(19) United States
(12) Patent Application Publication
(10) Pub. No.: US 2017/0137415 A1
(43) Pub. Date: May 18, 2017

(54) SULFONAMIDE COMPOUNDS AS VOLTAGE GATED SODIUM CHANNEL MODULATORS

(71) Applicant: LUPIN LIMITED, Mumbai (IN)

(72) Inventors: Vidya RAMDAS, Pune, Maharashtra (IN); Rajeshkumar Maganlal LORIYA, Pune, Maharashtra (IN); Deepak Sahebrao WALKE, Pune, Maharashtra (IN); Amit Kumar DAS, Pune, Maharashtra (IN); Talha Hussain KHAN, Pune, Maharashtra (IN); Moloy BANERJEE, Pune, Maharashtra (IN); Venkata P. PALLE, Pune, Maharashtra (IN); Rajender Kumar KAMBOJ, Pune, Maharashtra (IN)

(21) Appl. No.: 15/300,337

(22) PCT Filed: Mar. 27, 2015
(86) PCT No.: PCT/IB2015/052273

§ 371 Date: Sep. 29, 2016

(30) Foreign Application Priority Data
Mar. 29, 2014 (IN) 1170/MUM/2014
Feb. 20, 2015 (IN) 566/MUM/2015

(51) Int. Cl. C07D 417/12; C07D 417/14

(52) U.S. Cl. CPC C07D 417/12; C07D 417/14

(57) ABSTRACT

The present invention relates to the compound of Formula (I) wherein the substituents are as described herein, and their use in a medicine for the treatment of diseases, disorders associated with the inhibition of Voltage-gated sodium channels (VGSC) particularly NaV1.7. It further relates to the compounds herein and their pharmaceutically acceptable salts thereof, pharmaceutical compositions thereof useful in treating diseases, disorders, syndromes and/or conditions associated with the inhibition of Voltage-gated sodium channels (VGSC) particularly NaV1.7. The invention also relates to process for the preparation of the compounds of the invention.

### RELATED APPLICATIONS

The present application claims the benefit of priority to Indian Provisional Patent Application Nos. 1170/MUM/2014 filed on Mar. 29, 2014 and 0566/MUM/2015 filed on Feb. 20, 2015 and the entire provisional specifications are incorporated herein by reference.

### FIELD OF THE INVENTION

The present invention relates to sulfonamide compounds their pharmaceutically acceptable salts thereof, and pharmaceutical compositions for the treatment, management, and/or lessening severity of diseases, disorders, syndromes or conditions which are associated with the inhibition of voltage-gated sodium channels (VGSC) particularly Na_{V}1.7. The invention also relates to method of treating, managing and/or lessening the severity of diseases disorders, syndromes or conditions associated with the inhibition of voltage-gated sodium channels (VGSC) particularly Na_{V}1.7. The invention also relates to process for the preparation of the compounds of the invention.

### BACKGROUND OF THE INVENTION

Voltage-gated sodium channels play a crucial role in maintaining a specific membrane potential (intra- and extracellular ionic environments) across the mammalian cell membrane. The intracellular concentration of Na⁺ is kept low relative to the extracellular by active sodium pumps that eject three Na⁺ ions for every two K⁺ ions taken in. This generates a negative membrane potential (since more positive charge is pumped out and less taken in) and maintains the Na+ concentration of 6 and 140 mM in the intra and extracellular milieu. On opening of the voltage-gated sodium channels (VGSC), Na⁺ rushes in and leads to depolarization of the membrane because of the associated positive charge. The entry of Na⁺ via VGSC's occurs in cells of the heart, central and peripheral nervous system and is essential to initiate the firing of an action potential.

VGSCs consist of a pore-forming alpha subunit and a stabilizing beta subunit, 9 isoforms of the alpha subunit have been identified till date (Na_{V}1.1 to Na_{V}1.9). All nine members of the family have >50% identity in the amino acid sequence in the extracellular and transmembrane domain. The channels have also been further classified based on their sensitivity to the puffer fish toxin (tetrodotoxin, TTX). Channels Na_{V}1.8, Na_{V}1.9 and Na_{V}1.5 are TTX resistant (TTX-R) whereas the remaining channels are sensitive to TTX (TTX-S). (England and Rawson. Future Med. Chem. (2010), 2, 775-790). However, Na_{V}1.7gene is prominently responsible to cause to pain.

Loss of function mutations in the human Na_{V}1.7 gene lead to congenital insensitivity to pain which was observed for the first time in certain Pakistani families. Affected individuals displayed painless burns, fractures, and injuries of the lips and tongue. The patients did not have any autonomic or motor abnormalities, and reportedly had normal tear formation, sweating ability, reflexes, and intelligence. This genetic evidence clearly indicates that gain or loss of Na_{V}1.7 function can lead to exacerbation or loss of pain sensation respectively. Thus, it may be possible to treat chronic pain by pharmacologically blocking Na_{V}1.7. Moreover, Na_{V}1.7 has also been implicated in epilepsy. Small molecule Na_{V}1.7 blockers showed efficacy in in vivo epilepsy models. It has therefore been proposed that selective Na_{V}1.7 blockers may lead to therapeutic benefit in epilepsy (Hoyt et al. Bioorganic & Medicinal Chemistry Letters (2008), 18, 1963-1966).

Genetic evidence stems from the human gain of function as well as loss of function mutations that lead to inherited pain disorders and insensitivity to pain respectively. Non selective VGSC blockers have been shown to alleviate pain in animal models as well as in humans (e.g., Carbamazepine). Ralfinamide, another non-selective sodium channel blocker, is also being developed for the treatment of neuropathic pain.

Voltage-gated sodium channels are implicated in various diseases and disease conditions, including but not limited to chronic pain, visceral pain, arrhythmia, multiple sclerosis, epilepsy and related disorders as well as cancer. Thus, small molecules targeting one or more of the relevant VGSCs is likely to alleviate the suffering from these conditions.

International publication numbers WO 2006/110917, WO 2007/109324, WO 2008/046049, WO 2008/046084, WO 2008/046087, WO 2008/060789, WO 2009/012242, WO 2010/035166, WO 2010/045197, WO 2010/045251, WO 2010/053998, WO 2010/078307, WO2010/151595, WO2010/151597, WO 2011/002708, WO 2011/026240, WO 2011/103196, WO 2011/056985, WO 2011/058766, WO 2011/088201, WO2011/140425, US 2013/0289044, WO 2013/134518, WO 2014/201173, WO 2015/010065, and Bioorganic & Medicinal Chemistry Letters (2011), 21, 3676-681 disclose compounds related to voltage-gated sodium channel (VGSC) modulators for the treatment of various diseases mediated by VGSC modulation.

### SUMMARY OF THE INVENTION

In accordance with one aspect, the invention provides compounds having the structure of Formula (I),

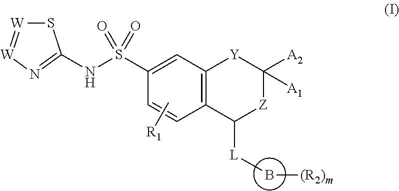

wherein,

Y is selected from CH₂, O and NR;

L is a bond or O;

R is hydrogen or substituted or unsubstituted alkyl;

A₁ and A₂ are independently hydrogen or substituted or unsubstituted alkyl; or

A₁ and A₂, together with the carbon atom to which they are attached, form a substituted or unsubstituted 3 to 6 membered cycloalkyl ring or 4-6 membered heterocyclyl ring;

Z is selected from CH₂ or —CH₂—CH₂;

R₁ is selected from hydrogen, halogen, cyano, substituted or unsubstituted alkyl and substituted or unsubstituted alkoxy;

ring B is substituted or unsubstituted aryl or substituted or unsubstituted heteroaryl;

R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;

W at each occurrence is independently selected from N or CR₃;

R₃ is selected from hydrogen, halogen, cyano, substituted or unsubstituted alkyl and substituted or unsubstituted alkoxy;

‘m’ is an integer ranging from 0 to 3, both inclusive;

wherein the substituents for substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are one or more same or different and independently selected from the group consisting of hydroxy, halogen, carboxy, cyano, nitro, oxo (═O), thio (═S), alkyl, haloalkyl, hydroxyalkyl, alkoxyalkyl, alkenyl, alkynyl, aryl, arylalkyl, cycloalkyl, cycloalkylalkyl, cycloalkenyl, heteroaryl, heterocyclic ring, heterocyclylalkyl, heteroarylalkyl, —C(O)ORₓ, —C(O)R_{y}, —C(S)R_{y}, —C(O)NRₓR_{z}, —NRₓC(O)NRₓR_{z}, —N(Rₓ)S(O)₂R_{y}, —NRₓR_{z}, —NRₓC(O)R_{y}, —NRₓC(S)R_{y}, —NRₓC(S)NRₓR_{z}, —S(O)₂NRₓR_{z}, —ORₓ, —OC(O)R_{y}, —C(RₐR_{b})₁₋₃C(O)ORₓ, —C(RₐR_{b})₁₋₃C(O)NRₓR_{z}, —OC(RₐR_{b})₂₋₃—ORₓ, —OC(RₐR_{b})₂₋₃—NRₓR_{z}, —OC(RₐR_{b})₂₋₃—S(O)₀₋₂R_{y}, —C(RₐR_{b})₁₋₃—NRₓR_{z}, —C(RₐR_{b})₁₋₃—S(O)₀₋₂R_{y}, —OC(RₐR_{b})₁₋₃—C(O)NRₓR_{z}, —OC(RₐR_{b})₁₋₃—C(O)ORₓ, and —S(O)₀₋₂R_{y};

Rₓ, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl;

R_{y}, which may be same or different at each occurrence, is independently selected from alkyl, haloalkyl, alkenyl, alkynyl, aryl, arylalkyl, cycloalkyl, cycloalkenyl, heteroaryl, heterocyclic ring, heterocyclylalkyl and heteroarylalkyl; and

R_{z}, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl; or Rₓ and R_{z} together with the nitrogen atom to which they are attached form a substituted or unsubstituted, saturated or unsaturated 4 to 8 membered cyclic ring, wherein the unsaturated cyclic ring may have one or two double bonds;

or N-oxides thereof or a pharmaceutically acceptable salt thereof.

In accordance with one aspect, the invention provides compounds having the structure of Formula (Ia),

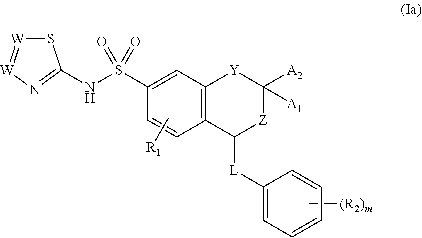

wherein,

Y is O or CH₂;

L is a bond or O;

Z is —CH₂—;

A₁ and A₂ are independently hydrogen or substituted or unsubstituted alkyl; or

A₁ and A₂, together with the carbon atom to which they are attached, form a substituted or unsubstituted 3 to 6 membered cycloalkyl ring or 4-6 membered heterocyclyl ring;

R₁ is selected from hydrogen, halogen, cyano, substituted or unsubstituted alkyl and substituted or unsubstituted alkoxy;

R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;

W at each occurrence is independently selected from N or CR₃;

R₃ is selected from hydrogen, halogen, cyano, substituted or unsubstituted alkyl and substituted or unsubstituted alkoxy;

‘m’ is an integer ranging from 0 to 3, both inclusive;

wherein the substituents for substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are one or more same or different and independently selected from the group consisting of hydroxy, halogen, carboxy, cyano, nitro, oxo (═O), thio (═S), alkyl, haloalkyl, hydroxyalkyl, alkoxyalkyl, alkenyl, alkynyl, aryl, arylalkyl, cycloalkyl, cycloalkylalkyl, cycloalkenyl, heteroaryl, heterocyclic ring, heterocyclylalkyl, heteroarylalkyl, —C(O)ORₓ, —C(O)R_{y}, —C(S)R_{y}, —C(O)NRₓR_{z}, —NRₓC(O)NRₓR_{z}, —N(Rₓ)S(O)₂R_{y}, —NRₓR_{z}, —NRₓC(O)R_{y}, —NRₓC(S)R_{y}, —NRₓC(S)NRₓR_{z}, —S(O)₂NRₓR_{z}, —ORₓ, —OC(O)R_{y}, —C(RₐR_{b})₁₋₃C(O)ORₓ, —C(RₐR_{b})₁₋₃C(O)NRₓR_{z}, —OC(RₐR_{b})₂₋₃—ORₓ, —OC(RₐR_{b})₂₋₃—NRₓR_{z}, —OC(RₐR_{b})₂₋₃—S(O)₀₋₂R_{y}, —C(RₐR_{b})₁₋₃—NRₓR_{z}, —C(RₐR_{b})₁₋₃—S(O)₀₋₂R_{y}, —OC(RₐR_{b})₁₋₃—C(O)NRₓR_{z}, —OC(RₐR_{b})₁₋₃—C(O)ORₓ, and —S(O)₀₋₂R_{y};

Rₓ, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl;

R_{y}, which may be same or different at each occurrence, is independently selected from alkyl, haloalkyl, alkenyl, alkynyl, aryl, arylalkyl, cycloalkyl, cycloalkenyl, heteroaryl, heterocyclic ring, heterocyclylalkyl and heteroarylalkyl; and

R_{z}, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl; or Rₓ and R_{z} together with the nitrogen atom to which they are attached form a substituted or unsubstituted, saturated or unsaturated 4 to 8 membered cyclic ring, wherein the unsaturated cyclic ring may have one or two double bonds;

or N-oxides thereof or a pharmaceutically acceptable salt thereof.

According to one embodiment, there are provided compounds having the structure of Formula (Ia) wherein Y is CH₂ or O.

According to another embodiment, there are provided compounds having the structure of Formula (Ia) wherein Y is O.

According to another embodiment, there are provided compounds having the structure of Formula (Ia) wherein Y is CH₂.

According to another embodiment, there are provided compounds having the structure of Formula (Ia) wherein L is a bond or O.

According to another embodiment, there are provided compounds having the structure of Formula (Ia) wherein L is a bond.

According to another embodiment, there are provided compounds having the structure of Formula (Ia) wherein L is a O.

According to another embodiment, there are provided compounds having the structure of Formula (Ia) wherein m is 1 to 3.

According to another embodiment, there are provided compounds having the structure of Formula (Ia) wherein m is 2.

According to another embodiment, there are provided compounds having the structure of Formula (Ia) wherein R₂ is same or different and are independently selected from halogen, cyano, substituted or unsubstituted (C₁-C₆)alkyl, substituted or unsubstituted (C₁-C₆)haloalkyl, substituted or unsubstituted (C₁-C₆)alkoxy, substituted or unsubstituted (C₃-C₁₂)cycloalkyl, substituted or unsubstituted C₆₋aryl, substituted or unsubstituted heteroaryl and substituted or unsubstituted heterocyclyl; ‘m’ is 1 to 3.

According to another embodiment, there are provided compounds having the structure of Formula (Ia) wherein R₂ is same or different at each occurrence, independently selected from halogen (e.g. chloro, fluoro or bromo), haloalkyl (e.g. trifluoromethyl), cyano, substituted or unsubstituted (C₁-C₆)alkyl (e.g. methyl, ethyl, isopropyl, —CH₂—C(O)N(CH₃)₂)(C₁-C₆)alkoxy (e.g. methoxy), substituted or unsubstituted (C₃-C₁₂)cycloalkyl, substituted or unsubstituted C₆-aryl, substituted or unsubstituted heteroaryl(e.g. a residue having the structure:

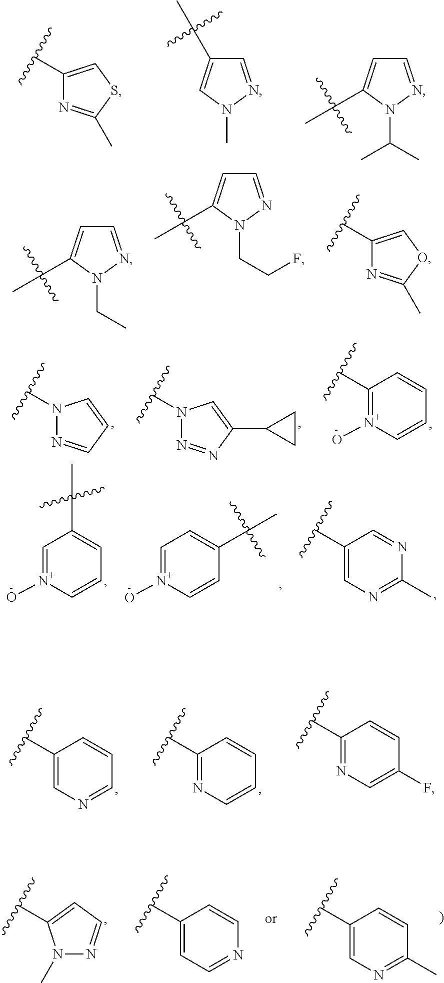

and substituted or unsubstituted heterocyclyl (e.g. a residue having the structure:

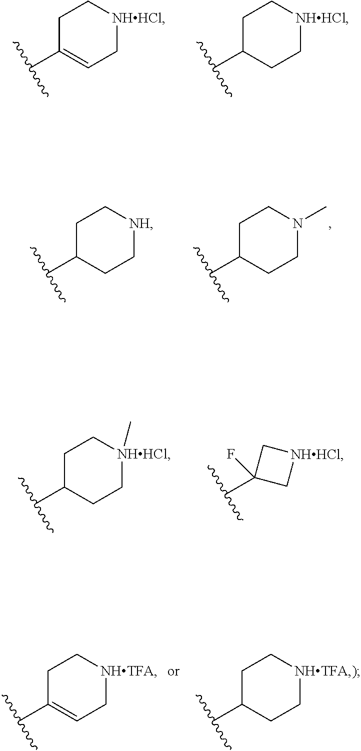

m is 1 or 2.

According to another embodiment, there are provided compounds having the structure of Formula (Ia) wherein R₂ is substituted or unsubstituted haloalkyl (e.g. CF₃); m is 1.

According to another embodiment, there are provided compounds having the structure of Formula (Ia) wherein m is 2, one R₂ is CF₃ and the other R₂ moiety is selected from the group consisting of: halogen (e.g. chloro or bromo), cyano, substituted or unsubstituted (C₁-C₆)alkyl (e.g. ethyl, isopropyl, —CH₂—C(O)N(CH₃)₂, substituted or unsubstituted (C₃-C₁₂)cycloalkyl (e.g. cyclopropyl), (C₁-C₆)alkoxy (e.g. methoxy), substituted or unsubstituted C₆-aryl

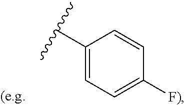

substituted or unsubstituted heteroaryl (e.g. a residue having the structure:

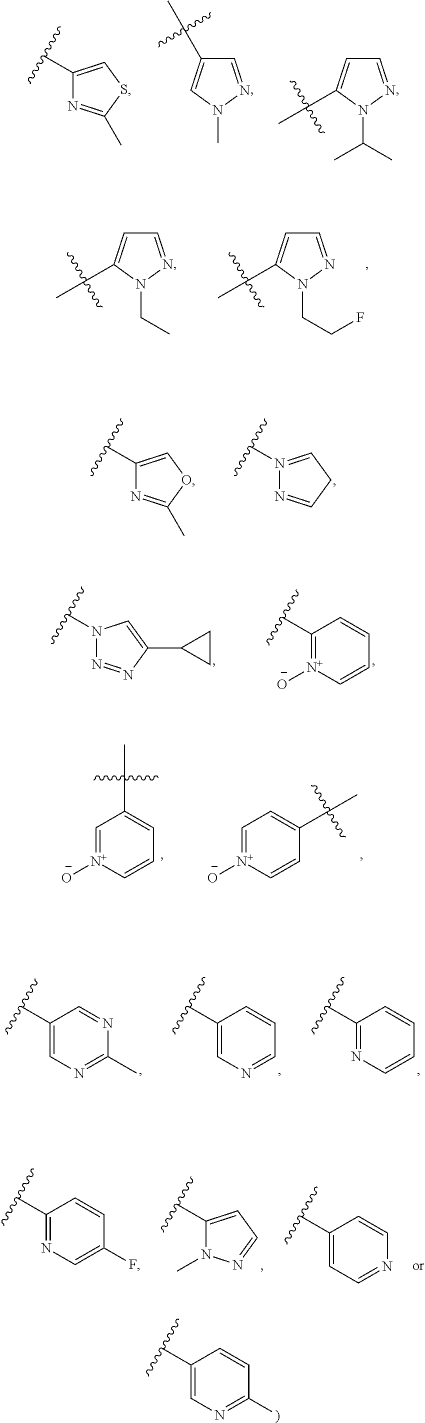

and substituted or unsubstituted heterocyclyl (e.g. a residue having the structure:

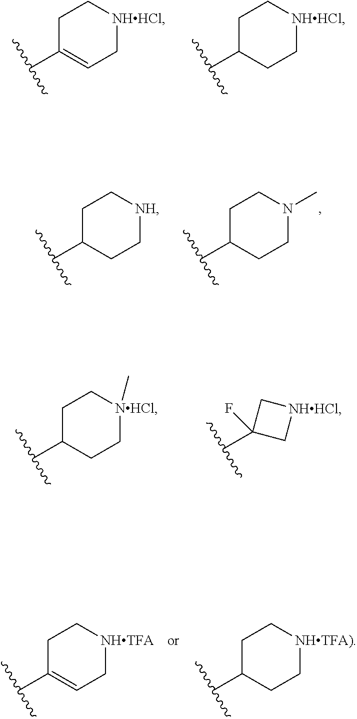

Preferably the CF₃ moiety is para to L. Preferably the R₂ moiety selected from the group consisting of: halogen (e.g. chloro), cyano, substituted or unsubstituted (C₁-C₆)alkyl (e.g. ethyl, isopropyl, —CH₂—C(O)N(CH₃)₂, substituted or unsubstituted (C₃-C₁₂)cycloalkyl (e.g. cyclopropyl), substituted or unsubstituted C₆-aryl

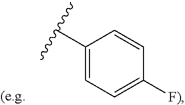

(C₁-C₆)alkoxy (e.g. methoxy), substituted or unsubstituted heteroaryl (e.g. a residue having the structure:

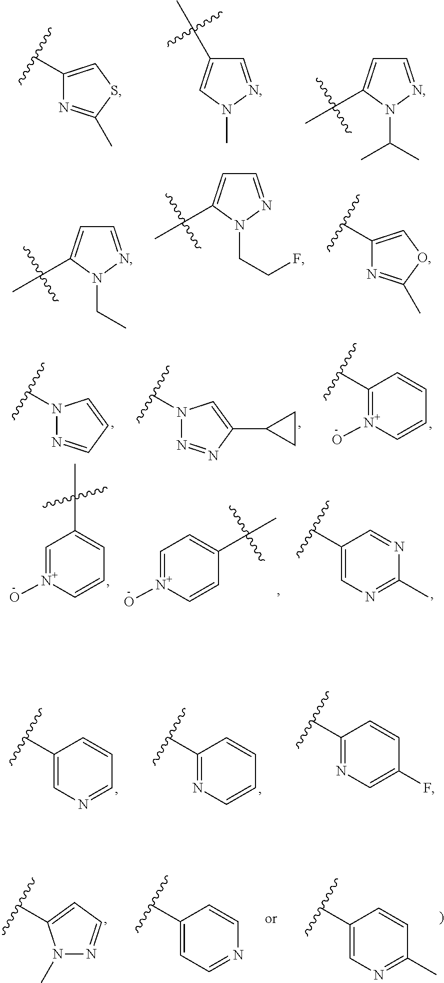

and substituted or unsubstituted heterocyclyl (e.g. a residue having the structure:

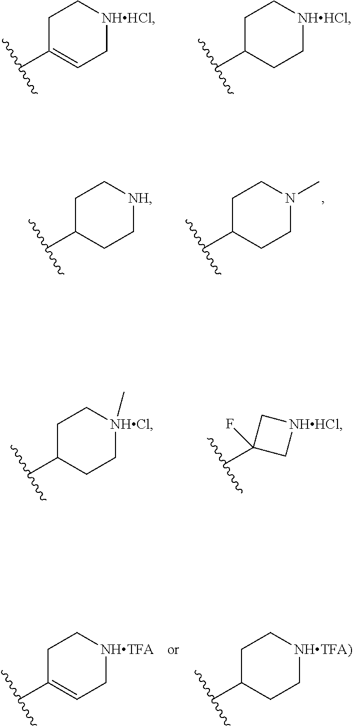

is ortho to L.

According to another embodiment, there are provided compounds having the structure of Formula (Ia) wherein m is 2, one R₂ is CF₃ and the other R₂ moiety is halogen (e.g. chloro, bromo).

According to another embodiment, there are provided compounds having the structure of Formula (Ia) wherein m is 2, one R₂ is halogen (e.g. Cl) and the other R₂ moiety is halogen (e.g. Cl). Preferably the halogen moieties are para to L and ortho to L.

According to another embodiment, there are provided compounds having the structure of Formula (Ia) wherein A₁ and A₂ are independently H or substituted or unsubstituted (C₁-C₆)alkyl.

According to another embodiment, there are provided compounds having the structure of Formula (Ia) wherein A₁ is Me.

According to another embodiment, there are provided compounds having the structure of Formula (Ia) wherein A₂ is Me.

According to another embodiment, there are provided compounds having the structure of Formula (Ia) wherein A₁ is H.

According to another embodiment, there are provided compounds having the structure of Formula (Ia) wherein A₂ is H.

According to another embodiment, there are provided compounds having the structure of Formula (Ia) wherein A₁ and A₂, together with the atoms to which they are attached, form a 4 membered cycloalkyl ring.

According to another embodiment, there are provided compounds having the structure of Formula (Ia) wherein A₂ is Et.

According to another embodiment, there are provided compounds having the structure of Formula (Ia) wherein R₁ is H.

According to another embodiment, there are provided compounds having the structure of Formula (Ia) wherein W at each occurrence is independently selected from N or CR₃; where R₃ is selected from hydrogen, halogen and substituted or unsubstituted (C₁-C₆)alkyl. Preferably R₃ is H. In an embodiment, the W adjacent to S is N. In an alternate embodiment, the W adjacent to S is CR₃. In an embodiment, the W adjacent to N is N. In an alternate embodiment, the W adjacent to N is CR₃. In an embodiment, the W adjacent to S is N and the W adjacent to N is CR₃. In an embodiment, the W adjacent to N is N and the W adjacent to S is CR₃. In an embodiment, the W adjacent to N is CR₃ and the W adjacent to S is CR₃.

In an embodiment, R₁ is H, Y is CH₂, L is O, m is 2, R₃ is H, one R₂ is CF₃, A₁ is H, A₂ is H, the W adjacent to S is CR₃ and the W adjacent to N is CR₃; Optionally, the other R₂ is selected from halogen (e.g. chloro, fluoro or bromo), substituted or unsubstituted heteroaryl (e.g. a residue having the structure:

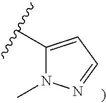 [SMILES: *c1ccnn1C]

and substituted or unsubstituted heterocyclyl (e.g. a residue having the structure:

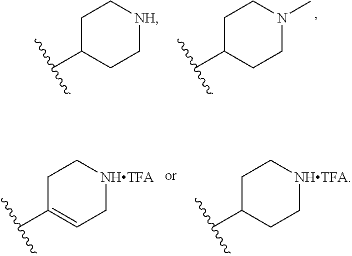

In an embodiment, R₁ is H, Y is CH₂, L is O, m is 2, R₃ is H, one R₂ is CF₃, A₁ is H, A₂ is H, the W adjacent to S is CR₃ and the W adjacent to N is N; Optionally, the other R₂ is selected from halogen (e.g. chloro or bromo), substituted or unsubstituted heteroaryl (e.g. a residue having the structure:

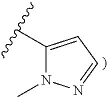 [SMILES: *c1ccnn1C]

and substituted or unsubstituted heterocyclyl (e.g. a residue having the structure:

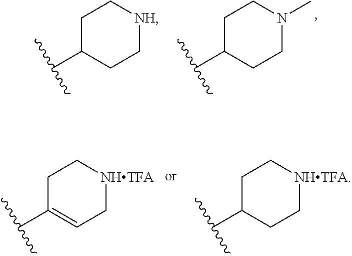

In an embodiment, R₁ is H, Y is CH₂, L is O, m is 2, R₃ is H, one R₂ is CF₃, A₁ is H, A₂ is H, the W adjacent to S is N and the W adjacent to N is CR₃; Optionally, the other R₂ is selected from halogen (e.g. chloro or bromo), substituted or unsubstituted heteroaryl (e.g. a residue having the structure:

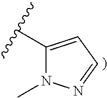 [SMILES: *c1ccnn1C]

and substituted or unsubstituted heterocyclyl (e.g. a residue having the structure:

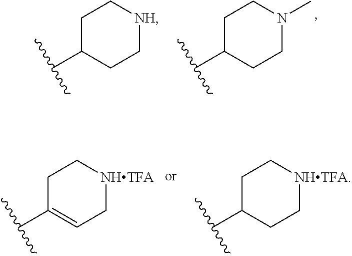

In an embodiment, R₁ is H, Y is O, L is a bond, m is 2, R₃ is H, one R₂ is CF₃, A₁ is Me, A₂ is Me, the W adjacent to S is N and the W adjacent to N is CR₃; Optionally, the other R₂ is selected from halogen (e.g. chloro), (C₁-C₆)alkoxy (e.g. methoxy) and substituted or unsubstituted heterocyclyl (e.g. a residue having the structure:

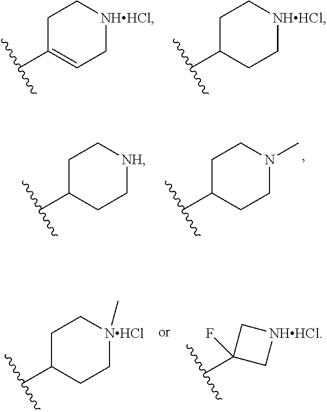

In an embodiment, R₁ is H, Y is O, L is a bond, m is 2, R₃ is H, one R₂ is CF₃, A₁ is Me, A₂ is Me, the W adjacent to S is CR₃ and the W adjacent to N is N; Optionally, the other R₂ is selected from halogen (e.g. chloro), (C₁-C₆)alkoxy (e.g. methoxy) and substituted or unsubstituted heterocyclyl (e.g. a residue having the structure:

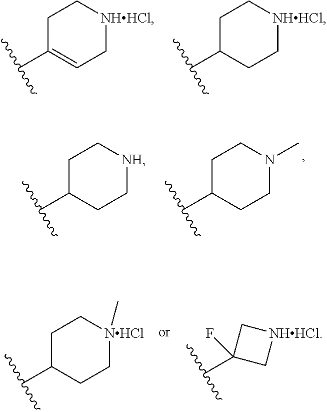

In an embodiment, R₁ is H, Y is O, L is a bond, m is 2, R₃ is H, one R₂ is CF₃, A₁ is Me, A₂ is Me, the W adjacent to S is CR₃ and the W adjacent to N is CR₃; Optionally, the other R₂ is selected from halogen (e.g. chloro), (C₁-C₆)alkoxy (e.g. methoxy), substituted or unsubstituted heteroaryl (e.g. a residue having the structure:

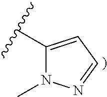 [SMILES: *c1ccnn1C]

and substituted or unsubstituted heterocyclyl (e.g. a residue having the structure:

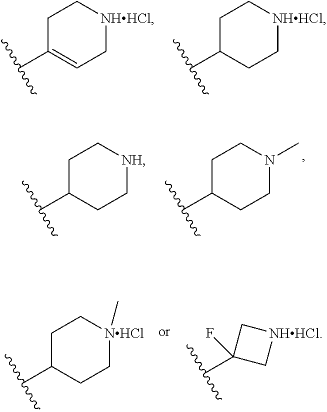

In an embodiment, R₁ is H, Y is O, L is a bond, m is 2, R₃ is H, one R₂ is CF₃, A₁ is H, A₂ is H, the W adjacent to S is CR₃ and the W adjacent to N is CR₃; Optionally, the other R₂ is selected from halogen (e.g. chloro), substituted or unsubstituted heteroaryl (e.g. a residue having the structure:

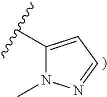 [SMILES: *c1ccnn1C]

and substituted or unsubstituted heterocyclyl (e.g. a residue having the structure:

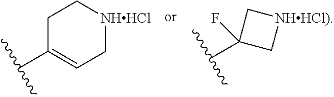

In an embodiment, R₁ is H, Y is O, L is a bond, m is 2, R₃ is H, one R₂ is CF₃, A₁ is H, A₂ is H, the W adjacent to S is N and the W adjacent to N is CR₃; Optionally, the other R₂ is selected from halogen (e.g. chloro), substituted or unsubstituted heteroaryl (e.g. a residue having the structure:

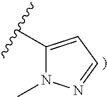 [SMILES: *c1ccnn1C]

and substituted or unsubstituted heterocyclyl (e.g. a residue having the structure:

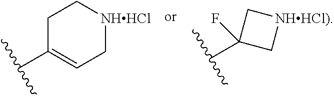

In an embodiment, R₁ is H, Y is O, L is a bond, m is 2, R₃ is H, one R₂ is CF₃, A₁ is H, A₂ is H, the W adjacent to S is CR₃ and the W adjacent to N is N; Optionally, the other R₂ is selected from halogen (e.g. chloro), substituted or unsubstituted heteroaryl (e.g. a residue having the structure:

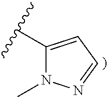 [SMILES: *c1ccnn1C]

and substituted or unsubstituted heterocyclyl (e.g. a residue having the structure:

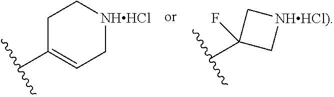

According to one embodiment, there are provided compounds having the structure of Formula (II)

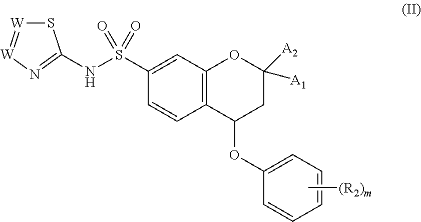

wherein,

A₁ and A₂ are independently hydrogen or substituted or unsubstituted alkyl; or

A₁ and A₂, together with the carbon atom to which they are attached, form a substituted or unsubstituted 3 to 6 membered cycloalkyl ring;

R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;

W at each occurrence is independently selected from N or CR₃;

R₃ is selected from hydrogen, halogen, cyano, substituted or unsubstituted alkyl and substituted or unsubstituted alkoxy; and

‘m’ is an integer ranging from 1 to 3, both inclusive;

wherein the substitutents for the substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are as defined in formula (Ia);

or N-oxides thereof or a pharmaceutically acceptable salt thereof.

According to another embodiment, there are provided compounds having the structure of Formula (II) wherein m is 1 to 3.

According to another embodiment, there are provided compounds having the structure of Formula (II) wherein R₂ same or different at each occurrence, is independently selected from halogen (e.g. chloro, Fluoro or bromo), haloalkyl (e.g. trifluoromethyl), cyno, substituted or unsubstituted (C₁-C₆)alkyl, (C₁-C₆)alkoxy (e.g. methoxy), substituted or unsubstituted (C₃-C₁₂)cycloalkyl, substituted or unsubstituted C₆-aryl, substituted or unsubstituted heteroaryl (e.g. a residue having the structure:

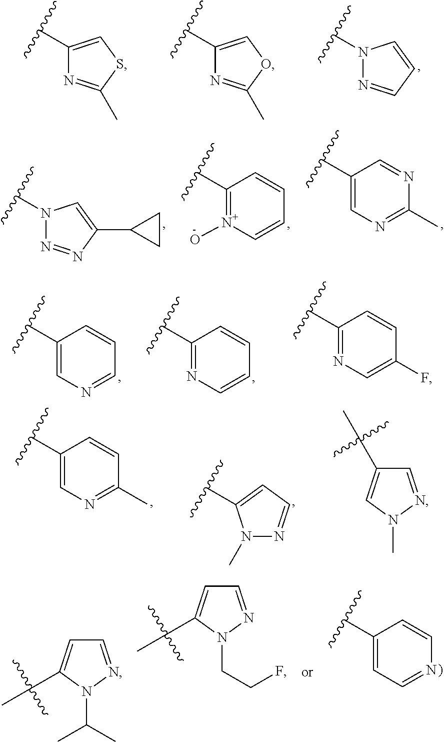

and substituted or unsubstituted heterocyclyl (e.g. a residue having the structure:

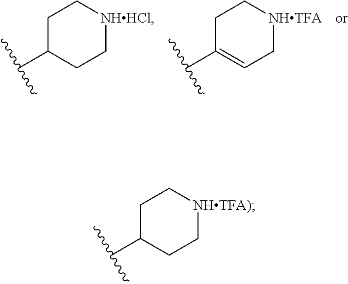

m is 1 or 2.

According to another embodiment, there are provided compounds having the structure of Formula (II) wherein m is 2, one R₂ is CF₃ and the other R₂ moiety is selected from the group consisting of: halogen (e.g. chloro or bromo), cyano, substituted or unsubstituted (C₁-C₆)alkyl (e.g. ethyl, isopropyl, —CH₂—C(O)N(CH₃)₂, substituted or unsubstituted (C₃-C₁₂)cycloalkyl (e.g. cyclopropyl), substituted or unsubstituted C₆-aryl, substituted or unsubstituted heteroaryl (e.g. a residue having the structure:

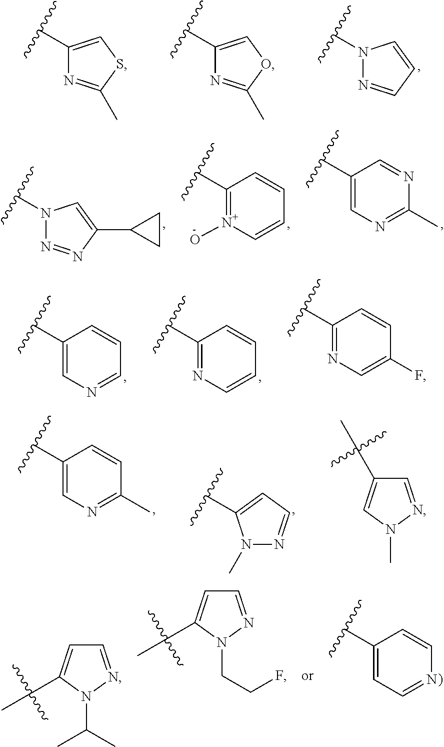

and substituted or unsubstituted heterocyclyl (e.g. a residue having the structure:

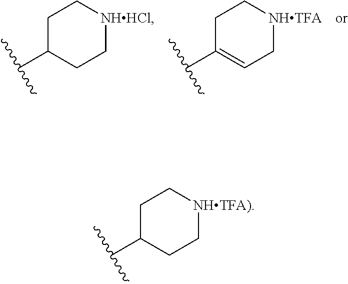

Preferably the CF₃ moiety is para to L. Preferably the R₂ moiety selected from the group consisting of: halogen (e.g. chloro), substituted or unsubstituted (C₁-C₆)alkyl (e.g. ethyl), substituted or unsubstituted heteroaryl (e.g. a residue having the structure:

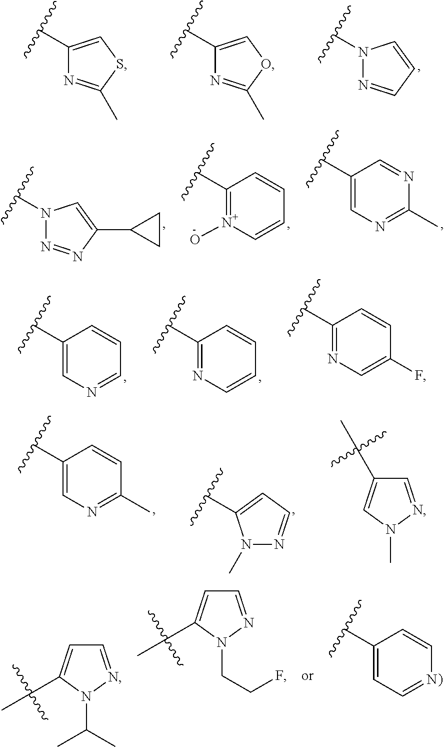

and substituted or unsubstituted heterocyclyl (e.g. a residue having the structure:

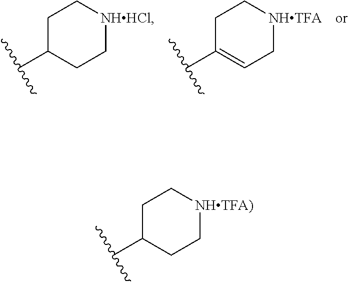

is ortho to L.

According to another embodiment, there are provided compounds having the structure of Formula (II) wherein m is 2, one R₂ is halogen (e.g. Cl) and the other R₂ moiety is halogen (e.g. Cl, F). Preferably the halogen moieties are para to L and ortho to L.

According to another embodiment, there are provided compounds having the structure of Formula (II) wherein A₁ is Me.

According to another embodiment, there are provided compounds having the structure of Formula (II) wherein A₂ is Me.

According to another embodiment, there are provided compounds having the structure of Formula (II) wherein A₁ is H.

According to another embodiment, there are provided compounds having the structure of Formula (II) wherein A₂ is H.

According to another embodiment, there are provided compounds having the structure of Formula (II) wherein A₁ and A₂, together with the atoms to which they are attached, form a 4 membered cycloalkyl ring.

According to another embodiment, there are provided compounds having the structure of Formula (II) wherein A₂ is Et.

According to another embodiment, there are provided compounds having the structure of Formula (II) wherein W at each occurrence is independently selected from N or CR₃; where R₃ is selected from hydrogen, halogen and substituted or unsubstituted (C₁-C₆)alkyl. Preferably R₃ is H. In an embodiment, the W adjacent to S is N. In an alternate embodiment, the W adjacent to S is CR₃. In an embodiment, the W adjacent to N is N. In an alternate embodiment, the W adjacent to N is CR₃. In an embodiment, the W adjacent to S is N and the W adjacent to N is CR₃. In an embodiment, the W adjacent to N is N and the W adjacent to S is CR₃. In an embodiment, the W adjacent to N is CR₃ and the W adjacent to S is CR₃.

In an embodiment, m is 2, R₃ is H, A₁ is H, A₂ is H, one R₂ is CF₃, the W adjacent to S is CR₃ and the W adjacent to N is N; Optionally, the other R₂ is selected from halogen (e.g. chloro), substituted or unsubstituted heteroaryl (e.g. a residue having the structure:

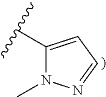 [SMILES: *c1ccnn1C]

and substituted or unsubstituted heterocyclyl (e.g. a residue having the structure:

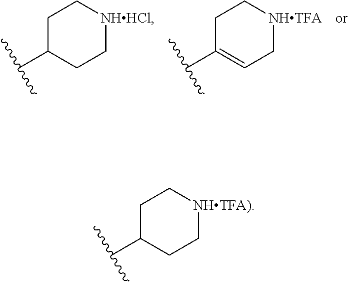

In an embodiment, m is 2, R₃ is H, A₁ is H, A₂ is H, one R₂ is CF₃, the W adjacent to S is CR₃ and the W adjacent to N is CR₃; Optionally, the other R₂ is selected from halogen (e.g. chloro), cyno, substituted or unsubstituted (C₁-C₆)alkyl (e.g. ethyl, isopropyl, —CH₂—C(O)N(CH₃)₂, substituted or unsubstituted (C₃-C₁₂)cycloalkyl (e.g. cyclopropyl), substituted or unsubstituted C₆-aryl

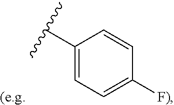

substituted or unsubstituted heteroaryl (e.g. a residue having the structure:

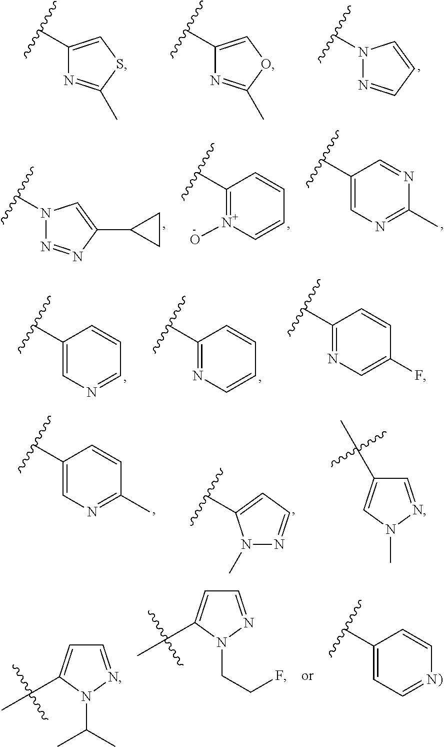

and substituted or unsubstituted heterocyclyl (e.g. a residue having the structure:

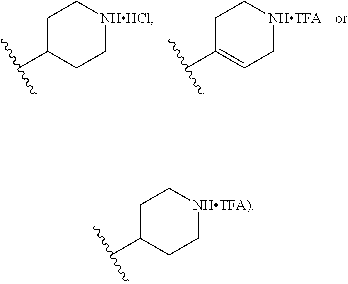

In an embodiment, m is 2, R₃ is H, A₁ is H, A₂ is H, one R₂ is CF₃, the W adjacent to S is N and the W adjacent to N is CR₃; Optionally, the other R₂ is selected from halogen (e.g. chloro), substituted or unsubstituted heteroaryl (e.g. a residue having the structure:

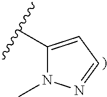 [SMILES: *c1ccnn1C]

and substituted or unsubstituted heterocyclyl (e.g. a residue having the structure:

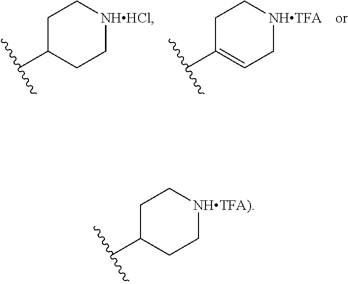

In an embodiment, m is 2 or 3, A₁ is H, A₂ is H, one R₂ is halogen (e.g. chloro or fluoro), the W adjacent to S is CR₃ and the W adjacent to N is CR₃ where R₃ is H; Optionally, the other R₂ is halogen (e.g. chloro or fluoro).

In an embodiment, m is 2, R₃ is H, A₁ is Me, A₂ is Me, one R₂ is CF₃ or Cl, the W adjacent to S is CR₃ and the W adjacent to N is N; Optionally, the other R₂ is selected from hydrogen, halogen (e.g. chloro) and substituted or unsubstituted heteroaryl (e.g. a residue having the structure:

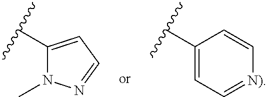

In an embodiment, m is 2, R₃ is H, A₁ is Me, A₂ is Me, one R₂ is CF₃ or Cl, the W adjacent to S is CR₃ and the W adjacent to N is CR₃; Optionally, the other R₂ is selected from hydrogen, halogen (e.g. chloro), cyano, substituted or unsubstituted (C₁-C₆)alkyl (e.g. ethyl), substituted or unsubstituted C₆-aryl

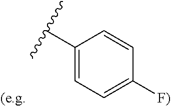

and substituted or unsubstituted heteroaryl (e.g. a residue having the structure:

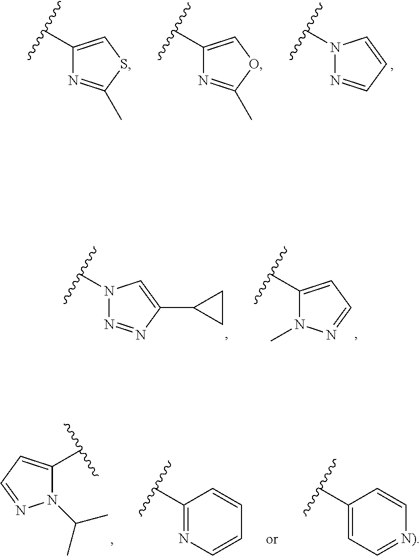

In an embodiment, m is 2, R₃ is H, A₁ is Me, A₂ is Me, one R₂ is CF₃ or Cl, the W adjacent to S is N and the W adjacent to N is CR₃; Optionally, the other R₂ is selected from halogen (e.g. chloro) and substituted or unsubstituted heteroaryl (e.g. a residue having the structure:

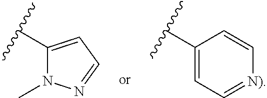

In an embodiment, m is 2, R₃ is H, A₁ and A₂ together form a 4 membered cycloalkyl ring, one R₂ is CF₃, the W adjacent to S is CR₃ and the W adjacent to N is N; Optionally, the other R₂ is selected from halogen (e.g. chloro) and substituted or unsubstituted heteroaryl (e.g. a residue having the structure:

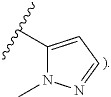 [SMILES: *c1ccnn1C]

In an embodiment, m is 2, R₃ is H, A₁ and A₂ together form a 4 membered cycloalkyl ring, one R₂ is CF₃, the W adjacent to S is CR₃ and the W adjacent to N is CR₃; Optionally, the other R₂ is selected from halogen (e.g. chloro) and substituted or unsubstituted heteroaryl (e.g. a residue having the structure:

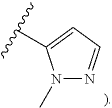 [SMILES: *c1ccnn1C]

In an embodiment, m is 2, R₃ is H, A₁ and A₂ together form a 4 membered cycloalkyl ring, one R₂ is CF₃, the W adjacent to S is N and the W adjacent to N is CR₃; Optionally, the other R₂ is selected from halogen (e.g. chloro) and substituted or unsubstituted heteroaryl (e.g. a residue having the structure:

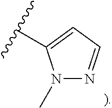 [SMILES: *c1ccnn1C]

In an embodiment, m is 2, R₃ is H, A₁ is H, A₂ is Et, one R₂ is CF₃, the W adjacent to S is CR₃ and the W adjacent to N is N; Optionally, the other R₂ is selected from halogen (e.g. chloro) and substituted or unsubstituted heteroaryl (e.g. a residue having the structure:

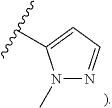 [SMILES: *c1ccnn1C]

In an embodiment, m is 2, R₃ is H, A₁ is H, A₂ is Et, one R₂ is CF₃, the W adjacent to S is CR₃ and the W adjacent to N is CR₃; Optionally, the other R₂ is selected from halogen (e.g. chloro) and substituted or unsubstituted heteroaryl (e.g. a residue having the structure:

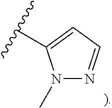 [SMILES: *c1ccnn1C]

In an embodiment, m is 2, R₃ is H, A₁ is H, A₂ is Et, one R₂ is CF₃, the W adjacent to S is N and the W adjacent to N is CR₃; Optionally, the other R₂ is selected from halogen (e.g. chloro) and substituted or unsubstituted heteroaryl (e.g. a residue having the structure:

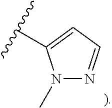 [SMILES: *c1ccnn1C]

In an embodiment, m is 2, R₃ is H, A₁ is H, A₂ is H, one R₂ is CF₃, the W adjacent to S is CR₃ and the W adjacent to N is CR₃; Optionally, the other R₂ is selected from halogen (e.g. chloro) and substituted or unsubstituted heteroaryl (e.g. a residue having the structure:

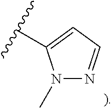 [SMILES: *c1ccnn1C]

In an embodiment, m is 2, R₃ is H, A₁ is Me, A₂ is Me, one R₂ is CF₃, the W adjacent to S is CR₃ and the W adjacent to N is CR₃; Optionally, the other R₂ is selected from halogen (e.g. chloro) and substituted or unsubstituted heteroaryl (e.g. a residue having the structure:

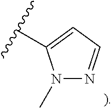 [SMILES: *c1ccnn1C]

According to another embodiment, there are provided compounds having the structure of Formula (III)

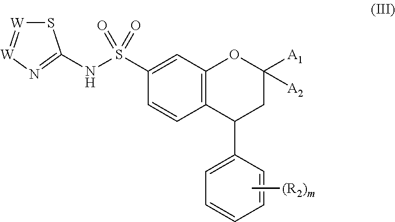

wherein,

A₁ and A₂ are independently hydrogen or substituted or unsubstituted alkyl; or

A₁ and A₂, together with the carbon atom to which they are attached, form a substituted or unsubstituted 3 to 6 membered cycloalkyl ring;

R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;

W at each occurrence is independently selected from N or CR₃;

R₃ is selected from hydrogen, halogen, cyano, substituted or unsubstituted alkyl and substituted or unsubstituted alkoxy; and

‘m’ is an integer ranging from 1 to 3, both inclusive;

wherein the substitutents for the substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are as defined in formula (Ia);

or N-oxides thereof or a pharmaceutically acceptable salt thereof.

According to another embodiment, there are provided compounds having the structure of Formula (IV)

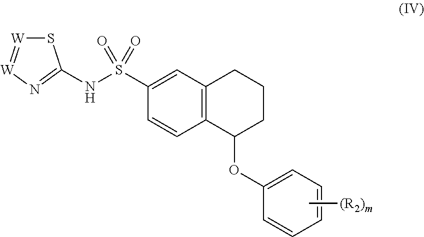

wherein,

R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;

W at each occurrence is independently selected from N or CR₃;

R₃ is selected from hydrogen, halogen, cyano, substituted or unsubstituted alkyl and substituted or unsubstituted alkoxy; and

‘m’ is an integer ranging from 1 to 3, both inclusive;

wherein the substitutents for the substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are as defined in formula (Ia);

or N-oxides thereof or a pharmaceutically acceptable salt thereof.

It should be understood that the Formula (I), Formula (Ia), Formula (II), Formula (III) and/or Formula (IV) structurally encompasses all tautomers, stereoisomers, enantiomers and diastereomers, including isotopes wherever applicable and pharmaceutically acceptable salts that may be contemplated from the chemical structures generally described herein.

It should be understood that the Formula (I), Formula (Ia), Formula (II), Formula (III) and/or Formula (IV) structurally encompasses, wherever applicable, N-oxide of all stereoisomers, enantiomers and diastereomers, and their pharmaceutically acceptable salts that may be contemplated from the chemical structures generally described herein.

The details of one or more embodiments of the invention set forth in the below are illustrative in nature only and not intended to limit to the scope of the invention. Other features, objects and advantages of the inventions will be apparent from the description and claims.

Compounds of the invention include, for example, compounds of the Formulae (I), (Ia), (II), (III) or (IV) or pharmaceutically acceptable salts thereof, wherein, unless otherwise stated, each of Y, L, Z, A₁, A₂, W, R₁, R₂, and ‘m’ has any of the meanings defined hereinbefore or independently in any of paragraphs (1) to (8):
(1) Y is O or CH₂.
(2) L is a bond or O.
(3) Z is —CH₂—.
(4) A₁ and A₂ are independently hydrogen or substituted or unsubstituted (C₁-C₆)alkyl; or
   A₁ and A₂, together with the carbon atom to which they are attached, form a substituted or unsubstituted 3 to 6 membered cycloalkyl ring or 4-6 membered heterocyclyl ring.
(5) R₁ is selected from hydrogen, halogen and substituted or unsubstituted (C₁-C₆)alkyl.
(6) R₂, which may be same or different at each occurrence, is independently selected from halogen, cyano, substituted or unsubstituted (C₁-C₆)alkyl, substituted or unsubstituted (C₁-C₆)haloalkyl, substituted or unsubstituted (C₁-C₆)alkoxy, substituted or unsubstituted (C₃-C₁₂)cycloalkyl, substituted or unsubstituted (C₆-C₁₀)aryl, substituted or unsubstituted heteroaryl and substituted or unsubstituted heterocyclyl.
(7) W at each occurrence is independently selected from N or CR₃;
   where R₃ is selected from hydrogen, halogen, cyano, substituted or unsubstituted (C₁-C₆)alkyl and substituted or unsubstituted (C₁-C₆)alkoxy.
(8) ‘m’ is an integer ranging from 0 to 3, both inclusive.

According to another embodiment, there are provided compounds having the structure of Formula (Ia)

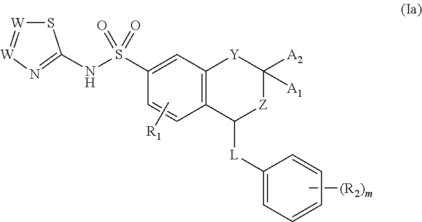

wherein Y is O or CH₂; L is O or bond; R₁ is hydrogen; A₁ and A₂ are hydrogen or substituted or unsubstituted (C₁-C₆)alkyl; Z is CH₂; or A₁ and A₂ together form a substituted or unsubstituted 3 to 6 membered cycloalkyl ring; at each occurrence R₂ is independently selected from halogen, cyano, substituted or unsubstituted (C₁-C₆)alkyl, substituted or unsubstituted (C₁-C₆)haloalkyl, substituted or unsubstituted (C₁-C₆)alkoxy, substituted or unsubstituted (C₃-C₁₂)cycloalkyl, substituted or unsubstituted C₆-aryl, substituted or unsubstituted heteroaryl and substituted or unsubstituted heterocyclyl; ‘m’ is 1 to 3 and W at each occurrence is independently selected from N or CR₃; where R₃ is hydrogen or halogen or substituted or unsubstituted (C₁-C₆)alkyl.

Below are the representative compounds, which are illustrative in nature only and are not intended to limit to the scope of the invention:
4-(2-Chloro-4-(trifluoromethyl)phenyl)-2,2-dimethyl-N-(1,2,4-thiadiazol-5-yl)chroman-7-sulfonamide,
4-(2-Chloro-4-(trifluoromethyl)phenyl)-2,2-dimethyl-N-(thiazol-2-yl)chroman-7-sulfonamide,
4-(2-Chloro-4-(trifluoromethyl)phenyl)-2,2-dimethyl-N-(1,3,4-thiadiazol-2-yl)chroman-7-sulfonamide,
4-(2-Methoxy-4-(trifluoromethyl)phenyl)-2,2-dimethyl-N-(1,2,4-thiadiazol-5-yl)chroman-7-sulfonamide,
4-(2-Methoxy-4-(trifluoromethyl)phenyl)-2,2-dimethyl-N-(thiazol-2-yl)chroman-7-sulfonamide,
4-(2-Methoxy-4-(trifluoromethyl)phenyl)-2,2-dimethyl-N-(1,3,4-thiadiazol-2-yl)chroman-7-sulfonamide,
2,2-Dimethyl-4-(2-(1,2,3,6-tetrahydropyridin-4-yl)-4-(trifluoromethyl)phenyl)-N-(thiazol-2-yl)chroman-7-sulfonamide hydrochloride,
2,2-Dimethyl-4-(2-(1,2,3,6-tetrahydropyridin-4-yl)-4-(trifluoromethyl)phenyl)-N-(1,2,4-thiadiazol-5-yl)chroman-7-sulfonamide hydrochloride,
2,2-Dimethyl-4-(2-(piperidin-4-yl)-4-(trifluoromethyl)phenyl)-N-(thiazol-2-yl)chroman-7-sulfonamide hydrochloride,
(R/S)-2,2-Dimethyl-4-(2-(piperidin-4-yl)-4-(trifluoromethyl)phenyl)-N-(thiazol-2-yl)chroman-7-sulfonamide hydrochloride,
2,2-Dimethyl-4-(2-(piperidin-4-yl)-4-(trifluoromethyl)phenyl)-N-(1,2,4-thiadiazol-5-yl)chroman-7-sulfonamide hydrochloride,
(R/S)-2,2-Dimethyl-4-(2-(piperidin-4-yl)-4-(trifluoromethyl)phenyl)-N-(1,2,4-thiadiazol-5-yl)chroman-7-sulfonamide,
2,2-Dimethyl-4-(2-(piperidin-4-yl)-4-(trifluoromethyl)phenyl)-N-(1,3,4-thiadiazol-2-yl)chroman-7-sulfonamide hydrochloride,
2,2-Dimethyl-4-(2-(1-methylpiperidin-4-yl)-4-(trifluoromethyl)phenyl)-N-(thiazol-2-yl)chroman-7-sulfonamide,
(R/S)-2,2-Dimethyl-4-(2-(1-methylpiperidin-4-yl)-4-(trifluoromethyl)phenyl)-N-(thiazol-2-yl)chroman-7-sulfonamide,
2,2-Dimethyl-4-(2-(1-methylpiperidin-4-yl)-4-(trifluoromethyl)phenyl)-N-(1,2,4-thiadiazol-5-yl)chroman-7-sulfonamide hydrochloride,
(R/S)-2,2-Dimethyl-4-(2-(1-methylpiperidin-4-yl)-4-(trifluoromethyl)phenyl)-N-(1,2,4-thiadiazol-5-yl)chroman-7-sulfonamide,
2,2-Dimethyl-4-(2-(1-methylpiperidin-4-yl)-4-(trifluoromethyl)phenyl)-N-(1,3,4-thiadiazol-2-yl)chroman-7-sulfonamide hydrochloride,
4-(2-(3-Fluoroazetidin-3-yl)-4-(trifluoromethyl)phenyl)-2,2-dimethyl-N-(thiazol-2-yl)chroman-7-sulfonamide hydrochloride,
4-(2-(3-Fluoroazetidin-3-yl)-4-(trifluoromethyl)phenyl)-2,2-dimethyl-N-(1,2,4-thiadiazol-5-yl)chroman-7-sulfonamide hydrochloride,
4-(2-Chloro-4-(trifluoromethyl)phenyl)-N-(1,2,4-thiadiazol-5-yl)chroman-7-sulfonamide,
4-(2-Chloro-4-(trifluoromethyl)phenyl)-N-(1,3,4-thiadiazol-2-yl)chroman-7-sulfonamide,
4-(2-Chloro-4-(trifluoromethyl)phenyl)-N-(thiazol-2-yl)chroman-7-sulfonamide,
(R/S)-4-(2-Chloro-4-(trifluoromethyl)phenyl)-N-(thiazol-2-yl)chroman-7-sulfonamide,
4-(2-(1,2,3,6-Tetrahydropyridin-4-yl)-4-(trifluoromethyl)phenyl)-N-(thiazol-2-yl) chroman-7-sulfonamide hydrochloride,
4-(2-(1,2,3,6-Tetrahydropyridin-4-yl)-4-(trifluoromethyl)phenyl)-N-(1,2,4-thiadiazol-5-yl)chroman-7-sulfonamide hydrochloride,
4-(2-(1,2,3,6-Tetrahydropyridin-4-yl)-4-(trifluoromethyl)phenyl)-N-(1,3,4-thiadiazol-2-yl)chroman-7-sulfonamide hydrochloride,
4-(2-(3-Fluoroazetidin-3-yl)-4-(trifluoromethyl)phenyl)-N-(thiazol-2-yl)chroman-7-sulfonamide hydrochloride,
4-(2-(1-Methyl-1H-pyrazol-5-yl)-4-(trifluoromethyl)phenyl)-N-(thiazol-2-yl)chroman-7-sulfonamide,
4-(2-Chloro-4-(trifluoromethyl)phenoxy)-N-(thiazol-2-yl)chroman-7-sulfonamide,
(R)-4-(2-Chloro-4-(trifluoromethyl)phenoxy)-N-(thiazol-2-yl)chroman-7-sulfonamide,
(R)-4-(2-Cyano-4-(trifluoromethyl)phenoxy)-N-(thiazol-2-yl)chroman-7-sulfonamide,
(R)-4-(2,4-Dichlorophenoxy)-N-(thiazol-2-yl)chroman-7-sulfonamide,
(R)-4-(3,4-Dichlorophenoxy)-N-(thiazol-2-yl)chroman-7-sulfonamide,
(R)—N-(Thiazol-2-yl)-4-(2,4,6-trifluorophenoxy)chroman-7-sulfonamide,
4-(2-Chloro-4-(trifluoromethyl)phenoxy)-N-(1,2,4-thiadiazol-5-yl)chroman-7-sulfonamide,
(R)-4-(2-Chloro-4-(trifluoromethyl)phenoxy)-N-(1,2,4-thiadiazol-5-yl)chroman-7-sulfonamide,
(R)-4-(2-(1-Methyl-1H-pyrazol-5-yl)-4-(trifluoromethyl)phenoxy)-N-(thiazol-2-yl)chroman-7-sulfonamide,
(S)-4-(2-(1-Methyl-1H-pyrazol-5-yl)-4-(trifluoromethyl)phenoxy)-N-(thiazol-2-yl)chroman-7-sulfonamide,
(R)-4-(2-(Pyridin-3-yl)-4-(trifluoromethyl)phenoxy)-N-(thiazol-2-yl)chroman-7-sulfonamide,
(R)-4-(2-(Pyridin-4-yl)-4-(trifluoromethyl)phenoxy)-N-(thiazol-2-yl)chroman-7-sulfonamide,
(R)-4-(2-(5-Fluoropyridin-2-yl)-4-(trifluoromethyl)phenoxy)-N-(thiazol-2-yl)chroman-7-sulfonamide,
(R)-4-(2-(6-Methylpyridin-3-yl)-4-(trifluoromethyl)phenoxy)-N-(thiazol-2-yl)chroman-7-sulfonamide,
(R)-4-(2-(2-Methylpyrimidin-5-yl)-4-(trifluoromethyl)phenoxy)-N-(thiazol-2-yl)chroman-7-sulfonamide,
(R)-4-(2-(1-Methyl-1H-pyrazol-4-yl)-4-(trifluoromethyl)phenoxy)-N-(thiazol-2-yl)chroman-7-sulfonamide,
(R)-4-(2-Ethyl-4-(trifluoromethyl)phenoxy)-N-(thiazol-2-yl)chroman-7-sulfonamide,
(R)-4-(2-Isopropyl-4-(trifluoromethyl)phenoxy)-N-(thiazol-2-yl)chroman-7-sulfonamide,
(R)-4-(2-Cyclopropyl-4-(trifluoromethyl)phenoxy)-N-(thiazol-2-yl)chroman-7-sulfonamide,
(R)-4-(2-(1-Isopropyl-1H-pyrazol-5-yl)-4-(trifluoromethyl)phenoxy)-N-(thiazol-2-yl)chroman-7-sulfonamide,
(R)-4-(2-(1-(2-Fluoroethyl)-1H-pyrazol-5-yl)-4-(trifluoromethyl)phenoxy)-N-(thiazol-2-yl)chroman-7-sulfonamide,
(R)-4-((4′-Fluoro-5-(trifluoromethyl)-[1,1′-biphenyl]-2-yl)oxy)-N-(thiazol-2-yl)chroman-7-sulfonamide,
(R)-4-(2-(Pyridin-2-yl)-4-(trifluoromethyl)phenoxy)-N-(thiazol-2-yl)chroman-7-sulfonamide,
(R)-4-(2-(4-Cyclopropyl-1H-1,2,3-triazol-1-yl)-4-(trifluoromethyl)phenoxy)-N-(thiazol-2-yl)chroman-7-sulfonamide,
(R)-4-(2-(1H-Pyrazol-1-yl)-4-(trifluoromethyl)phenoxy)-N-(thiazol-2-yl)chroman-7-sulfonamide,
(R)-4-(2-(2-Methylthiazol-4-yl)-4-(trifluoromethyl)phenoxy)-N-(thiazol-2-yl)chroman-7-sulfonamide,
(R)-4-(2-(2-Methyloxazol-4-yl)-4-(trifluoromethyl)phenoxy)-N-(thiazol-2-yl)chroman-7-sulfonamide,
(R)—N,N-Dimethyl-2-(2-((7-(N-(thiazol-2-yl)sulfamoyl)chroman-4-yl)oxy)-5-(trifluoromethyl)phenyl)acetamide,
(R)-2-(2-((7-(N-(Thiazol-2-yl)sulfamoyl)chroman-4-yl)oxy)-5-(trifluoromethyl)phenyl)pyridine 1-oxide,
4-(2-(1,2,3,6-Tetrahydropyridin-4-yl)-4-(trifluoromethyl)phenoxy)-N-(thiazol-2-yl)chroman-7-sulfonamide trifluoroacetic acid,
4-(2-(Piperidin-4-yl)-4-(trifluoromethyl)phenoxy)-N-(thiazol-2-yl)chroman-7-sulfonamide hydrochloride,
4-(2-(piperidin-4-yl)-4-(trifluoromethyl)phenoxy)-N-(1,2,4-thiadiazol-5-yl)chroman-7-sulfonamide trifluoroacetic acid,
(R)-4-(2-Chloro-4-(trifluoromethyl)phenoxy)-2,2-dimethyl-N-(thiazol-2-yl)chroman-7-sulfonamide,
(S)-4-(2-Chloro-4-(trifluoromethyl)phenoxy)-2,2-dimethyl-N-(thiazol-2-yl)chroman-7-sulfonamide,
(R)-2,2-Dimethyl-N-(thiazol-2-yl)-4-(4-(trifluoromethyl)phenoxy)chroman-7-sulfonamide,
(R)-2,2-Dimethyl-N-(thiazol-2-yl)-4-(3-(trifluoromethyl)phenoxy)chroman-7-sulfonamide,
(R)-4-(2,4-Dichlorophenoxy)-2,2-dimethyl-N-(thiazol-2-yl)chroman-7-sulfonamide,
(R)-4-(2-Cyano-4-(trifluoromethyl)phenoxy)-2,2-dimethyl-N-(thiazol-2-yl)chroman-7-sulfonamide,
(R)-4-(2-Ethyl-4-(trifluoromethyl)phenoxy)-2,2-dimethyl-N-(thiazol-2-yl)chroman-7-sulfonamide,
(R)-4-(2-(1-Isopropyl-1H-pyrazol-5-yl)-4-(trifluoromethyl)phenoxy)-2,2-dimethyl-N-(thiazol-2-yl)chroman-7-sulfonamide,
(R)-4-((4′-Fluoro-5-(trifluoromethyl)-[1,1′-biphenyl]-2-yl)oxy)-2,2-dimethyl-N-(thiazol-2-yl)chroman-7-sulfonamide,
(R)-4-(2-(4-Cyclopropyl-1H-1,2,3-triazol-1-yl)-4-(trifluoromethyl)phenoxy)-2,2-dimethyl-N-(thiazol-2-yl)chroman-7-sulfonamide,
(R)-4-(2-(1H-Pyrazol-1-yl)-4-(trifluoromethyl)phenoxy)-2,2-dimethyl-N-(thiazol-2-yl)chroman-7-sulfonamide,
(R)-2,2-Dimethyl-4-(2-(pyridin-2-yl)-4-(trifluoromethyl)phenoxy)-N-(thiazol-2-yl)chroman-7-sulfonamide,
(R)-2,2-Dimethyl-4-(2-(2-methylthiazol-4-yl)-4-(trifluoromethyl)phenoxy)-N-(thiazol-2-yl)chroman-7-sulfonamide,
(R)-2,2-Dimethyl-4-(2-(2-methyloxazol-4-yl)-4-(trifluoromethyl)phenoxy)-N-(thiazol-2-yl)chroman-7-sulfonamide,
(R)-2,2-Dimethyl-4-(2-(1-methyl-1H-pyrazol-5-yl)-4-(trifluoromethyl)phenoxy)-N-(thiazol-2-yl)chroman-7-sulfonamide,
(R)-2,2-Dimethyl-4-(2-(1-methyl-1H-pyrazol-5-yl)-5-(trifluoromethyl)phenoxy)-N-(thiazol-2-yl)chroman-7-sulfonamide,
(R)-2,2-Dimethyl-4-(2-(pyridin-4-yl)-4-(trifluoromethyl)phenoxy)-N-(thiazol-2-yl)chroman-7-sulfonamide,
(R)-4-(2-Chloro-4-(trifluoromethyl)phenoxy)-N-(thiazol-2-yl)spiro[chroman-2,1′-cyclobutane]-7-sulfonamide,
(R)-4-(2-(1-Methyl-1H-pyrazol-5-yl)-4-(trifluoromethyl)phenoxy)-N-(thiazol-2-yl)spiro[chroman-2,1′-cyclobutane]-7-sulfonamide,
(2R/S,4R)-4-(2-Chloro-4-(trifluoromethyl)phenoxy)-2-ethyl-N-(thiazol-2-yl)chroman-7-sulfonamide,
(2S/R,4R)-4-(2-Chloro-4-(trifluoromethyl)phenoxy)-2-ethyl-N-(thiazol-2-yl)chroman-7-sulfonamide,
(2S/R,4R)-2-Ethyl-4-(2-(1-methyl-1H-pyrazol-5-yl)-4-(trifluoromethyl) phenoxy)-N-(thiazol-2-yl)chroman-7-sulfonamide,
(2R/S,4R)-2-Ethyl-4-(2-(1-methyl-1H-pyrazol-5-yl)-4-(trifluoromethyl) phenoxy)-N-(thiazol-2-yl)chroman-7-sulfonamide,
5-(2-Chloro-4-(trifluoromethyl)phenoxy)-N-(thiazol-2-yl)-5,6,7,8-tetrahydronaphthalene-2-sulfonamide,
(R)-5-(2-Chloro-4-(trifluoromethyl)phenoxy)-N-(thiazol-2-yl)-5,6,7,8-tetrahydronaphthalene-2-sulfonamide,
5-(2-Bromo-4-(trifluoromethyl)phenoxy)-N-(thiazol-2-yl)-5,6,7,8-tetrahydronaphthalene-2-sulfonamide,
5-(2-Chloro-4-(trifluoromethyl)phenoxy)-N-(1,2,4-thiadiazol-5-yl)-5,6,7,8-tetrahydronaphthalene-2-sulfonamide,
5-(2-Bromo-4-(trifluoromethyl)phenoxy)-N-(1,2,4-thiadiazol-5-yl)-5,6,7,8-tetrahydronaphthalene-2-sulfonamide,
N-(Thiazol-2-yl)-5-(4-(trifluoromethyl)phenoxy)-5,6,7,8-tetrahydronaphthalene-2-sulfonamide,
5-(2-(1-Methyl-1H-pyrazol-5-yl)-4-(trifluoromethyl)phenoxy)-N-(thiazol-2-yl)-5,6,7,8-tetrahydronaphthalene-2-sulfonamide,
(R)-5-(2-(1-Methyl-1H-pyrazol-5-yl)-4-(trifluoromethyl)phenoxy)-N-(thiazol-2-yl)-5,6,7,8-tetrahydronaphthalene-2-sulfonamide,
5-(2-(1,2,3,6-Tetrahydropyridin-4-yl)-4-(trifluoromethyl)phenoxy)-N-(thiazol-2-yl)-5,6,7,8-tetrahydronaphthalene-2-sulfonamide trifluoroacetic acid,
(R/S)-5-(2-(1,2,3,6-Tetrahydropyridin-4-yl)-4-(trifluoromethyl)phenoxy)-N-(thiazol-2-yl)-5,6,7,8-tetrahydronaphthalene-2-sulfonamide trifluoroacetic acid,
5-(2-(Piperidin-4-yl)-4-(trifluoromethyl)phenoxy)-N-(thiazol-2-yl)-5,6,7,8-tetrahydronaphthalene-2-sulfonamide trifluoroacetic acid,
(R/S)-5-(2-(Piperidin-4-yl)-4-(trifluoromethyl)phenoxy)-N-(thiazol-2-yl)-5,6,7,8-tetrahydronaphthalene-2-sulfonamide,
5-(2-(Piperidin-4-yl)-4-(trifluoromethyl)phenoxy)-N-(1,2,4-thiadiazol-5-yl)-5,6,7,8-tetrahydronaphthalene-2-sulfonamide,
5-(2-(Piperidin-4-yl)-4-(trifluoromethyl)phenoxy)-N-(1,3,4-thiadiazol-2-yl)-5,6,7,8-tetrahydronaphthalene-2-sulfonamide trifluoroacetic acid,
5-(2-(1-Methylpiperidin-4-yl)-4-(trifluoromethyl)phenoxy)-N-(thiazol-2-yl)-5,6,7,8-tetrahydronaphthalene-2-sulfonamide,
5-(2-(1-Methylpiperidin-4-yl)-4-(trifluoromethyl)phenoxy)-N-(1,2,4-thiadiazol-5-yl)-5,6,7,8-tetrahydronaphthalene-2-sulfonamide,
5-(2-(1-Methylpiperidin-4-yl)-4-(trifluoromethyl)phenoxy)-N-(1,3,4-thiadiazol-2-yl)-5,6,7,8-tetrahydronaphthalene-2-sulfonamide,
(R)-4-(2-(2-Fluoroethyl)-4-(trifluoromethyl)phenoxy)-N-(thiazol-2-yl)chroman-7-sulfonamide,
(R)-4-(2-(1-Ethyl-1H-pyrazol-5-yl)-4-(trifluoromethyl)phenoxy)-N-(thiazol-2-yl)chroman-7-sulfonamide,
(R)-2-(2-((2,2-Dimethyl-7-(N-(thiazol-2-yl)sulfamoyl)chroman-4-yl)oxy)-5-(trifluoromethyl)phenyl)pyridine 1-oxide,
(R)-3-(2-((7-(N-(Thiazol-2-yl)sulfamoyl)chroman-4-yl)oxy)-5-(trifluoromethyl)phenyl)pyridine 1-oxide and
(R)-4-(2-((7-(N-(Thiazol-2-yl)sulfamoyl)chroman-4-yl)oxy)-5-(trifluoromethyl)phenyl)pyridine 1-oxide

or a free base thereof, N-oxide thereof, or stereoisomers thereof or a pharmaceutically acceptable salt thereof.

The details of one or more embodiments of the invention set forth in the below are illustrative in nature only and not intended to limit to the scope of the invention. Other features, objects and advantages of the inventions will be apparent from the description and claims.

In another aspect of the invention, there is provided a compound of Formulae (I), (Ia), (II), (III) or (IV), or a pharmaceutically acceptable salt thereof for use as a medicament.

In another aspect of the invention, there is provided a compound of Formulae (I), (Ia), (II), (III) or (IV), or a pharmaceutically acceptable salt thereof, for use in treating the diseases, disorders, syndromes or conditions associated with VGSC particularly Nav1.7.

In another aspect, the invention provides a pharmaceutical composition comprising at least one compound of Formulae (I), (Ia), (II), (III) or (IV), or a pharmaceutically acceptable salt thereof and at least one pharmaceutically acceptable excipient.

In another aspect, the invention provides a pharmaceutical composition comprising a compound of Formulae (I), (Ia), (II), (III) or (IV), or a pharmaceutically acceptable salt thereof, for use in treating, the diseases disorders, syndromes or conditions associated with VGSC particularly Nav1.7 in a subject, in need thereof by administering to the subject, one or more compounds described herein in a therapeutically effective amount to cause modulation of such receptor.

In another aspect, the invention provides a pharmaceutical composition comprising a compound of Formulae (I), (Ia), (II), (III) or (IV), or a pharmaceutically acceptable salt thereof, N-oxide thereof, or a pharmaceutically acceptable stereoisomer, thereof together with a pharmaceutically acceptable excipient.

In another aspect of the invention, there is provided use of a compound of Formulae (I), (Ia), (II), (III) or (IV), or a pharmaceutically acceptable salt thereof, for the manufacture of a medicament for treating, the diseases, disorders, syndromes or conditions associated with VGSC.

In another aspect, there are provided process for the preparation of compounds of Formula (Ib):

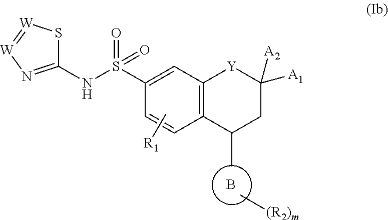

wherein ring B, W, Y, A₁, A₂, R₁, R₂ and m are as defined herein above;

the process comprising the steps of:
a) reducing a double bond of compound of formula (5) followed by deprotection using suitable reagents to obtain compound of formula (6)

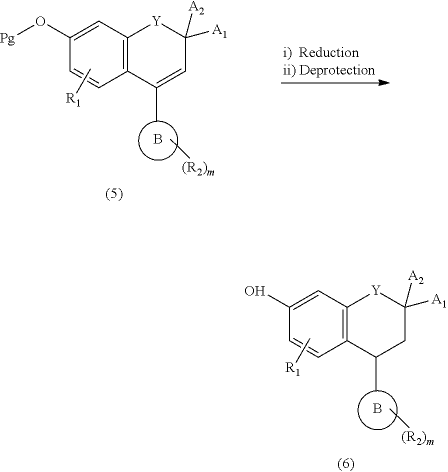

b) reacting a hydroxy group in compound of formula (6) with suitable triflating agents to obtain compound of formula (7)

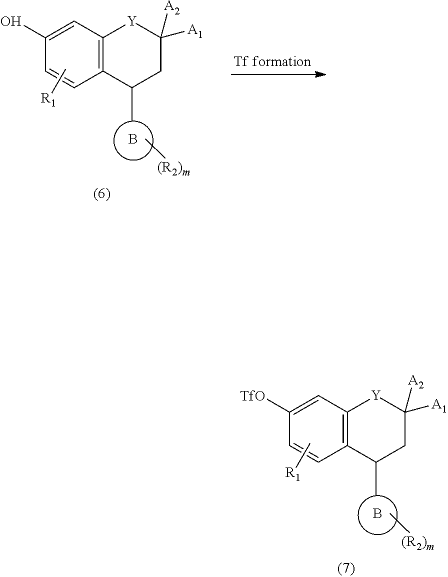

c) reacting a triflate compound of formula (7) with benzyl mercaptan in the presence of Pd catalyst to give the compound of formula (8)

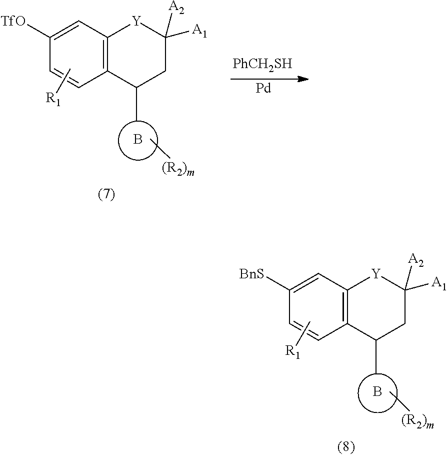

d) converting a compound of formula (8) to compound of formula (9) using pentafluorophenol

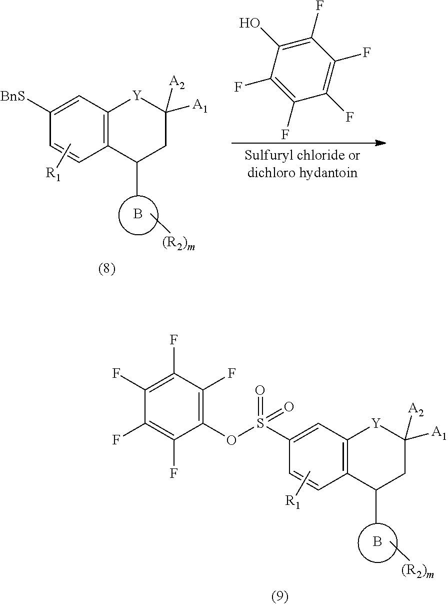

e) reacting a compound of formula (9) with an amino compound of formula (10) in presence of suitable base to afford compound of formula (Ib)

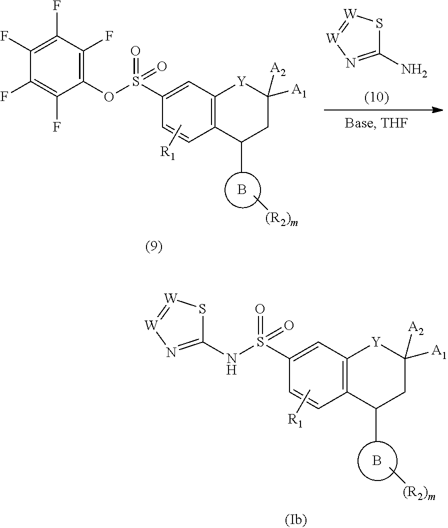

In another aspect, there are provided process for the preparation of compounds of Formula (Ic):

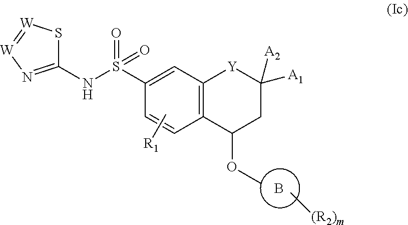

wherein ring B, Y, W, A₁, A₂, R₁, R₂ and m are as defined herein above;

the process comprising the steps of:
a) reducing a keto group of compound of formula (13) using suitable reducing agents to obtain compound of formula (18)

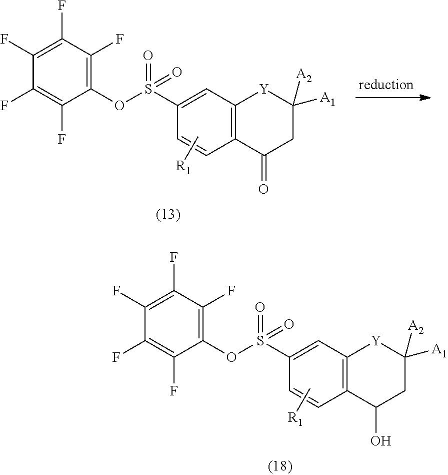

b) reacting a hydroxyl group in compound of formula (18) with phenol of formula (19) under Mitsunobu reaction conditions gives the compound of formula (20).

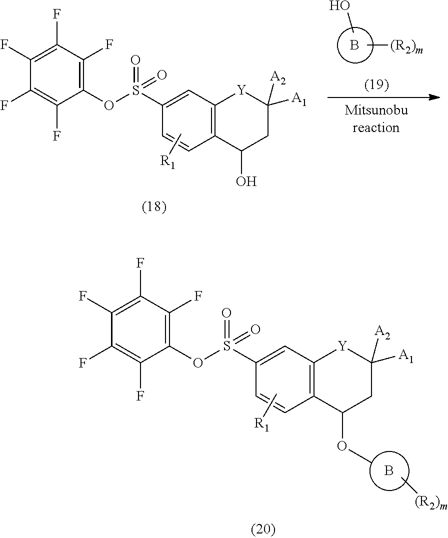

c) reacting a compound of formula (20) with an amino compound of formula (10) in presence of suitable base to afford compound of formula (Ic)

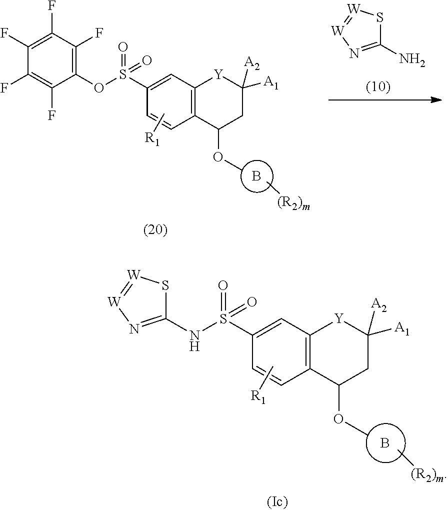

### DETAILED DESCRIPTION OF THE INVENTION

### Definitions and Abbreviations

Unless otherwise stated, the following terms used in the specification and claims have the meanings given below.

For purposes of interpreting the specification, the following definitions will apply and whenever appropriate, terms used in the singular will also include the plural and vice versa.

The terms “halogen” or “halo” means fluorine, chlorine, bromine, or iodine.

Unless otherwise stated, in the present application “oxo” means C(═O) group. Such an oxo group may be a part of either a cycle or a chain in the compounds of the present invention.

The term “alkyl” refers to an alkane derived hydrocarbon radical that includes solely carbon and hydrogen atoms in the backbone, contains no unsaturation, has from one to six carbon atoms, and is attached to the remainder of the molecule by a single bond, for example (C₁-C₆)alkyl or (C₁-C₄)alkyl, representative groups include e.g., methyl, ethyl, n-propyl, 1-methylethyl (isopropyl), n-butyl, n-pentyl, 1,1-dimethylethyl (t-butyl) and the like. Unless set forth or recited to the contrary, all alkyl groups described or claimed herein may be straight chain or branched.

The term “alkenyl” refers to a hydrocarbon radical containing from 2 to 10 carbon atoms and including at least one carbon-carbon double bond. Non-limiting Examples of alkenyl groups include, for example (C₂-C₆)alkenyl, (C₂-C₄)alkenyl, ethenyl, 1-propenyl, 2-propenyl (allyl), iso-propenyl, 2-methyl-1-propenyl, 1-butenyl, 2-butenyl and the like. Unless set forth or recited to the contrary, all alkenyl groups described or claimed herein may be straight chain or branched.

The term “alkynyl” refers to a hydrocarbon radical containing 2 to 10 carbon atoms and including at least one carbon-carbon triple bond. Non-limiting Examples of alkynyl groups include, for example (C₂-C₆)alkynyl, (C₂-C₄)alkynyl, ethynyl, propynyl, butynyl and the like. Unless set forth or recited to the contrary, all alkynyl groups described or claimed herein may be straight chain or branched.

The term “alkoxy” refers to an alkyl group attached via an oxygen linkage. Non-limiting Examples of such groups include, for example (C₁-C₆)alkoxy, (C₁-C₄)alkoxy, methoxy, ethoxy and propoxy and the like. Unless set forth or recited to the contrary, all alkoxy groups described or claimed herein may be straight chain or branched.

The term “alkoxyalkyl” refers to an alkoxy group as defined above directly bonded to an alkyl group as defined above, for example (C₁-C₆)alkoxy-(C₁-C₆)alkyl, (C₁-C₄)alkoxy-(C₁-C₄)alkyl, —CH₂—O—CH₃, —CH₂—O—CH₂CH₃, —CH₂CH₂—O—CH₃ and the like.

The term “haloalkyl” refers to an alkyl group as defined above that is substituted by one or more halogen atoms as defined above. For example (C₁-C₆)haloalkyl or (C₁-C₄)haloalkyl. Suitably, the haloalkyl may be monohaloalkyl, dihaloalkyl or polyhaloalkyl including perhaloalkyl. A monohaloalkyl can have one iodine, bromine, chlorine or fluorine atom. Dihaloalkyl and polyhaloalkyl groups can be substituted with two or more of the same halogen atoms or a combination of different halogen atoms. Suitably, a polyhaloalkyl is substituted with up to 12 halogen atoms. Non-limiting Examples of a haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, dichloropropyl and the like. A perhaloalkyl refers to an alkyl having all hydrogen atoms replaced with halogen atoms. Unless set forth or recited to the contrary, all haloalkyl groups described or claimed herein may be straight chain or branched.

The term “hydroxyalkyl” refers to an alkyl group, as defined above that is substituted by one or more hydroxy groups. Preferably, the hydroxyalkyl is monohydroxyalkyl or dihydroxyalkyl. Non-limiting examples of a hydroxyalkyl include 2-hydroxyethyl, 3-hydroxypropyl, 2-hydroxypropyl, and the like.

The term “haloalkoxy” refers to a haloalkyl, defined herein, group attached via an oxygen linkage. Non-limiting examples of such groups are monohaloalkoxy, dihaloalkoxy or polyhaloalkoxy including perhaloalkoxy. Unless set forth or recited to the contrary, all haloalkoxy group described or claimed herein may be straight chain or branched, substituted or unsubstituted.

The term “cycloalkyl” refers to a non-aromatic mono or multicyclic ring system having 3 to 12 carbon atoms, such as (C₃-C₁₀)cycloalkyl, (C₃-C₆)cycloalkyl, cyclopropyl, cyclobutyl, cyclopentyl, cyclohexyl and the like. Examples of multicycliccycloalkyl groups include, but are not limited to, perhydronaphththyl, adamantyl and norbornyl groups, bridged cyclic groups or spirobicyclic groups, e.g., spiro(4,4)non-2-yl and the like.

The term “cycloalkenyl” refers to a non-aromatic mono or multicyclic ring system having 3 to 12 carbon atoms and including at least one carbon-carbon double bond, such as cyclopentenyl, cyclohexenyl, cycloheptenyl and the like. Unless set forth or recited to the contrary, all cycloalkenyl groups described or claimed herein may be substituted or unsubstituted.

The term “cycloalkylalkyl” refers to a cycloalkyl group as defined above, directly bonded to an alkyl group as defined above, e.g., cyclopropylmethyl, cyclobutylmethyl, cyclopentylmethyl, cyclohexylmethyl, cyclohexylethyl, etc. Unless set forth or recited to the contrary, all cycloalkylalkyl groups described or claimed herein may be substituted or unsubstituted.

The term “aryl” refers to an aromatic radical having 6- to 14-carbon atoms, including monocyclic, bicyclic and tricyclic aromatic systems, such as phenyl, naphthyl, tetrahydronaphthyl, indanyl, and biphenyl and the like.

The term “arylalkyl” refers to an aryl group as defined above directly bonded to an alkyl group as defined above, e.g., —CH₂C₆H₅ and —C₂H₄C₆H₅. Unless set forth or recited to the contrary, all arylalkyl groups described or claimed herein may be substituted or unsubstituted.

The term “heterocyclic ring” or “heterocyclyl ring” or “heterocyclyl”, unless otherwise specified, refers to substituted or unsubstituted non-aromatic 3- to 15-membered ring which consists of carbon atoms and with one or more heteroatom(s) independently selected from N, O or S. The heterocyclic ring may be a mono-, bi- or tricyclic ring system, which may include fused, bridged or spiro ring systems and the nitrogen, carbon, oxygen or sulfur atoms in the heterocyclic ring may be optionally oxidized to various oxidation states. In addition, the nitrogen atom may be optionally quaternized, the heterocyclic ring or heterocyclyl may optionally contain one or more olefinic bond(s), and one or two carbon atoms(s) in the heterocyclic ring or heterocyclyl may be interrupted with —CF₂—, —C(O)—, —S(O)—, S(O)₂, —C(═N—(C₁-C₆)alkyl)-, or —C(═N—(C₃-C₆)cycloalkyl), etc. In addition heterocyclic ring may also be fused with aromatic ring. Non-limiting examples of heterocyclic rings include azetidinyl, benzopyranyl, chromanyl, decahydroisoquinolyl, indanyl, indolinyl, isoindolinyl, isochromanyl, isothiazolidinyl, isoxazolidinyl, morpholinyl, oxazolinyl, oxazolidinyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl, tetrahydropyranyl, thiazolinyl, thiazolidinyl, thiamorpholinyl, thiamorpholinyl sulfoxide, thiamorpholinyl sulfone indoline, benzodioxole, tetrahydroquinoline, tetrahydrobenzopyran and the like. The heterocyclic ring may be attached to the main structure at any heteroatom or carbon atom that results in the creation of a stable structure. Unless set forth or recited to the contrary, all heterocyclyl groups described or claimed herein may be substituted or unsubstituted; substituents may be on same or different ring atom.

The term “heteroaryl” unless otherwise specified, refers to a substituted or unsubstituted 5- to 14-membered aromatic heterocyclic ring with one or more heteroatom(s) independently selected from N, O or S. In addition the nitrogen atom in a heteroaryl ring is optionally quaternized to form corresponding N-oxide. The heteroaryl may be a mono-, bi- or tricyclic ring system. The heteroaryl ring may be attached to the main structure at any heteroatom or carbon atom that results in the creation of a stable structure. Non-limiting examples of a heteroaryl ring include oxazolyl, isoxazolyl, imidazolyl, furyl, indolyl, isoindolyl, pyrrolyl, triazolyl, triazinyl, tetrazolyl, thienyl, thiazolyl, isothiazolyl, pyridyl, pyrimidinyl, pyrazinyl, pyridazinyl, benzofuranyl, benzothiazolyl, benzoxazolyl, benzimidazolyl, benzothienyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indolizinyl, acridinyl, phenazinyl, phthalazinyl and the like. Unless set forth or recited to the contrary, all heteroaryl groups described or claimed herein may be substituted or unsubstituted.

The term “heterocyclylalkyl” refers to a heterocyclic ring radical directly bonded to an alkyl group. The heterocyclylalkyl radical may be attached to the main structure at any carbon atom in the alkyl group that results in the creation of a stable structure. Unless set forth or recited to the contrary, all heterocyclylalkyl groups described or claimed herein may be substituted or unsubstituted.

The term “heteroarylalkyl” refers to a heteroaryl ring radical directly bonded to an alkyl group. The heteroarylalkyl radical may be attached to the main structure at any carbon atom in the alkyl group that results in the creation of a stable structure. Unless set forth or recited to the contrary, all heteroarylalkyl groups described or claimed herein may be substituted or unsubstituted.

Unless otherwise specified, the term “substituted” as used herein refers to a group or moiety having one or more substituents attached to the structural skeleton of the group or moiety. Such substituents include, but are not limited to hydroxy, halogen, carboxyl, cyano, nitro, oxo (═O), thio (═S), (C₁-C₆)alkyl, (C₁-C₆)haloalkyl, (C₂-C₆)alkenyl, (C₂-C₆)alkynyl, aryl, aryl(C₁-C₆)alkyl, (C₃-C₁₂)cycloalkyl, heteroaryl, heterocyclic ring, heterocyclyl(C₁-C₆)alkyl, heteroaryl(C₁-C₆)alkyl, —C(O)OR^{x}, —C(O)R^{x}, —C(S)R^{x}, —C(O)NR^{x}R^{y}, —NR^{x}C(O)NR^{y}R^{z}, —N(R^{x})S(O)R^{y}, —N(R^{x})S(O)₂R^{y}, —NR^{x}R^{y}, —NR^{x}C(O)R^{y}, —NR^{x}C(S)R^{y}, —NR^{x}C(S)NR^{y}R^{z}, —S(O)₂NR^{x}R^{y}, —OR^{x}, —OC(O)R^{x}, —OC(O)NR^{x}R^{y}, —R^{x}C(O)OR^{y}, —R^{x}C(O)NR^{y}R^{z}, —R^{x}C(O)R^{y}, —SR^{x}, and —S(O)₂R^{x}; wherein each occurrence of R^{x}, R^{y} and R^{z} are independently selected from hydrogen, halogen, (C₁-C₄)alkyl, (C₁-C₄)haloalkyl, (C₂-C₄)alkenyl, (C₂-C₆)alkynyl, (C₃-C₁₂)cycloalkyl and aryl.

For example one representative group of moieties which may be a “substituent” is selected from hydroxy, halogen, cyano, nitro, oxo (═O), thio (═S), (C₁-C₄)alkyl, (C₁-C₄)haloalkyl, (C₃-C₇)cycloalkyl, —C(O)OR^{x}, —C(O)R^{x}, —C(O)NR^{x}R^{y}, —NR^{x}R^{y}, —NR^{x}C(O)R^{y}, —S(O)₂NR^{x}R^{y}, —OR^{x}, —OC(O)R^{x}, —SR^{x} and —S(O)₂R^{x}; wherein each occurrence of R^{x} and R^{y} are independently selected from hydrogen, halogen, (C₁-C₄)alkyl, (C₁-C₄)haloalkyl and (C₃-C₆)cycloalkyl.

It is to be understood that the aforementioned “substituted” groups cannot be further substituted. For Example, when the substituent on “substituted alkyl” is “aryl” or “alkenyl”, the aryl or alkenyl cannot be substituted aryl or substituted alkenyl respectively.

The compounds of the present invention may have one or more chiral centers. The absolute stereochemistry at each chiral centre may be ‘R’ or ‘S’. The compounds of the invention include all diastereomers and enantiomers and mixtures thereof. Unless specifically mentioned otherwise, reference to one stereoisomer applies to any of the possible stereoisomers. Whenever the stereoisomeric composition is unspecified, it is to be understood that all possible stereoisomers are included.

The term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. The terms “racemate” or “racemic mixture” refer to a mixture of equal parts of enantiomers.

The term “treating” or “treatment” of a state, disease, disorder, condition or syndrome includes: (a) preventing or delaying the appearance of clinical symptoms of the state, disease, disorder, condition or syndrome developing in a subject that may be afflicted with or predisposed to the state, disease, disorder, condition or syndrome but does not yet experience or display clinical or subclinical symptoms of the state, disease, disorder, condition or syndrome; (b) inhibiting the state, disease, disorder, condition or syndrome, i.e., arresting or reducing the development of the disease or at least one clinical or subclinical symptom thereof; c) lessening the severity of a disease disorder or condition or at least one of its clinical or subclinical symptoms thereof; and/or (d) relieving the disease, i.e., causing regression of the state, disorder or condition or at least one of its clinical or subclinical symptoms.

The term “modulate” or “modulating” or “modulation” refers to a decrease or inhibition in the amount, quality, or effect of a particular activity, function or molecule; by way of illustration that antagonists of a voltage-gated sodium channels are modulators of VGSC. Any such modulation, whether it is partial or complete inhibition or prevention of ion flux, is sometimes referred to herein as “blocking” and corresponding compounds as “blockers”. For example, the compounds of invention are useful as modulators of the Na_{V}1.7. In general, the compounds of the invention modulates the activity of a sodium channel downwards, inhibits the voltage-dependent activity of the sodium channel, and/or reduces or prevents sodium ion flux across a cell membrane by preventing sodium channel activity such as ion flux.

The term “subject” includes mammals, preferably humans and other animals, such as domestic animals; e.g., household pets including cats and dogs.

A “therapeutically effective amount” refers to the amount of a compound that, when administered to a subject in need thereof, is sufficient to cause a desired effect. The “therapeutically effective amount” will vary depending on the compound, the disease and its severity, age, weight, physical condition and responsiveness of the subject to be treated.

### Pharmaceutically Acceptable Salts:

The compounds of the invention may form salts with acid or base. The compounds of invention may be sufficiently basic or acidic to form stable nontoxic acid or base salts, administration of the compound as a pharmaceutically acceptable salt may be appropriate. Non-limiting examples of pharmaceutically acceptable salts are inorganic, organic acid addition salts formed by addition of acids including hydrochloride salts. Non-limiting examples of pharmaceutically acceptable salts are inorganic, organic base addition salts formed by addition of bases. The compounds of the invention may also form salts with amino acids. Pharmaceutically acceptable salts may be obtained using standard procedures well known in the art, for example by reacting a sufficiently basic compound such as an amine with a suitable acid affording a physiologically acceptable anion.

With respect to the overall compounds described by the Formulae (I) to (IV) herein, the invention extends to these stereoisomeric forms and to mixtures thereof. To the extent prior art teaches synthesis or separation of particular stereoisomers, the different stereoisomeric forms of the invention may be separated from one another by a method known in the art, or a given isomer may be obtained by stereospecific or asymmetric synthesis or chiral HPLC (high performance liquid chromatography. Tautomeric forms and mixtures of compounds described herein are also contemplated.

### Compound Screening:

The screening of compounds of the invention for VGSC modulatory activity particularly Nav1.7 can be achieved by using various in vitro and in vivo protocols. Some of the methods include measuring current (electrophysiology), estimating membrane potential (using membrane potential dyes or voltage specific dye pairs), measuring ion flux (e.g., Sodium or Guanidium), measuring second messenger and transcription factor levels, measuring sodium concentration or by Rubidium efflux assay. These assays can be performed in tissue slices or cell lines that endogenously express sodium channels (e.g. ND7/23, SHSY-5Y). Alternatively, one can also use cell lines stably expressing the Nay of interest (e.g., stable cell lines generated in HEK293 cells or CHO cells).

### Pharmaceutical Compositions:

The invention relates to pharmaceutical compositions containing the compounds of the Formulae (I) to (IV), or pharmaceutically acceptable salts thereof disclosed herein. In particular, the pharmaceutical compositions contain a therapeutically effective amount of at least one compound of Formula (I) and at least one pharmaceutically acceptable excipient (such as a carrier or diluent). Preferably, the pharmaceutical compositions include the compound(s) described herein in an amount sufficient to modulate the ion flux through a voltage-dependent sodium channel to treat sodium channel mediated diseases such as pain when administered to a subject.

The compound of the invention may be incorporated with a pharmaceutically acceptable excipient (such as a carrier or a diluent) or be diluted by a carrier, or enclosed within a carrier which can be in the form of a capsule, sachet, paper or other container. The pharmaceutically acceptable excipient includes a pharmaceutical agent that does not itself induce the production of antibodies harmful to the individual receiving the composition, and which may be administered without undue toxicity.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, salicylic acid, fatty acids, fatty acid amines, fatty acid monoglycerides and diglycerides, pentaerythritol fatty acid esters, polyoxyethylene, hydroxymethylcellulose and polyvinylpyrrolidone.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared by conventional techniques known in the art. For example, the active compound can be mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound can be adsorbed on a granular solid container, for example, in a sachet.

The pharmaceutical compositions may be administered in conventional forms, for example, capsules, tablets, aerosols, solutions, suspensions or products for topical application.

The route of administration may be any route which effectively transports the active compound of the invention to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment).

Solid oral Formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Liquid Formulations include, but are not limited to, syrups, emulsions, soft gelatin and sterile injectable liquids, such as aqueous or non-aqueous liquid suspensions or solutions. For parenteral application, particularly suitable are injectable solutions or suspensions Formulation.

Liquid Formulations include, but are not limited to, syrups, emulsions, suspensions, solutions, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

For administration to human patients, the total daily dose of the compounds of the invention depends, of course, on the mode of administration. For example, oral administration may require a higher total daily dose, than an intravenous (direct into blood).

The quantity of active component in a unit dose preparation may be varied or adjusted from 0.1 mg to 10000 mg, more typically 1.0 mg to 1000 mg, and most typically 10 mg to 500 mg, according to the potency of the active component or mode of administration.

Suitable doses of the compounds for use in treating the diseases disorders, syndromes and conditions described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects for the patient. For example, the daily dosage of the Sodium channel modulator can range from about 0.1 to about 30.0 mg/kg. Mode of administration, dosage forms, suitable pharmaceutical excipients, diluents or carriers can also be well used and adjusted by those skilled in the art. All changes and modifications are envisioned within the scope of the invention.

### Methods of Treatment:

In an embodiment the invention are provided compounds and pharmaceutical compositions that are useful in the treatment of diseases, disorders, syndromes and/or conditions modulated by Nav1.7 channel. The invention further provides a method of treating a disease, condition and/or disorder modulated by Nav1.7 channel in a subject in need thereof by administering to the subject a therapeutically effective amount of a compound or a pharmaceutical composition of the invention.

One aspect of the invention provides methods for decreasing ion flow through voltage-gated sodium channels in a cell, comprising contacting a cell containing the target ion channels with a compound, associated to voltage-dependent gated ion channel, described herein.

In another aspect of the invention, the methods are also useful for the diagnosis of conditions that can be treated by acting on ion flux through voltage-dependent gated ion channel, for determining if a patient will be responsible to therapeutic agents.

In still another aspect of the invention provides a method for the treatment of a disorder or condition through modulating voltage-gated sodium channel particularly Nav1.7. In this method, a subject in need of such treatment is administered an effective amount of a compound described herein and/or according to Formulae (I), (Ia), (II), (III) or (IV), or a pharmaceutically acceptable salt thereof described herein.

The compound of Formula Formulae (I), (Ia), (II), (III) or (IV), or a pharmaceutically acceptable salt thereof, being a Nav1.7, is potentially useful in the treating, managing and/or lessening of diseases, disorders, syndromes or conditions including but not limited to pain, erythromelalgia, neurological disorders, cardiovascular conditions, neuromuscular conditions, multiple sclerosis, cancer, pruritus, benign prostatic hyperplasia (BPH) and the like.

Pain includes, but is not limited to, acute pain, musculoskeletal pain, post-operative pain, chronic pain, persistent pain, peripherally mediated pain, centrally mediated pain.

The compounds, compositions and methods of the invention are of particular use in treating, managing and/or lessening of pain including inflammatory, neuropathic, nociceptive and idiopathic pain.

The compounds, compositions and methods of the invention are of particular use in treating, managing and/or lessening of pain including but not limited to postoperative pain, arthritis pain, osteoarthritis pain, pain associated with cancer including chemotherapy pain, neuropathic pain secondary to metastatic inflammation, neuralgic, orofacial pain, burn pain, somatic pain, dental pain, sciatica pain, intestinal obstruction pain, visceral pain, coliky pain, myofacial pain, trauma pain, labour pain, trigeminal neuralgia, glossopharangyl neuralgia, adiposis dolorosa, acute herpetic and postherpetic neuralgia, diabetic neuropathy, causalgia, brachial plexus avulsion, occipital neuralgia, reflex sympathetic dystrophy, fibromyalgia, gout, phantom limb pain, pain following stroke, thalamic lesions, radiculopathy, chronic headache, migraine pain, familial hemiplegic migraine, conditions associated with cephalic pain, sinus headache, tension headache, cardiac pain arising from an ischemic myocardium, pain following stroke, neuropathy secondary to metastatic inflammation, pain due to connective tissue damage, and other forms of neuralgic, neuropathic, and idiopathic pain syndromes.

Idiopathic pain is pain of unknown origin, for example, phantom limb pain. Neuropathic pain is generally caused by injury or infection of the peripheral sensory nerves generally it includes, but is not limited to, pain from peripheral nerve trauma, herpes virus infection, diabetes mellitus, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies.

The compounds of the invention may be useful for treating certain types of inflammatory disease such as pancreatitis, which includes acute pancreatitis and chronic pancreatitis, is characterized by recurring or persistent abdominal pain with or without steatorrhea or diabetes mellitus, hereditary pancreatitis, pancreatic dysfunction. And it may also useful for treating the pain associated with pancreatitis and its related disorders.

The compounds of the invention may be useful for treating cardiovascular conditions such as arrhythmias, atrial fibrillation and ventricular fibrillation.

Although no mutations in humans have been detected, Nav1.6 is thought to play a role in the manifestation of the symptoms associated with multiple sclerosis and has been considered as a target for the treatment of this disease (Craner, M. J., et al. Proc. Natl. Acad. Sci. USA (2004), 101, 8168-73). Nav1.7 was first cloned from the pheochromocytoma PC12 cell line (Toledo-Aral, J. J., et al. Proc. Natl. Acad. Sci. USA (1997), 94, 1527-1532). Its presence at high levels in the growth cones of small-diameter neurons suggested that it could play a role in the transmission of nociceptive information. Although this has been challenged by experts in the field as Nav1.7 is also expressed in neuroendocrine cells associated with the autonomic system (Klugbauer, N., et al. EMBO J. (1995), 14, 1084-90) and as such has been implicated in autonomic processes. The compounds of the invention may be useful for treating Crohns disease, multiple sclerosis (MS) and pain associated with multiple sclerosis (MS), amyotrophic lateral sclerosis (ALS), disseminated sclerosis, motor failure, ataxia, tremor, muscle weakness, and dystonia. Epilepsy and cardiac arrhythmias are often targets of sodium channel blockers. Recent evidence from animal models suggests that sodium channel blockers may also be useful for neuroprotection under ischemic conditions caused by stroke or neural trauma and in patients with multiple sclerosis (MS).

The compounds of the invention may be useful for treating certain type of cancers for example prostate cancer, breast cancer, ovarian cancer, testicular cancer, thyroid neoplasia. The VGSC's are reported to have been expressed in prostate and breast cancer cells. Nav1.5 has been identified in breast cancer cells and the enhanced expression of this isoform was associated with strong metastatic potential in vitro and breast cancer progression in vivo. (Fraser et al. Clin. Cancer Res. (2005), 11, 5381-5389). Expression of Na_{V}1.7 is upregulated ˜20 fold in prostate cancer. Moreover, the expression correlates with high metastatic potential in vitro. (Current Pharmaceutical Design (2006), 12, 3681-3695; Prostate Cancer and Prostatic Diseases (2005), 8, 266-273).

The compounds of invention may be useful in the treatment of epilepsy, partial and general tonic seizures, arrhythmias, fibromyalgia, neuroprotection under ischaemic conditions caused by stroke, glaucoma or neural trauma, neuromuscular conditions such as restless leg syndrome and muscle paralysis or tetanus.

The compounds of invention may be useful in the treatment of pruritus and related diseases such as psoriatic pruritus, itch due to hemodialysis, aquagenic pruritus, itching caused by skin disorders, allergic itch, insect bite itch, itch caused by hypersensitivity such as dry skin, acne, eczema, psoriasis or injury, itch caused by vulvar vestibulitis and the similar itch.

The compounds of the invention may be useful in treating the symptoms associated with BPH (benign prostate hyperplasia) including but not limited to acute urinary retention and urinary tract infection.

It is to be understood that the invention encompasses any of the compounds of Formulae (I), (Ia), (II), (III) or (IV), or pharmaceutically acceptable salts thereof for use in the treatment of any of the conditions disclosed herein.

It is to be understood that the invention encompasses the use of any of the compounds of Formulae (I), (Ia), (II), (III) or (IV), or pharmaceutically acceptable salts thereof for the manufacture of a medicament for the treatment of any of the conditions disclosed herein.

### General Methods of Preparation

The compounds described herein may be prepared by techniques known in the art. In addition, the compounds described herein may be prepared by following the reaction sequence as depicted in Scheme-1 to 3 wherein ring B, A₁, A₂, R₁, R₂, W, Y and m are as described herein above. Further, in the following schemes, where specific bases, acids, reagents, solvents, coupling agents, etc. are mentioned, it is understood that other bases, acids, reagents, solvents, coupling agents etc., known in the art may also be used and are therefore included within the scope of the present invention. Variations in reaction conditions, for example, temperature and/or duration of the reaction, which may be used as known in the art, are also within the scope of the present invention. All the isomers of the compounds described in these schemes, unless otherwise specified, are also encompassed within the scope of this invention.

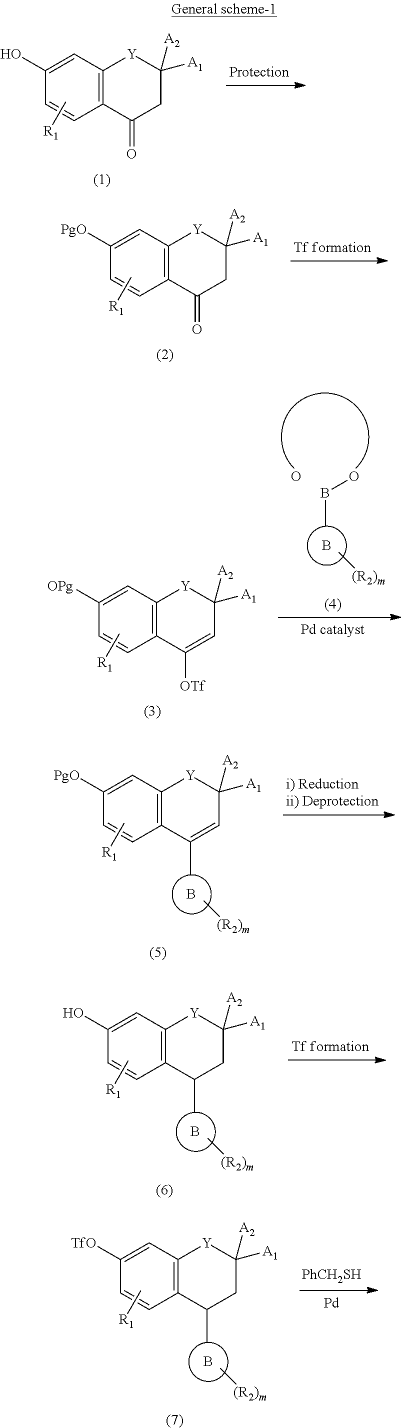

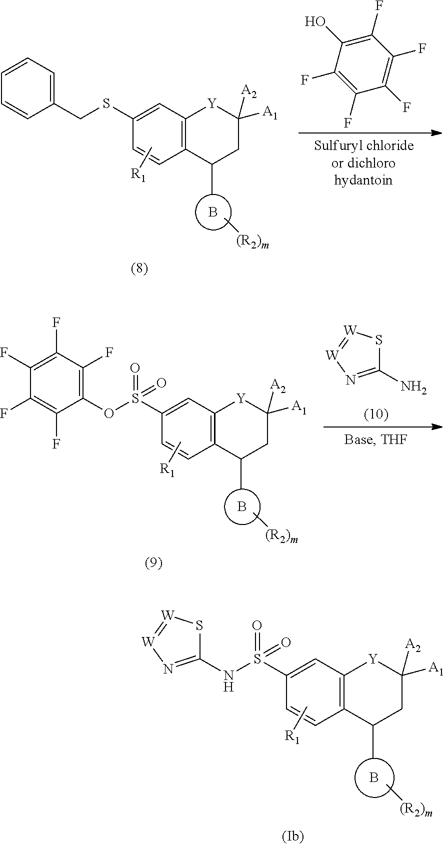

Compound of Formula (Ib), where A₁, A₂, W, Y, ring B, R₁, R₂ and ‘m’ are as described in herein above, is prepared as depicted in Scheme-1. The suitably protected compound of formula (2), which obtained from Formula (1) is converted to compound of formula (3) in presence of triflating agents like triflic anhydride or 1,1,1-trifluoro-N-phenyl-N-((trifluoromethyl)sulfonyl)methanesulfonamide in the presence of a suitable base. Suzuki coupling with boronic acid or boronic ester of formula (4) followed by reduction of double bond and deprotection of hydroxyl protection group gives the compound of formula (6). The hydroxyl group in compound of formula (6) reacts with suitable triflating agents in the presence of base to give the triflate of formula (7) which reacts with benzyl mercaptan in the presence of Pd catalyst to give the compound of formula (8). The compound of formula (8) reacts with sulfuryl chloride or dichlorohydantoin in DCM, water and acetic acid to give sulfonyl chloride which on treatment with pentafluorophenol in the presence of organic base like TEA gives the pentafluoro ester of formula (9). Treatment of amines of formula (10) with compound of formula (9) in the presence of a suitable base such as LiHMDS in suitable solvent like THF gives the compound of formula (Ib).

When one of the R₂ substitutions on ring B is bromo or iodo in compound of formula (9), It may further reacts with different boronic acid or boronic ester of formula (4), followed by reaction with amine of formula (10) to give the compound of formula (Ib).

The compound of formula (Ib) is also prepared by following general scheme-2.

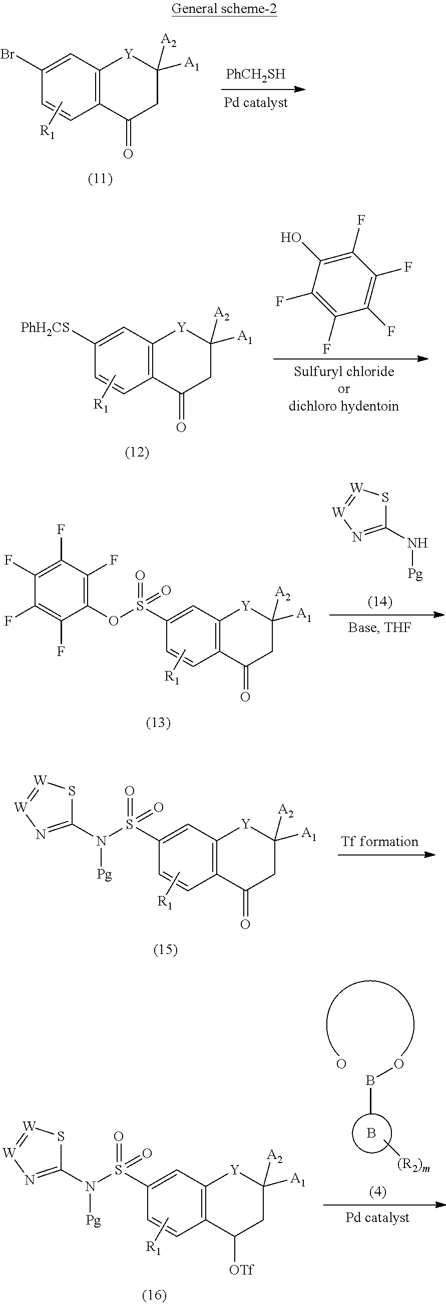

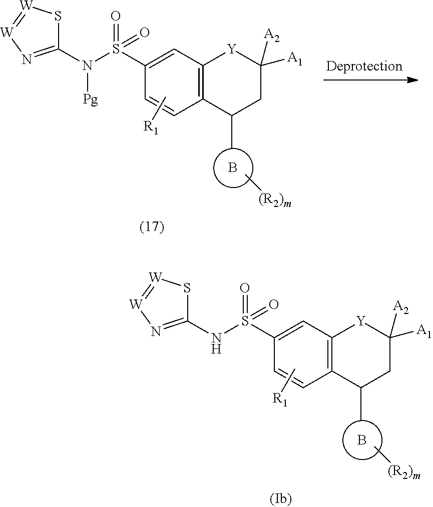

Pentafluoro ester of formula (13) is prepared by converting the bromo of formula (11) into thiobenzyl derivative of formula (12) followed by reaction with sulfuryl chloride or dichlorohydantoin in DCM:water:Acetic acid and then with pentafluorophenol in the presence of a base like TEA or DIPEA. The compound of formula (13) is converted into sulfonamide of formula (15) using compound of formula (14) in the presence of a base like LiHMDS and in suitable solvent such as THF. The compound of formula (15) is converted into triflate of formula (16) by reacting with suitable triflating agents, which are known in the art in the presence of a suitable base. Suzuki coupling of compound of formula (16) with boronic acid or boronic ester of formula (4) followed by reduction of double bond with suitable reducing agents gives the compound of formula (17). Finally removal of protecting group with suitable deprotecting agent to give the compound of formula (Ib).

When one of the R₂ substitutions on ring B is bromo or iodo in compound of formula (17), It further reacts with different boronic acid or boronic ester of formula (4), followed by deprotection of protection group also gives the compound of formula (Ib).

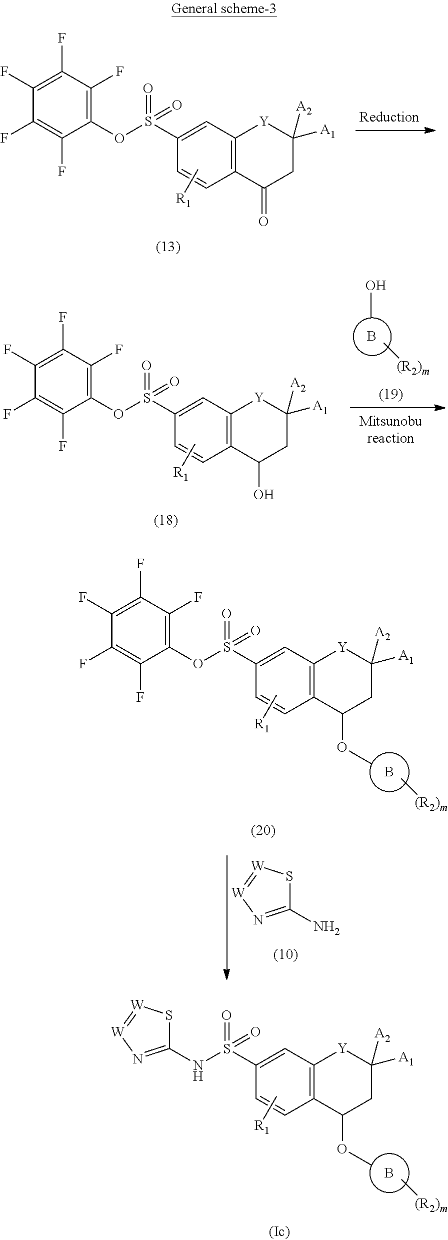

Reduction of keto group of formula (13) gives the compound of formula (18) which on reaction with phenol of formula (19) under Mitshunobu reaction condition gives the compound of formula (20). The pentafluoro ester of formula (20) on reaction with the amine of formula (10) in the presence of a base like LHMDS in suitable solvent gives the compound of formula (Ic).

When one of the R₂ substitutions on ring B is bromo or iodo in compound of formula (20), It is further reacts with different boronic acid or boronic ester of formula (4) followed by reaction with amine of formula (10) in the presence of suitable base like LiHMDS and solvent like THF to give the compound of formula (Ic).

### EXPERIMENTAL

The invention is further illustrated by the following examples which are provided merely to be exemplary of the invention and do not limit the scope of the invention. The examples set forth below demonstrate the synthetic procedures for the preparation of the representative compounds. Certain modifications and equivalents will be apparent to those skilled in the art and are intended to be included within the scope of the invention. The aforementioned patents and patent applications are incorporated herein by reference.

Unless otherwise stated, work-up implies the following operations: distribution of the reaction mixture between the organic and aqueous phase, separation of layers, drying the organic layer over sodium sulfate, filtration and evaporation of the organic solvent. Purification, unless otherwise mentioned, implies purification by silica gel chromatographic techniques, generally using ethyl acetate/petroleum ether mixture of a suitable polarity as the mobile phase.

### INTERMEDIATES

### Intermediate-1: Perfluorophenyl 4-(2-chloro-4-(trifluoromethyl)phenyl)-2,2-dimethyl chroman-7-sulfonate

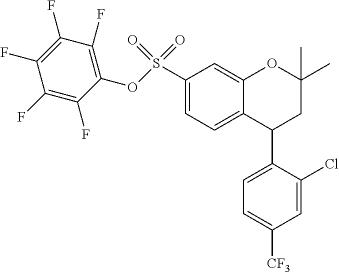 [SMILES: CC1(C)CC(c2ccc(C(F)(F)F)cc2Cl)c2ccc(S(=O)(=O)Oc3c(F)c(F)c(F)c(F)c3F)cc2O1]

### Step-1: tert-Butyl((4-(2-chloro-4-(trifluoromethyl)phenyl)-2,2-dimethyl-2H-chromen-7-yl) oxy)dimethylsilane

A solution of 7-((tert-Butyldimethylsilyl)oxy)-2,2-dimethyl-2H-chromen-4-yl trifluoro methanesulfonate (2.00 g, 4.56 mmol) (prepared as described in EP 2179994 A1), (2-chloro-4-(trifluoromethyl)phenyl)boronic acid (1.23 g, 5.47 mmol) and potassium phosphate (2.90 g, 13.68 mmol) in 1,4-dioxane (20 ml) was purged with N₂ for 10 minute then PdCl₂(dppf)-CH₂Cl₂ adduct (0.37 g, 0.45 mmol) was added and heated at 80° C. for 6 h. After completion of reaction, the reaction mixture was cooled to room temperature, diluted with ethyl acetate and filtered through celite. The filtrate was washed with brine, dried over Na₂SO₄ and evaporated under vacuum. The crude product was purified by flash column chromatography to obtain the title compound as pale yellow gummy liquid (1.80 g, 84%). ¹H NMR (400 MHz, Chloroform-d) δ 7.77-7.66 (m, 1H), 7.61-7.52 (m, 1H), 7.41 (d, J=8.0 Hz, 1H), 6.42 (dd, J=5.2, 3.0 Hz, 2H), 6.29 (d, J=2.3 Hz, 1H), 5.46 (s, 1H), 1.53 (s, 6H), 0.98 (s, 9H), 0.21 (s, 6H).

### Step-2:4-(2-Chloro-4-(trifluoromethyl)phenyl)-2,2-dimethylchroman-7-ol

To a stirred solution of tert-Butyl((4-(2-chloro-4-(trifluoromethyl)phenyl)-2,2-dimethyl-2H-chromen-7-yl)oxy)dimethylsilane (1.7 g, 3.62 mmol) in ethanol (30 ml), chlorobenzene (3.69 ml, 36.20 mmol), acetic acid (2.08 ml, 36.20 mmol), and 10% Pd/C (0.39 g, 0.36 mmol) were added at room temperature and stirred under hydrogen balloon pressure. After completion of reaction, the catalyst was filtered through celite bed and the filtrate was evaporated under vacuum to obtain the title compound as pale yellow liquid (1.1 g, 85%). m/z: 357.1 (M+H)⁺; ¹H NMR (400 MHz, Chloroform-d) δ 7.74-7.66 (m, 1H), 7.51-7.40 (m, 1H), 7.27-7.13 (m, 1H), 6.63-6.52 (m, 1H), 6.41-6.28 (m, 2H), 4.83-4.65 (m, 1H), 2.19-2.09 (m, 1H), 1.90-1.65 (m, 1H), 1.46 (s, 3H), 1.39 (s, 3H).

### Step-3:4-(2-Chloro-4-(trifluoromethyl)phenyl)-2,2-dimethylchroman-7-yl trifluoro methanesulfonate

To a solution of 4-(2-Chloro-4-(trifluoromethyl)phenyl)-2,2-dimethylchroman-7-ol (1 g, 2.80 mmol) and 2,6-lutidine (0.45 g, 4.20 mmol) in DCM (15 ml) was added triflic anhydride (0.70 ml, 4.20 mmol) at −30° C. in a drop wise manner and stirred for 1 h allowing it to come to room temperature. After completion of reaction, as indicated by TLC, ice cold water was added to reaction mixture and the compound was extracted with ethyl acetate. The combined organic layer was washed with brine, dried over Na₂SO₄ and concentrated under vacuum. The crude product was purified by flash column chromatography (10% ethyl acetate:Petroleum ether) to obtain white solid (1.2 g, 88%). LCMS(ESI): m/z 488.9 (M+H)⁺; ¹H NMR (400 MHz, Chloroform-d) δ 7.72 (d, J=2.1 Hz, 1H), 7.54-7.44 (m, 1H), 7.19 (s, 1H), 6.82 (d, J=2.5 Hz, 1H), 6.78 (dd, J=8.7, 1.2 Hz, 1H), 6.71 (dd, J=8.6, 2.5 Hz, 1H), 4.92-4.66 (m, 1H), 2.25-2.07 (m, 1H), 1.98-1.73 (m, 1H), 1.49 (s, 3H), 1.41 (s, 3H).

### Step-4:7-(Benzylthio)-4-(2-chloro-4-(trifluoromethyl)phenyl)-2,2-dimethylchroman

4-(2-Chloro-4-(trifluoromethyl)phenyl)-2,2-dimethylchroman-7-yl trifluoromethanesulfonate (1.2 g, 2.455 mmol) was dissolved in 1,4-dioxane (15 ml) and purged with N₂. Xantphos (0.071 g, 0.12 mmol), Pd₂(dba)₃ (0.056 g, 0.061 mmol), Hunig's base (0.85 ml, 4.91 mmol), and benzyl mercaptan (0.30 ml, 2.58 mmol) were added and heated at 80° C. for 6 h. After completion of reaction, the reaction mixture was diluted with water and the compound was extracted with ethyl acetate. The combined organic layer was washed with brine, dried over Na₂SO₄ and evaporated under vacuum. Purification was done using flash column chromatography to obtain the title compound as off white solid (0.98 g, 86% yield). ¹H NMR (400 MHz, Chloroform-d) δ 7.76-7.63 (m, 1H), 7.51-7.41 (m, 1H), 7.39-7.15 (m, 6H), 6.87 (d, J=1.9 Hz, 1H), 6.79-6.70 (m, 1H), 6.61 (dd, J=8.1, 1.1 Hz, 1H), 4.80-4.70 (m, 1H), 4.15 (s, 2H), 2.17-2.10 (m, 1H), 1.85-1.70 (m, 1H), 1.47 (s, 3H), 1.39 (s, 3H).

### Step-5: Perfluorophenyl 4-(2-chloro-4-(trifluoromethyl)phenyl)-2,2-dimethylchroman-7-sulfonate (Intermediate-1)

To an ice-cold solution of 7-(Benzylthio)-4-(2-chloro-4-(trifluoromethyl)phenyl)-2,2-dimethylchroman (1.2 g, 2.59 mmol) in DCM-AcOH—H₂O (20:1:1 ml) was added sulfuryl chloride (0.25 ml, 3.11 mmol) at 0-5° C. temperature and stirred at room temperature for 2 h. The reaction mixture was quenched with water, and the product was extracted with DCM. The organic layer was dried over Na₂SO₄, and concentrated under vacuum. The residue was dissolved in DCM (20 ml) and then 2,3,4,5,6-pentafluorophenol (0.57 g, 3.11 mmol) and TEA (1.80 ml, 12.96 mmol) were added at 0° C. and stirred for 30 min. The reaction mixture was quenched with water and extracted with DCM. The combined organic layer was washed with brine, dried over Na₂SO₄, and concentrated under vacuum. Purification was done using flash column chromatography to obtain off white solid (1.10 g, 72%). ¹H NMR (400 MHz, Chloroform-d) δ 7.78-7.70 (m, 1H), 7.56-7.45 (m, 2H), 7.41-7.32 (m, 1H), 7.23-7.06 (m, 1H), 7.01-6.93 (m, 1H), 4.99-4.72 (m, 1H), 2.36-2.15 (m, 1H), 2.02-1.75 (m, 1H), 1.52 (s, 3H), 1.42 (s, 3H).

### Intermediate-2: Perfluorophenyl 4-(2-methoxy-4-(trifluoromethyl)phenyl)-2,2-dimethyl-2H-chromene-7-sulfonate

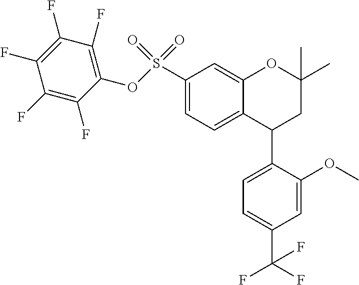 [SMILES: COc1cc(C(F)(F)F)ccc1C1CC(C)(C)Oc2cc(S(=O)(=O)Oc3c(F)c(F)c(F)c(F)c3F)ccc21]

The title compound was prepared by following similar procedure as described in Intermediate-1. ¹H NMR (400 MHz, Chloroform-d) δ 7.45 (d, J=2.0 Hz, 1H), 7.30 (dd, J=8.2, 2.1 Hz, 1H), 7.24-7.19 (m, 1H), 7.15 (m, 2H), 6.95 (dd, J=8.2, 1.1 Hz, 1H), 4.70-4.60 (m, 1H), 3.86 (s, 3H), 2.10-2.00 (m, 2H), 1.50 (s, 3H), 1.39 (s, 3H).

### Intermediate-3: tert-Butyl 4-(2-(2,2-dimethyl-7-((perfluorophenoxy) sulfonyl)chroman-4-yl)-5-(trifluoromethyl)phenyl)-5,6-dihydropyridine-1(2H)-carboxylate

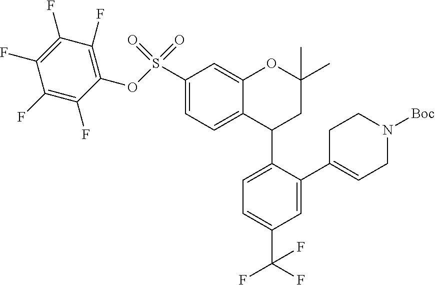 [SMILES: CC(C)(C)OC(=O)N1CC=C(c2cc(C(F)(F)F)ccc2C2CC(C)(C)Oc3cc(S(=O)(=O)Oc4c(F)c(F)c(F)c(F)c4F)ccc32)CC1]

A mixture of Perfluorophenyl 4-(2-chloro-4-(trifluoromethyl)phenyl)-2,2-dimethylchroman-7-sulfonate (0.30 g, 0.51 mmol) (Intermediate-1), tert-butyl 4-(4,4,5,5-tetramethyl-1,3,2-dioxaborolan-2-yl)-5,6-dihydropyridine-1(2H)-carboxylate (0.19 g, 0.61 mmol) and potassium phosphate (0.27 g, 1.28 mmol), in 1,4-dioxane (10 ml) was purged with N₂ for 10 minute. After that bis(di-tert-butyl(4-dimethylaminophenyl)phosphine) dichloropalladium(II) (0.036 g, 0.051 mmol) was added to the reaction vial and heated at 110° C. for 1 h under microwave irradiation. After completion of reaction, it was diluted with ethyl acetate and filtered through a pad of celite. Filtrate was washed with brine, dried over Na₂SO₄ and evaporated to dryness. Purification was done by flash column chromatography to obtain title compound as off white solid (0.18 g, 48%). ¹H NMR (400 MHz, Chloroform-d) δ 7.46 (m, 3H), 7.35-7.29 (m, 1H), 7.05 (d, J=8.1 Hz, 1H), 6.90-6.74 (m, 1H), 5.74 (m, 1H), 4.42 (m, 1H), 4.20-4.10 (m, 2H), 3.75-3.54 (m, 2H), 2.50-2.41 (m, 2H), 2.03-1.98 (m, 2H), 1.51 (s, 3H), 1.50 (s, 9H), 1.34 (s, 3H).

### Intermediate-4: tert-Butyl 4-(2-(4,4,5,5-tetramethyl-1,3,2-dioxaborolan-2-yl)-5-(trifluoro methyl)phenyl)piperidine-1-carboxylate

### Step-1: 4-(2-Bromo-5-(trifluoromethyl)phenyl)piperidine-2,6-dione

To a solution of 2-Bromo-5-(trifluoromethyl)benzaldehyde (11 g, 43.5 mmol) and ethyl 3-oxobutanoate (11.88 g, 91 mmol) in ethanol (100 ml), piperidine (0.861 ml, 8.70 mmol) was added and stirred the mixture at room temperature for overnight. 12N NaOH (20 ml) and 20 ml EtOH was added to the reaction mixture and was refluxed for 4 h. The solvent was removed under vacuum and reaction mixture was cooled, acidified carefully with HCl (pH 2) and extracted with ethyl acetate. The combined organic layer was washed with brine, dried over Na₂SO₄ and evaporated under vacuum. The crude solid was triturated with ether: hexane (1:1) to give di-acid as pale yellow solid. The di-acid solid and urea (7.83 g, 130 mmol) was heated at 160° C. for 2 h. TLC indicated completion of reaction. Reaction mixture was cooled to room temperature and quenched with water. The solid was filtered and dried to give the title compound as brown solid (11.9 g, 81%). LCMS(ESI): m/z 336.22 & 338.28 (M⁺& M+2)⁺.

### Step-2:4-(2-Bromo-5-(trifluoromethyl)phenyl)piperidine

To a solution of 4-(2-Bromo-5-(trifluoromethyl)phenyl)piperidine-2,6-dione (11 g, 32.7 mmol) in THF (55 ml) was added BH₃.DMS (13.99 ml, 147 mmol) at 0° C. and then stirred at 65° C. for overnight. TLC shown completion of reaction. The reaction mixture was cooled to ° C. and then slowly quenched with con. HCl (Up to pH 2). Again it was heated at 85° C. for 3 h. The reaction mixture was cooled to 0° C., neutralized with 2N NaOH and extracted with ethyl acetate. The combined organic layer was washed with brine, dried over Na₂SO₄ and evaporated under vacuum to obtain the title compound as brown oil (10 g, 98%). LCMS(ESI): m/z 308 & 310 (M⁺& M+2)⁺

### Step-3: tert-Butyl 4-(2-bromo-5-(trifluoromethyl)phenyl)piperidine-1-carboxylate

To a solution of 4-(2-Bromo-5-(trifluoromethyl)phenyl)piperidine (9 g, 29.2 mmol) in acetonitrile (100 ml) was added TEA (12.21 ml, 88 mmol), DMAP (4-Dimethylaminopyridine) (0.35 g, 2.92 mmol), BOC-anhydride (16.95 ml, 73 mmol) and heated at 60° C. for overnight. TLC shown completion of reaction. The solvent was removed under vacuum and the reaction mixture was diluted with ethyl acetate. The combined organic layer was washed with 2N HCl, brine, dried over Na₂SO₄ and evaporated to dryness under vacuum. The crude product was purified with flash column chromatography (18% ethyl acetate/Petroleum ether) to obtain title compound as an off white solid (4 g, 33%). 1H NMR (400 MHz, Chloroform-d) δ 7.70 (dd, J=8.3, 1.0 Hz, 1H), 7.46 (d, J=2.2 Hz, 1H), 7.35 (ddd, J=8.5, 2.3, 0.9 Hz, 1H), 4.42-4.25 (m, 2H), 3.19 (tt, J=12.2, 3.4 Hz, 1H), 2.87 (td, J=12.7, 2.5 Hz, 2H), 1.96-1.83 (m, 2H), 1.65-1.56 (m, 2H), 1.50 (s, 9H).

### Step-4: tert-Butyl 4-(2-(4,4,5,5-tetramethyl-1,3,2-dioxaborolan-2-yl)-5-(trifluoromethyl) phenyl)piperidine-1-carboxylate (Intermediate-4)

A mixture of tert-Butyl 4-(2-bromo-5-(trifluoromethyl)phenyl)piperidine-1-carboxylate (2.00 g, 4.90 mmol), 4,4,4′,4′,5,5,5,5′,5′-octamethyl-2,2′-bi(1,3,2-dioxaborolane) (1.86 g, 7.35 mmol) and potassium acetate (1.34 g, 13.72 mmol) in 1,4-dioxane (10 ml) was purged with nitrogen for 30 minutes after which PdCl₂(dppf)-DCM adduct (0.40 g, 0.490 mmol) was added and the reaction mixture was heated at 120° C. for 2 h under microwave irradiation. The reaction mixture was diluted with ethyl acetate and filtered through celite. The combined filtrate was washed with brine, dried over Na₂SO₄ and purified by flash column chromatography (5% EtOAc/Petroleum ether) to obtain the title compound as pale yellow gummy solid (1.7 g, 76%). ¹H NMR (400 MHz, Chloroform-d) δ 7.87 (dt, J=7.6, 0.9 Hz, 1H), 7.46 (s, 2H), 4.28 (d, J=13.0 Hz, 2H), 3.48 (tt, J=12.2, 3.4 Hz, 1H), 2.90-2.73 (m, 2H), 1.84-1.79 (m, 2H), 1.65-1.58 (m, 2H), 1.50 (s, 9H), 1.37 (s, 12H).

### Intermediate-5: tert-Butyl 3-fluoro-3-(2-(4,4,5,5-tetramethyl-1,3,2-dioxaborolan-2-yl)-5-(trifluoromethyl)phenyl)azetidine-1-carboxylate

### Step-1: tert-Butyl 3-(2-bromo-5-(trifluoromethyl)phenyl)-3-fluoroazetidine-1-carboxylate

To a stirred solution of 1-Bromo-2-iodo-4-(trifluoromethyl)benzene (1.62 ml, 9.97 mmol) in THF (50 ml), isopropyl magnesium chloride (5.98 ml, 11.97 mmol) was added and stirred for 30 min. at room temperature. The reaction mixture was then cooled to 0° C. and tert-butyl 3-oxoazetidine-1-carboxylate (2.05 g, 11.97 mmol) was added and stirred for 2 h allowing it to come to room temperature. After completion of reaction as indicated by TLC, the reaction mixture was poured into saturated solution of ammonium chloride and extracted with ethyl acetate. The combined organic layer was washed with brine, dried over Na₂SO₄ and evaporated under vacuum to obtain tert-butyl 3-(2-bromo-5-(trifluoromethyl)phenyl)-3-hydroxyazetidine-1-carboxylate, which was dissolved in DCM (50.0 ml,) and cooled to 0° C. and DAST (Diethylaminosulfur trifluoride) (1.58 ml, 11.97 mmol) was added to it and stirred for 12 h at room temperature. The reaction mixture was poured into water and extracted with DCM, dried over Na₂SO₄ and concentrated under vacuum. The crude product was purified by flash column chromatography (25-40% ethyl acetate/Petroleum ether) to obtain tert-Butyl 3-(2-bromo-5-(trifluoromethyl)phenyl)-3-fluoroazetidine-1-carboxylate (3 g, 76%).

LCMS(ESI): m/z 420 (M+Na)⁺; ¹H NMR (400 MHz, Chloroform-d) δ 7.82 (dt, J=8.4, 0.9 Hz, 1H), 7.66 (t, J=2.1 Hz, 1H), 7.58-7.50 (m, 1H), 4.71-4.57 (m, 2H), 4.50 (ddd, J=21.9, 10.9, 1.4 Hz, 2H), 1.49 (s, 9H).

### Step-2: tert-Butyl 3-fluoro-3-(2-(4,4,5,5-tetramethyl-1,3,2-dioxaborolan-2-yl)-5-(trifluoro methyl)phenyl)azetidine-1-carboxylate (Intermediate-5)

A mixture of tert-Butyl 3-(2-bromo-5-(trifluoromethyl)phenyl)-3-fluoroazetidine-1-carboxylate (1.5 g, 3.77 mmol), 4,4,4′,4′,5,5,5′,5′-octamethyl-2,2′-bi(1,3,2-dioxaborolane) (1.43 g, 5.65 mmol) and potassium acetate (1.03 g, 10.55 mmol) in 1,4-dioxane (10 ml) was purged with nitrogen for 30 min. after which PdCl₂(dppf)-DCM adduct (0.308 g, 0.377 mmol) was added and the reaction mixture was heated at 120° C. for 3 h under microwave irradiation. The reaction mixture was diluted with ethyl acetate and filtered through celite. The combined filtrate was washed with brine, dried over Na₂SO₄ and purified by flash chromatography (50% ethyl acetate/Petroleum ether) to obtain the title compound as off-white gummy solid (0.9 g, 53%). LCMS(ESI): m/z 446.17 (M+H)⁺; ¹H NMR (400 MHz, Chloroform-d) δ 7.84-7.60 (m, 3H), 4.53-4.38 (m, 4H), 1.50 (s, 12H), 1.38 (s, 9H).

### Intermediate-6: tert-Butyl 4-(2-(2,2-dimethyl-7-((perfluorophenoxy)sulfonyl)chroman-4-yl)-5-(trifluoromethyl)phenyl)piperidine-1-carboxylate

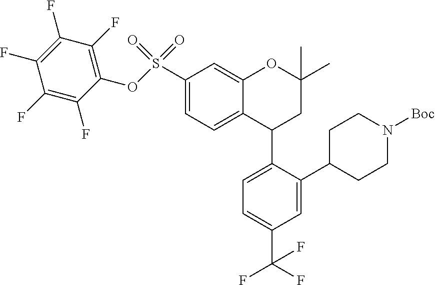 [SMILES: CC(C)(C)OC(=O)N1CCC(c2cc(C(F)(F)F)ccc2C2CC(C)(C)Oc3cc(S(=O)(=O)Oc4c(F)c(F)c(F)c(F)c4F)ccc32)CC1]

The title compound was prepared by following similar procedure as described for Intermediate-1 using Intermediate-4.

¹H NMR (400 MHz, Chloroform-d) δ 7.60-7.40 (m, 3H), 7.33 (d, J=8.3 Hz, 1H), 7.01 (d, J=8.1 Hz, 1H), 6.85 (d, J=8.3 Hz, 1H), 4.65-4.57 (m, 1H), 4.35 (s, 2H), 3.10-3.00 (m, 1H), 2.90-2.80 (m, 2H), 2.10-1.70 (m, 6H), 1.60 (s, 3H), 1.53 (s, 3H), 1.51 (s, 9H).

### Intermediate-7: tert-Butyl 4-(2-(2,2-dimethyl-7-(N-(thiazol-2-yl)sulfamoyl)chroman-4-yl)-5-(trifluoromethyl)phenyl)piperidine-1-carboxylate

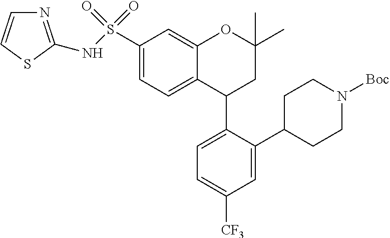 [SMILES: CC(C)(C)OC(=O)N1CCC(c2cc(C(F)(F)F)ccc2C2CC(C)(C)Oc3cc(S(=O)(=O)Nc4nccs4)ccc32)CC1]

To a solution of thiazol-2-amine (0.018 g, 0.177 mmol) and tert-Butyl 4-(2-(2,2-dimethyl-7-((perfluorophenoxy)sulfonyl)chroman-4-yl)-5-(trifluoromethyl)phenyl)piperidine-1-carboxylate (0.10 g, 0.136 mmol) (Intermediate-6) in THF (5 ml) was added LiHMDS (1M THF solution) (0.204 ml, 0.204 mmol) at 0° C. under nitrogen. The reaction mixture was stirred for 4 h at ambient temperature then poured into 2N HCl solution and extracted with ethyl acetate. The combined organic layer was washed with brine, dried over Na₂SO₄ and evaporated to dryness. The crude was purified by flash column chromatography to obtain the title compound as white solid (0.070 g, 79%). LCMS(ESI): m/z 651.71 (M+H)⁺; Enantiomers of Intermediate-7 were separated using chiral preparative HPLC [Column: Chiral pak IC, Mobile phase: (n-hexane:0.1% DEA):(ETOH:DCM, 1:1) 70:30], Intermediate-7a: Retention time: 7.42 min.; Intermediate-7b Retention time: 9.56 min.

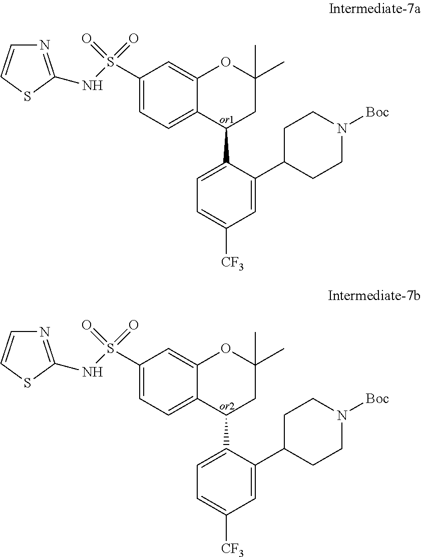 [SMILES: CC(C)(C)OC(=O)N1CCC(c2cc(C(F)(F)F)ccc2[C@@H]2CC(C)(C)Oc3cc(S(=O)(=O)Nc4nccs4)ccc32)CC1 ; CC(C)(C)OC(=O)N1CCC(c2cc(C(F)(F)F)ccc2[C@H]2CC(C)(C)Oc3cc(S(=O)(=O)Nc4nccs4)ccc32)CC1]

Similarly Intermediates-8, 8a and 8b were prepared by following similar procedure as described in Intermediate-7 using Intermediate-6 and 1,2,4-thiadiazole 5-amine.

### Intermediate-8: tert-Butyl 4-(2-(7-(N-(1,2,4-thiadiazol-5-yl)sulfamoyl)-2,2-dimethyl chroman-4-yl)-5-(trifluoromethyl)phenyl)piperidine-1-carboxylate

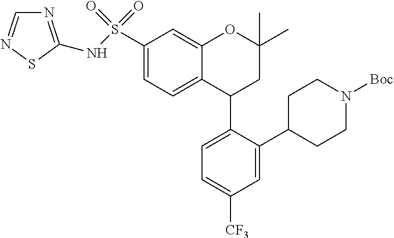 [SMILES: CC(C)(C)OC(=O)N1CCC(c2cc(C(F)(F)F)ccc2C2CC(C)(C)Oc3cc(S(=O)(=O)Nc4ncns4)ccc32)CC1]

Enantiomers of Intermediate-8 were separated using chiral preparative HPLC [Column: Chiral pak IC, Mobile phase: (n-hexane:0.1% DEA):(ETOH:DCM, 1:1) 60:40], Isomer-8a retention time: 6.50 min.; Isomer-8b retention time: 8.91 min.

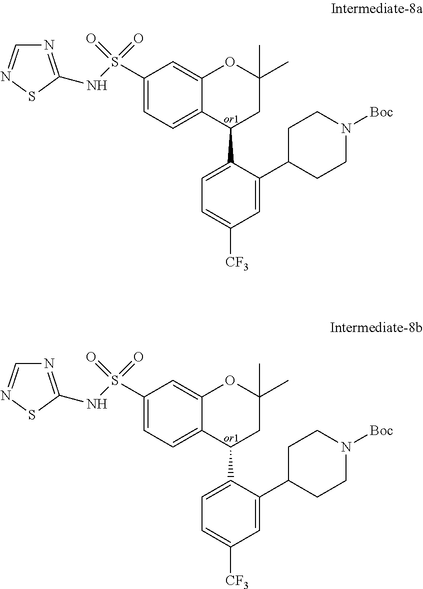 [SMILES: CC(C)(C)OC(=O)N1CCC(c2cc(C(F)(F)F)ccc2[C@@H]2CC(C)(C)Oc3cc(S(=O)(=O)Nc4ncns4)ccc32)CC1 ; CC(C)(C)OC(=O)N1CCC(c2cc(C(F)(F)F)ccc2[C@H]2CC(C)(C)Oc3cc(S(=O)(=O)Nc4ncns4)ccc32)CC1]

LCMS(ESI): m/z 675.18 (M+Na)+ LCMS(ESI): m/z 675.18 (M+Na)+

### Intermediate-9: tert-Butyl 4-(2-(7-(N-(1,3,4-thiadiazol-2-yl)sulfamoyl)-2,2-dimethyl chroman-4-yl)-5-(trifluoromethyl)phenyl)piperidine-1-carboxylate

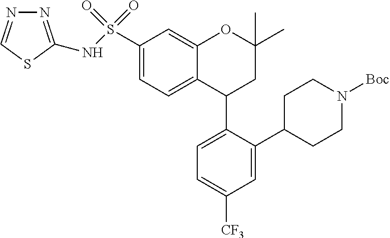 [SMILES: CC(C)(C)OC(=O)N1CCC(c2cc(C(F)(F)F)ccc2C2CC(C)(C)Oc3cc(S(=O)(=O)Nc4nncs4)ccc32)CC1]

The title compound was prepared by following similar procedure as described in Intermediate-8 using Intermediate-6 and 1,3,4-thiadiazol-2-amine. LCMS(ESI): m/z 652.59 (M+H)⁺.

### Intermediate-10: Perfluorophenyl 4-(2-chloro-4-(trifluoromethyl)phenyl)chroman-7-sulfonate

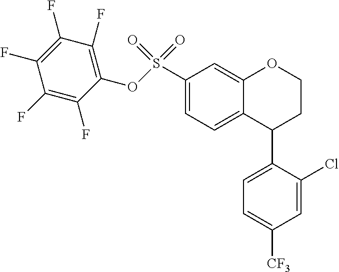 [SMILES: O=S(=O)(Oc1c(F)c(F)c(F)c(F)c1F)c1ccc2c(c1)OCCC2c1ccc(C(F)(F)F)cc1Cl]

The title compound was prepared by similar procedure as described in Intermediate-1 using 7-((tert-butyldimethylsilyl)oxy)-2H-chromen-4-yl trifluoromethanesulfonate (Prepared by following similar procedure as described in EP 2179994 from 7-hydroxychroman-4-one) and (2-Chloro-4-(trifluoromethyl)phenyl)boronic acid

Yield: 65%; LCMS(ESI): m/z 580.95 (M+Na)⁺; ¹H NMR (400 MHz, Chloroform-d) δ 7.77-7.72 (m, 1H), 7.54 (d, J=2.0 Hz, 1H), 7.50-7.44 (m, 1H), 7.43-7.37 (m, 1H), 7.08-7.03 (m, 1H), 6.96 (d, J=8.2 Hz, 1H), 4.81 (t, J=6.1 Hz, 1H), 4.41-4.28 (m, 1H), 4.28-4.13 (m, 1H), 2.56-2.37 (m, 1H), 2.26-2.15 (m, 1H).

### Intermediate-11: tert-Butyl 4-(2-(7-((perfluorophenoxy)sulfonyl)chroman-4-yl)-5-(trifluoromethyl)phenyl)-5,6-dihydropyridine-1 (2H)-carboxylate

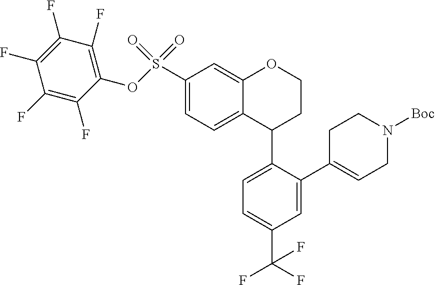 [SMILES: CC(C)(C)OC(=O)N1CC=C(c2cc(C(F)(F)F)ccc2C2CCOc3cc(S(=O)(=O)Oc4c(F)c(F)c(F)c(F)c4F)ccc32)CC1]

The title compound was prepared by similar procedure as described in Intermediate-3 using Intermediate-10.

Yield: 42%; ¹H NMR (400 MHz, Chloroform-d) δ 7.77-7.73 (m, 1H), 7.54 (d, J=2.0 Hz, 1H), 7.51-7.45 (m, 1H), 7.41 (dd, J=8.2, 2.0 Hz, 1H), 7.05 (dd, J=8.1, 1.0 Hz, 1H), 6.95 (d, J=8.2 Hz, 1H), 5.80-5.70 (m, 1H), 4.81 (t, J=6.1 Hz, 1H), 4.39-4.16 (m, 2H), 4.02-3.80 (m, 2H), 3.52-3.49 (m, 2H), 2.47-2.22 (m, 1H), 2.27-2.11 (m, 3H), 1.48 (s, 9H).

### Intermediate-12: Perfluorophenyl 4-hydroxychroman-7-sulfonate

### Step-1: 7-(Benzylthio)chroman-4-one

A solution of 7-Bromochroman-4-one (Prepared as described in US2013/18055A1) (3 g, 13.21 mmol) in 1,4-dioxane (5 ml) was purged with N₂ for 15 min., and then benzyl mercaptan (1.64 ml, 13.87 mmol), xantphos (0.3 g, 0.66 mmol), Pd₂(dba)₃ (0.30 g, 0.33 mmol), and Hunig's base (4.62 ml, 26.4 mmol) were added and heated at 80° C. for 2 h. After completion of reaction, the reaction mixture was diluted with water and the compound was extracted with ethyl acetate. The combined organic layer was washed with brine, dried over Na₂SO₄ and evaporated under vacuum. The crude product was purified by flash column chromatography (20% ethyl acetate/Petroleum ether) to obtain off white solid (1.5 g, 42%). ¹H NMR (400 MHz, Chloroform-d) δ 7.78 (d, J=8.3 Hz, 1H), 7.49-7.23 (m, 5H), 6.90 (dd, J=8.3, 1.8 Hz, 1H), 6.85-6.75 (m, 1H), 4.52 (m, 2H), 4.20 (s, 2H), 2.78 (m, 2H).

### Step-2: Perfluorophenyl 4-oxochroman-7-sulfonate

To an ice-cold solution of 7-(Benzylthio)chroman-4-one (1.5 g, 5.55 mmol) in DCM-AcOH—H₂O (5 ml-1 ml-1 ml) was added sulfuryl chloride (0.54 ml, 6.66 mmol) and stirred at 0° C. for 2 h. After completion of reaction, it was quenched by water and extracted with DCM. The combined organic layer was washed with brine, dried over Na₂SO₄ and evaporated under vacuum. The product sulfonyl chloride was taken in DCM (10 ml) and 2,3,4,5,6-pentafluorophenol (1.12 g, 6.10 mmol) and TEA (1.54 ml, 11.10 mmol) were added at 0° C. and stirred for 1 h. After completion of reaction, the reaction mixture was diluted with DCM, washed with water, brine, dried over Na₂SO₄ and evaporated under vacuum. Purification was done using flash column chromatography (17% ethyl acetate/Petroleum ether) (1 g, 46%). LCMS(ESI): m/z 394.94 (M+H)⁺; ¹H NMR (400 MHz, Chloroform-d) δ 8.13 (d, J=8.3 Hz, 1H), 7.75-7.56 (m, 2H), 4.68 (m, 2H), 3.00-2.90 (m, 2H).

### Step-3: Perfluorophenyl 4-hydroxychroman-7-sulfonate (Intermediate-12)

Perfluorophenyl 4-oxochroman-7-sulfonate (1 g, 2.54 mmol) was dissolved in MeOH (15 ml) and NaBH₄ (0.12 g, 3.04 mmol) was added to it at 0° C. The reaction mixture was stirred at reaction mixture for 1 h. After which, solvent was evaporated under vacuum and the reaction mixture was quenched with ammonium chloride. The product was extracted with ethyl acetate. The combined organic layer was washed with brine and dried over Na₂SO₄ and evaporated under vacuum to obtain the title compound as off white solid (1 g, 99% yield). ¹HNMR (400 MHz, Chloroform-d) δ 7.60 (d, J=8.1 Hz, 1H), 7.51 (dd, J=8.1, 1.9 Hz, 1H), 7.47 (d, J=1.8 Hz, 1H), 4.90 (m, 1H), 4.41-4.31 (m, 2H), 2.32-2.07 (m, 2H).

### Intermediate-12a: (S)-Perfluorophenyl 4-hydroxychroman-7-sulfonate

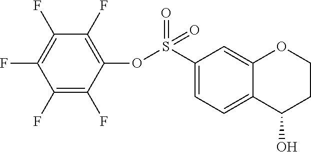 [SMILES: O=S(=O)(Oc1c(F)c(F)c(F)c(F)c1F)c1ccc2c(c1)OCC[C@@H]2O]

To a solution of (R)-1-Methyl-3,3-diphenylhexahydropyrrolo[1,2-c][1,3,2]oxazaborole (5.58 ml, 5.58 mmol) in THF (50 ml) was added BH₃.DMS (3.44 ml, 36.3 mmol) at room temperature and stirred for 30 min. Added into perfluorophenyl 4-oxochroman-7-sulfonate (step-2 of Intermediate-12) (11.00 g, 27.9 mmol) in THF (50 ml) drop wise and stirred at room temperature for another 30 min. TLC shown completion of reaction. Reaction mixture was then quenched with MeOH and poured into water and extract with ethyl acetate.

The combined organic layer was dried over Na₂SO₄ and conc. under vacuum. The crude product was purified with flash column chromatography (30% ethyl acetate/Petroleum ether) to obtained title compound as colorless oil (9.5 g 86%). ee 98.90% (chiral pak IF, Mobile phase: (n-hexane:0.1% DEA):(ETOH:DCM, 1:1) 80:20, Retention time: 8.16 min). LCMS(ESI): m/z 418.87 (M+Na)⁺; ¹H NMR (400 MHz, Chloroform-d) δ 7.60 (d, J=8.1 Hz, 1H), 7.51 (dd, J=8.1, 1.9 Hz, 1H), 7.46 (d, J=1.9 Hz, 1H), 4.92 (t, J=4.8 Hz, 1H), 4.45-4.32 (m, 2H), 2.29-2.10 (m, 2H).

### Intermediate-12b: (R)-Perfluorophenyl 4-hydroxychroman-7-sulfonate

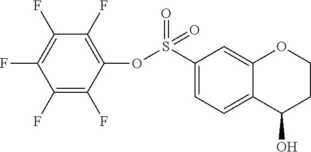 [SMILES: O=S(=O)(Oc1c(F)c(F)c(F)c(F)c1F)c1ccc2c(c1)OCC[C@H]2O]

Intermediate-12b was prepared similarly as described for Intermediate-12a using (S)-1-Methyl-3,3-diphenylhexahydropyrrolo[1,2-c][1,3,2]oxazaborole.

LCMS(ESI): m/z 418.88 (M+Na)⁺; ee 99.20 (chiral pakIF, Mobile phase: (n-hexane:0.1% DEA):(ETOH:DCM, 1:1) 80:20, Retention time: 7.31 min).

### Intermediate-13: Perfluorophenyl 4-(2-chloro-4-(trifluoromethyl) phenoxy)chroman-7-sulfonate

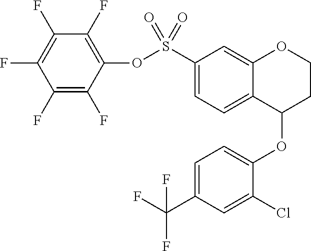 [SMILES: O=S(=O)(Oc1c(F)c(F)c(F)c(F)c1F)c1ccc2c(c1)OCCC2Oc1ccc(C(F)(F)F)cc1Cl]

To a solution of Perfluorophenyl 4-hydroxychroman-7-sulfonate (Intermediate-12) (0.50 g, 1.26 mmol), 2-Chloro-4-(trifluoromethyl)phenol (0.30 g, 1.51 mmol) and triphenylphosphine (0.43 g, 1.64 mmol) in THF (3 ml) was added DEAD (Diethyl azodicarboxylate) (0.30 ml, 1.89 mmol) at room temperature and stirred for 1 h. The reaction mixture was diluted with ethyl acetate and washed with water, brine, dried over Na₂SO₄. The organic layer was evaporated under vacuum and purified by flash column chromatography (10% ethyl acetate/Petroleum ether) to obtain title compound as off white solid (0.28, 39%). ¹H NMR (400 MHz, Chloroform-d) δ 7.75-7.65 (m, 1H), 7.60-7.36 (m, 4H), 7.19 (d, J=8.5 Hz, 1H), 5.49 (t, J=3.9 Hz, 1H), 4.59-4.39 (m, 2H), 2.45-2.26 (m, 2H).

### Intermediate-13a: (R)-Perfluorophenyl 4-(2-chloro-4-(trifluoromethyl) phenoxy)chroman-7-sulfonate

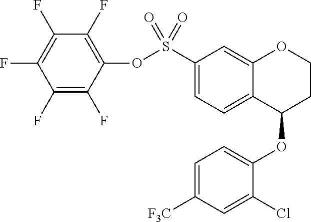 [SMILES: O=S(=O)(Oc1c(F)c(F)c(F)c(F)c1F)c1ccc2c(c1)OCC[C@H]2Oc1ccc(C(F)(F)F)cc1Cl]

To a solution of (S)-Perfluorophenyl 4-hydroxychroman-7-sulfonate (Intermediate-12a) (0.300 g, 0.757 mmol), triphenylphosphine (0.40 g, 1.514 mmol) and 2-Chloro-4-(trifluoromethyl)phenol (0.18 g, 0.908 mmol) in THF (25 ml), DIAD (Diisopropyl azodicarboxylate) (0.294 ml, 1.514 mmol) was added and the mixture was stirred for 2 h. The reaction mixture was poured into water and extracted with ethyl acetate. The combined organic layer was washed with brine, dried over Na₂SO₄ and concentrated under vacuum. The crude product was purified by flash column chromatography (15% ethyl acetate/pet/ether) to obtain title compound as colorless gummy liquid (0.2 g, 46%). ¹H NMR (400 MHz, Chloroform-d) δ 7.75-7.69 (m, 1H), 7.60-7.36 (m, 4H), 7.20 (d, J=8.6 Hz, 1H), 5.49 (t, J=4.0 Hz, 1H), 4.59-4.40 (m, 2H), 2.45-2.26 (m, 2H).

### Intermediate-14a: (R)-Perfluorophenyl 4-(2-(1-methyl-1H-pyrazol-5-yl)-4-(trifluoromethyl) phenoxy)chroman-7-sulfonate

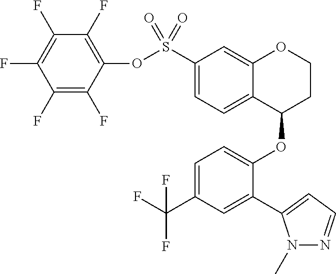 [SMILES: Cn1nccc1-c1cc(C(F)(F)F)ccc1O[C@@H]1CCOc2cc(S(=O)(=O)Oc3c(F)c(F)c(F)c(F)c3F)ccc21]

### Step-1: (R)-Perfluorophenyl 4-(2-bromo-4-(trifluoromethyl)phenoxy)chroman-7-sulfonate

The title compound was prepared by following similar procedure as described for Intermediate-13a using Intermediate-12a and 2-Bromo-4-(trifluoromethyl)phenol.

Yield 26%, LCMS(ESI): m/z 491.94 (M+H)⁺; 1H NMR (400 MHz, Chloroform-d) δ 7.88 (dd, J=2.1, 0.9 Hz, 1H), 7.65-7.41 (m, 4H), 7.17 (d, J=8.6 Hz, 1H), 5.51 (t, J=4.0 Hz, 1H), 4.60-4.40 (m, 2H), 2.45-2.26 (m, 2H).

### Step-2: (R)-Perfluorophenyl 4-(2-(1-methyl-1H-pyrazol-5-yl)-4-(trifluoromethyl) phenoxy)chroman-7-sulfonate (Intermediate-14a)

A solution of (R)-Perfluorophenyl 4-(2-bromo-4-(trifluoromethyl)phenoxy)chroman-7-sulfonate (step-1) (0.350 g, 0.565 mmol), 1-Methyl-5-(4,4,5,5-tetramethyl-1,3,2-dioxaborolan-2-yl)-1H-pyrazole (0.141 g, 0.678 mmol) and K₃PO₄ (0.240 g, 1.130 mmol) in 1,4-dioxane (10 ml) was purged with N₂ for 10 min. and then Amphos (0.040 g, 0.057 mmol) was added and heated the reaction mixture under microwave irradiation at 90° C. for 15 min. After completion of reaction as indicated by TLC, the mixture was diluted with ethyl acetate and filtered through celite. The filtrate was then washed with brine, dried over Na₂SO₄ and evaporated to dryness. The crude product was purified by flash column chromatography to obtain title compound as off-white solid (0.21 g, 59%). LCMS(ESI): m/z 621.06 (M+H)⁺; ¹H NMR (400 MHz, Chloroform-d) δ 7.76 (dd, J=8.8, 2.4 Hz, 1H), 7.63 (d, J=2.4 Hz, 1H), 7.53 (d, J=1.9 Hz, 1H), 7.47 (d, J=1.9 Hz, 1H), 7.43 (dd, J=8.0, 2.0 Hz, 1H), 7.32 (d, J=8.6 Hz, 1H), 7.18 (d, J=8.1 Hz, 1H), 6.20 (d, J=1.9 Hz, 1H), 5.32-5.28 (m, 1H), 4.31 (dt, J=11.3, 4.0 Hz, 1H), 4.07 (ddd, J=11.3, 8.8, 5.3 Hz, 1H), 3.67 (s, 3H), 2.27-2.10 (m, 2H).

### Intermediate-14b: (S)-Perfluorophenyl 4-(2-(1-methyl-1H-pyrazol-5-yl)-4-(trifluoromethyl) phenoxy)chroman-7-sulfonate

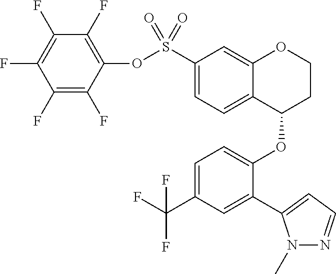 [SMILES: Cn1nccc1-c1cc(C(F)(F)F)ccc1O[C@H]1CCOc2cc(S(=O)(=O)Oc3c(F)c(F)c(F)c(F)c3F)ccc21]

The title compound was prepared by following the similar procedure as described in Intermediate-14a from Intermediate-12b. Yield: 80%; LCMS(ESI): m/z 620.46 (M+H)⁺; ¹H NMR (400 MHz, Chloroform-d) δ 7.76 (ddd, J=8.7, 2.5, 0.8 Hz, 1H), 7.63 (d, J=2.4 Hz, 1H), 7.53 (d, J=2.0 Hz, 1H), 7.50-7.39 (m, 2H), 7.32 (d, J=8.7 Hz, 1H), 7.25-7.14 (m, 1H), 6.20 (d, J=1.9 Hz, 1H), 5.30 (t, J=3.7 Hz, 1H), 4.36-4.26 (m, 1H), 4.12-3.97 (m, 1H), 3.67 (s, 3H), 2.17 (dt, J=8.4, 4.0 Hz, 2H).

### Intermediate-15: tert-Butyl 4-(2-hydroxy-5-(trifluoromethyl) phenyl)piperidine-1-carboxylate

### Step-1: tert-Butyl 4-(2-((tert-butyldimethylsilyl)oxy)-5-(trifluoromethyl)phenyl)-5,6-dihydropyridine-1(2H)-carboxylate

A solution of (2-Bromo-4-(trifluoromethyl)phenoxy)(tert-butyl)dimethylsilane(1 g, 2.81 mmol), tert-Butyl 4-(4,4,5,5-tetramethyl-1,3,2-dioxaborolan-2-yl)-5,6-dihydropyridine-1(2H)-carboxylate (0.87 g, 2.81 mmol) and potassium phosphate dibasic (1.2 g, 7.04 mmol) in 1,4-dioxane (3 ml) was purged with N₂ for 10 min. then added into PdCl₂(dppf)-DCM-adduct (0.230 g, 0.281 mmol) and heated at 110° C. for 1 h. The reaction mixture was diluted with ethyl acetate and filtered through celite. Filtrate was washed with brine, dried over Na₂SO₄ and evaporated to dryness under vacuum. The crude product was purified by flash column chromatography (20% ethyl acetate/Petroleum ether) to obtain title compound as colorless oil (0.7 g, 54%). ¹H NMR (400 MHz, Chloroform-d) δ 7.44-7.35 (m, 2H), 6.86 (d, J=8.3 Hz, 1H), 5.76 (s, 1H), 4.05 (s, 2H), 3.61 (d, J=6.3 Hz, 2H), 2.46 (s, 2H), 1.51 (s, 9H), 0.98 (s, 9H), 0.21 (s, 6H).

### Step-2: tert-Butyl 4-(2-((tert-butyldimethylsilyl)oxy)-5-(trifluoromethyl)phenyl) piperidine-1-carboxylate

To a solution of tert-Butyl 4-(2-((tert-butyldimethylsilyl)oxy)-5-(trifluoromethyl)phenyl)-5,6-dihydropyridine-1(2H)-carboxylate (0.6 g, 1.311 mmol) in MeOH (15 ml) was added 10% Pd/C (0.028 g, 0.262 mmol) and stirred at room temperature for 16 h under hydrogen balloon atmosphere. Reaction mixture was filtered through celite and solvent was removed under reduced pressure to obtain title compound as colorless gummy solid. ¹H NMR (400 MHz, Chloroform-d) δ 7.40-7.30 (m, 2H), 6.86 (d, J=8.3 Hz, 1H), 4.35-4.25 (m, 2H), 3.19-3.03 (m, 1H), 2.84-2.77 (m, 2H), 1.85-1.75 (m, 2H), 1.65-1.55 (m, 2H), 1.50 (s, 9H), 1.04 (s, 9H), 0.29 (s, 6H).

### Step-3: tert-Butyl 4-(2-hydroxy-5-(trifluoromethyl)phenyl)piperidine-1-carboxylate (Intermediate-15)

To a solution of tert-Butyl 4-(2-((tert-butyldimethylsilyl)oxy)-5-(trifluoromethyl) phenyl) piperidine-1-carboxylate (0.5 g, 1.088 mmol) in THF (5 ml) was added TBAF (1M in THF) (2.72 ml, 2.72 mmol) at room temperature and stirred for 3 h. Reaction mixture was poured into water and extracted with ethyl acetate. The combined organic layer was washed with brine, dried over Na₂SO₄ and evaporated under vacuum. It was purified with flash column chromatography (25% ethyl acetate/Petroleum ether) to obtain title compound as colorless oil. (0.3 g, 80%)¹H NMR (400 MHz, Chloroform-d) δ 7.46-7.33 (m, 2H), 6.85 (d, J=8.3 Hz, 1H), 4.35-4.20 (m, 2H), 3.06 (tt, J=12.1, 3.4 Hz, 1H), 2.95-2.80 (m, 2H), 1.90-1.80 (m, 2H), 1.70-1.62 (m, 2H), 1.51 (s, 9H).

### Intermediate-16: tert-Butyl 4-(2-((7-((perfluorophenoxy)sulfonyl)chroman-4-yl)oxy)-5-(trifluoromethyl)phenyl)piperidine-1-carboxylate

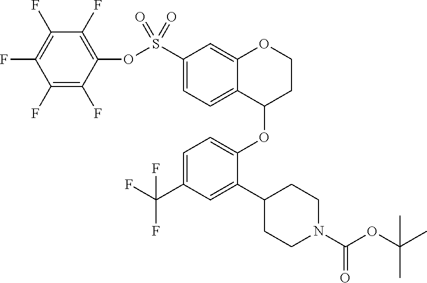 [SMILES: CC(C)(C)OC(=O)N1CCC(c2cc(C(F)(F)F)ccc2OC2CCOc3cc(S(=O)(=O)Oc4c(F)c(F)c(F)c(F)c4F)ccc32)CC1]

### Step-1: tert-Butyl 4-(2-((7-bromochroman-4-yl)oxy)-5-(trifluoromethyl)phenyl) piperidine-1-carboxylate

To a solution of 7-Bromochroman-4-ol (Prepared as described in US2013/18055)(0.64 g, 2.78 mmol), tert-Butyl 4-(2-hydroxy-5-(trifluoromethyl)phenyl)piperidine-1-carboxylate (Intermediate-15) (0.80 g, 2.316 mmol) and triphenylphosphine (0.91 g, 3.47 mmol) in THF (25 ml) was added DIAD (0.901 ml, 4.63 mmol) stirred at room temperature for 16 h. Reaction mixture was diluted with ethyl acetate and washed with aqueous NaOH solution, brine, dried over Na₂SO₄ and evaporated. The crude product was purified by flash column chromatography (10% ethyl acetate/Petroleum ether) to obtain title compound (0.5 g, 39%). LCMS(ESI): m/z 578.12 (M+Na)⁺.

### Step-2: tert-Butyl 4-(2-((7-(benzylthio)chroman-4-yl)oxy)-5-(trifluoromethyl)phenyl) piperidine-1-carboxylate

A solution of tert-Butyl 4-(2-((7-bromochroman-4-yl)oxy)-5-(trifluoromethyl)phenyl) piperidine-1-carboxylate (0.60 g, 1.078 mmol) in 1,4-dioxane (25 ml) and was purged with N₂ for 15 min then Xantphos (0.031 g, 0.054 mmol), Pd₂(dba)₃ (0.025 g, 0.027 mmol), Hunig's base (0.377 ml, 2.157 mmol) and benzyl mercaptan(0.134 ml, 1.132 mmol) were added. The reaction mixture was heated at 80° C. for 1 h. After completion of reaction as indicated by TLC, it was diluted with ethyl acetate and washed with brine, dried over Na₂SO₄ and evaporated under vacuum. The crude product was purified by flash column chromatography to obtain title compound (0.45 g, 69%). LCMS(ESI): m/z 622.2 (M+Na)⁺.

### Step-3: tert-Butyl 4-(2-((7-((perfluorophenoxy)sulfonyl)chroman-4-yl)oxy)-5-(trifluoro methyl)phenyl)piperidine-1-carboxylate

To an ice-cold solution of tert-Butyl 4-(2-((7-(benzylthio)chroman-4-yl)oxy)-5-(trifluoromethyl)phenyl)piperidine-1-carboxylate (0.40 g, 0.667 mmol) in ACN—AcOH—H₂O (10 ml-0.5 ml-0.5 ml) was added portion wise 1,3-dichloro-5,5-dimethylimidazolidine-2,4-dione (0.26 g, 1.334 mmol) at 0° C. The reaction mixture was then allowed to stir at room temperature for 2 h, then diluted with DCM (50 ml) and washed with water. The organic layer was dried over Na₂SO₄, and evaporated under vacuum to obtain sulfonyl chloride intermediate which was added to a solution of 2,3,4,5,6-pentafluorophenol (0.147 g, 0.800 mmol) and TEA (0.465 ml, 3.33 mmol) in DCM (15 ml) at 0° C. The reaction mixture was stirred for 1 h then quenched with water and extracted with DCM. The combined organic layer was washed with brine, dried over Na₂SO₄, and evaporated under vacuum. The crude was purified with flash column chromatography to obtain title compound as colorless gummy solid (0.32 g, 66%). ¹H NMR (400 MHz, Chloroform-d) δ 7.61 (d, J=2.0 Hz, 1H), 7.61-7.52 (m, 2H), 7.47-7.37 (m, 2H), 7.16 (d, J=8.6 Hz, 1H), 5.51 (t, J=4.2 Hz, 1H), 4.57-4.35 (m, 2H), 4.30-4.10 (m, 2H), 3.06-2.90 (m, 1H), 2.80-2.60 (m, 2H), 2.40-2.35 (m, 2H), 1.75-1.55 (m, 4H), 1.47 (s, 9H).

### Intermediate-17a: (S)-Perfluorophenyl 4-hydroxy-2,2-dimethylchroman-7-sulfonate

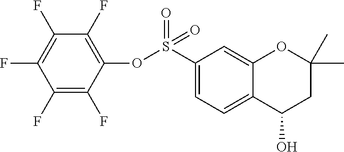 [SMILES: CC1(C)C[C@H](O)c2ccc(S(=O)(=O)Oc3c(F)c(F)c(F)c(F)c3F)cc2O1]

The title compound was prepared by following similar procedure as described in Intermediate-12a using Perfluorophenyl 2,2-dimethyl-4-oxochroman-7-sulfonate (Prepared from 7-bromo-2,2-dimethylchroman-4-one (US2002/82264A1) and R-CBS as catalyst.

Enantiomeric Excess (ee): 98.5%, Chiral pak IA, Mobile phase: (n-hexane:0.1% DEA):(ETOH:DCM, 1:1) 90:10, Retention time: 8.39 min).

Yield 63%; LCMS(ESI): m/z 446.89 (M+Na)⁺; ¹H NMR (400 MHz, Chloroform-d) δ 7.85-7.70 (m, 1H), 7.55-7.48 (m, 1H), 7.40 (d, J=1.9 Hz, 1H), 5.01-4.86 (m, 1H), 2.33-2.21 (m, 1H), 2.01-1.86 (m, 1H), 1.50 (s, 3H), 1.36 (s, 3H).

### Intermediate-17b: (R)-Perfluorophenyl 4-hydroxy-2,2-dimethylchroman-7-sulfonate

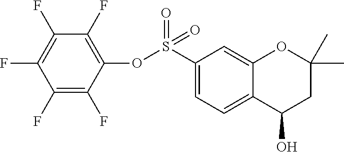 [SMILES: CC1(C)C[C@@H](O)c2ccc(S(=O)(=O)Oc3c(F)c(F)c(F)c(F)c3F)cc2O1]

The title compound was prepared by following similar procedure as described in Intermediate-12a using Perfluorophenyl 2,2-dimethyl-4-oxochroman-7-sulfonate and S-CBS as catalyst. Yield 91%; LCMS(ESI): m/z 446.88 (M+Na)⁺; ¹H NMR (400 MHz, Chloroform-d) δ 7.80-7.66 (m, 1H), 7.57-7.47 (m, 1H), 7.41 (d, J=1.9 Hz, 1H), 5.03-4.84 (m, 1H), 2.33-2.21 (m, 1H), 1.99-1.85 (m, 1H), 1.50 (s, 3H), 1.36 (s, 3H).

Below Intermediate 18a and 18b were prepared by following similar procedure as described in Intermediate-13a using Intermediate 17a and 17b respectively.

### Intermediate-18a: (R)-Perfluorophenyl 4-(2-chloro-4-(trifluoromethyl)phenoxy)-2,2-dimethylchroman-7-sulfonate

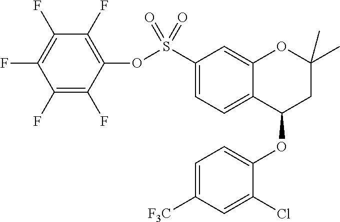 [SMILES: CC1(C)C[C@@H](Oc2ccc(C(F)(F)F)cc2Cl)c2ccc(S(=O)(=O)Oc3c(F)c(F)c(F)c(F)c3F)cc2O1]

Yield 35%; LCMS(ESI): m/z 625.00 (M+Na)⁺; 1H NMR (400 MHz, Chloroform-d) δ 7.74 (t, J=1.4 Hz, 1H), 7.70-7.65 (m, 1H), 7.60-7.46 (m, 3H), 7.17 (d, J=8.6 Hz, 1H), 5.58 (t, J=6.6 Hz, 1H), 2.38-2.22 (m, 2H), 1.51 (s, 3H), 1.46 (s, 3H).

### Intermediate-18b: (S)-Perfluorophenyl 4-(2-chloro-4-(trifluoromethyl)phenoxy)-2,2-dimethylchroman-7-sulfonate

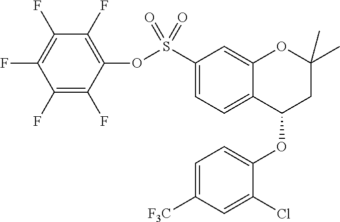 [SMILES: CC1(C)C[C@H](Oc2ccc(C(F)(F)F)cc2Cl)c2ccc(S(=O)(=O)Oc3c(F)c(F)c(F)c(F)c3F)cc2O1]

Yield 38%; LCMS(ESI): m/z 625.00 (M+Na)⁺; ¹H NMR (400 MHz, Chloroform-d) δ 7.74 (d, J=2.3 Hz, 1H), 7.67 (d, J=8.1 Hz, 1H), 7.59-7.50 (m, 3H), 7.17 (d, J=8.6 Hz, 1H), 5.58 (t, J=6.4 Hz, 1H), 2.49-2.10 (m, 2H), 1.52 (s, 3H), 1.47 (s, 2H).

### Intermediate-19a/19b: (R/S)-Perfluorophenyl 2-ethyl-4-oxochroman-7-sulfonate

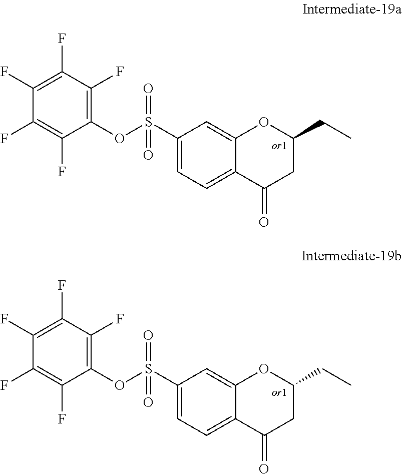 [SMILES: CC[C@H]1CC(=O)c2ccc(S(=O)(=O)Oc3c(F)c(F)c(F)c(F)c3F)cc2O1 ; CC[C@@H]1CC(=O)c2ccc(S(=O)(=O)Oc3c(F)c(F)c(F)c(F)c3F)cc2O1]

### Step-1: 7-Bromo-2-ethylchroman-4-one

To a solution of 1-(4-bromo-2-hydroxyphenyl)ethanone (15 g, 69.8 mmol) in toluene (200 ml), pyrrolidine (5.77 ml, 69.8 mmol) was added followed by propionaldehyde (4.86 g, 84 mmol) and heated at 90° C. for 18 h. After completion as shown in TLC the mixture was evaporated to dryness. The residue was poured into 1N HCl solution and extracted with DCM. The combined organic layer was washed with brine, dried over Na₂SO₄ and evaporated under vacuum. The crude product was purified by column chromatography to obtain title compound as off white solid (8 g, 45%). GCMS: 254.09.

### Step-2: 7-(Benzylthio)-2-ethylchroman-4-one

7-Bromo-2-ethylchroman-4-one (4 g, 15.68 mmol) was dissolved in 1,4-dioxane (100 ml) and purged with N₂ and then Xantphos (0.45 g, 0.784 mmol), Pd₂(dba)₃ (0.36 g, 0.392 mmol), Hunig's Base (5.48 ml, 31.4 mmol) and benzyl mercaptan(1.94 ml, 16.46 mmol) were added. The reaction mixture was heated at 80° C. for 1 h then black reaction mixture was extracted with ethyl acetate and washed with water, brine and dried over Na₂SO₄ and concentrated under vacuum. The crude was purified by flash column chromatography to obtain title compound as pale yellow oil (3.5 g, 74). ¹H NMR (400 MHz, Chloroform-d) δ 7.76 (d, J=8.3 Hz, 1H), 7.47-7.29 (m, 5H), 6.92-6.83 (m, 2H), 4.46-4.29 (m, 1H), 4.23 (s, 2H), 2.71-2.63 (m, 2H), 1.92-1.71 (m, 2H), 1.08 (t, J=7.5 Hz, 3H).

### Step-3: (R/S)-Perfluorophenyl 2-ethyl-4-oxochroman-7-sulfonate

To an ice-cold solution of 7-(Benzylthio)-2-ethylchroman-4-one (2.5 g, 8.38 mmol) in DCM-HOAc—H₂O (40 ml-2 ml 2 ml) was added sulfuryl chloride (0.817 ml, 10.05 mmol) in a drop wise manner. The reaction mixture was stirred at 0° C. for 4 h. The reaction mixture was diluted with DCM and washed with water, brine, dried over Na₂SO₄ and concentrated under vacuum. It was dissolved in DCM (15 ml) and added drop wise to the solution of 2,3,4,5,6-pentafluorophenol (1.696 g, 9.22 mmol) and TEA (3.50 ml, 25.1 mmol) in DCM (30 ml) at 0° C. and stirred for 30 min at room temperature. The reaction mixture was poured in to water and extracted with DCM. The combined organic layer was dried over Na₂SO₄, and concentrated under vacuum. The crude product purified by column chromatography to obtain Perfluorophenyl 2-ethyl-4-oxochroman-7-sulfonate as colorless oil (1 g, 42%). ¹H NMR (400 MHz, Chloroform-d) δ 8.10 (d, J=8.3 Hz, 1H), 7.66 (d, J=1.7 Hz, 1H), 7.58 (dd, J=8.3, 1.8 Hz, 1H), 4.52 (ddt, J=10.4, 7.2, 5.3 Hz, 1H), 2.88-2.74 (m, 2H), 2.01-1.80 (m, 2H), 1.12 (t, J=7.5 Hz, 3H).

The enantiomers were separated by chiral preparative (Chiral PAK IA, mobile phase: (n-hexane:0.1% DEA):(ETOH:DCM, 1:1) 70:30, Intermediate-19a: Retention time 10.05 min.; Intermediate 19b: Retention time 12.59 min.

### Intermediate-20a: (2R/S,4S)-Perfluorophenyl 2-ethyl-4-hydroxychroman-7-sulfonate

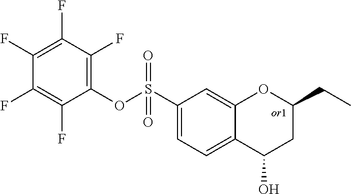 [SMILES: CC[C@H]1C[C@H](O)c2ccc(S(=O)(=O)Oc3c(F)c(F)c(F)c(F)c3F)cc2O1]

To a solution of (R)-1-Methyl-3,3-diphenylhexahydropyrrolo[1,2-c][1,3,2]oxazaborole (0.046 g, 0.166 mmol) in THF (20 ml) was added BH₃.DMS (0.102 ml, 1.077 mmol) at room temperature and stirred for 30 min. Added in to (Intermediate-19a)(0.350 g, 0.829 mmol) in THF (10 ml) and stirred at room temperature for 30 min. After completion of reaction as indicated by TLC, reaction mixture was quenched with MeOH and poured in to water and extract with ethyl acetate. The combined organic layer was dried over Na₂SO₄ and concentrated under vacuum. The crude product was purified by flash column chromatography (30% ethyl acetate/Petroleum ether) to obtain colorless oil (0.2 g, 57%). ¹H NMR (400 MHz, Chloroform-d) δ 7.58-7.46 (m, 3H), 4.88 (t, J=3.1 Hz, 1H), 4.31-4.20 (m, 1H), 2.22-2.09 (m, 1H), 1.95-1.84 (m, 1H), 1.84-1.72 (m, 2H), 1.11 (t, J=7.5 Hz, 4H).

### Intermediate-20b: (2R/S,4S)-Perfluorophenyl 2-ethyl-4-hydroxychroman-7-sulfonate

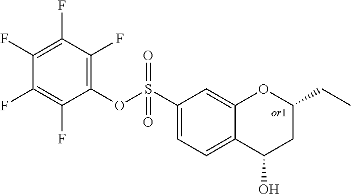 [SMILES: CC[C@@H]1C[C@H](O)c2ccc(S(=O)(=O)Oc3c(F)c(F)c(F)c(F)c3F)cc2O1]

The title compound was prepared by following similar procedure as described for Intermediate-20a using Intermediate-19b and R-CBS catalyst. Yield: 67%; ¹H NMR (400 MHz, Chloroform-d) δ 7.74 (dd, J=8.2, 1.1 Hz, 1H), 7.50 (dd, J=8.2, 2.0 Hz, 1H), 7.42 (d, J=1.9 Hz, 1H), 5.18-4.92 (m, 1H), 4.28-4.17 (m, 1H), 2.39 (ddd, J=13.0, 6.2, 1.8 Hz, 1H), 1.88-1.75 (m, 3H), 1.08 (t, J=7.5 Hz, 3H).

### Intermediate-21: Perfluorophenyl 5-(2-chloro-4-(trifluoromethyl)phenoxy)-5,6,7,8-tetrahydro naphthalene-2-sulfonate

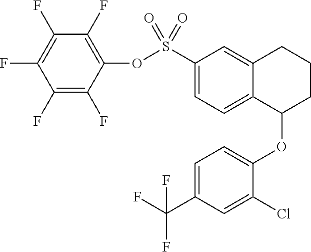 [SMILES: O=S(=O)(Oc1c(F)c(F)c(F)c(F)c1F)c1ccc2c(c1)CCCC2Oc1ccc(C(F)(F)F)cc1Cl]

### Step-1: 6-(Benzylthio)-3,4-dihydronaphthalen-1 (2H)-one

6-Bromo-3,4-dihydronaphthalen-1(2H)-one (10 g, 44.4 mmol) was dissolved in 1,4-dioxane (100 ml) and the solution was purged with nitrogen for 15 min and then added xanthphos (1.285 g, 2.221 mmol), Pd₂(dba)₃ (1.1 g, 1.11 mmol), Hunig's base (15.52 ml, 89 mmol) and benzyl mercaptan(5.52 ml, 46.6 mmol). The reaction mixture was heated at 80° C. for 1 h. After completion of reaction, the reaction mixture was diluted with ethyl acetate and washed with water, brine, dried over Na₂SO₄ and evaporated under vacuum. The crude product was purified by flash column chromatography to obtain 6-(benzylthio)-3,4-dihydronaphthalen-1(2H)-one (8.5 g, 71%). LCMS(ESI): m/z 268.13 (M+H)⁺; ¹H NMR (400 MHz, Chloroform-d) δ 7.93 (d, J=8.3 Hz, 1H), 7.42-7.26 (m, 5H), 7.22-7.17 (m, 1H), 7.12 (d, J=1.9 Hz, 1H), 4.23 (s, 2H), 2.90 (t, J=6.1 Hz, 2H), 2.72-2.59 (m, 2H), 2.20-2.05 (m, 2H).

### Step-2: Perfluorophenyl 5-oxo-5,6,7,8-tetrahydronaphthalene-2-sulfonate

To an ice-cold solution of 6-(benzylthio)-3,4-dihydronaphthalen-1(2H)-one (3.2 g, 11.92 mmol) in DCM-AcOH—H₂O (5 ml, 1 ml, 1 ml) was added sulfuryl chloride (1.20 ml, 14.31 mmol) drop wise. The reaction mixture was stirred at 0° C. for 2 h, and then concentrated to near dryness under vacuum. The crude product was diluted with DCM (50 ml), washed with water, brine, dried over Na₂SO₄ and evaporated under vacuum. The sulfonyl chloride derivative thus obtained was taken in DCM (25 ml) and 2,3,4,5,6-pentafluorophenol (2.41 g, 13.12 mmol) and TEA (5 ml, 35.8 mmol) were added. Stirred the mixture at 0-5° C. for 30 min and then poured into water and extracted with DCM. The combined organic layer was washed with brine, dried over Na₂SO₄, and concentrated under vacuum. Purification was done by flash column chromatography to obtain the title compound as white solid (3 g, 64%). ¹H NMR (400 MHz, Chloroform-d) δ 8.25 (d, J=8.2 Hz, 1H), 8.00-7.86 (m, 2H), 3.11 (t, J=6.1 Hz, 2H), 2.78 (dd, J=7.3, 5.8 Hz, 2H), 2.32-2.19 (m, 2H).

### Step-3: Perfluorophenyl 5-hydroxy-5,6,7,8-tetrahydronaphthalene-2-sulfonate

To a solution of Perfluorophenyl 5-oxo-5,6,7,8-tetrahydronaphthalene-2-sulfonate (0.70 g, 1.78 mmol) in MeOH (15 ml) at 0° C. was added NaBH₄ (0.08 g, 2.14 mmol) and stirred at room temperature for 1 h. TLC indicated completion of reaction. The solvent was removed under vacuum and quenched with saturated NH₄Cl solution. The product was extracted with ethyl acetate and the combined organic layer was washed with brine, dried over Na₂SO₄ and evaporated under vacuum to obtain the title compound as off-white solid (0.70 g, 99%). ¹H NMR (400 MHz, Chloroform-d) δ 7.60 (d, J=8.1 Hz, 1H), 7.54-7.50 (m, 1H), 7.47 (d, J=1.8 Hz, 1H), 5.00-4.90 (m, 1H), 4.41-4.33 (m, 2H), 2.29-2.08 (m, 2H).

### Step-4: Perfluorophenyl 5-(2-chloro-4-(trifluoromethyl)phenoxy)-5,6,7,8-tetrahydro naphthalene-2-sulfonate (Intermediate-21)

To a solution of Perfluorophenyl 5-hydroxy-5,6,7,8-tetrahydronaphthalene-2-sulfonate (0.35 g, 0.888 mmol), 2-Chloro-4-(trifluoromethyl)phenol (0.21 g, 1.06 mmol), triphenylphosphine (0.3 g, 1.15 mmol) in THF (3 ml) was added DEAD (0.21 ml, 1.33 mmol) and stirred at room temperature for overnight. The reaction mixture was diluted with ethyl acetate and washed with water, brine, dried over Na₂SO₄ and evaporated under vacuum. The crude was purified by flash column chromatography (10% ethyl acetate/Petroleum ether) to obtain title compound as off-white solid. ¹H NMR (400 MHz, Chloroform-d) δ 7.86-7.78 (m, 2H), 7.73-7.64 (m, 2H), 7.58-7.52 (m, 1H), 7.18 (d, J=8.6 Hz, 1H), 5.55-5.45 (m, 1H), 3.20-2.85 (m, 2H), 2.25-1.85 (m, 4H).

### Intermediate-21a: (R)-Perfluorophenyl 5-(2-chloro-4-(trifluoromethyl)phenoxy)-5,6,7,8-tetrahydronaphthalene-2-sulfonate

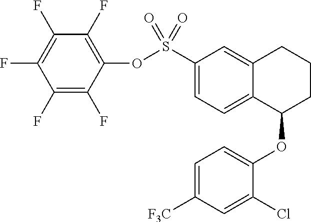 [SMILES: O=S(=O)(Oc1c(F)c(F)c(F)c(F)c1F)c1ccc2c(c1)CCC[C@H]2Oc1ccc(C(F)(F)F)cc1Cl]

### Step-1: (S)-Perfluorophenyl 5-hydroxy-5,6,7,8-tetrahydronaphthalene-2-sulfonate

To a solution of (R)-1-Methyl-3,3-diphenylhexahydropyrrolo[1,2-c][1,3,2]oxazaborole (1.28 ml, 1.28 mmol) in THF (30 ml) was added BH₃.DMS (0.80 ml, 8.28 mmol) at room temperature and stirred for 30 min. Added into Perfluorophenyl 5-oxo-5,6,7,8-tetrahydronaphthalene-2-sulfonate (step-2, Intermediate-21) (2.5 g, 6.37 mmol) in THF (20 ml) drop wise and stirred at room temperature for another 30 min. TLC shown completion of reaction. Reaction mixture was then quenched with MeOH, and poured in to water and extract with ethyl acetate. The combined organic layer was dried over Na₂SO₄, and concentrated under vacuum. It was purified with flash column chromatography (30% ethyl acetate/Petroleum ether) to obtain title compound as colorless oil (2.1 g, 84%). LCMS(ESI): m/z 416.92 (M+H)⁺.

### Step-2: (R)-Perfluorophenyl 5-(2-chloro-4-(trifluoromethyl)phenoxy)-5,6,7,8-tetrahydro naphthalene-2-sulfonate

To a solution of (S)-perfluorophenyl 5-hydroxy-5,6,7,8-tetrahydronaphthalene-2-sulfonate (step-1)(0.20 g, 0.507 mmol), triphenylphosphine (0.27 g, 1.014 mmol), 2-chloro-4-(trifluoromethyl)phenol (0.12 g, 0.609 mmol in THF (25 ml) was added diisopropyl azodicarboxylate (0.20 ml, 1.01 mmol) and stirred at room temperature for 1 h. After completion of reaction, the solvent was removed under vacuum and crude was purified by flash column chromatography to obtain title compound as colorless gummy solid (0.1 g, 35%). ¹H NMR (400 MHz, Chloroform-d) δ 7.90-7.78 (m, 2H), 7.73-7.53 (m, 3H), 7.18 (d, J=8.6 Hz, 1H), 5.51 (q, J=7.1, 6.4 Hz, 1H), 3.20-2.80 (m, 2H), 2.29-1.80 (m, 4H).

### Intermediate-22: Perfluorophenyl 5-(2-bromo-4-(trifluoromethyl)phenoxy)-5,6,7,8-tetrahydronaphthalene-2-sulfonate

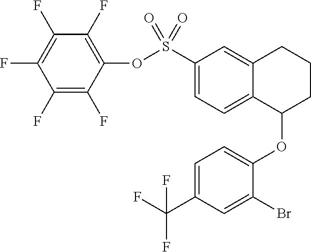 [SMILES: O=S(=O)(Oc1c(F)c(F)c(F)c(F)c1F)c1ccc2c(c1)CCCC2Oc1ccc(C(F)(F)F)cc1Br]

The title compound was prepared by following the similar procedure as described in Intermediate-21 using 2-Bromo-4-(trifluoro methyl)phenol. ¹H NMR (400 MHz, Chloroform-d) δ 7.91-7.72 (m, 3H), 7.70-7.60 (m, 2H), 7.14 (d, J=8.7 Hz, 1H), 5.53 (t, J=5.4 Hz, 1H), 3.10-3.00 (m, 1H), 2.95-2.85 (m, 1H), 2.26-2.12 (m, 3H), 2.00-1.86 (m, 1H).

### Intermediate-22a: (R)-Perfluorophenyl 5-(2-bromo-4-(trifluoromethyl)phenoxy)-5,6,7,8-tetrahydronaphthalene-2-sulfonate

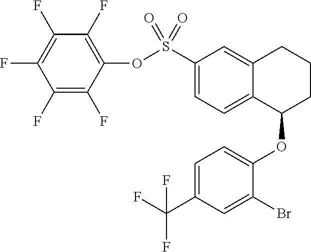 [SMILES: O=S(=O)(Oc1c(F)c(F)c(F)c(F)c1F)c1ccc2c(c1)CCC[C@H]2Oc1ccc(C(F)(F)F)cc1Br]

The title compound was prepared by following the similar procedure as described in Intermediate-21a using 2-Bromo-4-(trifluoro methyl)phenol. Yield: 32%; ¹H NMR (400 MHz, Chloroform-d) δ 7.92-7.76 (m, 3H), 7.67 (d, J=8.2 Hz, 1H), 7.60 (ddd, J=8.6, 2.4, 0.8 Hz, 1H), 7.14 (d, J=8.6 Hz, 1H), 5.53 (t, J=5.3 Hz, 1H), 3.15-2.84 (m, 2H), 2.28-1.86 (m, 4H).

### Intermediate-23: Perfluorophenyl 5-(2-(1-methyl-1H-pyrazol-5-yl)-4-(trifluoromethyl) phenoxy)-5,6,7,8-tetrahydronaphthalene-2-sulfonate

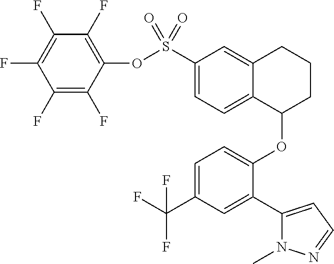 [SMILES: Cn1nccc1-c1cc(C(F)(F)F)ccc1OC1CCCc2cc(S(=O)(=O)Oc3c(F)c(F)c(F)c(F)c3F)ccc21]

The title compound was prepared by following similar procedure as described in Intermediate-14a using Intermediate-22 and 1-Methyl-5-(4,4,5,5-tetramethyl-1,3,2-dioxaborolan-2-yl)-1H-pyrazole. Yield: 60%; LCMS(ESI): m/z 618.56 (M+H)⁺; ¹H NMR (400 MHz, Chloroform-d) δ 7.81-7.68 (m, 3H), 7.65-7.58 (m, 1H), 7.54 (d, J=2.0 Hz, 1H), 7.32-7.30 (m, 2H), 6.25 (d, J=2.0 Hz, 1H), 5.40 (t, J=5.1 Hz, 1H), 3.69 (s, 3H), 2.99-2.76 (m, 2H), 2.06 (dt, J=6.5, 5.3 Hz, 2H), 1.96-1.78 (m, 2H).

### Intermediate-23a: (R)-Perfluorophenyl 5-(2-(1-methyl-1H-pyrazol-5-yl)-4-(trifluoro methyl)phenoxy)-5,6,7,8-tetrahydronaphthalene-2-sulfonate

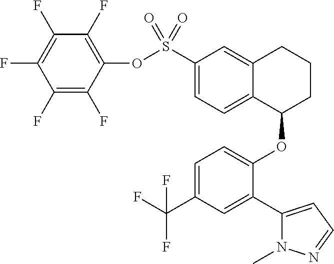 [SMILES: Cn1nccc1-c1cc(C(F)(F)F)ccc1O[C@@H]1CCCc2cc(S(=O)(=O)Oc3c(F)c(F)c(F)c(F)c3F)ccc21]

The title compound was prepared by following similar procedure as described in Intermediate-23 using Intermediate-22a and 1-Methyl-5-(4,4,5,5-tetramethyl-1,3,2-dioxaborolan-2-yl)-1H-pyrazole. Yield: 44%; LCMS(ESI): m/z 618.33 (M+H)⁺; ¹H NMR (400 MHz, Chloroform-d) δ 7.78-7.70 (m, 3H), 7.61 (d, J=2.4 Hz, 1H), 7.51 (d, J=1.9 Hz, 1H), 7.30-7.25 (m, 2H), 6.23 (d, J=1.9 Hz, 1H), 5.39 (t, 1H), 3.67 (s, 3H), 2.95-2.77 (m, 2H), 2.11-2.02 (m, 2H), 1.90-1.75 (m, 2H).

### Intermediate-24: tert-Butyl 4-(2-((6-((perfluorophenoxy)sulfonyl)-1,2,3,4-tetrahydro naphthalen-1-yl)oxy)-5-(trifluoromethyl)phenyl)-5,6-dihydropyridine-1(2H)-carboxylate

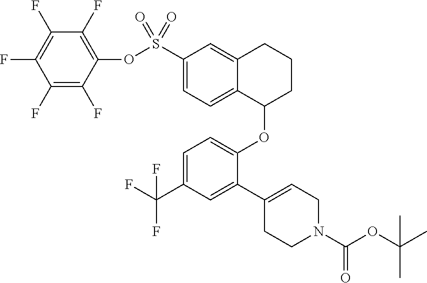 [SMILES: CC(C)(C)OC(=O)N1CC=C(c2cc(C(F)(F)F)ccc2OC2CCCc3cc(S(=O)(=O)Oc4c(F)c(F)c(F)c(F)c4F)ccc32)CC1]

A solution of Perfluorophenyl 5-(2-bromo-4-(trifluoromethyl)phenoxy)-5,6,7,8-tetrahydro naphthalene-2-sulfonate (Intermediate-22) (0.10 g, 0.16 mmol), tert-butyl 4-(4,4,5,5-tetramethyl-1,3,2-dioxaborolan-2-yl)-5,6-dihydropyridine-1 (2H)-carboxylate (0.05 g, 0.16 mmol), and potassium phosphate (0.017 g, 0.097 mmol) in 1,4-dioxane:water (1 ml, 0.3 ml) was purged with N₂ for 10 min and then bis(di-tert-butyl(4-dimethylaminophenyl) phosphine)dichloropalladium(II) (0.006 g, 0.008 mmol) was added. The reaction vial was heated at 110° C. for 30 min under microwave irradiation then cooled to room temperature; the reaction mixture was diluted with ethyl acetate and washed with water and brine. The combined organic layer was dried over Na₂SO₄ and evaporated under vacuum. The crude product was purified by flash column chromatography to obtain title compound as off-white solid (0.10 g, 86%). ¹H NMR (400 MHz, Chloroform-d) δ 7.83 (d, J=2.0 Hz, 1H), 7.80-7.72 (m, 1H), 7.57-7.49 (m, 2H), 7.46 (d, J=2.4 Hz, 1H), 7.11 (d, J=8.6 Hz, 1H), 5.75-5.70 (m, 1H), 5.48 (t, J=5.5 Hz, 1H), 4.05-3.95 (m, 2H), 3.50-3.40 (m, 2H), 3.07-2.85 (m, 2H), 2.40-2.30 (m, 1H), 2.21-2.00 (m, 4H), 1.99-1.89 (m, 1H), 1.46 (s, 9H).

The enantiomers were separated using chiral prep (Column: Chiral pak IA; Mobile phase: (n-hexane:0.1% DEA):(ETOH:DCM, 1:1) 95:5, affording Isomer-A, retention time 9.58 min and Isomer-B retention time 11.2 min.

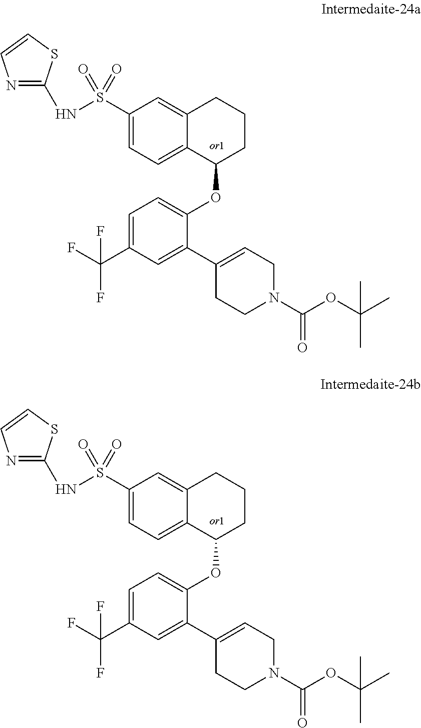 [SMILES: CC(C)(C)OC(=O)N1CC=C(c2cc(C(F)(F)F)ccc2O[C@@H]2CCCc3cc(S(=O)(=O)Nc4nccs4)ccc32)CC1 ; CC(C)(C)OC(=O)N1CC=C(c2cc(C(F)(F)F)ccc2O[C@H]2CCCc3cc(S(=O)(=O)Nc4nccs4)ccc32)CC1]

### Intermediate-25: tert-Butyl 4-(2-((6-((perfluorophenoxy)sulfonyl)-1,2,3,4-tetrahydronaphthalen-1-yl)oxy)-5-(trifluoromethyl)phenyl)piperidine-1-carboxylate

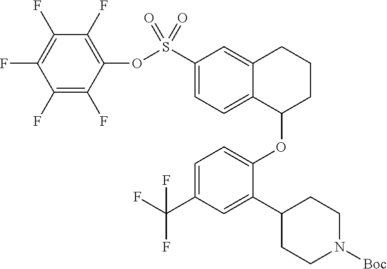 [SMILES: CC(C)(C)OC(=O)N1CCC(c2cc(C(F)(F)F)ccc2OC2CCCc3cc(S(=O)(=O)Oc4c(F)c(F)c(F)c(F)c4F)ccc32)CC1]

To a solution of Perfluorophenyl 5-hydroxy-5,6,7,8-tetrahydronaphthalene-2-sulfonate (0.4 g, 1.014 mmol) (Step-3, Intermediate-21), tert-Butyl 4-(2-hydroxy-5-(trifluoromethyl)phenyl)piperidine-1-carboxylate (Intermediate-15)(0.350 g, 1.014 mmol) and triphenylphosphine (0.35 g, 1.32 mmol) in THF (25 ml) was added DEAD (0.241 ml, 1.522 mmol) at room temperature and stirred for 16 h. Reaction mixture was diluted with ethyl acetate and washed with water, brine, dried over Na₂SO₄ and evaporated under vacuum. The crude product was purified by flash column chromatography (10% ethyl acetate/Petroleum ether) to obtain title compound as off-white gummy solid (0.35 g, 47%). LCMS(ESI): m/z 744.1 (M+Na)⁺; ¹H NMR (400 MHz, Chloroform-d) δ 7.91-7.74 (m, 2H), 7.62-7.41 (m, 3H), 7.12 (d, J=8.6 Hz, 1H), 5.55 (t, J=5.4 Hz, 1H), 4.30-4.10 (m, 2H), 3.08-2.87 (m, 3H), 2.80-2.60 (m, 2H), 2.23-1.90 (m, 4H), 1.80-1.50 (m, 4H), 1.47 (s, 9H).

### Intermediate-26: 2-Ethyl-4-(trifluoromethyl)phenol

### Step-1: 1-(2-(Benzyloxy)-5-(trifluoromethyl)phenyl)ethanone

A solution of 1-(benzyloxy)-2-bromo-4-(trifluoromethyl)benzene (10 g, 30.2 mmol) (Prepared according to procedure described in WO2005/18529) in dioxane (40 ml) was purged with N₂ for 15 min. Bis(triphenylphosphine)palladium(II) dichloride (2.120 g, 3.02 mmol) and tributyl(1-ethoxyvinyl)stannane (12.35 ml, 36.2 mmol) was added and heated the mixture at 100° C. for overnight. The Solvent was removed under vacuum and 3M HCl (40 ml) was added and stirred at for additional 1 h. It was extracted with ethyl acetate and The combined organic layer was washed with brine, dried over Na₂SO₄ and evaporated to dryness under reduced pressure. The crude product was purified with flash column chromatography (5% ethyl acetate/petroleum ether) to obtain title compound as white solid (8 g, 90%). LCMS(ESI): m/z 294.93 (M+H)⁺.

### Step-2: 1-(Benzyloxy)-2-ethyl-4-(trifluoromethyl)benzene

Heated the mixture of 1-(2-(benzyloxy)-5-(trifluoromethyl)phenyl)ethanone (1.2 g, 4.08 mmol), NaOH (0.33 g, 8.16 mmol) and hydrazinehydrate (0.512 ml, 16.31 mmol) in triethylene glycol (6 ml) at 170° C. for 8 h. TLC shown completion of reaction. Reaction mixture was allowed to come at room temperature and then poured into aq. HCl and then extracted with ethyl acetate. The combined organic layer was washed with brine, dried over Na₂SO₄ and evaporated under vacuum. The crude product was purified with flash column chromatography (10% ethyl acetate/petroleum ether) to obtain title compound as colorless oil (0.75, 65%). ¹H NMR (400 MHz, Chloroform-d) δ 7.53-7.33 (m, 7H), 6.95 (d, J=9.1 Hz, 1H), 5.15 (s, 2H), 2.76 (q, J=7.5 Hz, 2H), 1.26 (t, J=7.5 Hz, 3H).

### Step-3: 2-Ethyl-4-(trifluoromethyl)phenol

To a solution of 1-(benzyloxy)-2-ethyl-4-(trifluoromethyl)benzene (0.75 g, 2.68 mmol) in MeOH (10 ml) was added 10% Pd/C (0.28 g, 2.68 mmol) (50% wet) and stirred under hydrogen balloon atmosphere for overnight. TLC shown completion of reaction. Reaction mixture was filtered through celite and filtrate was evaporated under vacuum to obtain title compound as colorless oil (0.5 g, 98%). ¹H NMR (400 MHz, Chloroform-d) δ 7.41 (d, J=2.3 Hz, 1H), 7.36 (dd, J=8.4, 2.3 Hz, 1H), 6.84 (d, J=8.3 Hz, 1H), 2.69 (q, J=7.6 Hz, 2H), 1.27 (t, J=8.3, 3H).

### Intermediate-27: 2-Isopropyl-4-(trifluoromethyl)phenol

### Step-1: 2-(2-(benzyloxy)-5-(trifluoromethyl)phenyl)propan-2-ol

To a solution of 1-(2-(benzyloxy)-5-(trifluoromethyl)phenyl)ethanone (0.60 g, 2.039 mmol) in diethyl ether (5 ml), methylmagnesium bromide (3M in THF, 1.019 ml, 3.06 mmol) was added at 0° C. and stirred for 1 h. Reaction mixture was then poured into saturated ammonium chloride solution and extracted with diethyl ether. The combined organic layer was washed with brine, dried over Na₂SO₄ and evaporated under vacuum to give title compound as colorless oil (0.6 g, 95%). LCMS(ESI): m/z 311.14 (M+H)⁺

### Step-2: 2-(2-hydroxypropan-2-yl)-4-(trifluoromethyl)phenol

To a stirred solution of 2-(2-(benzyloxy)-5-(trifluoromethyl)phenyl)propan-2-ol (0.60 g, 1.934 mmol) in Ethanol (15 ml), 10% Pd/C (20.58 mg, 0.193 mmol) and 1 drop acetic acid was added and stirred under H₂ pressure for overnight. Reaction mixture was filtered through celite bed and filtrate was concentrated under vacuum to give title compound as colorless oil (0.39 g, 93%). ¹H NMR (400 MHz, Chloroform-d) δ 7.45 (ddd, J=8.5, 2.3, 0.8 Hz, 1H), 7.37-7.30 (m, 1H), 6.96 (dd, J=8.5, 0.9 Hz, 1H), 1.73 (s, 6H).

### Step-3: 2-(prop-1-en-2-yl)-4-(trifluoromethyl)phenol

To a stirred solution of 2-(2-hydroxypropan-2-yl)-4-(trifluoromethyl)phenol (0.35 g, 1.590 mmol) in THF (5 ml), BF₃.OEt₂ (0.90 ml, 7.15 mmol) and triethylsilane (1.90 ml, 11.92 mmol) was added and stirred at room temperature for 18 h. Reaction mixture then poured into bicarbonate solution and extracted with ethyl acetate. The combined organic layer was washed with water, brine, dried over Na₂SO₄ and evaporated to dryness under reduced pressure. The crude product was purified by flash column chromatography (5% ethyl acetate/petroleum ether) to give title compound (0.27, 83%). ¹H NMR (400 MHz, Chloroform-d) δ 7.47-7.38 (m, 2H), 7.01 (d, J=8.4 Hz, 1H), 5.49 (p, J=1.6 Hz, 1H), 5.20 (q, J=1.1 Hz, 1H), 2.15 (s, 3H).

### Step-4: 2-isopropyl-4-(trifluoromethyl)phenol

To a stirred solution of 2-(prop-1-en-2-yl)-4-(trifluoromethyl)phenol (0.28 g, 1.385 mmol) in methanol (10 ml), 10% Pd/C (0.15 g, 1.385 mmol) was added and stirred under H₂ pressure for overnight over night. TLC shown completion of reaction. Reaction mixture was filtered through celite and filtrate was evaporated under vacuum to obtain title compound as colorless oil (0.26, 92%) GCMS: m/z 204.09.

### Intermediate-28: 2-Cyclopropyl-4-(trifluoromethyl)phenol

### Step-1: 1-(benzyloxy)-2-cyclopropyl-4-(trifluoromethyl)benzene

To a nitrogen flushed solution of 1-(benzyloxy)-2-bromo-4-(trifluoromethyl)benzene (0.60 g, 1.812 mmol), cyclopropylboronic acid (0.23 g, 2.72 mmol) and potassium phosphate tribasic (1.15 g, 5.44 mmol) was added dicyclohexyl(2′,6′-diisopropoxy-[1,1′-biphenyl]-2-yl)phosphine (0.085 g, 0.181 mmol) followed by palladium(ii) acetate (0.040 g, 0.181 mmol) and reaction mixture was stirred for 7 h at 85° C. under nitrogen atmosphere. Reaction progress was monitored by TLC. Reaction mixture was diluted with ethyl acetate and partitioned between ethyl acetate and water. Organic layer was washed with brine, dried over Na₂SO₄ and concentrated under vacuum. Crude product was purified by flash column chromatography to obtain title compound (0.50 g, 94%). ¹H NMR (400 MHz, Chloroform-d): δ 7.52-7.34 (m, 6H), 7.12 (d, J=2.3 Hz, 1H), 6.95 (d, J=8.5 Hz, 1H), 5.19 (s, 2H), 2.32-2.21 (m, 1H), 1.05-0.96 (m, 2H), 0.77-0.68 (m, 2H).

### Step-2: 2-cyclopropyl-4-(trifluoromethyl)phenol

To a solution of 1-(benzyloxy)-2-cyclopropyl-4-(trifluoromethyl)benzene (0.50 g, 1.711 mmol) in ethyl acetate (25 ml) was added Pd—C(10%) (0.10 g, 0.940 mmol) very cautiously and reaction mixture was stirred under H₂ atmosphere for 2 hr. Reaction was monitored by TLC. Reaction mixture was filtered through celite and filtrate was concentrated to get crude comp which was used without further purification. ¹H NMR (400 MHz, Chloroform-d): δ 7.41 (m, 1H), 7.36 (d, J=2.3 Hz, 1H), 6.95 (dd, J=8.4, 0.9 Hz, 1H), 5.79 (s, 1H), 1.84 (m, 1H), 1.09-1.01 (m, 2H), 0.74-0.67 (m, 2H).

### Intermediate-29: 2-(1-isopropyl-1H-pyrazol-5-yl)-4-(trifluoromethyl)phenol

### Step-1: (E)-1-(2-(benzyloxy)-5-(trifluoromethyl)phenyl)-3-(dimethylamino)prop-2-en-1-one

Heated the mixture of 1-(2-(benzyloxy)-5-(trifluoromethyl)phenyl)ethanone (4 g, 13.59 mmol) and DMF.DMA (10 ml,) at 100° C. for overnight. TLC shown completion of reaction. Reaction mixture was cooled to room temperature and then poured into ice water. The yellow solid was filtered and dried under vacuum (4.5 g, 95%)

### Step-2: 5-(2-(benzyloxy)-5-(trifluoromethyl)phenyl)-1-isopropyl-1H-pyrazole

Heated the mixture of (E)-1-(2-(benzyloxy)-5-(trifluoromethyl)phenyl)-3-(dimethylamino)prop-2-en-1-one (2.4 g, 6.78 mmol), isopropylhydrazine hydrochloride (It was prepared according to procedure described in Synlett, 2004, 2355-2356)(0.90 g, 8.14 mmol) and K₂CO₃ (1.12 g, 8.14 mmol) in EtOH (25 ml) at 80° C. for overnight. After completion of reaction as indicated by TLC, reaction mixture was evaporated under vacuum, quenched with water and extracted with ethyl acetate. The combined organic layer was washed with brine, dried over Na₂SO₄ and evaporated under vacuum. The crude product was purified with flash column chromatography (20% ethyl acetate/petroleum ether) to obtain title compound as off white solid. LCMS(ESI): m/z 361.35 (M+H)⁺

### Step-3: 2-(1-isopropyl-1H-pyrazol-5-yl)-4-(trifluoromethyl)phenol

To a stirred solution of 5-(2-(benzyloxy)-5-(trifluoromethyl)phenyl)-1-isopropyl-1H-pyrazole (0.80 g, 2.220 mmol) in EtOH (30 ml), 10% Pd/C (0.24 g, 2.22 mmol) was and and stirred under H₂ balloon pressure at room temperature for overnight. TLC confirmed completion of reaction. Reaction mixture was filtered through celite bed and filtrate was concentrated under vacuum (0.6 g, 100%). ¹H NMR (400 MHz, Chloroform-d) δ 7.74 (d, J=1.9 Hz, 1H), 7.64 (ddd, J=8.7, 2.4, 0.7 Hz, 1H), 7.48 (d, J=2.3 Hz, 1H), 7.18 (d, J=8.6 Hz, 1H), 6.38 (d, J=1.9 Hz, 1H), 4.34 (hept, J=6.6 Hz, 1H), 1.48 (d, J=6.6 Hz, 6H).

### Intermediate-30:2-(1-(2-fluoroethyl)-1H-pyrazol-5-yl)-4-(trifluoromethyl)phenol

The title compound was prepared similarly as described for Intermediate-29.

¹H NMR (400 MHz, Chloroform-d) δ 7.80 (d, J=1.9 Hz, 1H), 7.72-7.61 (m, 1H), 7.55 (d, J=2.3 Hz, 1H), 7.22 (d, J=8.6 Hz, 1H), 6.48 (d, J=1.8 Hz, 1H), 4.78 (dd, J=46.8, 4.8 Hz, 2H), 4.47-4.33 (m, 2H).

### Intermediate-31: 4′-fluoro-5-(trifluoromethyl)-[1,1′-biphenyl]-2-ol

Nitrogen was purged in a microwave vial containing 2-bromo-4-(trifluoromethyl)phenol (1.00 g, 4.15 mmol), (4-fluorophenyl)boronic acid (0.7 g, 4.98 mmol), Na₂CO₃ (1.1 g, 10.37 mmol) in water (2 ml) for 10 minutes. TetrakistriphenylphosphinePd(0) was added to the reaction mixture and heated at 90° C. for 90 min under microwave irradiation. After completion of reaction it was cooled to room temperature and reaction mass was concentrated under vacuum. The crude was purified by flash column chromatography to obtain title compound as off white solid (0.15 g, 14%). GCMS: m/z 256.04

### Intermediate-32:2-(pyridin-2-yl)-4-(trifluoromethyl)phenol

### Step-1: 2-(2-(benzyloxy)-5-(trifluoromethyl)phenyl)-4,4,5,5-tetramethyl-1,3,2-dioxaborolane

A solution of 1-(benzyloxy)-2-bromo-4-(trifluoromethyl)benzene (5.00 g, 15.10 mmol) in dioxane (60 ml) was purged with N₂ for 10 min. Bis(pinacolato)diboron (5.75 g, 22.65 mmol), PdCl₂(dppf)-CH₂Cl₂Adduct (1.23 g, 1.510 mmol) and potassium acetate (4.15 g, 42.3 mmol) was added to the reaction mixture and heated at 110° C. for 4 h. Reaction progress was monitored by TLC. The solvent was removed under vacuum and the crude was purified by column chromatography to obtain title compound as off white solid. (2.8 g, 49.0%)

### Step-2: 2-(2-(benzyloxy)-5-(trifluoromethyl)phenyl)pyridine

A solution of 2-(2-(benzyloxy)-5-(trifluoromethyl)phenyl)-4,4,5,5-tetramethyl-1,3,2-dioxaborolane (1.2 g, 3.17 mmol), 2-bromopyridine (0.6 g, 3.81 mmol) and Na₂CO₃ (0.841 g, 7.93 mmol) in acetonitrile (10 ml)&water (10 ml) was purged with N₂ for 10 minutes. TetrakistriphenylphosphinePd(0)(0.36 g, 0.31 mmol) was added and heated the reaction mixture at 100° C. for 12 h. After completion of reaction as indicated by TLC, the solvent was removed under vacuum and crude was purified by column chromatography to obtain title compound as off white solid.

### Step-3: 2-(pyridin-2-yl)-4-(trifluoromethyl)phenol

2-(2-(benzyloxy)-5-(trifluoromethyl)phenyl)pyridine (1 g, 3.04 mmol) and ammonium formate (1.92 g, 30.4 mmol), was dissolved in MeOH (30 ml) and 10% Pd/C (0.65 g, 0.607 mmol) was added to it and the mixture was heated at 80° C. for 2 h. The reaction mixture was filtered through celite bed and washed with ethyl acetate. The organic layer was washed with water, dried over Na₂SO₄ and evaporated under vacuum. The crude was purified with column chromatography to obtain title compound as a white solid (0.6 g, 83%). ¹H NMR (400 MHz, Chloroform-d) δ 8.63-8.52 (m, 1H), 8.11-8.04 (m, 1H), 8.05-7.97 (m, 1H), 7.97-7.89 (m, 1H), 7.60-7.53 (m, 1H), 7.40-7.32 (m, 1H), 7.17-7.09 (m, 1H).

### Intermediate-33: 2-(4-cyclopropyl-1H-1,2,3-triazol-1-yl)-4-(trifluoromethyl)phenol

### Step-1: 2-azido-4-(trifluoromethyl)phenol

2-amino-4-(trifluoromethyl)phenol (0.45 g, 2.54 mmol) was dissolved in a mixture of 20 ml water and 20 ml acetonitrile into which HCl (1.930 ml, 63.5 mmol) was added. The solution was cooled to −5° C. and a solution of sodium nitrite (0.26 g, 3.81 mmol) in water was added slowly. The solution was stirred at that temperature for 30 min after which a solution of sodium azide (0.25 g, 3.81 mmol) was added to this mixture. The solution was warmed to room temperature over another 30 min and the reaction mixture was extracted with ethyl acetate. The combined organic layer was dried over Na₂SO₄ and conc under vacuum to obtain title compound as a viscous dark oil (0.50 g, 97%). ¹H NMR (400 MHz, Chloroform-d) δ 7.38-7.31 (m, 2H), 7.06-6.99 (m, 1H), 5.75 (bs, 1H).

### Step-2: 2-(4-cyclopropyl-1H-1,2,3-triazol-1-yl)-4-(trifluoromethyl)phenol

2-azido-4-(trifluoromethyl)phenol (0.50 g, 2.462 mmol), ethynylcyclopropane (0.195 g, 2.95 mmol), and sodium 2-((S)-1,2-dihydroxyethyl)-4-hydroxy-5-oxo-2,5-dihydrofuran-3-olate (0.195 g, 0.985 mmol) was dissolved in a mixture of 5 ml t-Butanol and 5 ml water and copper sulfate pentahydrate (0.123 g, 0.492 mmol) was added to it. After some time ppt was observed and the reaction mixture was stirred for 1 h after which it was dissolved in ethyl acetate and washed with water. The ethyl acetate layer was dried over Na₂SO₄, concentrated under vacuum and the crude product was purified by flash column chromatography to obtain the product as a pale brown solid (0.50 g, 75%). LCMS(ESI): m/z 270.33 (M+H)⁺.

### Intermediate-34: 2-(1H-pyrazol-1-yl)-4-(trifluoromethyl)phenol

### Step 1: 1-(2-(benzyloxy)-5-(trifluoromethyl)phenyl)-1H-pyrazole

1-(benzyloxy)-2-bromo-4-(trifluoromethyl)benzene (0.75 g, 2.265 mmol), 1H-pyrazole (0.185 g, 2.72 mmol), N1,N2-dimethylethane-1,2-diamine (0.122 ml, 1.132 mmol), cesium carbonate (1.62 g, 4.98 mmol) was added to 15 ml degassed dioxane in a sealed tube and copper(I) iodide (0.043 g, 0.226 mmol) was added. The tube was sealed and heated at 120° C. for 12 h. After the solution was cooled, the mixture was passed through a pad of celite and the filtrate was extracted with ethyl acetate. The ethyl acetate layer was washed with brine, dried over Na₂SO₄ and concentrated under vacuum. The crude was purified by flash column chromatography to obtain the product as off white solid (0. 455 g, 63%). LCMS(ESI): m/z 319.40 (M+H)⁺

### Step 2: 2-(1H-pyrazol-1-yl)-4-(trifluoromethyl)phenol

1-(2-(benzyloxy)-5-(trifluoromethyl)phenyl)-1H-pyrazole (0.45 g, 1.414 mmol) was dissolved in MeOH (20 ml) and ammonium formate (2.67 g, 42.4 mmol) was added followed by 10% Pd/C (0.30 g, 0.283 mmol). The mixture was refluxed for 1 h after which it was cooled and filtered through a pad of celite. The filtrate was conc and the crude purified on column to obtain the product as a white solid (0.3 g, 93%). ¹H NMR (400 MHz, Chloroform-d) δ 8.09 (dd, J=2.6, 0.6 Hz, 1H), 7.81-7.77 (m, 1H), 7.67-7.62 (m, 1H), 7.47-4.43 (m, 1H), 7.22-7.18 (m, 1H), 6.59 (t, J=2.6 Hz, 1H).

### Intermediate-35: 2-(2-methylthiazol-4-yl)-4-(trifluoromethyl)phenol

### Step-1: 1-(2-(benzyloxy)-5-(trifluoromethyl)phenyl)-2-bromoethanone

To a solution of 1-(2-(benzyloxy)-5-(trifluoromethyl)phenyl)ethanone (0.20 g, 0.680 mmol) in DCM (15 ml) was added activated molecular sieves followed by phenyl trimethylammonium tribromide (1 g, 2.66 mmol) and stirred at room temperature for 30 min. After completion of reaction as indicated by TLC, reaction mixture was filtered through celite and washed with DCM. The combined organic layer was washed with water, brine, dried over Na₂SO₄ and evaporated under vacuum. The crude product was purified by column chromatography (10% ethyl acetate/petroleum ether) to obtain title compound (0.54 g, 60%). ¹H NMR (400 MHz, Chloroform-d) δ 8.12 (dd, J=2.4, 0.8 Hz, 1H), 7.82-7.73 (m, 1H), 7.52-7.39 (m, 5H), 7.17 (d, J=8.8 Hz, 1H), 5.27 (s, 2H), 4.51 (s, 2H).

### Step-2: 4-(2-(benzyloxy)-5-(trifluoromethyl)phenyl)-2-methylthiazole

To a solution of 1-(2-(benzyloxy)-5-(trifluoromethyl)phenyl)-2-bromoethanone (0.24 g, 0.64 mmol) in ethanol (15 ml) was added thioacetamide (0.048 g, 0.643 mmol) and stirred at 60° C. for 4 h. Reaction mixture then evaporated under vacuum and crude mixture was purified by flash chromatography (10% ethyl acetate/petroleum ether) to obtain title compound (0.14 g, 62%). LCMS(ESI): m/z 350.10 (M+H)⁺

### Step-3: 2-(2-methylthiazol-4-yl)-4-(trifluoromethyl)phenol

To a solution of 4-(2-(benzyloxy)-5-(trifluoromethyl)phenyl)-2-methylthiazole (0.14 g, 0.401 mmol) in MeOH (15 ml) was added ammonium formate (0.025 g, 0.401 mmol), 10% Pd/C (0.043 g, 0.401 mmol) and heated at 60° C. for 1 h. After completion of reaction as indicated by TLC, reaction mixture was filtered through celite and washed with ethyl acetate. The combined organic layer was washed with water, brine, dried over Na₂SO₄ and evaporated under vacuum. The crude product was purified by flash column chromatography (10% ethyl acetate/petroleum ether) to obtain title compound (0.09 g, 99%). LCMS(ESI): m/z 259.95 (M+H)⁺

### Intermediate-36: 2-(2-methyloxazol-4-yl)-4-(trifluoromethyl)phenol

The title compound was prepared by following similar procedure as described for Intermediate-35. LCMS(ESI): m/z 242.93 (M+H)⁺

### Intermediate-37: 2-(2-hydroxy-5-(trifluoromethyl)phenyl)-N,N-dimethylacetamide

### Step-1: 2-allyl-1-(benzyloxy)-4-(trifluoromethyl)benzene

2-allyl-4-(trifluoromethyl)phenol (2.8 g, 13.85 mmol) was dissolved in acetone (50 ml) and cesium carbonate (6.77 g, 20.77 mmol) was added followed by benzyl bromide (2.47 ml, 20.77 mmol) and potassium iodide (0.23 g, 1.385 mmol). The mixture was heated at 50° C. overnight, after which it was filtered and the filtrate was concentrated and purified by flash column chromatography to obtain the product as a pale yellow oil (3 g, 74%). ¹H NMR (400 MHz, Chloroform-d) δ 7.49-7.32 (m, 7H), 6.97 (d, J=8.4 Hz, 1H), 6.12-5.92 (m, 1H), 5.19-5.05 (m, 4H), 3.49 (dd, J=6.6, 1.5 Hz, 2H).

### Step-2: 2-(2-(benzyloxy)-5-(trifluoromethyl)phenyl)acetic acid

2-allyl-1-(benzyloxy)-4-(trifluoromethyl)benzene (2.5 g, 8.55 mmol) was dissolved in a mixture of acetonitrile:water (1:1, 60 ml) and a mixture of sodium periodate (9.15 g, 42.8 mmol) and ruthenium(III) chloride hydrate (0.193 g, 0.855 mmol) was added to it portionwise. The mixture turned dark brown and after 15 min, a large amount of precipitation was observed. The reaction mixture was stirred at room temperature for 1 h and then was passed through a pad of celite. The filtrate was extracted with ethyl acetate and the ethyl acetate layer was washed with sodium thiosulfate solution. The combined organic layer was dried over Na₂SO₄ and evaporated under vacuum to obtain the acid as a pale brown solid (2.5 g, 94%). ¹H NMR (400 MHz, Chloroform-d) δ 7.58-7.32 (m, 7H), 7.00 (d, J=8.6 Hz, 1H), 5.14 (s, 2H), 3.77 (s, 2H).

### Step-3: 2-(2-(benzyloxy)-5-(trifluoromethyl)phenyl)-N,N-dimethylacetamide

2-(2-(benzyloxy)-5-(trifluoromethyl)phenyl)acetic acid (0.66 g, 2.127 mmol), dimethylamine hydrochloride (0.87 g, 10.64 mmol) and HATU (1.618 g, 4.25 mmol) was dissolved in DMF (5 ml) and Hunig's Base (3.34 ml, 19.14 mmol) was added to it dropwise at 0° C. and the reaction allowed to stir overnight at room temperature. The reaction was quenched by the addition of water and then extracted with ethyl acetate. The organic layer was dried over Na₂SO₄ and concentrated under vacuum and the crude was purified by column chromatography to obtain the product as a pale yellow solid (0.50 mg, 70%). ¹H NMR (400 MHz, Chloroform-d) δ 7.54-7.47 (m, 2H), 7.47-7.33 (m, 5H), 6.99 (d, J=9.0 Hz, 1H), 5.12 (s, 2H), 3.72 (s, 2H), 2.96 (s, 3H), 2.83 (s, 3H).

### Step-4: 2-(2-hydroxy-5-(trifluoromethyl)phenyl)-N,N-dimethylacetamide

2-(2-(benzyloxy)-5-(trifluoromethyl)phenyl)-N,N-dimethylacetamide (0.45 g, 1.334 mmol) was dissolved in MeOH (25 ml) and ammonium formate (1.68 g, 26.7 mmol) was added followed by 10% Pd/C (0.28 g, 0.267 mmol). The mixture was refluxed for 1 h and then cooled and the solution filtered through a pad of celite. The filtrate was concentrated under vacuum and the crude was purified by column chromatography to obtain the product as a white solid (0.33 g, 100%). ¹H NMR (400 MHz, Chloroform-d) δ 7.46 (dd, J=8.5, 2.3 Hz, 1H), 7.30 (d, J=2.3 Hz, 1H), 7.06 (d, J=8.5 Hz, 1H), 3.81 (s, 2H), 3.27 (s, 3H), 3.03 (s, 3H).

### Intermediate-38: 2-(1-ethyl-1H-pyrazol-5-yl)-4-(trifluoromethyl)phenol

The title compound was prepared by following procedure as described for Intermediate-29. ¹H NMR (400 MHz, Chloroform-d) δ 7.71-7.56 (m, 2H), 7.50 (d, J=2.3 Hz, 1H), 7.22-7.15 (m, 1H), 6.40 (d, J=2.0 Hz, 1H), 4.10 (q, J=7.2 Hz, 2H), 1.38 (t, J=7.2 Hz, 3H).

### Intermediate-39: 2-(2-fluoroethyl)-4-(trifluoromethyl) phenol

### Step-1: 2-(2-(benzyloxy)-5-(trifluoromethyl)phenyl)ethanol

2-(2-(benzyloxy)-5-(trifluoromethyl)phenyl)acetic acid (1 g, 3.22 mmol) was dissolved in THF (20 ml) and borane-dimethyl sulfide complex (0.918 ml, 9.67 mmol) was added to it dropwise and the solution stirred at room temperature for 1 h. After the starting material was consumed, the reaction mixture was quenched by addition of MeOH and then diluted with ethyl acetate. The ethyl acetate layer was washed with water, dried over Na₂SO₄ and concentrated under vacuum to obtain alcohol as viscous oil (0. 90 g, 94%). ¹H NMR (400 MHz, Chloroform-d) δ 7.50-7.40 (m, 7H), 7.00 (d, J=9.0 Hz, 1H), 5.16 (s, 2H), 3.90 (t, J=6.5 Hz, 2H), 3.01 (t, J=6.5 Hz, 2H).

### Step-2: 1-(benzyloxy)-2-(2-fluoroethyl)-4-(trifluoromethyl)benzene

2-(2-(benzyloxy)-5-(trifluoromethyl)phenyl)ethanol (0.80 g, 2.70 mmol) was dissolved in DCM (10 ml) and DAST (1.070 ml, 8.10 mmol) was added to it. The solution was stirred at room temperature for 1 h. After completion of reaction as indicated by TLC, the reaction mixture was diluted with DCM and washed with 5% sodium bicarbonate solution. The DCM layer was dried over Na₂SO₄ and concentrated and the crude was purified by flash column chromatography to obtain title compound as a white oil (0.31 g, 38%). GCMS: m/z 298.10 (M)⁺; ¹H NMR (400 MHz, Chloroform-d) δ 7.45-7.38 (m, 7H), 7.00 (d, J=9.1 Hz, 1H), 5.16 (s, 2H), 4.74 (t, J=6.4 Hz, 1H), 4.62 (t, J=6.4 Hz, 1H), 3.17 (t, J=6.5 Hz, 1H), 3.11 (t, J=6.4 Hz, 1H).

### Step-3: 2-(2-fluoroethyl)-4-(trifluoromethyl)phenol

1-(benzyloxy)-2-(2-fluoroethyl)-4-(trifluoromethyl)benzene (0.30 g, 1.006 mmol) was dissolved in degassed ethyl acetate (10 ml) and 10% Pd/C (0.107 g, 0.101 mmol) was added to it and the mixture was stirred for 6 h under hydrogen atmosphere by using a gas balloon. The mixture was then passed through a pad of celite and the filtrate was concentrated to obtain the product as a white viscous oil (0.20 g, 96%). GCMS: m/z 206.70 (M−H)⁺

### EXAMPLES

### Example-1: 4-(2-Chloro-4-(trifluoromethyl)phenyl)-2,2-dimethyl-N-(1,2,4-thiadiazol-5-yl) chroman-7-sulfonamide

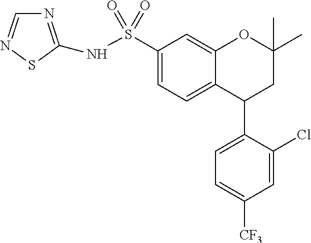 [SMILES: CC1(C)CC(c2ccc(C(F)(F)F)cc2Cl)c2ccc(S(=O)(=O)Nc3ncns3)cc2O1]

To a solution of 1,2,4-thiadiazol-5-amine (0.02 g, 0.20 mmol) in THF (10 ml) was added LiHMDS (1M THF solution, 0.25 ml, 0.25 mmol) at −78° C. under N₂ and stirred for 1 h allowing it to come to room temperature. The reaction mixture was cooled again to −30° C. and perfluorophenyl 4-(2-Chloro-4-(trifluoromethyl)phenyl)-2,2-dimethylchroman-7-sulfonate (0.10 g, 0.17 mmol) (Intermediate-1) in THF was added drop wise and stirred at room temperature for 1 h. The reaction mixture was quenched with 2N HCl and the compound was extracted with ethyl acetate. The combined organic layer was washed with brine, dried over Na₂SO₄, and concentrated under vacuum. The crude product was purified by preparative HPLC to obtain the title compound as white solid (0.008 g, 10%). LCMS(ESI): m/z 503.97 (M+H)⁺; ¹H NMR (400 MHz, Chloroform-d) δ 8.03 (s, 1H), 7.72 (s, 1H), 7.46 (d, J=8.2 Hz, 1H), 7.40 (d, J=1.9 Hz, 1H), 7.31 (dd, J=8.1, 2.0 Hz, 1H), 7.14 (s, 1H), 6.92-6.80 (m, 1H), 4.75-4.85 (m, 1H), 2.22-2.10 (m, 1H), 1.90-1.80 (m, 1H), 1.49 (s, 3H), 1.40 (s, 3H).

Below Example-2 and 3 were prepared by following similar procedure as described in Example-1 using Intermediate-1 and suitable amine.

### Example-2: 4-(2-Chloro-4-(trifluoromethyl)phenyl)-2,2-dimethyl-N-(thiazol-2-yl)chroman-7-sulfonamide

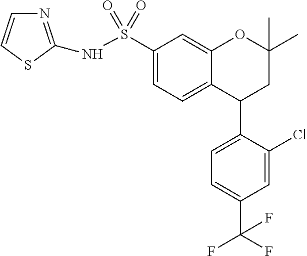 [SMILES: CC1(C)CC(c2ccc(C(F)(F)F)cc2Cl)c2ccc(S(=O)(=O)Nc3nccs3)cc2O1]

Yield: 18%; LCMS(ESI): m/z 503.00 (M+H)⁺; 1H NMR (400 MHz, Chloroform-d) δ 7.71 (s, 1H), 7.42 (m, 2H), 7.37-7.29 (m, 1H), 7.17 (d, J=4.6 Hz, 2H), 6.86-6.80 (m, 1H), 6.54 (d, J=4.6 Hz, 1H), 4.89-4.71 (m, 1H), 2.22-2.10 (m, 1H), 1.92-1.69 (m, 1H), 1.48 (s, 3H), 1.39 (s, 3H).

### Example-3: 4-(2-Chloro-4-(trifluoromethyl)phenyl)-2,2-dimethyl-N-(1,3,4-thiadiazol-2-yl)chroman-7-sulfonamide

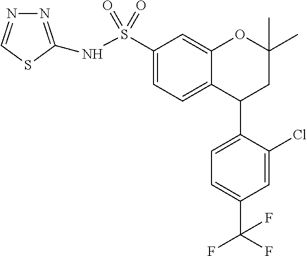 [SMILES: CC1(C)CC(c2ccc(C(F)(F)F)cc2Cl)c2ccc(S(=O)(=O)Nc3nncs3)cc2O1]

Yield: 12%; LCMS(ESI): m/z 504.00 (M+H)⁺; ¹H NMR (400 MHz, Chloroform-d) δ 8.24 (s, 1H), 7.71 (d, J=2.0 Hz, 1H), 7.57-7.42 (m, 2H), 7.34 (dd, J=8.1, 2.0 Hz, 1H), 7.15 (m, 1H), 6.84 (dd, J=8.1, 1.1 Hz, 1H), 4.91-4.72 (m, 1H), 2.26-2.10 (m, 1H), 1.93-1.61 (m, 1H), 1.48 (s, 3H), 1.39 (s, 3H).

Below Example-4 to 6 were prepared by following similar procedure as described in Example-1 using Intermediate-2 and suitable amine.

### Example-4: 4-(2-Methoxy-4-(trifluoromethyl)phenyl)-2,2-dimethyl-N-(1,2,4-thiadiazol-5-yl)chroman-7-sulfonamide

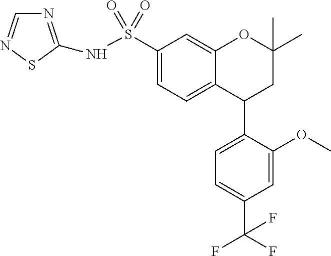 [SMILES: COc1cc(C(F)(F)F)ccc1C1CC(C)(C)Oc2cc(S(=O)(=O)Nc3ncns3)ccc21]

Yield: 17%; LCMS(ESI): m/z 500.04 (M+H)⁺; 1H NMR (400 MHz, Chloroform-d) δ 7.99 (s, 1H), 7.36 (d, J=1.9 Hz, 1H), 7.26 (d, J=2.0 Hz, 1H), 7.22-7.05 (m, 3H), 6.87 (dd, J=8.1, 1.1 Hz, 1H), 4.75-4.65 (m, 1H), 3.88 (s, 3H), 2.20-1.75 (m, 2H), 1.47 (s, 3H), 1.38 (s, 3H).

### Example-5: 4-(2-Methoxy-4-(trifluoromethyl)phenyl)-2,2-dimethyl-N-(thiazol-2-yl) chroman-7-sulfonamide

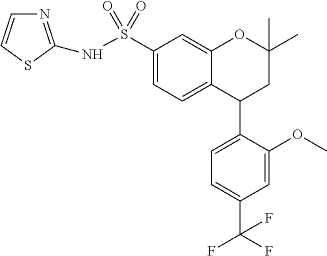 [SMILES: COc1cc(C(F)(F)F)ccc1C1CC(C)(C)Oc2cc(S(=O)(=O)Nc3nccs3)ccc21]

Yield: 12%; LCMS(ESI): m/z 498.81 (M+H)⁺; 1H NMR (400 MHz, Chloroform-d) δ 7.40 (d, J=1.9 Hz, 1H), 7.31 (dd, J=8.1, 2.0 Hz, 1H), 7.23-7.05 (m, 4H), 6.83 (dd, J=8.1, 1.2 Hz, 1H), 6.56 (d, J=4.4 Hz, 1H), 4.75-4.60 (m, 1H), 3.88 (s, 3H), 2.16-1.84 (m, 2H), 1.47 (s, 3H), 1.38 (s, 3H).

### Example-6: 4-(2-Methoxy-4-(trifluoromethyl)phenyl)-2,2-dimethyl-N-(1,3,4-thiadiazol-2-yl)chroman-7-sulfonamide

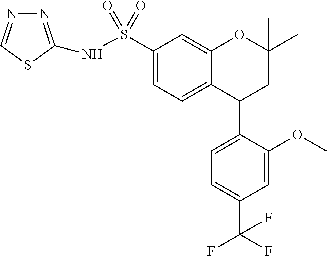 [SMILES: COc1cc(C(F)(F)F)ccc1C1CC(C)(C)Oc2cc(S(=O)(=O)Nc3nncs3)ccc21]

Yield: 15%; LCMS(ESI): m/z 499.81 (M+H)⁺; 1H NMR (400 MHz, Chloroform-d) δ 8.23 (s, 1H), 7.40 (d, J=1.9 Hz, 1H), 7.33-7.29 (m, 1H), 7.20-7.06 (m, 3H), 6.90-6.80 (m, 1H), 4.72-4.67 (m, 1H), 3.88 (s, 3H), 2.12-1.87 (m, 2H), 1.46 (s, 3H), 1.37 (s, 3H).

### Example-7: 2,2-Dimethyl-4-(2-(1,2,3,6-tetrahydropyridin-4-yl)-4-(trifluoromethyl) phenyl)-N-(thiazol-2-yl)chroman-7-sulfonamide hydrochloride

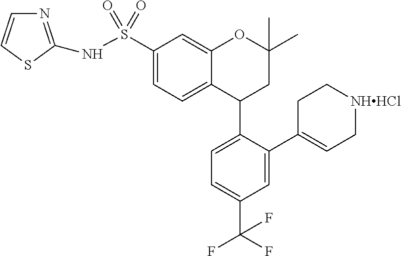

To a solution of thiazol-2-amine (0.011 g, 0.11 mmol) in THF (5 ml) was added LiHMDS (1M in THF, 0.12 ml, 0.12 mmol) at −78° C. under nitrogen and stirred for 1 h allowing it to come to room temperature. The reaction mixture was cooled again at −30° C. and then tert-butyl 4-(2-(2,2-dimethyl-7-((perfluorophenoxy)sulfonyl)chroman-4-yl)-5-(trifluoromethyl) phenyl)-5,6-dihydropyridine-1(2H)-carboxylate (0.06 g, 0.08 mmol) (Intermediate-3) in THF was added. The reaction mixture was then allowed to stir at room temperature for 1 h, quenched with 2N HCl and the product was extracted with ethyl acetate. The combined organic layer was washed with brine, dried over Na₂SO₄, concentrated under vacuum and purified by flash column chromatography. Boc protection was removed by taking this Boc compound in DCM (5 ml) and treated with 2N HCl in diethyl ether (0.20 ml) at room temperature for 15 h. The solvent was removed under vacuum and the solid was triturated with ether to obtain the title compound as hydrochloride salt (0.015 g, 31%). LCMS(ESI): m/z: 549.82 (M+H)⁺; ¹H NMR (400 MHz, DMSO-d6) δ 12.76 (s, 1H), 9.12 (bs, 2H), 7.61 (dd, J=8.5, 2.0 Hz, 1H), 7.54 (d, J=2.0 Hz, 1H), 7.27 (d, J=4.6 Hz, 1H), 7.22 (d, J=8.3 Hz, 1H), 7.18-7.11 (m, 2H), 6.84 (d, J=4.6 Hz, 1H), 6.65 (d, J=8.0 Hz, 1H), 5.79 (m, 1H), 4.43 (m, 1H), 3.75-3.65 (m, 2H), 3.40-3.20 (m, 2H), 2.65-2.55 (m, 2H), 2.06 (d, 1H), 1.98-1.85 (m, 1H), 1.42 (s, 3H), 1.32 (s, 3H).

### Example-8: 2,2-Dimethyl-4-(2-(1,2,3,6-tetrahydropyridin-4-yl)-4-(trifluoromethyl) phenyl)-N-(1,2,4-thiadiazol-5-yl)chroman-7-sulfonamide hydrochloride

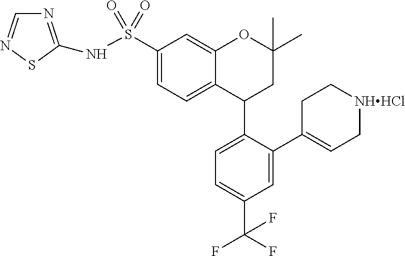

Example-8 was prepared following similar procedure as described in Example-7 using Intermediate-3 and 1,2,4-thiadiazol-5-amine. Yield: 17%; ¹H NMR (400 MHz, DMSO-d₆) δ 9.14 (bs, 2H), 8.48 (s, 1H), 7.65-7.52 (m, 2H), 7.26-7.09 (m, 3H), 6.69 (d, J=8.1 Hz, 1H), 5.79 (m, 1H), 4.55-4.38 (m, 1H), 3.80-3.64 (m, 2H), 2.50-2.43 (m, 4H), 2.09 (m, 1H), 1.94 (m, 1H), 1.43 (s, 3H), 1.33 (s, 3H).

### Example-9:2,2-Dimethyl-4-(2-(piperidin-4-yl)-4-(trifluoromethyl)phenyl)-N-(thiazol-2-yl)chroman-7-sulfonamide hydrochloride

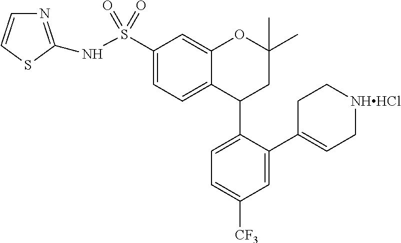

To a solution of Intermediate-7 in DCM (2 ml), 2N HCl in diethyl ether (2 ml, 4.00 mmol) was added at 25° C. and stirred for 15 h. The solvent was removed under vacuum and solid was washed with diethyl ether to obtain the title compound as white solid (0.055 g, 87%). LCMS(ESI): m/z 552.00 (M+H)⁺; ¹H NMR (400 MHz, DMSO-d₆) δ 12.77 (bs, 1H), 8.98 (bs, 1H), 8.64 (bs, 1H), 7.60-7.54 (m, 2H), 7.27 (d, J=4.4 Hz, 1H), 7.20-7.10 (m, 3H), 6.84 (d, J=4.4 Hz, 1H), 6.68 (d, J=8.0 Hz, 1H), 4.65-4.47 (m, 1H), 3.40-3.35 (m, 2H), 3.20-3.00 (m, 2H), 2.14-1.80 (m, 7H), 1.43 (s, 3H), 1.38 (s, 3H).

Below Examples-10 to 15 were prepared by following similar procedure as described in Example-9 using Intermediate-7a, 7b, 8, 8a, 8b and Intermediate-9 respectively.

### Example-10: (R/S)-2,2-Dimethyl-4-(2-(piperidin-4-yl)-4-(trifluoromethyl)phen yl)-N-(thiazol-2-yl)chroman-7-sulfonamide hydrochloride

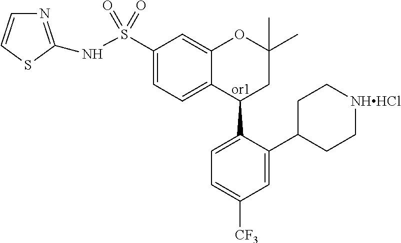

Yield: 42%; LCMS(ESI): m/z 552.00 (M+H)⁺; ¹H NMR (400 MHz, DMSO-d₆) δ 12.78 (bs, 1H), 8.93 (bs, 1H), 8.62 (bs, 1H), 7.60-7.54 (m, 2H), 7.27 (d, J=4.6 Hz, 1H), 7.20-7.10 (m, 3H), 6.84 (d, J=4.6 Hz, 1H), 6.67 (d, J=8.0 Hz, 1H), 4.65-4.47 (m, 1H), 3.40-3.30 (m, 2H), 3.20-3.05 (m, 2H), 2.20-1.91 (m, 7H), 1.43 (s, 3H), 1.38 (s, 3H).

### Example-11: (R/S)-2,2-Dimethyl-4-(2-(piperidin-4-yl)-4-(trifluoromethyl)phenyl)-N-(thiazol-2-yl)chroman-7-sulfonamide hydrochloride

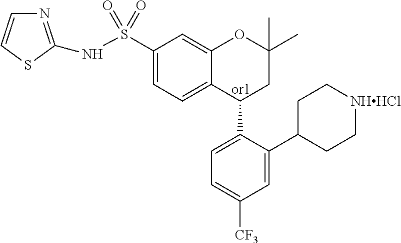

Yield: 43%; LCMS(ESI): m/z 552.10 (M+H)⁺; ¹H NMR (400 MHz, DMSO-d₆) δ 12.77 (bs, 1H), 8.88 (bs, 1H), 8.59 (bs, 1H), 7.60-7.50 (m, 2H), 7.27 (d, J=4.6 Hz, 1H), 7.22-7.12 (m, 3H), 6.84 (d, J=4.6 Hz, 1H), 6.67 (d, J=8.0 Hz, 1H), 4.70-4.47 (m, 1H), 3.40-3.30 (m, 2H), 3.20-3.05 (m, 2H), 2.20-1.86 (m, 7H), 1.43 (s, 3H), 1.38 (s, 3H).

### Example-12: 2,2-Dimethyl-4-(2-(piperidin-4-yl)-4-(trifluoromethyl)phenyl)-N-(1,2,4-thiadiazol-5-yl)chroman-7-sulfonamide hydrochloride

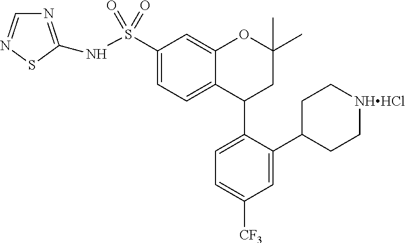

Yield: 31%; LCMS(ESI): m/z 552.82 (M+H)⁺; ¹H NMR (400 MHz, DMSO-d₆) δ 8.88 (bs, 1H), 8.60-8.50 (m, 2H), 7.60-7.50 (m, 2H), 7.37-7.05 (m, 3H), 6.75-6.60 (m, 1H), 4.70-4.55 (m, 1H), 3.39-3.30 (m, 2H), 3.20-3.09 (m, 2H), 2.18-1.78 (m, 7H), 1.44 (s, 3H), 1.39 (s, 3H).

### Example-13: (R/S)-2,2-Dimethyl-4-(2-(piperidin-4-yl)-4-(trifluoromethyl)phenyl)-N-(1,2,4-thiadiazol-5-yl)chroman-7-sulfonamide

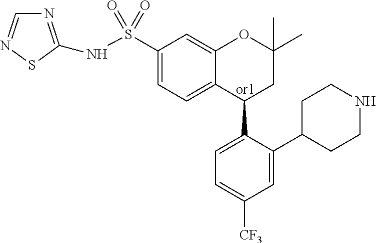 [SMILES: CC1(C)C[C@@H](c2ccc(C(F)(F)F)cc2C2CCNCC2)c2ccc(S(=O)(=O)Nc3ncns3)cc2O1]

Yield: 36%; LCMS(ESI): m/z 553.11 (M+H)⁺; ¹H NMR (400 MHz, DMSO-d₆) δ 7.84 (s, 1H), 7.60-7.45 (m, 2H), 7.20-7.15 (m, 3H), 6.60-6.50 (m, 1H), 4.65-4.50 (m, 1H), 3.40-30 (m, 2H), 3.10-2.95 (m, 2H), 2.18-1.71 (m, 7H), 1.41 (s, 3H), 1.39 (s, 3H).

### Example-14: (R/S)-2,2-Dimethyl-4-(2-(piperidin-4-yl)-4-(trifluoromethyl)phenyl)-N-(1,2,4-thiadiazol-5-yl)chroman-7-sulfonamide

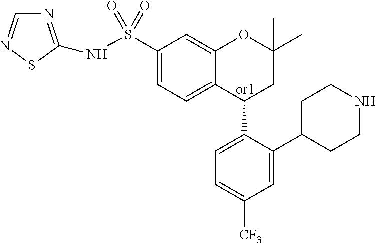 [SMILES: CC1(C)C[C@H](c2ccc(C(F)(F)F)cc2C2CCNCC2)c2ccc(S(=O)(=O)Nc3ncns3)cc2O1]

Yield: 45%; LCMS(ESI): m/z: 553.10 (M+H)⁺; ¹H NMR (400 MHz, DMSO-d₆) δ 7.84 (s, 1H), 7.60-7.50 (m, 2H), 7.20-7.10 (m, 3H), 6.60-6.50 (m, 1H), 4.62-4.55 (m, 1H), 3.39-3.20 (m, 2H), 3.10-2.95 (m, 2H), 2.14-1.76 (m, 7H), 1.42 (s, 3H), 1.39 (s, 3H).

### Example-15: 2,2-Dimethyl-4-(2-(piperidin-4-yl)-4-(trifluoromethyl)phenyl)-N-(1,3,4-thiadiazol-2-yl)chroman-7-sulfonamide hydrochloride

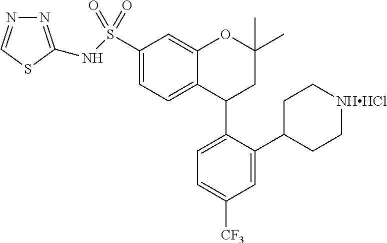

Yield: 32%; LCMS(ESI): m/z: 552.82 (M+H)⁺; ¹H NMR (400 MHz, DMSO-d₆) δ 14.38 (bs, 1H), 8.89 (bs, 1H), 8.77 (s, 1H), 8.59 (bs, 1H), 7.60-7.54 (m, 2H), 7.30-7.01 (m, 3H), 6.69 (d, J=8.1 Hz, 1H), 4.70-4.62 (m, 1H), 3.40-3.35 (m, 2H), 3.20-3.00 (m, 2H), 2.23-1.72 (m, 7H), 1.43 (s, 3H), 1.39 (s, 3H).

### Example-16: 2,2-Dimethyl-4-(2-(1-methylpiperidin-4-yl)-4-(trifluoromethyl) phenyl)-N-(thiazol-2-yl)chroman-7-sulfonamide

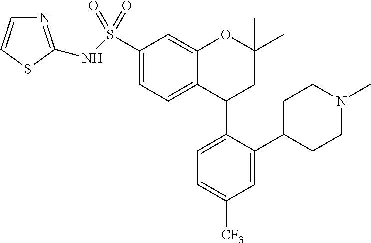 [SMILES: CN1CCC(c2cc(C(F)(F)F)ccc2C2CC(C)(C)Oc3cc(S(=O)(=O)Nc4nccs4)ccc32)CC1]

To a solution of 2,2-dimethyl-4-(2-(piperidin-4-yl)-4-(trifluoromethyl)phenyl)-N-(thiazol-2-yl)chroman-7-sulfonamide hydrochloride (Example-9) (0.030 g, 0.051 mmol) in MeOH (1 ml):DCM (10 ml) was added formaldehyde solution (0.15 ml, 1.530 mmol) and acetic acid (0.15 ml, 2.55 mmol), stirred for 30 minute at ambient temperature, then sodium triacetoxyborohydride (0.054 g, 0.255 mmol) was added portion wise. The reaction mixture was stirred at room temperature for another 30 min. The reaction mixture was poured into water and basified with saturated solution of sodium carbonate and extracted with DCM. The combined organic layers were washed with brine, dried over Na₂SO₄, and concentrated under vacuum. It was washed with diethyl ether to obtain title compound as white solid (0.015 g, 52%). LCMS(ESI): m/z 565.82 (M+H)⁺; ¹H NMR (400 MHz, Chloroform-d) δ 7.65-7.55 (m, 1H), 7.50-7.39 (m, 3H), 7.17-7.10 (m, 1H), 7.07-7.00 (m, 1H), 6.77-6.65 (m, 1H), 6.54-6.65 (m, 1H), 4.60-4.45 (m, 1H), 3.20-3.00 (m, 2H), 2.85-2.60 (m, 2H), 2.36 (s, 3H), 2.18-1.73 (m, 7H), 1.50 (s, 3H), 1.39 (s, 3H).

Below Examples-17 to 20 were prepared by following similar procedure as described in Example-16 using Example-11, 12, 13 and 15 respectively.

### Example-17: (R/S)-2,2-Dimethyl-4-(2-(1-methylpiperidin-4-yl)-4-(trifluoromethyl) phenyl)-N-(thiazol-2-yl)chroman-7-sulfonamide

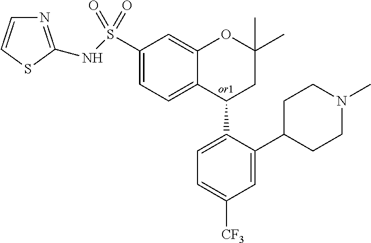 [SMILES: CN1CCC(c2cc(C(F)(F)F)ccc2[C@H]2CC(C)(C)Oc3cc(S(=O)(=O)Nc4nccs4)ccc32)CC1]

Yield: 32%; LCMS(ESI): m/z 565.82 (M+H)⁺; ¹H NMR (400 MHz, DMSO-d₆) δ 7.60-7.48 (m, 3H), 7.40-7.00 (m, 3H), 6.90-6.50 (m, 2H), 4.60-4.40 (m, 1H), 3.60-3.40 (m, 2H), 3.15-2.85 (m, 2H), 2.35 (s, 3H), 2.21-1.57 (m, 7H), 1.42 (s, 3H), 1.38 (s, 3H).

### Example-18: 2,2-Dimethyl-4-(2-(1-methylpiperidin-4-yl)-4-(trifluoromethyl)phenyl)-N-(1,2,4-thiadiazol-5-yl)chroman-7-sulfonamide hydrochloride

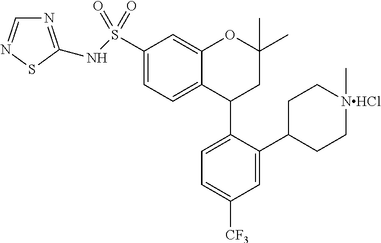

Yield: 71%; LCMS(ESI): m/z 567.17 (M+H)⁺; ¹H NMR (400 MHz, DMSO-d₆) δ 10.24 (bs, 1H), 8.47 (s, 1H), 7.60-7.50 (m, 2H), 7.37-7.04 (m, 3H), 6.85-6.57 (m, 1H), 4.70-4.59 (m, 1H), 3.56-3.30 (m, 4H), 2.78 (s, 3H), 2.16-1.85 (m, 7H), 1.41 (s, 3H), 1.38 (s, 3H).

### Example-19: (R/S)-2,2-Dimethyl-4-(2-(1-methylpiperidin-4-yl)-4-(trifluoromethyl) phenyl)-N-(1,2,4-thiadiazol-5-yl)chroman-7-sulfonamide

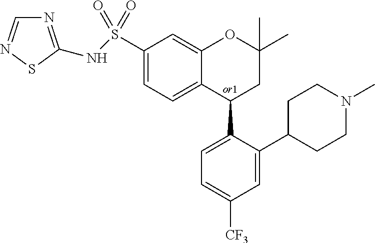 [SMILES: CN1CCC(c2cc(C(F)(F)F)ccc2[C@@H]2CC(C)(C)Oc3cc(S(=O)(=O)Nc4ncns4)ccc32)CC1]

Yield: 25%; LCMS(ESI): m/z 566.70 (M+H)⁺; ¹H NMR (400 MHz, DMSO-d₆) δ 7.84 (s, 1H), 7.60-7.45 (m, 2H), 7.20-7.05 (m, 3H), 6.60-6.50 (m, 1H), 4.60-4.40 (m, 1H), 3.30-3.00 (m, 7H), 2.24-1.66 (m, 7H), 1.42 (s, 3H), 1.38 (s, 3H).

### Example-20: 2,2-Dimethyl-4-(2-(1-methylpiperidin-4-yl)-4-(trifluoromethyl)phenyl)-N-(1,3,4-thiadiazol-2-yl)chroman-7-sulfonamide hydrochloride

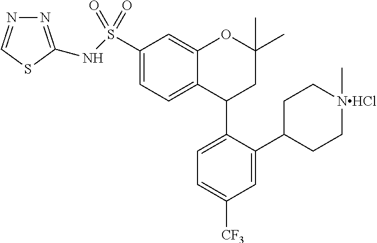

Yield: 73%; LCMS(ESI): m/z 566.82 (M+H)⁺; ¹H NMR (400 MHz, DMSO-d₆) δ 8.78 (s, 1H), 8.00-7.55 (m, 3H), 7.48-7.00 (m, 2H), 6.85-6.65 (m, 1H), 4.65-4.50 (m, 1H), 3.50-3.00 (m, 4H), 2.77 (s, 3H), 2.40-1.60 (m, 7H), 1.44 (s, 3H), 1.39 (s, 3H).

### Example-21: 4-(2-(3-Fluoroazetidin-3-yl)-4-(trifluoromethyl)phenyl)-2,2-dimethyl-N-(thiazol-2-yl)chroman-7-sulfonamide hydrochloride

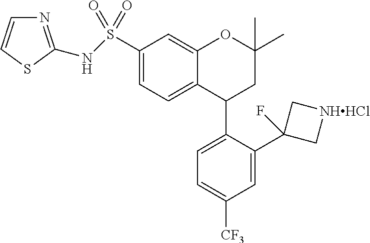

The title compound was prepared following similar procedure as described in Example-9 using Intermediate-5.

LCMS(ESI): m/z 542.02 (M+H)⁺; ¹H NMR (400 MHz, DMSO-d₆) δ 12.77 (bs, 1H), 9.93 (bs, 1H), 9.29 (bs, 1H), 7.93 (s, 1H), 7.81 (d, J=8.3 Hz, 1H), 7.40-7.25 (m, 2H), 7.20-7.05 (m, 2H), 6.85 (d, J=4.5 Hz, 1H), 6.67 (d, J=8.1 Hz, 1H), 5.25-4.47 (m, 4H), 4.06-4.00 (m, 1H), 2.11 (dd, J=13.5, 5.6 Hz, 1H), 1.97 (d, J=12.9 Hz, 1H), 1.43 (s, 3H), 1.34 (s, 3H).

### Example-22: 4-(2-(3-Fluoroazetidin-3-yl)-4-(trifluoromethyl)phenyl)-2,2-dimethyl-N-(1,2,4-thiadiazol-5-yl)chroman-7-sulfonamide hydrochloride

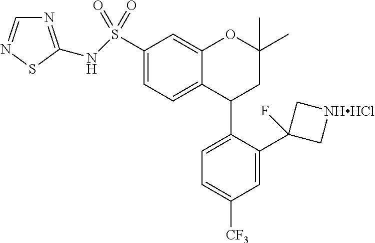

The title compound was prepared by following similar procedure as described in Example-9 using Intermediate-5 and 1,2,4-thiadiazol-5-amine. LCMS(ESI): m/z 543.69 (M+H)⁺; ¹H NMR (400 MHz, DMSO-d₆) δ 9.93 (bs, 1H), 9.27 (bs, 1H), 8.48 (s, 1H), 7.93 (s, 1H), 7.81 (d, J=8.2 Hz, 1H), 7.35 (d, J=8.1 Hz, 1H), 7.22-7.12 (m, 2H), 6.70 (d, J=8.0 Hz, 1H), 5.10-4.50 (m, 4H), 4.04 (dd, J=12.4, 5.5 Hz, 1H), 2.12 (dd, J=13.6, 5.6 Hz, 1H), 2.01-1.89 (m, 1H), 1.44 (s, 3H), 1.29 (s, 3H).

Below Examples-23 &24 were prepared by following similar procedure as described in Example-1 using Intermediate-10 and suitable amine.

### Example-23: 4-(2-Chloro-4-(trifluoromethyl)phenyl)-N-(1,2,4-thiadiazol-5-yl)chroman-7-sulfonamide

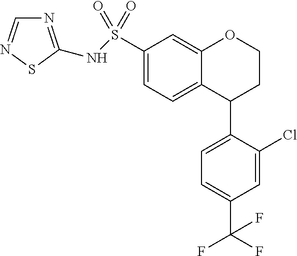 [SMILES: O=S(=O)(Nc1ncns1)c1ccc2c(c1)OCCC2c1ccc(C(F)(F)F)cc1Cl]

Yield: 18%; LCMS(ESI): m/z 476.05 (M+H)⁺; 1H NMR (400 MHz, Chloroform-d) δ 8.05 (s, 1H), 7.82-7.67 (m, 1H), 7.54-7.29 (m, 3H), 7.10-6.86 (m, 2H), 4.74 (t, J=6.1 Hz, 1H), 4.36-4.06 (m, 2H), 2.47-2.31 (m, 1H), 2.20-2.10 (m, 1H).

### Example-24: 4-(2-Chloro-4-(trifluoromethyl)phenyl)-N-(1,3,4-thiadiazol-2-yl)chroman-7-sulfonamide

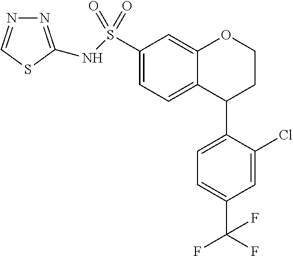 [SMILES: O=S(=O)(Nc1nncs1)c1ccc2c(c1)OCCC2c1ccc(C(F)(F)F)cc1Cl]

Yield: 24%; LCMS(ESI): m/z 475.98 (M+H)⁺; 1H NMR (400 MHz, Chloroform-d) δ 8.28 (s, 1H), 7.80-7.67 (m, 1H), 7.49 (d, J=1.9 Hz, 1H), 7.46-7.38 (m, 2H), 6.99 (d, J=8.1 Hz, 1H), 6.95-6.90 (m, 1H), 4.73 (t, J=6.0 Hz, 1H), 4.35-4.06 (m, 2H), 2.50-2.30 (m, 1H), 2.24-2.05 (m, 1H).

### Example-25: 4-(2-Chloro-4-(trifluoromethyl)phenyl)-N-(thiazol-2-yl)chroman-7-sulfonamide

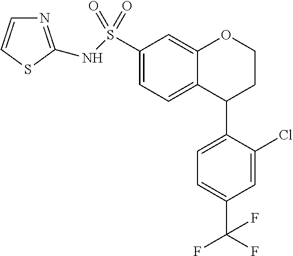 [SMILES: O=S(=O)(Nc1nccs1)c1ccc2c(c1)OCCC2c1ccc(C(F)(F)F)cc1Cl]

To a solution of thiazol-2-amine (0.058 g, 0.580 mmol) and perfluorophenyl 4-(2-chloro-4-(trifluoromethyl)phenyl)chroman-7-sulfonate (0.270 g, 0.483 mmol) (Intermediate-10) in THF (10 ml) was added LiHMDS (1M in THF, 0.725 ml, 0.725 mmol) at 0° C. under nitrogen atm. The reaction mixture was stirred for 1 h allowing it to come at room temperature. After completion of reaction as indicated by TLC, reaction mixture was poured into 2N HCl solution and extracted with ethyl acetate. The combined organic layer was washed with brine, dried over Na₂SO₄, and concentrated under vacuum. The crude product was purified by preparative HPLC to obtain (R/S)-4-(2-chloro-4-(trifluoromethyl)phenyl)-N-(thiazol-2-yl)chroman-7-sulfonamide. LCMS(ESI): m/z 475.05 (M+H)⁺; 1H NMR (400 MHz, Chloroform-d) δ 7.75-7.68 (m, 1H), 7.48 (d, J=1.9 Hz, 1H), 7.47-7.42 (m, 1H), 7.41-7.35 (m, 1H), 7.19 (d, J=4.6 Hz, 1H), 7.00 (d, J=8.1 Hz, 1H), 6.94-6.87 (m, 1H), 6.57 (d, J=4.5 Hz, 1H), 4.73 (t, J=6.0 Hz, 1H), 4.33-4.09 (m, 2H), 2.45-2.28 (m, 1H), 2.19-2.04 (m, 1H).

The enantiomers of Example-25 were separated using chiral preparative HPLC (Column: Chiral pak IA; Mobile phase: (n-hexane:EtOH, 9:1+0.1% DEA &0.1% TFA,):(ETOH:DCM, 1:1) 7:3, affording Example-26 (retention time 5.21 min) and Example-27 (retention time 7.12 min).

### Example-26: (R/S)-4-(2-Chloro-4-(trifluoromethyl)phenyl)-N-(thiazol-2-yl)chroman-7-sulfonamide

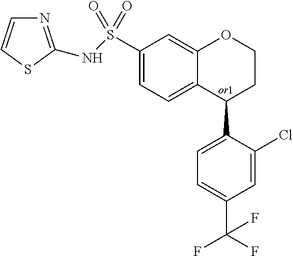 [SMILES: O=S(=O)(Nc1nccs1)c1ccc2c(c1)OCC[C@H]2c1ccc(C(F)(F)F)cc1Cl]

Yield: 8%; LCMS(ESI): m/z 474.92 (M+H)⁺; ¹H NMR (400 MHz, DMSO-d₆) δ 12.79 (bs, 1H), 7.98-7.90 (m, 1H), 7.70-7.61 (m, 1H), 7.33-7.13 (m, 4H), 6.96-6.81 (m, 2H), 4.72 (t, J=6.4 Hz, 1H), 4.32-4.21 (m, 1H), 4.18-4.07 (m, 1H), 2.35-2.24 (m, 1H), 2.13-2.03 (m, 1H).

### Example-27:—(R/S)-4-(2-Chloro-4-(trifluoromethyl)phenyl)-N-(thiazol-2-yl)chroman-7-sulfonamide

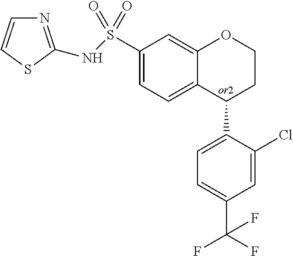 [SMILES: O=S(=O)(Nc1nccs1)c1ccc2c(c1)OCC[C@@H]2c1ccc(C(F)(F)F)cc1Cl]

Yield: 9%; LCMS(ESI): m/z 474.92 (M+H)⁺; ¹H NMR (400 MHz, DMSO-d₆) δ 12.79 (bs, 1H), 7.95 (d, J=1.9 Hz, 1H), 7.70-7.60 (m, 1H), 7.34-7.15 (m, 4H), 6.94-6.81 (m, 2H), 4.72 (t, J=6.4 Hz, 1H), 4.32-4.21 (m, 1H), 4.18-4.07 (m, 1H), 2.37-2.23 (m, 1H), 2.14-2.03 (m, 1H). Below Examples-28 to 30 were prepared by following similar procedure as described in Example-7 using Intermediate-11.

### Example-28: 4-(2-(1,2,3,6-Tetrahydropyridin-4-yl)-4-(trifluoromethyl)phenyl)-N-(thiazol-2-yl)chroman-7-sulfonamide hydrochloride

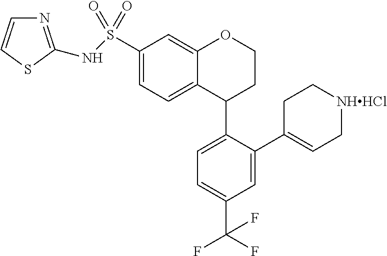

Yield: 10%; LCMS(ESI): m/z 521.92 (M+H)⁺; ¹H NMR (400 MHz, DMSO-d₆) δ 12.78 (bs, 1H), 9.19-8.99 (m, 2H), 7.65-7.57 (m, 1H), 7.51 (d, J=2.0 Hz, 1H), 7.27 (d, J=4.6 Hz, 1H), 7.24-7.12 (m, 3H), 6.84 (d, J=4.6 Hz, 1H), 6.71 (d, J=8.0 Hz, 1H), 5.80 (bs, 1H), 4.55 (t, J=9.4, 5.7 Hz, 1H), 4.41-4.22 (m, 2H), 3.81-3.64 (m, 2H), 3.37-3.30 (m, 2H), 2.60-2.50 (m, 2H), 2.24-1.98 (m, 2H).

### Example-29: 4-(2-(1,2,3,6-Tetrahydropyridin-4-yl)-4-(trifluoromethyl)phenyl)-N-(1,2,4-thiadiazol-5-yl)chroman-7-sulfonamide hydrochloride

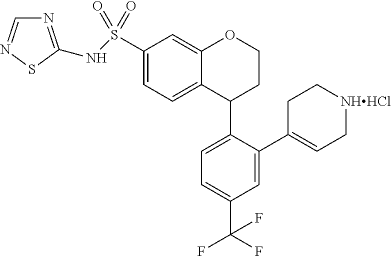

Yield: 16%; LCMS(ESI): m/z 523.00 (M+H)⁺; ¹H NMR (400 MHz, DMSO-d₆) δ 9.20-8.99 (m, 2H), 8.48 (s, 1H), 7.69-7.58 (m, 1H), 7.52 (d, J=2.0 Hz, 1H), 7.29-7.11 (m, 3H), 6.75 (d, J=8.6 Hz, 1H), 5.79 (d, J=3.6 Hz, 1H), 4.57 (t, 1H), 4.42-4.24 (m, 2H), 3.77-3.64 (m, 2H), 3.58-3.44 (m, 2H), 2.64-2.56 (m, 2H), 2.27-1.97 (m, 2H).

### Example-30: 4-(2-(1,2,3,6-Tetrahydropyridin-4-yl)-4-(trifluoromethyl)phenyl)-N-(1,3,4-thiadiazol-2-yl)chroman-7-sulfonamide hydrochloride

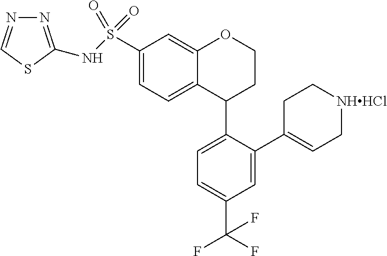

Yield: 11%; LCMS(ESI): m/z 523.00 (M+H)⁺; ¹H NMR (400 MHz, DMSO-d₆) δ 14.38 (bs, 1H), 9.16 (bs, 2H), 8.77 (s, 1H), 7.78-7.56 (m, 1H), 7.51 (d, J=2.0 Hz, 1H), 7.27-7.09 (m, 3H), 6.73 (d, J=8.3 Hz, 1H), 5.78 (d, J=11.0 Hz, 1H), 4.68-4.50 (m, 1H), 4.48-4.21 (m, 2H), 3.84-3.64 (m, 2H), 3.40-3.34 (m, 2H), 2.66-2.55 (m, 2H), 2.22-1.95 (m, 2H).

### Example-31: 4-(2-(3-Fluoroazetidin-3-yl)-4-(trifluoromethyl)phenyl)-N-(thiazol-2-yl) chroman-7-sulfonamide hydrochloride

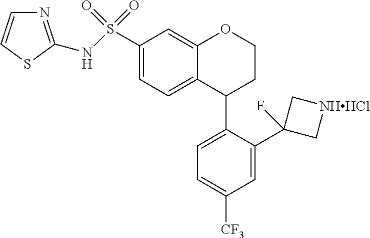

The title compound was prepared by following the similar procedure as described in Example-9 using Intermediate-5 and 7-((tert-butyldimethylsilyl)oxy)-2H-chromen-4-yl trifluoromethanesulfonate (Prepared by following similar procedure as described in EP 2179994 from 7-hydroxychroman-4-one).

Yield: 16%; LCMS(ESI): m/z 514.00 (M+H)⁺; ¹H NMR (400 MHz, DMSO-d₆) δ 12.79 (bs, 1H), 10.00 (s, 1H), 9.18 (s, 1H), 7.90 (s, 1H), 7.80 (d, J=8.3 Hz, 1H), 7.36-7.26 (m, 2H), 7.23-7.14 (m, 2H), 6.84 (d, J=4.4 Hz, 1H), 6.68 (d, J=8.1 Hz, 1H), 5.00-4.80 (m, 2H), 4.80-4.65 (m, 2H), 4.40-4.36 (m, 2H), 4.30-4.15 (m, 1H), 2.27-2.18 (m, 1H), 2.14-2.00 (m, 1H)

### Example-32: 4-(2-(1-Methyl-1H-pyrazol-5-yl)-4-(trifluoromethyl)phenyl)-N-(thiazol-2-yl)chroman-7-sulfonamide

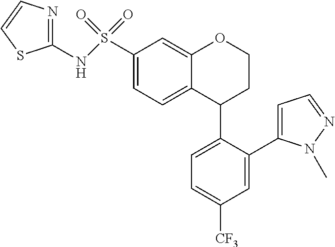 [SMILES: Cn1nccc1-c1cc(C(F)(F)F)ccc1C1CCOc2cc(S(=O)(=O)Nc3nccs3)ccc21]

### Step-1: 4-(2-Chloro-4-(trifluoromethyl)phenyl)-N-(2,4-dimethoxybenzyl)-N-(thiazol-2-yl)chroman-7-sulfonamide

To a solution of perfluorophenyl 4-(2-chloro-4-(trifluoromethyl)phenyl)chroman-7-sulfonate (0.20 g, 0.358 mmol)(Intermediate-10) and N-(2,4-dimethoxybenzyl)thiazol-2-amine (prepared as described in WO2013/63459) (0.1 g, 0.394 mmol) in THF (10 ml) was added LiHMDS (0.090 g, 0.537 mmol) at 0° C. under nitrogen dropwise. The reaction mixture was stirred for 2 h at ambient temperature. The reaction mixture was quenched with water and extracted with ethyl acetate. The combined organic layer was washed with brine, dried over Na₂SO₄ and concentrated under vacuum. The crude was purified by flash column chromatography to obtain title compound as off white solid (0.1 g, 44%). ¹H NMR (400 MHz, Chloroform-d) δ 7.76-7.68 (m, 1H), 7.50-7.38 (m, 3H), 7.31-7.17 (m, 2H), 7.06-6.94 (m, 2H), 6.89 (dd, J=8.2, 1.0 Hz, 1H), 6.44-6.34 (m, 2H), 5.11 (s, 2H), 4.73 (t, J=6.1 Hz, 1H), 4.27 (ddd, J=10.3, 6.7, 3.2 Hz, 1H), 4.15 (ddd, J=11.3, 8.3, 2.9 Hz, 1H), 3.77 (d, J=6.3 Hz, 6H), 2.45-2.33 (m, 1H), 2.16-2.07 (m, 1H).

### Step-2: N-(2,4-Dimethoxybenzyl)-4-(2-(1-methyl-1H-pyrazol-5-yl)-4-(trifluoromethyl) phenyl)-N-(thiazol-2-yl)chroman-7-sulfonamide

Nitrogen was purged in a microwave vial containing 4-(2-chloro-4-(trifluoromethyl)phenyl)-N-(2,4-dimethoxybenzyl)-N-(thiazol-2-yl)chroman-7-sulfonamide (0.10 g, 0.160 mmol) and 1-methyl-5-(4,4,5,5-tetramethyl-1,3,2-dioxaborolan-2-yl)-1H-pyrazole (0.066 g, 0.320 mmol) in dioxane (8 ml) and water (1 ml), for 10 minutes. AMPHOS (0.011 g, 0.016 mmol) and K₃PO₄.3H₂O (0.85 g, 0.400 mmol) were added to the reaction mixture and heated at 110° C. for 15 min. under microwave irradiation. After completion of reaction as indicated by TLC, the solvent was removed under vacuum and the crude mixture was purified by flash column chromatography obtain title compound as off white solid. LCMS(ESI): m/z 671.12 (M+H)⁺;

### Step-3: 4-(2-(1-Methyl-1H-pyrazol-5-yl)-4-(trifluoromethyl)phenyl)-N-(thiazol-2-yl)chroman-7-sulfonamide

To a solution of N-(2,4-Dimethoxybenzyl)-4-(2-(1-methyl-1H-pyrazol-5-yl)-4-(trifluoromethyl)phenyl)-N-(thiazol-2-yl)chroman-7-sulfonamide (0.060 g, 0.089 mmol) in DCM (10 ml) was added TFA (0.138 ml, 1.789 mmol) at room temperature and stirred for 1 h. TLC shown completion of reaction. After completion of reaction, the mixture was evaporated to dryness under reduced pressure to get crude which was purified by column chromatography (50% ethyl acetate/petroleum ether). (0.025 g, 53%). LCMS(ESI): m/z 520.94 (M+H)⁺; ¹H NMR (400 MHz, DMSO-d₆) δ 7.82-7.72 (m, 2H), 7.55 (d, J=1.9 Hz, 1H), 7.32-7.11 (m, 4H), 6.89-6.79 (m, 2H), 6.47 (d, J=1.9 Hz, 1H), 4.23 (ddd, J=9.6, 5.4, 3.4 Hz, 1H), 4.11 (dd, J=10.3, 7.3 Hz, 2H), 3.70 (s, 3H), 2.18-1.93 (m, 2H).

### Example-33: 4-(2-Chloro-4-(trifluoromethyl)phenoxy)-N-(thiazol-2-yl)chroman-7-sulfonamide

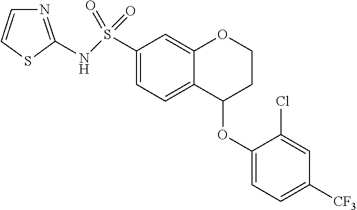 [SMILES: O=S(=O)(Nc1nccs1)c1ccc2c(c1)OCCC2Oc1ccc(C(F)(F)F)cc1Cl]

To a solution of thiazol-2-amine (0.016 g, 0.16 mmol) and perfluorophenyl 4-(2-chloro-4-(trifluoromethyl)phenoxy)chroman-7-sulfonate (Intermediate-13) (0.09 g, 0.16 mmol) in THF (3 ml) was added LiHMDS (1M in THF, 0.17 ml, 0.17 mmol) at 0° C. under nitrogen. The reaction mixture was stirred for 2 h allowing it to come to room temperature, then quenched with 2N HCl and the compound was extracted with dichloromethane. The combined organic layer was washed with brine, dried over Na₂SO₄, and concentrated under vacuum. The crude product was then purified by preparative HPLC to obtain the title compound as white solid (0.007 g, 9%). LCMS(ESI): m/z 490.99 (M+H)⁺; ¹H NMR (400 MHz, Chloroform-d) δ 7.74-7.68 (m, 1H), 7.57-7.42 (m, 3H), 7.35 (d, J=8.1 Hz, 1H), 7.22-7.13 (m, 2H), 6.57 (d, J=4.6 Hz, 1H), 5.44 (t, J=3.8 Hz, 1H), 4.45-4.35 (m, 2H), 2.35-2.17 (m, 2H).

### Example-34: (R)-4-(2-Chloro-4-(trifluoromethyl)phenoxy)-N-(thiazol-2-yl)chroman-7-sulfonamide

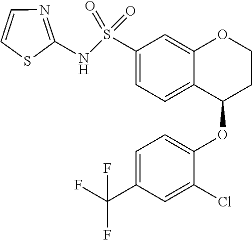 [SMILES: O=S(=O)(Nc1nccs1)c1ccc2c(c1)OCC[C@H]2Oc1ccc(C(F)(F)F)cc1Cl]

To a solution of thiazol-2-amine (0.059 g, 0.588 mmol) and (R)-perfluorophenyl 4-(2-chloro-4-(trifluoromethyl)phenoxy)chroman-7-sulfonate (Intermediate-13a) (0.26 g, 0.452 mmol) in THF (5 ml) was added LiHMDS (1M, in THF, 0.588 ml, 0.588 mmol) at 0° C. under nitrogen. The reaction mixture was stirred for 4 h room temperature then poured into aqueous 2N HCl solution and extracted with ethyl acetate. The combined organic layers were washed with brine, dried over Na₂SO₄, and concentrated under vacuum. The crude product was purified by column chromatography to obtain title compound as white solid (0.070 g, 31%). LCMS(ESI): m/z 490.86 (M+H)⁺; ¹H NMR (400 MHz, Chloroform-d) δ 7.74-7.68 (m, 1H), 7.58-7.44 (m, 3H), 7.36 (d, J=8.0 Hz, 1H), 7.21-7.15 (m, 2H), 6.58 (d, J=4.5 Hz, 1H), 5.44 (t, J=3.8 Hz, 1H), 4.45-4.30 (m, 2H), 2.43-2.18 (m, 2H).

Following Example-35 to 38 were prepared by following similar procedure as described in Example-34 using commercially available different phenols.

### Example-35: (R)-4-(2-Cyano-4-(trifluoromethyl)phenoxy)-N-(thiazol-2-yl)chroman-7-sulfonamide

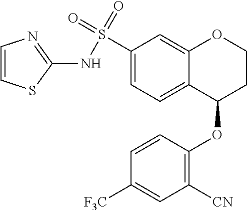 [SMILES: N#Cc1cc(C(F)(F)F)ccc1O[C@@H]1CCOc2cc(S(=O)(=O)Nc3nccs3)ccc21]

LCMS(ESI): m/z 481.96 (M+H)⁺; ¹H NMR (400 MHz, DMSO-d₆) δ 12.84 (bs, 1H), 8.29 (d, J=2.4 Hz, 1H), 8.11 (dd, J=9.2, 2.4 Hz, 1H), 7.82 (d, J=9.0 Hz, 1H), 7.54 (d, J=8.1 Hz, 1H), 7.35 (dd, J=8.1, 1.9 Hz, 1H), 7.28 (d, J=4.6 Hz, 1H), 7.24 (d, J=1.8 Hz, 1H), 6.86 (d, J=4.6 Hz, 1H), 5.95 (t, J=4.0 Hz, 1H), 4.38 (dt, J=11.4, 4.2 Hz, 1H), 4.23 (td, J=10.9, 2.9 Hz, 1H), 2.35-2.15 (m, 2H).

### Example-36: (R)-4-(2,4-Dichlorophenoxy)-N-(thiazol-2-yl)chroman-7-sulfonamide

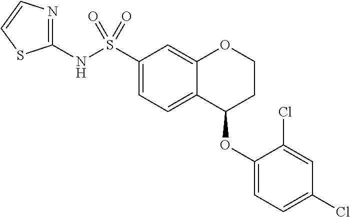 [SMILES: O=S(=O)(Nc1nccs1)c1ccc2c(c1)OCC[C@H]2Oc1ccc(Cl)cc1Cl]

LCMS(ESI): m/z 456.90 (M+H)⁺; ¹H NMR (400 MHz, DMSO-d₆) δ 12.85 (bs, 1H), 7.61 (d, J=2.5 Hz, 1H), 7.53 (d, J=9.0 Hz, 1H), 7.49-7.41 (m, 2H), 7.31 (dd, J=8.0, 1.8 Hz, 1H), 7.28 (d, J=4.6 Hz, 1H), 7.22 (d, J=1.8 Hz, 1H), 6.85 (d, J=4.6 Hz, 1H), 5.67-5.60 (m, 1H), 4.41-4.32 (m, 1H), 4.28-4.23 (m, 1H), 2.24-2.05 (m, 2H).

### Example-37: (R)-4-(3,4-Dichlorophenoxy)-N-(thiazol-2-yl)chroman-7-sulfonamide

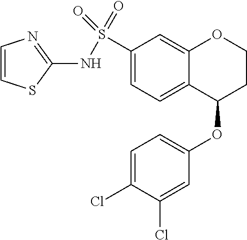 [SMILES: O=S(=O)(Nc1nccs1)c1ccc2c(c1)OCC[C@H]2Oc1ccc(Cl)c(Cl)c1]

LCMS(ESI): m/z 456.96 (M+H)⁺; ¹H NMR (400 MHz, DMSO-d₆) δ 12.84 (bs, 1H), 7.58 (d, J=8.9 Hz, 1H), 7.48 (dd, J=5.5, 2.6 Hz, 2H), 7.36-7.27 (m, 2H), 7.21 (d, J=1.8 Hz, 1H), 7.14 (dd, J=9.0, 2.9 Hz, 1H), 6.86 (d, J=4.6 Hz, 1H), 5.64 (s, 1H), 4.42-4.14 (m, 2H), 2.28-2.07 (m, 2H).

### Example-38: (R)—N-(Thiazol-2-yl)-4-(2,4,6-trifluorophenoxy)chroman-7-sulfonamide

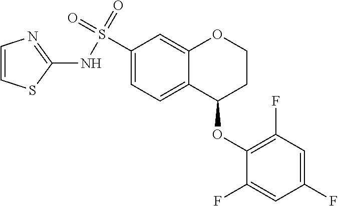 [SMILES: O=S(=O)(Nc1nccs1)c1ccc2c(c1)OCC[C@H]2Oc1c(F)cc(F)cc1F]

LCMS(ESI): m/z 443.11 (M+H)⁺; ¹H NMR (400 MHz, DMSO-d6) δ 12.84 (bs, 1H), 7.39-7.26 (m, 5H), 7.21 (d, J=1.3 Hz, 1H), 6.85 (d, J=4.6 Hz, 1H), 5.15 (s, 1H), 4.45-4.27 (m, 2H), 2.19-2.09 (m, 2H).

Below Example-39 & 40 were prepared by following similar procedure as described in Example-33/34 using Intermediate-13 and 13a respectively and 1,2,4-thiadiazol-5-amine.

### Example-39: 4-(2-Chloro-4-(trifluoromethyl)phenoxy)-N-(1,2,4-thiadiazol-5-yl)chroman-7-sulfonamide

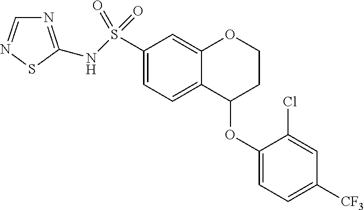 [SMILES: O=S(=O)(Nc1ncns1)c1ccc2c(c1)OCCC2Oc1ccc(C(F)(F)F)cc1Cl]

Yield 11%; LCMS(ESI): m/z 491.92 (M+H)⁺; ¹H NMR (400 MHz, Chloroform-d) δ 8.03 (s, 1H), 7.70 (dd, J=8.1, 2.3 Hz, 1H), 7.60-7.36 (m, 4H), 7.27-7.15 (m, 1H), 5.49-5.41 (m, 1H), 4.52-4.34 (m, 2H), 2.29 (m, 2H).

### Example-40: (R)-4-(2-Chloro-4-(trifluoromethyl)phenoxy)-N-(1,2,4-thiadiazol-5-yl) chroman-7-sulfonamide

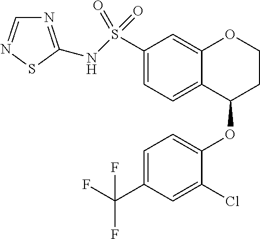 [SMILES: O=S(=O)(Nc1ncns1)c1ccc2c(c1)OCC[C@H]2Oc1ccc(C(F)(F)F)cc1Cl]

Yield 30%, LCMS(ESI): m/z 491.94 (M+H)⁺; ¹H NMR (400 MHz, DMSO-d₆) δ 8.50 (s, 1H), 7.89 (d, J=2.2 Hz, 1H), 7.80-7.67 (m, 2H), 7.55 (d, J=8.1 Hz, 1H), 7.35 (dd, J=8.0, 1.9 Hz, 1H), 7.24 (d, J=1.8 Hz, 1H), 5.85 (t, J=3.8 Hz, 1H), 4.38 (dt, J=11.3, 4.2 Hz, 1H), 4.24 (td, J=11.0, 2.9 Hz, 1H), 2.32-2.10 (m, 2H).

### Example-41: (R)-4-(2-(1-Methyl-1H-pyrazol-5-yl)-4-(trifluoromethyl)phenoxy)-N-(thiazol-2-yl)chroman-7-sulfonamide

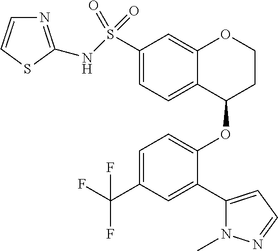 [SMILES: Cn1nccc1-c1cc(C(F)(F)F)ccc1O[C@@H]1CCOc2cc(S(=O)(=O)Nc3nccs3)ccc21]

To a solution of thiazol-2-amine (0.78 g, 0.778 mmol) and (R)-perfluorophenyl 4-(2-(1-methyl-1H-pyrazol-5-yl)-4-(trifluoromethyl)phenoxy)chroman-7-sulfonate (Intermediate-14a) (0.210 g, 0.338 mmol) in THF (15 ml), LiHMDS (1M in THF, 0.508 ml, 0.508 mmol) was added at 0° C. under nitrogen dropwise. The reaction mixture was stirred for 4 h allowing it to come at room temperature. The reaction mixture was poured into aqueous 2N HCl solution and extracted with ethyl acetate. The combined organic layer was washed with brine, dried over Na₂SO₄, and concentrated under vacuum. The crude product was purified by column chromatography to obtain title compound as white solid. (0.105 g, 57%); LCMS(ESI): m/z 536.69 (M+H)⁺; ¹H NMR (400 MHz, DMSO-d₆) δ 12.82 (bs, 1H), 7.89 (dd, J=8.8, 2.4 Hz, 1H), 7.74 (d, J=8.8 Hz, 1H), 7.63 (d, J=2.4 Hz, 1H), 7.42-7.32 (m, 2H), 7.32-7.22 (m, 2H), 7.15 (d, J=1.8 Hz, 1H), 6.85 (d, J=4.6 Hz, 1H), 6.21 (d, J=1.9 Hz, 1H), 5.73 (t, J=3.9 Hz, 1H), 4.26 (dt, J=11.0, 4.0 Hz, 1H), 3.87 (td, J=11.1, 2.8 Hz, 1H), 3.50 (s, 3H), 2.25-1.97 (m, 2H).

### Example-42: (S)-4-(2-(1-Methyl-1H-pyrazol-5-yl)-4-(trifluoromethyl)phenoxy)-N-(thiazol-2-yl)chroman-7-sulfonamide

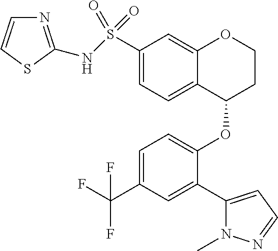 [SMILES: Cn1nccc1-c1cc(C(F)(F)F)ccc1O[C@H]1CCOc2cc(S(=O)(=O)Nc3nccs3)ccc21]

The title compound was prepared similarly as described in Example-41 using Intermediate-14b and 2-amino-thiazole

Yield: 44%; LCMS(ESI): m/z 536.69 (M+H)⁺; ¹H NMR (400 MHz, DMSO-d₆) δ 12.82 (s, 1H), 7.89 (dd, J=8.8, 2.4 Hz, 1H), 7.74 (d, J=8.8 Hz, 1H), 7.64 (d, J=2.4 Hz, 1H), 7.43-7.33 (m, 2H), 7.31-7.23 (m, 2H), 7.15 (d, J=1.8 Hz, 1H), 6.86 (d, J=4.6 Hz, 1H), 6.21 (d, J=1.9 Hz, 1H), 5.74 (t, J=3.9 Hz, 1H), 4.26 (dt, J=11.3, 4.1 Hz, 1H), 3.87 (td, J=11.0, 2.8 Hz, 1H), 3.50 (s, 3H), 2.25-2.04 (m, 2H).

Below Examples 43 to 48 were prepared similarly as described in Example-41 using (R)-perfluorophenyl 4-(2-bromo-4-(trifluoromethyl)phenoxy)chroman-7-sulfonate (Step-1, Intermediate-14a) and commercially available boronic acid or boronate ester.

### Example-43: (R)-4-(2-(Pyridin-3-yl)-4-(trifluoromethyl)phenoxy)-N-(thiazol-2-yl)chroman-7-sulfonamide

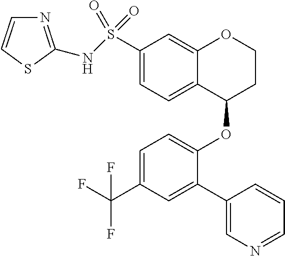 [SMILES: O=S(=O)(Nc1nccs1)c1ccc2c(c1)OCC[C@H]2Oc1ccc(C(F)(F)F)cc1-c1cccnc1]

LCMS(ESI): m/z 534 (M+H)⁺; ¹H NMR (400 MHz, DMSO-d₆) δ 12.83 (bs, 1H), 8.63 (d, J=2.3 Hz, 1H), 8.49 (dd, J=4.9, 1.7 Hz, 1H), 7.90-7.79 (m, 2H), 7.77-7.69 (m, 2H), 7.45-7.34 (m, 2H), 7.31-7.22 (m, 2H), 7.14 (d, J=1.9 Hz, 1H), 6.85 (d, J=4.6 Hz, 1H), 5.76 (t, J=4.2 Hz, 1H), 4.28 (dd, J=11.2, 4.5 Hz, 1H), 4.02-3.93 (m, 1H), 2.28-2.07 (m, 2H).

### Example-44: (R)-4-(2-(Pyridin-4-yl)-4-(trifluoromethyl)phenoxy)-N-(thiazol-2-yl)chroman-7-sulfonamide

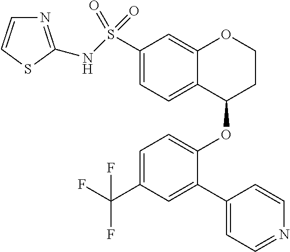 [SMILES: O=S(=O)(Nc1nccs1)c1ccc2c(c1)OCC[C@H]2Oc1ccc(C(F)(F)F)cc1-c1ccncc1]

LCMS(ESI): m/z 534.0 (M+H)⁺; ¹H NMR (400 MHz, DMSO-d₆) δ 12.82 (bs, 1H), 8.59-8.49 (m, 2H), 7.90-7.83 (m, 1H), 7.79-7.69 (m, 2H), 7.46-7.37 (m, 3H), 7.31-7.24 (m, 2H), 7.14 (d, J=1.8 Hz, 1H), 6.85 (d, J=4.6 Hz, 1H), 5.89-5.65 (m, 1H), 4.29 (d, J=11.4 Hz, 1H), 4.17-3.91 (m, 1H), 2.35-2.01 (m, 2H).

### Example-45: (R)-4-(2-(5-Fluoropyridin-2-yl)-4-(trifluoromethyl)phenoxy)-N-(thiazol-2-yl)chroman-7-sulfonamide

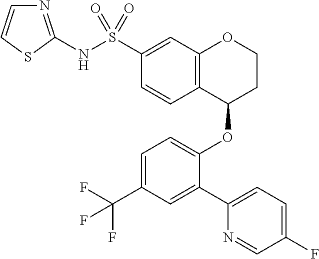 [SMILES: O=S(=O)(Nc1nccs1)c1ccc2c(c1)OCC[C@H]2Oc1ccc(C(F)(F)F)cc1-c1ccc(F)cn1]

LCMS(ESI): m/z 552.0 (M+H)⁺; ¹H NMR (400 MHz, DMSO-d₆) δ 12.81 (bs, 1H), 8.56 (d, J=1.7 Hz, 1H), 8.40 (d, J=4.8 Hz, 1H), 7.92 (dd, J=8.8, 2.4 Hz, 1H), 7.79-7.69 (m, 2H), 7.44-7.20 (m, 4H), 7.12 (d, J=1.8 Hz, 1H), 6.85 (d, J=4.6 Hz, 1H), 5.77 (t, J=4.0 Hz, 1H), 4.29 (dt, J=11.1, 4.3 Hz, 1H), 3.95 (td, J=10.9, 2.7 Hz, 1H), 2.29-2.05 (m, 2H).

### Example-46: (R)-4-(2-(6-Methylpyridin-3-yl)-4-(trifluoromethyl)phenoxy)-N-(thiazol-2-yl)chroman-7-sulfonamide

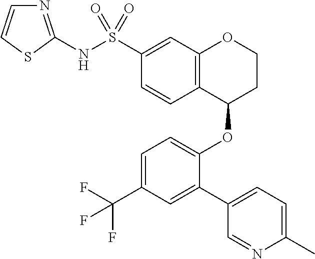 [SMILES: Cc1ccc(-c2cc(C(F)(F)F)ccc2O[C@@H]2CCOc3cc(S(=O)(=O)Nc4nccs4)ccc32)cn1]

LCMS(ESI): m/z 548.02 (M+H)⁺; ¹H NMR (400 MHz, DMSO-d₆) δ 12.82 (bs, 1H), 8.51 (d, J=2.4 Hz, 1H), 7.80 (d, J=2.4 Hz, 1H), 7.76-7.66 (m, 3H), 7.43 (d, J=8.0 Hz, 1H), 7.30-7.25 (m, 2H), 7.23 (d, J=8.1 Hz, 1H), 7.15 (d, J=1.9 Hz, 1H), 6.85 (d, J=4.6 Hz, 1H), 5.77 (s, 1H), 4.29 (dt, J=11.1, 4.2 Hz, 1H), 3.99 (d, J=2.8 Hz, 1H), 2.45 (s, 3H), 2.32-2.08 (m, 2H).

### Example-47: (R)-4-(2-(2-Methylpyrimidin-5-yl)-4-(trifluoromethyl)phenoxy)-N-(thiazol-2-yl)chroman-7-sulfonamide

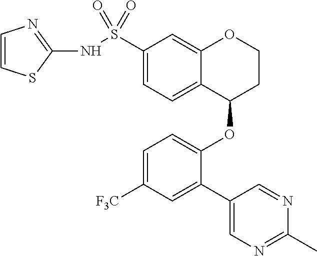 [SMILES: Cc1ncc(-c2cc(C(F)(F)F)ccc2O[C@@H]2CCOc3cc(S(=O)(=O)Nc4nccs4)ccc32)cn1]

LCMS(ESI): m/z 548.99 (M+H)⁺; ¹H NMR (400 MHz, DMSO-d₆) δ 12.82 (bs, 1H), 8.77 (s, 2H), 7.89-7.81 (m, 2H), 7.71 (d, J=8.7 Hz, 1H), 7.47 (d, J=8.1 Hz, 1H), 7.31-7.25 (m, 2H), 7.17 (d, J=1.8 Hz, 1H), 6.86 (d, J=4.6 Hz, 1H), 5.81 (t, J=4.2 Hz, 1H), 4.30 (dt, J=11.4, 4.3 Hz, 1H), 4.02-3.96 (m, 1H), 2.60 (s, 3H), 2.37-2.07 (m, 2H).

### Example-48: (R)-4-(2-(1-Methyl-1H-pyrazol-4-yl)-4-(trifluoromethyl)phenoxy)-N-(thiazol-2-yl)chroman-7-sulfonamide

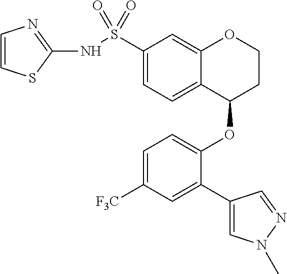 [SMILES: Cn1cc(-c2cc(C(F)(F)F)ccc2O[C@@H]2CCOc3cc(S(=O)(=O)Nc4nccs4)ccc32)cn1]

LCMS(ESI): m/z 536.82 (M+H)⁺; ¹H NMR (400 MHz, DMSO-d₆) δ 8.03 (s, 1H), 7.90 (d, J=2.0 Hz, 1H), 7.79 (d, J=0.8 Hz, 1H), 7.61-7.54 (m, 2H), 7.48 (d, J=8.1 Hz, 1H), 7.33 (dd, J=8.0, 1.8 Hz, 1H), 7.30-7.23 (m, 2H), 6.85 (d, J=4.6 Hz, 1H), 5.84 (t, J=4.4 Hz, 1H), 4.34 (dt, J=9.3, 4.4 Hz, 1H), 4.24-4.12 (m, 1H), 3.74 (s, 3H), 2.38-2.15 (m, 2H).

Below Examples 49 to 60 were prepared by following similar procedure as described in Example-34 using different phenols which were prepared as shown in Intermediate preparation.

### Example-49: (R)-4-(2-Ethyl-4-(trifluoromethyl)phenoxy)-N-(thiazol-2-yl)chroman-7-sulfonamide

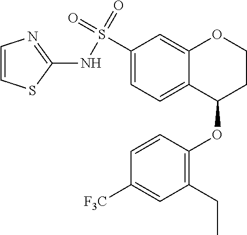 [SMILES: CCc1cc(C(F)(F)F)ccc1O[C@@H]1CCOc2cc(S(=O)(=O)Nc3nccs3)ccc21]

LCMS(ESI): m/z 484.99 (M+H)⁺; ¹H NMR (400 MHz, DMSO-d₆) δ 7.59 (dd, J=8.7, 2.4 Hz, 1H), 7.53-7.44 (m, 3H), 7.35-7.26 (m, 2H), 7.23 (d, J=1.8 Hz, 1H), 6.85 (d, J=4.6 Hz, 1H), 5.73 (t, J=4.1 Hz, 1H), 4.37 (dt, J=11.2, 4.3 Hz, 1H), 4.21 (td, J=10.9, 2.8 Hz, 1H), 2.58-2.52 (m, 2H), 2.34-2.12 (m, 2H), 1.04 (t, J=7.5 Hz, 3H).

### Example-50: (R)-4-(2-Isopropyl-4-(trifluoromethyl)phenoxy)-N-(thiazol-2-yl)chroman-7-sulfonamide

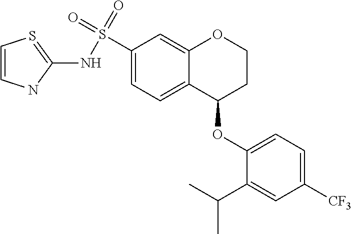

LCMS(ESI): m/z 499.17 (M+H)⁺; ¹H NMR (400 MHz, DMSO-d₆) δ 7.60-7.56 (m, 1H), 7.50-7.45 (m, 2H), 7.42 (d, J=8.1 Hz, 1H), 7.30 (dd, J=8.0, 1.8 Hz, 1H), 7.22 (d, J=1.7 Hz, 1H), 6.79 (d, J=4.5 Hz, 1H), 5.73 (t, 1H), 4.37 (dt, J=11.2, 4.2 Hz, 1H), 4.20 (td, J=10.9, 2.9 Hz, 1H), 3.15 (p, J=6.8 Hz, 1H), 2.34-2.13 (m, 2H), 1.12 (d, J=6.9 Hz, 3H), 1.05 (d, J=6.9 Hz, 3H).

### Example-51: (R)-4-(2-Cyclopropyl-4-(trifluoromethyl)phenoxy)-N-(thiazol-2-yl)chroman-7-sulfonamide

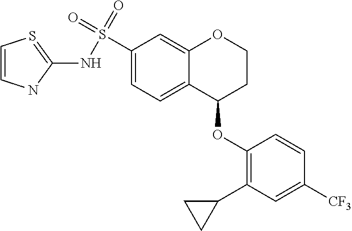

LCMS(ESI): m/z 497.17 (M+H)⁺; ¹H NMR (400 MHz, DMSO-d₆) δ 12.83 (bs, 1H), 7.54 (dd, J=8.9, 2.3 Hz, 1H), 7.50-7.44 (m, 2H), 7.32 (dd, J=8.0, 1.9 Hz, 1H), 7.28 (d, J=4.6 Hz, 1H), 7.22 (d, J=1.8 Hz, 1H), 7.13 (d, J=2.3 Hz, 1H), 6.85 (d, J=4.6 Hz, 1H), 5.73 (t, J=4.0 Hz, 1H), 4.41-4.32 (m, 1H), 4.26 (m, 1H), 2.29-2.12 (m, 2H), 2.07-1.95 (m, 1H), 0.92-0.78 (m, 2H), 0.62 (dd, J=6.6, 4.1 Hz, 2H).

### Example-52: (R)-4-(2-(1-Isopropyl-1H-pyrazol-5-yl)-4-(trifluoromethyl)phenoxy)-N-(thiazol-2-yl)chroman-7-sulfonamide

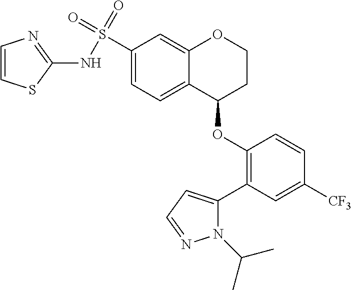 [SMILES: CC(C)n1nccc1-c1cc(C(F)(F)F)ccc1O[C@@H]1CCOc2cc(S(=O)(=O)Nc3nccs3)ccc21]

LCMS(ESI): m/z 564.82 (M+H)⁺; ¹H NMR (400 MHz, DMSO-d₆) δ 12.82 (bs, 1H), 7.90 (dd, J=8.9, 2.4 Hz, 1H), 7.76 (d, J=8.8 Hz, 1H), 7.57 (d, J=2.4 Hz, 1H), 7.45 (d, J=1.8 Hz, 1H), 7.38 (d, J=8.1 Hz, 1H), 7.31-7.23 (m, 2H), 7.14 (d, J=1.8 Hz, 1H), 6.85 (d, J=4.6 Hz, 1H), 6.15 (d, J=1.8 Hz, 1H), 5.78 (s, 1H), 4.25-4.20 (m, 1H), 4.07-3.98 (m, 1H), 3.85-3.72 (m, 1H), 2.20-2.12 (m, 1H), 2.08-2.01 (m, 1H), 1.10 (d, J=6.5 Hz, 3H), 0.97 (d, J=6.5 Hz, 3H).

### Example-53: (R)-4-(2-(1-(2-Fluoroethyl)-1H-pyrazol-5-yl)-4-(trifluoromethyl)phenoxy)-N-(thiazol-2-yl)chroman-7-sulfonamide

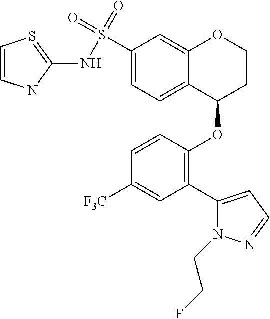

LCMS(ESI): m/z 568.95 (M+H)⁺; ¹H NMR (400 MHz, DMSO-d₆) δ 12.80 (bs, 1H), 7.89 (dd, J=8.9, 2.5 Hz, 1H), 7.73 (d, J=8.9 Hz, 1H), 7.58 (d, J=2.4 Hz, 1H), 7.49 (d, J=1.8 Hz, 1H), 7.37 (d, J=8.1 Hz, 1H), 7.30-7.23 (m, 2H), 7.14 (d, J=1.8 Hz, 1H), 6.85 (d, J=4.6 Hz, 1H), 6.23 (d, J=1.8 Hz, 1H), 5.72 (t, 1H), 4.82-4.72 (m, 1H), 4.62 (q, J=4.7, 4.2 Hz, 1H), 4.50 (q, J=4.6, 4.1 Hz, 1H), 4.30-3.95 (m, 2H), 3.92-3.83 (m, 1H), 2.21-2.04 (m, 2H),

### Example-54: (R)-4-((4′-Fluoro-5-(trifluoromethyl)-[1,1′-biphenyl]-2-yl)oxy)-N-(thiazol-2-yl)chroman-7-sulfonamide

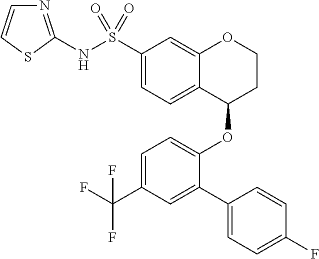 [SMILES: O=S(=O)(Nc1nccs1)c1ccc2c(c1)OCC[C@H]2Oc1ccc(C(F)(F)F)cc1-c1ccc(F)cc1]

LCMS(ESI): m/z 551.0 (M+H)⁺; ¹H NMR (400 MHz, DMSO-d₆) δ 12.82 (bs, 1H), 7.78 (dd, J=8.6, 2.2 Hz, 1H), 7.69 (d, J=8.8 Hz, 1H), 7.60 (d, J=2.4 Hz, 1H), 7.48-7.34 (m, 3H), 7.30-7.23 (m, 2H), 7.20-7.11 (m, 3H), 6.85 (d, J=4.6 Hz, 1H), 5.70 (t, J=4.0 Hz, 1H), 4.36-4.19 (m, 1H), 4.02-3.92 (m, 1H), 2.27-2.02 (m, 2H).

### Example-55: (R)-4-(2-(Pyridin-2-yl)-4-(trifluoromethyl)phenoxy)-N-(thiazol-2-yl)chroman-7-sulfonamide

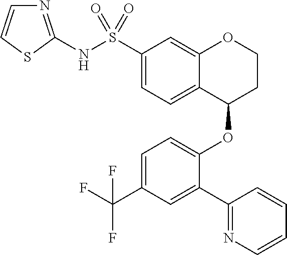 [SMILES: O=S(=O)(Nc1nccs1)c1ccc2c(c1)OCC[C@H]2Oc1ccc(C(F)(F)F)cc1-c1ccccn1]

LCMS(ESI): m/z 534.04 (M+H)⁺; ¹H NMR (400 MHz, DMSO-d₆) δ 12.81 (s, 1H), 8.65 (dt, J=4.8, 1.5 Hz, 1H), 8.05 (d, J=2.5 Hz, 1H), 7.84 (dd, J=8.9, 2.6 Hz, 1H), 7.77-7.63 (m, 3H), 7.47 (d, J=8.1 Hz, 1H), 7.38-7.25 (m, 3H), 7.18 (d, J=1.8 Hz, 1H), 6.86 (d, J=4.6 Hz, 1H), 5.83 (t, J=4.2 Hz, 1H), 4.31 (dd, J=10.5, 5.2 Hz, 1H), 4.15-4.06 (m, 1H), 2.29-2.15 (m, 2H).

### Example-56: (R)-4-(2-(4-Cyclopropyl-1H-1,2,3-triazol-1-yl)-4-(trifluoromethyl)phenoxy)-N-(thiazol-2-yl)chroman-7-sulfonamide

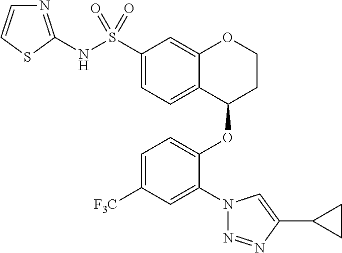 [SMILES: O=S(=O)(Nc1nccs1)c1ccc2c(c1)OCC[C@H]2Oc1ccc(C(F)(F)F)cc1-n1cc(C2CC2)nn1]

LCMS(ESI): m/z 564.06 (M+H)⁺; ¹H NMR (400 MHz, DMSO-d6) δ 12.82 (bs, 1H), 8.03-7.93 (m, 3H), 7.86 (d, J=8.6 Hz, 1H), 7.47 (d, J=8.1 Hz, 1H), 7.32-7.25 (m, 2H), 7.19 (d, J=1.8 Hz, 1H), 6.86 (d, J=4.6 Hz, 1H), 5.84 (t, J=4.3 Hz, 1H), 4.34-4.26 (m, 1H), 4.11-3.96 (m, 1H), 2.36-2.20 (m, 1H), 2.20-2.08 (m, 1H), 1.98-1.91 (m, 1H), 0.90-0.85 (m, 2H), 0.65-0.55 (m, 2H).

### Example-57: (R)-4-(2-(1H-Pyrazol-1-yl)-4-(trifluoromethyl)phenoxy)-N-(thiazol-2-yl)chroman-7-sulfonamide

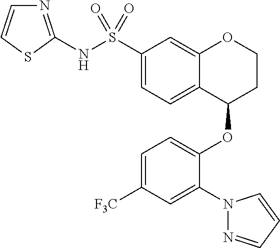 [SMILES: O=S(=O)(Nc1nccs1)c1ccc2c(c1)OCC[C@H]2Oc1ccc(C(F)(F)F)cc1-n1cccn1]

LCMS(ESI): m/z 522.82 (M+H)⁺; ¹H NMR (400 MHz, DMSO-d6) δ 12.83 (bs, 1H), 8.08-8.03 (m, 1H), 7.98 (d, J=1.9 Hz, 1H), 7.82-7.79 (m, 2H), 7.71 (d, J=1.8 Hz, 1H), 7.51 (d, J=8.1 Hz, 1H), 7.33-7.26 (m, 2H), 7.20 (d, J=1.8 Hz, 1H), 6.86 (d, J=4.6 Hz, 1H), 6.43 (dd, J=2.5, 1.8 Hz, 1H), 5.93-5.76 (m, 1H), 4.36-4.25 (m, 1H), 4.12-4.03 (m, 1H), 2.32-2.12 (m, 2H).

### Example-58:—(R)-4-(2-(2-Methylthiazol-4-yl)-4-(trifluoromethyl)phenoxy)-N-(thiazol-2-yl)chroman-7-sulfonamide

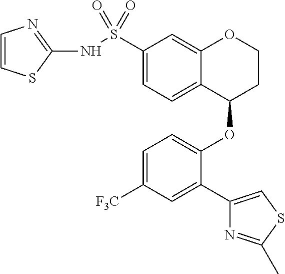 [SMILES: Cc1nc(-c2cc(C(F)(F)F)ccc2O[C@@H]2CCOc3cc(S(=O)(=O)Nc4nccs4)ccc32)cs1]

LCMS(ESI): m/z 553.82 (M+H)⁺; ¹H NMR (400 MHz, DMSO-d₆) δ 8.48 (d, J=2.4 Hz, 1H), 7.78-7.64 (m, 3H), 7.52 (d, J=8.1 Hz, 1H), 7.33 (dd, J=8.1, 1.8 Hz, 1H), 7.29 (d, J=4.6 Hz, 1H), 7.25 (d, J=1.9 Hz, 1H), 6.86 (d, J=4.6 Hz, 1H), 5.94 (t, J=4.2 Hz, 1H), 4.42-4.30 (m, 1H), 4.27-4.14 (m, 1H), 2.70 (s, 3H), 2.37-2.18 (m, 2H).

### Example-59: (R)-4-(2-(2-Methyloxazol-4-yl)-4-(trifluoromethyl)phenoxy)-N-(thiazol-2-yl)chroman-7-sulfonamide

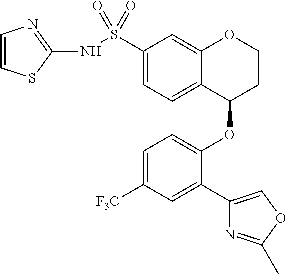 [SMILES: Cc1nc(-c2cc(C(F)(F)F)ccc2O[C@@H]2CCOc3cc(S(=O)(=O)Nc4nccs4)ccc32)co1]

LCMS(ESI): m/z 537.98 (M+H)⁺; ¹H NMR (400 MHz, DMSO-d₆) δ 12.84 (bs, 1H), 8.26 (d, J=2.4 Hz, 1H), 7.82 (s, 1H), 7.74 (dd, J=8.9, 2.5 Hz, 1H), 7.63 (d, J=8.8 Hz, 1H), 7.51 (d, J=8.1 Hz, 1H), 7.35 (dd, J=8.0, 1.9 Hz, 1H), 7.29 (d, J=4.6 Hz, 1H), 7.26 (d, J=1.8 Hz, 1H), 6.87 (d, J=4.6 Hz, 1H), 5.94 (t, J=4.4 Hz, 1H), 4.43-4.14 (m, 2H), 2.45 (s, 3H), 2.38-2.20 (m, 2H).

### Example-60: (R)—N,N-Dimethyl-2-(2-((7-(N-(thiazol-2-yl)sulfamoyl)chroman-4-yl)oxy)-5-(trifluoromethyl)phenyl)acetamide

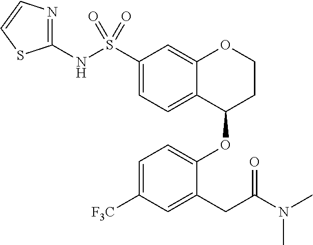 [SMILES: CN(C)C(=O)Cc1cc(C(F)(F)F)ccc1O[C@@H]1CCOc2cc(S(=O)(=O)Nc3nccs3)ccc21]

LCMS(ESI): m/z 542.32 (M+H)⁺; 1H NMR (400 MHz, DMSO-d6) δ 12.85 (s, 1H), 7.63 (dd, J=8.7, 2.4 Hz, 1H), 7.53 (d, J=2.4 Hz, 1H), 7.42 (dd, J=8.4, 3.5 Hz, 2H), 7.34-7.26 (m, 2H), 7.22 (d, J=1.8 Hz, 1H), 6.86 (d, J=4.6 Hz, 1H), 5.69 (t, J=4.0 Hz, 1H), 4.30 (dd, J=10.2, 5.3 Hz, 1H), 4.15 (dd, J=10.9, 2.8 Hz, 1H), 3.56 (s, 2H), 2.66 (s, 3H), 2.63 (s, 3H), 2.27-2.16 (m, 1H), 2.13 (d, J=3.7 Hz, 1H).

### Example-61: (R)-2-(2-((7-(N-(Thiazol-2-yl)sulfamoyl)chroman-4-yl)oxy)-5-(trifluoromethyl) phenyl)pyridine 1-oxide

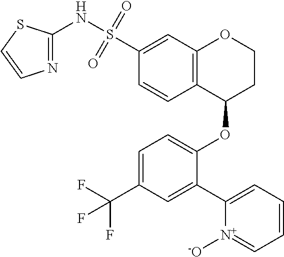

### Step-1: (R)-Perfluorophenyl 4-(2-(pyridin-2-yl)-4-(trifluoromethyl)phenoxy)chroman-7-sulfonate

The title compound was prepared by following similar procedure as described in Intermediate-13a using 2-(pyridin-2-yl)-4-(trifluoromethyl)phenol. LCMS(ESI): m/z 618.08 (M+H)⁺.

### Step-2: (R)-2-(2-((7-((Perfluorophenoxy)sulfonyl)chroman-4-yl)oxy)-5-(trifluoromethyl)phenyl)pyridine 1-oxide

To a solution of (R)-perfluorophenyl 4-(2-(pyridin-2-yl)-4-(trifluoromethyl)phenoxy)chroman-7-sulfonate (step-4 of Example-55) (0.23 g, 0.372 mmol) in CHCl₃ (10 ml), m-CPBA (0.14 g, 0.819 mmol) was added and stirred for 1 h at room temperature. After completion of reaction, Solvent was removed under vacuum and crude reaction mixture was purified by flash column chromatography to obtain title compound as white solid (0.17 g, 72.0%). LCMS(ESI): m/z 633.97 (M+H)⁺.

### Step-3: (R)-2-(2-((7-(N-(Thiazol-2-yl)sulfamoyl)chroman-4-yl)oxy)-5-(trifluoromethyl) phenyl)pyridine 1-oxide

The title compound was prepared by following similar procedure as described in Example-34 using Step-2 intermediate and 2-amino thiazole.

Yield: 33%; LCMS(ESI): m/z 549.98 (M+H)⁺; ¹H NMR (400 MHz, DMSO-d₆) δ 12.80 (bs, 1H), 8.35-8.27 (m, 1H), 7.92-7.84 (m, 1H), 7.76-7.64 (m, 2H), 7.51 (d, J=8.1 Hz, 1H), 7.46-7.33 (m, 2H), 7.31-7.19 (m, 3H), 7.11 (d, J=1.8 Hz, 1H), 6.84 (d, J=4.6 Hz, 1H), 5.72 (t, J=4.6 Hz, 1H), 4.26 (dt, J=9.7, 4.0 Hz, 1H), 4.12-4.01 (m, 1H), 2.30-2.20 (m, 2H), 2.11-2.05 (m, 1H).

### Example-62: 4-(2-(1,2,3,6-Tetrahydropyridin-4-yl)-4-(trifluoromethyl)phenoxy)-N-(thiazol-2-yl)chroman-7-sulfonamide

### Step-1: tert-Butyl 4-(2-((7-((perfluorophenoxy)sulfonyl)chroman-4-yl)oxy)-5-(trifluoro methyl)phenyl)-5,6-dihydropyridine-1(2H)-carboxylate

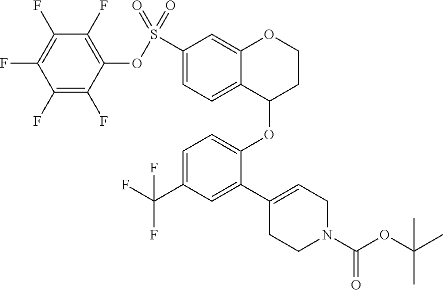 [SMILES: CC(C)(C)OC(=O)N1CC=C(c2cc(C(F)(F)F)ccc2OC2CCOc3cc(S(=O)(=O)Oc4c(F)c(F)c(F)c(F)c4F)ccc32)CC1]

A solution of perfluorophenyl 4-(2-chloro-4-(trifluoromethyl)phenoxy)chroman-7-sulfonate) (Intermediate-13)(0.15 g, 0.26 mmol), tert-butyl 4-(4,4,5,5-tetramethyl-1,3,2-dioxaborolan-2-yl)-5,6-dihydropyridine-1(2H)-carboxylate (0.16 g, 0.52 mmol), potassium phosphate (0.14 g, 0.78 mmol) in 1,4-dioxane (1 ml) & water (0.30 ml) was purged with N₂ for 15 min. Bis(di-tert-butyl(4-dimethylaminophenyl)phosphine) dichloropalladium(II) (0.007 g, 0.010 mmol) was added to the reaction mixture and heated under microwave irradiation for 1 h at 110° C. The reaction mixture was cooled to room temperature, diluted with ethyl acetate, washed with water, brine, dried over Na₂SO₄ and evaporated under vacuum. Purification was done by flash column chromatography to obtain the title compound as off-white solid (0.14 g, 77%). LCMS(ESI): m/z 744.1 (M+Na)⁺; ¹H NMR (400 MHz, Chloroform-d) δ 7.57 (dd, J=8.4, 2.6 Hz, 1H), 7.53 (d, J=1.9 Hz, 1H), 7.48-7.43 (m, 2H), 7.35 (d, J=8.1 Hz, 1H), 7.14 (d, J=8.6 Hz, 1H), 5.70-5.60 (m, 1H), 5.45 (t, J=4.0 Hz, 1H), 4.47-4.31 (m, 2H), 4.01-3.94 (m, 2H), 3.47 (m, 2H), 2.35-2.26 (m, 4H), 1.46 (s, 9H).

### Step-2: 4-(2-(1,2,3,6-Tetrahydropyridin-4-yl)-4-(trifluoromethyl)phenoxy)-N-(thiazol-2-yl) chroman-7-sulfonamide trifluoroacetic acid

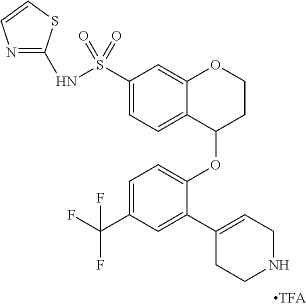 [SMILES: O=S(=O)(Nc1nccs1)c1ccc2c(c1)OCCC2Oc1ccc(C(F)(F)F)cc1C1=CCNCC1]

To a solution of thiazole-2-amine (0.01 g, 0.09 mmol) and tert-Butyl 4-(2-((7-((perfluoro phenoxy)sulfonyl)chroman-4-yl)oxy)-5-(trifluoromethyl)phenyl)-5,6-dihydro pyridine-1(2H)-carboxylate (step-1)(0.06 g, 0.08 mmol) in THF (3 ml) was added LiHMDS (1M in THF, 0.17 ml,) at −78° C. under nitrogen. The reaction mixture was stirred for 2 h allowing it to come at room temperature, then poured into saturated aqueous ammonium chloride solution and extracted with DCM. The combined organic layers was washed with brine, dried over anhydrous Na₂SO₄, and concentrated under vacuum. The crude product was purified by column chromatography (30% ethyl acetate/Petroleum ether) to obtain title compound with Boc protection as off white solid. This product was taken in DCM (2 ml), added TFA (0.2 ml) and stirred at room temperature for 1 h. After completion of reaction, the solvent was removed under vacuum and triturated with ether and decanted the solvent to obtain title compound as off-white solid (0.006 g, 13%). LCMS(ESI): m/z 538.03 (M+H)⁺; ¹H NMR (400 MHz, Methanol-d₄) δ 7.71-7.66 (m, 1H), 7.52-7.47 (m, 2H), 7.40-7.31 (m, 3H), 7.13 (d, J=4.6 Hz, 1H), 6.76 (d, J=4.7 Hz, 1H), 5.72-5.70 (m, 1H), 5.70-5.65 (m, 1H), 4.44-4.36 (m, 1H), 4.34-4.25 (m, 1H), 3.75-3.70 (m, 2H), 3.28-3.22 (m, 2H), 2.70-2.53 (m, 2H), 2.44-2.21 (m, 2H).

### Example-63: 4-(2-(Piperidin-4-yl)-4-(trifluoromethyl)phenoxy)-N-(thiazol-2-yl)chroman-7-sulfonamide hydrochloride

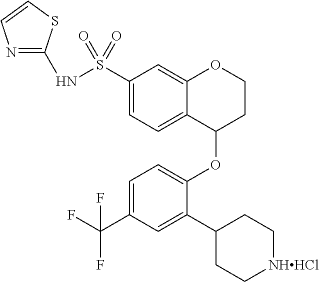

To a solution of thiazol-2-amine (0.018 g, 0.180 mmol), tert-Butyl 4-(2-((7-((perfluorophenoxy)sulfonyl)chroman-4-yl)oxy)-5-(trifluoromethyl)phenyl)piperidine-1-carboxylate (0.10 g, 0.138 mmol)(Intermediate-16) in THF (5 ml) was added LiHMDS (1M in THF)(0.207 ml, 0.207 mmol) at 0° C. under nitrogen atm. The reaction mixture was stirred for 4 h at room temperature then poured into 2N HCl solution and extracted with ethyl acetate. The combined organic layer was washed with brine, dried over Na₂SO₄, and evaporated under vacuum. This crude was purified by preparative HPLC to obtain Boc protected intermediate which was taken in DCM (2 ml), and stirred with 2N HCl in diethyl ether ether (0.213 ml, 2.76 mmol) for 2 h. TLC indicated completion of reaction. The solvent was removed under vacuum and triturated with ether to obtain title compound as white solid (0.015 g, 20.12%). LCMS(ESI): m/z 540.08 (M+H)⁺; ¹H NMR (400 MHz, Methanol-d₄) δ 7.64 (dd, J=8.8, 2.3 Hz, 1H), 7.52-7.45 (m, 2H), 7.45-7.30 (m, 3H), 7.13 (d, J=4.7 Hz, 1H), 6.76 (d, J=4.7 Hz, 1H), 5.71 (t, J=3.8 Hz, 1H), 4.42-4.37 (m, 2H), 3.49-3.33 (m, 2H), 3.20-2.90 (m 3H), 2.40-2.30 (m, 2H), 2.05-1.70 (m, 4H).

### Example-64: 4-(2-(Piperidin-4-yl)-4-(trifluoromethyl)phenoxy)-N-(1,2,4-thiadiazol-5-yl) chroman-7-sulfonamide trifluoroacetic acid

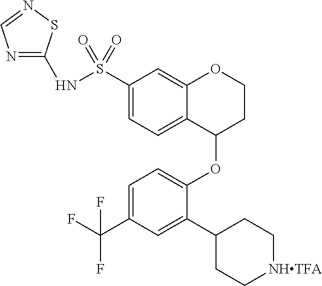

To a solution of 1,2,4-thiadiazol-5-amine (0.023 g, 0.226 mmol) in THF (5 ml) was added LiHMDS (1M in THF) (0.260 ml, 0.260 mmol) at −78° C. under nitrogen atm. The reaction mixture was allowed to stir slowly at ambient temperature then cooled again −30° C. before the drop wise addition of tert-Butyl 4-(2-((7-(chlorosulfonyl)chroman-4-yl)oxy)-5-(trifluoromethyl)phenyl)piperidine-1-carboxylate (0.10 g, 0.174 mmol)(Intermediate-16) in THF. The reaction mixture was then stirred at room temperature for 1 h. After completion of reaction as indicated by TLC, reaction mixture was poured into 2N HCl solution and extracted with ethyl acetate. The combined organic layer was washed with brine, dried over Na₂SO₄, and concentrated under vacuum. The crude product was purified by preparative HPLC to obtain Boc protected intermediate which was taken in DCM (2 ml) and treated with TFA (0.267 ml, 3.47 mmol) for 1 h. The solvent was removed under vacuum, triturated with MeOH (3 ml), filtered and filtrate was evaporated under vacuum to obtain title compound as white solid (0.021 g, 22%). LCMS(ESI): m/z 541.08 (M+H)⁺; ¹H NMR (400 MHz, DMSO-d₆) δ 8.57 (bs, 1H), 8.29 (s, 1H), 8.14 (bs, 1H), 7.78-7.13 (m, 6H), 5.81 (d, J=5.0 Hz, 1H), 4.50-4.22 (m, 2H), 3.57-3.36 (m, 2H), 3.19-2.78 (m, 3H), 2.40-2.10 (m, 2H), 1.96-1.64 (m, 4H).

Below Example-65 and 66 were prepared by following similar procedure as described in Example-34 using Intermediate 18a and 18b respectively.

### Example-65: (R)-4-(2-Chloro-4-(trifluoromethyl)phenoxy)-2,2-dimethyl-N-(thiazol-2-yl) chroman-7-sulfonamide

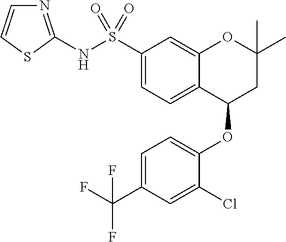 [SMILES: CC1(C)C[C@@H](Oc2ccc(C(F)(F)F)cc2Cl)c2ccc(S(=O)(=O)Nc3nccs3)cc2O1]

Yield 50%; LCMS(ESI): m/z 518.98 (M+H)⁺; ¹H NMR (400 MHz, Chloroform-d) δ 7.71 (d, J=2.3 Hz, 1H), 7.58-7.47 (m, 3H), 7.45 (d, J=1.7 Hz, 1H), 7.21-7.11 (m, 2H), 6.57 (d, J=4.6 Hz, 1H), 5.53 (t, J=6.2 Hz, 1H), 2.28 (dd, J=14.1, 5.7 Hz, 1H), 2.18 (dd, J=14.0, 6.8 Hz, 1H), 1.49 (s, 3H), 1.43 (s, 3H).

### Example-66: (S)-4-(2-Chloro-4-(trifluoromethyl)phenoxy)-2,2-dimethyl-N-(thiazol-2-yl) chroman-7-sulfonamide

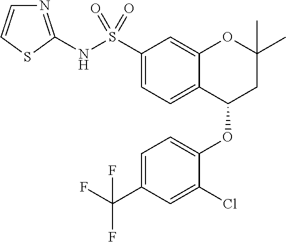 [SMILES: CC1(C)C[C@H](Oc2ccc(C(F)(F)F)cc2Cl)c2ccc(S(=O)(=O)Nc3nccs3)cc2O1]

Yield 15%; LCMS(ESI): m/z 518.63 (M+H)⁺; 1H NMR (400 MHz, Chloroform-d) δ 7.71 (d, J=2.2 Hz, 1H), 7.59-7.41 (m, 4H), 7.22-7.09 (m, 2H), 6.57 (d, J=4.5 Hz, 1H), 5.53 (t, J=6.2 Hz, 1H), 2.43-2.10 (m, 2H), 1.49 (s, 3H), 1.43 (s, 3H).

Below Examples 67 to 70 were prepared by following similar procedure as described in Example-34 using Intermediate-17a and different commercially available phenols.

### Example-67: (R)-2,2-Dimethyl-N-(thiazol-2-yl)-4-(4-(trifluoromethyl)phenoxy)chroman-7-sulfonamide

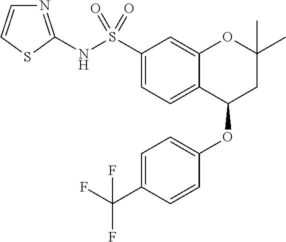 [SMILES: CC1(C)C[C@@H](Oc2ccc(C(F)(F)F)cc2)c2ccc(S(=O)(=O)Nc3nccs3)cc2O1]

LCMS(ESI): m/z 485.00 (M+H)⁺; ¹H NMR (400 MHz, Chloroform-d) δ 7.61 (d, J=8.6 Hz, 2H), 7.52-7.41 (m, 3H), 7.17 (d, J=4.6 Hz, 1H), 7.06 (d, J=8.5 Hz, 2H), 6.56 (d, J=4.6 Hz, 1H), 5.50 (t, J=6.1 Hz, 1H), 2.25 (d, J=5.6 Hz, 1H), 2.21-2.08 (m, 1H), 1.45 (s, 3H), 1.42 (s, 3H).

### Example-68: (R)-2,2-Dimethyl-N-(thiazol-2-yl)-4-(3-(trifluoromethyl)phenoxy)chroman-7-sulfonamide

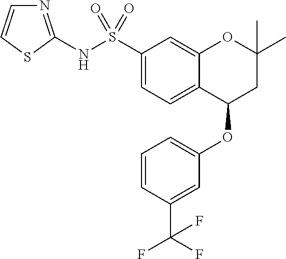 [SMILES: CC1(C)C[C@@H](Oc2cccc(C(F)(F)F)c2)c2ccc(S(=O)(=O)Nc3nccs3)cc2O1]

LCMS(ESI): m/z 484.80 (M+H)⁺; ¹H NMR (400 MHz, Chloroform-d) δ 7.57-7.43 (m, 4H), 7.32-7.28 (m, 1H), 7.25-7.15 (m, 3H), 6.58 (d, J=4.4 Hz, 1H), 5.48 (t, J=6.3 Hz, 1H), 2.30-2.10 (m, 2H), 1.47 (s, 3H), 1.42 (s, 3H).

### Example-69: (R)-4-(2,4-Dichlorophenoxy)-2,2-dimethyl-N-(thiazol-2-yl)chroman-7-sulfonamide

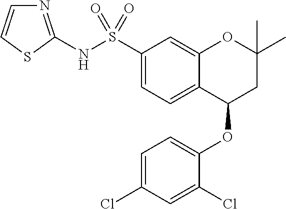 [SMILES: CC1(C)C[C@@H](Oc2ccc(Cl)cc2Cl)c2ccc(S(=O)(=O)Nc3nccs3)cc2O1]

LCMS(ESI): m/z 485.00 (M+H)⁺; ¹H NMR (400 MHz, DMSO-d₆) δ 12.81 (bs, 1H), 7.64 (d, J=2.5 Hz, 1H), 7.54-7.40 (m, 3H), 7.37-7.25 (m, 2H), 7.15 (d, J=1.8 Hz, 1H), 6.85 (d, J=4.6 Hz, 1H), 5.70 (t, J=5.9 Hz, 1H), 2.35-2.25 (m, 1H), 2.05 (dd, J=14.3, 6.2 Hz, 1H), 1.39 (s, 3H), 1.34 (s, 3H).

### Example-70: (R)-4-(2-Cyano-4-(trifluoromethyl)phenoxy)-2,2-dimethyl-N-(thiazol-2-yl)chroman-7-sulfonamide

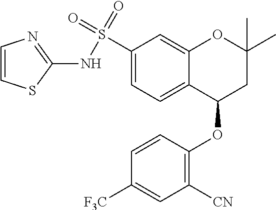 [SMILES: CC1(C)C[C@@H](Oc2ccc(C(F)(F)F)cc2C#N)c2ccc(S(=O)(=O)Nc3nccs3)cc2O1]

LCMS(ESI): m/z 510.04 (M+H)⁺; ¹H NMR (400 MHz, DMSO-d₆) δ 12.82 (bs, 1H), 8.35-8.28 (m, 1H), 8.10 (dd, J=9.0, 2.4 Hz, 1H), 7.77 (d, J=9.1 Hz, 1H), 7.54 (d, J=8.2 Hz, 1H), 7.36 (dd, J=8.1, 1.8 Hz, 1H), 7.29 (d, J=4.7 Hz, 1H), 7.18 (d, J=1.9 Hz, 1H), 6.86 (d, J=4.6 Hz, 1H), 5.97 (t, J=5.4 Hz, 1H), 2.35 (dd, J=14.6, 5.3 Hz, 1H), 2.15 (dd, J=14.5, 5.5 Hz, 1H), 1.39 (d, J=2.7 Hz, 6H).

Below Examples-71 to 78 to were prepared by following similar procedure as described in Example-34 using Intermediate-17a and different phenols which were prepared as shown in Intermediate procedure.

### Example-71: (R)-4-(2-Ethyl-4-(trifluoromethyl)phenoxy)-2,2-dimethyl-N-(thiazol-2-yl)chroman-7-sulfonamide

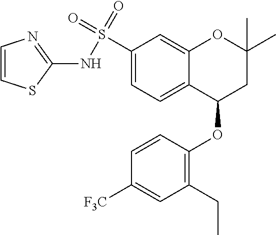 [SMILES: CCc1cc(C(F)(F)F)ccc1O[C@@H]1CC(C)(C)Oc2cc(S(=O)(=O)Nc3nccs3)ccc21]

LCMS(ESI): m/z 513.07 (M+H)⁺; ¹H NMR (400 MHz, DMSO-d₆) δ 12.81 (bs, 1H), 7.58 (d, J=8.6 Hz, 1H), 7.53 (d, J=2.4 Hz, 1H), 7.48 (d, J=8.2 Hz, 1H), 7.41 (d, J=8.7 Hz, 1H), 7.34 (dd, J=8.1, 1.9 Hz, 1H), 7.29 (d, J=4.7 Hz, 1H), 7.17 (d, J=1.8 Hz, 1H), 6.86 (d, J=4.6 Hz, 1H), 5.76 (t, J=5.9 Hz, 1H), 2.57 (q, J=7.4 Hz, 2H), 2.37 (dd, J=14.2, 5.7 Hz, 1H), 2.05 (dd, J=14.2, 6.3 Hz, 1H), 1.39 (d, J=2.3 Hz, 6H), 1.09 (t, J=7.5 Hz, 3H).

### Example-72: (R)-4-(2-(1-Isopropyl-1H-pyrazol-5-yl)-4-(trifluoromethyl)phenoxy)-2,2-dimethyl-N-(thiazol-2-yl)chroman-7-sulfonamide

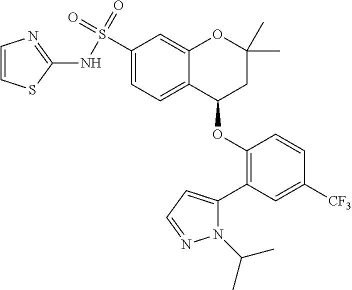 [SMILES: CC(C)n1nccc1-c1cc(C(F)(F)F)ccc1O[C@@H]1CC(C)(C)Oc2cc(S(=O)(=O)Nc3nccs3)ccc21]

LCMS(ESI): m/z 593.11 (M+H)⁺; ¹H NMR (400 MHz, DMSO-d₆) δ 12.81 (bs, 1H), 7.89 (d, J=8.8 Hz, 1H), 7.70 (d, J=9.0 Hz, 1H), 7.58 (d, J=2.4 Hz, 1H), 7.43 (d, J=1.8 Hz, 1H), 7.37 (d, J=8.1 Hz, 1H), 7.32-7.22 (m, 2H), 7.08 (d, J=1.8 Hz, 1H), 6.85 (d, J=4.6 Hz, 1H), 6.18 (d, J=1.8 Hz, 1H), 5.85 (t, J=5.6 Hz, 1H), 4.12 (p, J=6.6 Hz, 1H), 2.31-2.23 (m, 1H), 1.97-1.88 (m, 1H), 1.32 (s, 3H), 1.24 (s, 3H), 1.11 (d, J=6.5 Hz, 3H), 1.06 (d, J=6.5 Hz, 3H).

### Example-73: (R)-4-((4′-Fluoro-5-(trifluoromethyl)-[1,1′-biphenyl]-2-yl)oxy)-2,2-dimethyl-N-(thiazol-2-yl)chroman-7-sulfonamide

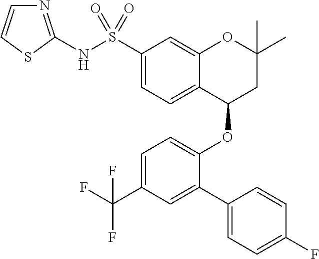 [SMILES: CC1(C)C[C@@H](Oc2ccc(C(F)(F)F)cc2-c2ccc(F)cc2)c2ccc(S(=O)(=O)Nc3nccs3)cc2O1]

LCMS(ESI): m/z 578.82 (M+H)⁺; ¹H NMR (400 MHz, DMSO-d₆) δ 12.79 (bs, 1H), 7.82-7.73 (m, 1H), 7.66-7.57 (m, 2H), 7.56-7.47 (m, 2H), 7.43 (d, J=8.1 Hz, 1H), 7.32-7.25 (m, 2H), 7.23-7.13 (m, 2H), 7.09 (d, J=1.8 Hz, 1H), 6.84 (d, J=4.5 Hz, 1H), 5.80 (t, J=6.3 Hz, 1H), 2.42-2.29 (m, 1H), 1.96-1.85 (m, 1H), 1.34 (s, 3H), 1.20 (s, 3H).

### Example-74: (R)-4-(2-(4-Cyclopropyl-1H-1,2,3-triazol-1-yl)-4-(trifluoromethyl)phenoxy)-2,2-dimethyl-N-(thiazol-2-yl)chroman-7-sulfonamide

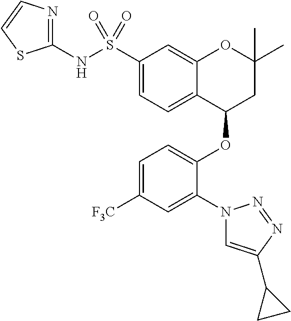 [SMILES: CC1(C)C[C@@H](Oc2ccc(C(F)(F)F)cc2-n2cc(C3CC3)nn2)c2ccc(S(=O)(=O)Nc3nccs3)cc2O1]

LCMS(ESI): m/z 591.83 (M+H)⁺; ¹H NMR (400 MHz, DMSO-d6) δ 12.81 (bs, 1H), 8.05 (s, 1H), 8.02-7.90 (m, 2H), 7.79 (d, J=8.9 Hz, 1H), 7.47 (d, J=8.1 Hz, 1H), 7.33-7.24 (m, 2H), 7.12 (d, J=1.9 Hz, 1H), 6.85 (d, J=4.6 Hz, 1H), 5.90 (t, J=6.3 Hz, 1H), 2.40 (dd, J=14.1, 5.8 Hz, 1H), 2.02-1.95 (m, 1H), 1.94-1.83 (m, 1H), 1.35 (s, 3H), 1.20 (s, 3H), 0.87-0.81 (m, 2H), 0.67-0.49 (m, 2H).

### Example-75: (R)-4-(2-(1H-Pyrazol-1-yl)-4-(trifluoromethyl)phenoxy)-2,2-dimethyl-N-(thiazol-2-yl)chroman-7-sulfonamide

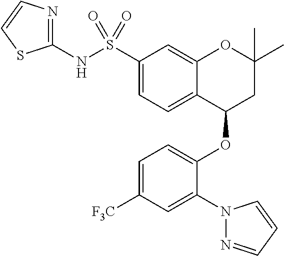 [SMILES: CC1(C)C[C@@H](Oc2ccc(C(F)(F)F)cc2-n2cccn2)c2ccc(S(=O)(=O)Nc3nccs3)cc2O1]

LCMS(ESI): m/z 573.07 (M+Na)⁺; ¹H NMR (400 MHz, DMSO-d6) δ 8.10 (d, J=2.5 Hz, 1H), 8.00 (d, J=2.2 Hz, 1H), 7.81-7.69 (m, 3H), 7.54 (d, J=8.2 Hz, 1H), 7.35-7.26 (m, 2H), 7.14 (d, J=1.8 Hz, 1H), 6.86 (d, J=4.6 Hz, 1H), 6.43 (dd, J=2.5, 1.8 Hz, 1H), 5.92 (t, J=6.2 Hz, 1H), 2.39 (dd, J=14.1, 5.7 Hz, 1H), 2.13-2.01 (m, 1H), 1.35 (s, 3H), 1.24 (s, 3H).

### Example-76: —(R)-2,2-Dimethyl-4-(2-(pyridin-2-yl)-4-(trifluoromethyl)phenoxy)-N-(thiazol-2-yl)chroman-7-sulfonamide

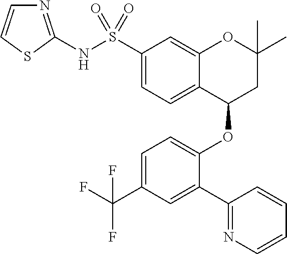 [SMILES: CC1(C)C[C@@H](Oc2ccc(C(F)(F)F)cc2-c2ccccn2)c2ccc(S(=O)(=O)Nc3nccs3)cc2O1]

LCMS(ESI): m/z 562.0 (M+H)⁺; ¹H NMR (400 MHz, DMSO-d₆) δ 12.80 (bs, 1H), 8.75-8.61 (m, 1H), 8.08 (d, J=2.5 Hz, 1H), 7.90-7.79 (m, 2H), 7.77-7.59 (m, 2H), 7.51 (d, J=8.1 Hz, 1H), 7.39-7.22 (m, 3H), 7.13 (d, J=1.8 Hz, 1H), 6.86 (d, J=4.6 Hz, 1H), 5.90 (t, J=6.4 Hz, 1H), 2.43-2.33 (m, 1H), 2.07-1.96 (m, 1H), 1.36 (s, 3H), 1.25 (s, 3H).

### Example-77: (R)-2,2-Dimethyl-4-(2-(2-methylthiazol-4-yl)-4-(trifluoromethyl)phenoxy)-N-(thiazol-2-yl)chroman-7-sulfonamide

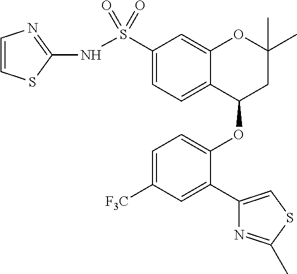 [SMILES: Cc1nc(-c2cc(C(F)(F)F)ccc2O[C@@H]2CC(C)(C)Oc3cc(S(=O)(=O)Nc4nccs4)ccc32)cs1]

LCMS(ESI): m/z 581.83 (M+H)⁺; ¹H NMR (400 MHz, DMSO-d₆) δ 12.81 (bs, 1H), 8.50 (d, J=2.5 Hz, 1H), 7.73 (d, J=8.7 Hz, 2H), 7.64 (d, J=8.8 Hz, 1H), 7.54 (d, J=8.1 Hz, 1H), 7.39-7.26 (m, 2H), 7.18 (d, J=1.8 Hz, 1H), 6.86 (d, J=4.6 Hz, 1H), 5.97 (t, J=6.5 Hz, 1H), 2.46-2.41 (m, 1H), 2.16 (dd, J=13.9, 7.2 Hz, 1H), 1.39 (s, 3H), 1.35 (s, 3H).

### Example-78: (R)-2,2-Dimethyl-4-(2-(2-methyloxazol-4-yl)-4-(trifluoromethyl)phenoxy)-N-(thiazol-2-yl)chroman-7-sulfonamide

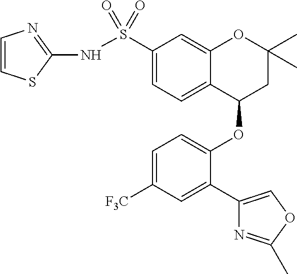 [SMILES: Cc1nc(-c2cc(C(F)(F)F)ccc2O[C@@H]2CC(C)(C)Oc3cc(S(=O)(=O)Nc4nccs4)ccc32)co1]

LCMS(ESI): m/z 566.08 (M+H)⁺; ¹H NMR (400 MHz, DMSO-d₆) δ 12.82 (bs, 1H), 8.29 (d, J=2.4 Hz, 1H), 7.91 (s, 1H), 7.72 (dd, J=9.1, 2.5 Hz, 1H), 7.60 (d, J=8.8 Hz, 1H), 7.52 (d, J=8.1 Hz, 1H), 7.35 (dd, J=8.1, 1.8 Hz, 1H), 7.29 (d, J=4.6 Hz, 1H), 7.19 (d, J=1.8 Hz, 1H), 6.87 (d, J=4.6 Hz, 1H), 5.95 (t, J=6.6 Hz, 1H), 2.46 (s, 3H), 2.42-2.40 (m, 1H) 2.19 (dd, J=13.9, 7.5 Hz, 1H), 1.39 (s, 3H), 1.37 (s, 3H).

### Example-79: (R)-2,2-Dimethyl-4-(2-(1-methyl-1H-pyrazol-5-yl)-4-(trifluoromethyl) phenoxy)-N-(thiazol-2-yl)chroman-7-sulfonamide

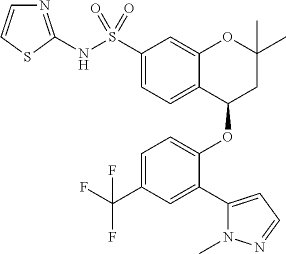 [SMILES: Cn1nccc1-c1cc(C(F)(F)F)ccc1O[C@@H]1CC(C)(C)Oc2cc(S(=O)(=O)Nc3nccs3)ccc21]

The title compound was prepared by following similar procedure as described in Example-41 using Intermediate-17a.

LCMS(ESI): m/z 564.57 (M+H)⁺; ¹H NMR (400 MHz, DMSO-d₆) δ 12.80 (bs, 1H), 7.96-7.85 (m, 1H), 7.77-7.61 (m, 2H), 7.44-7.34 (m, 2H), 7.31-7.24 (m, 2H), 7.10 (d, J=1.8 Hz, 1H), 6.85 (d, J=4.6 Hz, 1H), 6.26 (d, J=1.9 Hz, 1H), 5.83 (s, 1H), 3.56 (s, 3H), 2.35-2.25 (m, 1H), 2.04-1.90 (m, 1H), 1.33 (s, 3H), 1.11 (s, 3H).

### Example-80: (R)-2,2-Dimethyl-4-(2-(1-methyl-1H-pyrazol-5-yl)-5-(trifluoromethyl) phenoxy)-N-(thiazol-2-yl)chroman-7-sulfonamide

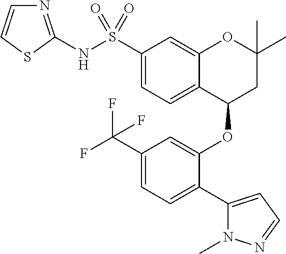 [SMILES: Cn1nccc1-c1ccc(C(F)(F)F)cc1O[C@@H]1CC(C)(C)Oc2cc(S(=O)(=O)Nc3nccs3)ccc21]

The title compound was prepared by following similar procedure as described in Example-41, Mitsunobu reaction of Intermediate-17a with 2-bromo-5-(trifluoromethyl)phenol followed by coupling with 1-Methyl-5-(4,4,5,5-tetramethyl-1,3,2-dioxaborolan-2-yl)-1H-pyrazole and then reaction with 2-aminothiazole.

LCMS(ESI): m/z 565.05 (M+H)⁺; ¹H NMR (400 MHz, DMSO-d₆) δ 12.80 (bs, 1H), 7.81 (s, 1H), 7.56 (d, J=7.9 Hz, 1H), 7.48 (d, J=1.5 Hz, 1H), 7.45-7.35 (m, 2H), 7.33-7.24 (m, 2H), 7.10 (d, J=1.8 Hz, 1H), 6.85 (d, J=4.6 Hz, 1H), 6.23 (d, J=1.9 Hz, 1H), 5.86 (t, 1H), 3.56 (s, 3H), 2.25-2.20 (m, 1H), 1.97-1.90 (m, 1H), 1.32 (s, 3H), 1.24 (s, 3H).

### Example-81: (R)-2,2-Dimethyl-4-(2-(pyridin-4-yl)-4-(trifluoromethyl)phenoxy)-N-(thiazol-2-yl)chroman-7-sulfonamide

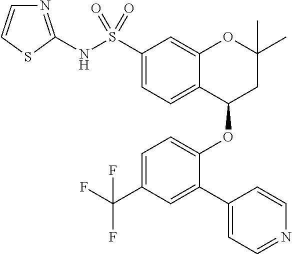 [SMILES: CC1(C)C[C@@H](Oc2ccc(C(F)(F)F)cc2-c2ccncc2)c2ccc(S(=O)(=O)Nc3nccs3)cc2O1]

The title compound was prepared by following similar procedure as described in Example-41 using Intermediate-17a.

LCMS(ESI): m/z 562.00 (M+H)⁺; ¹H NMR (400 MHz, DMSO-d₆) δ 12.80 (bs, 1H), 8.60-8.50 (m, 2H), 7.85 (dd, J=8.8, 2.4 Hz, 1H), 7.76-7.64 (m, 2H), 7.57-7.39 (m, 3H), 7.36-7.25 (m, 2H), 7.10 (d, J=1.8 Hz, 1H), 6.85 (d, J=4.6 Hz, 1H), 5.86 (t, J=6.3 Hz, 1H), 2.43-2.27 (m, 1H), 2.18 (t, J=8.1 Hz, 1H), 1.35 (s, 3H), 1.21 (s, 3H).

### Example-82: (R)-4-(2-Chloro-4-(trifluoromethyl)phenoxy)-N-(thiazol-2-yl)spiro [chroman-2,1′-cyclobutane]-7-sulfonamide

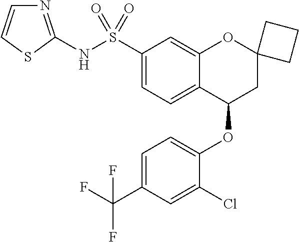 [SMILES: O=S(=O)(Nc1nccs1)c1ccc2c(c1)OC1(CCC1)C[C@H]2Oc1ccc(C(F)(F)F)cc1Cl]

### Step-1: (S)-Perfluorophenyl 4-hydroxyspiro[chroman-2,1′-cyclobutane]-7-sulfonate

The title compound was prepared by following similar procedure as described in Intermediate-12a using 7-Bromospiro[chroman-2,1′-cyclobutan]-4-one (Prepared as described in US2002/82264 A1). Yield 74%; ¹H NMR (400 MHz, Chloroform-d) δ 7.69 (dd, J=8.2, 0.9 Hz, 1H), 7.51 (dd, J=8.1, 1.9 Hz, 1H), 7.45 (d, J=1.9 Hz, 1H), 4.94 (dd, J=9.0, 5.6 Hz, 1H), 2.53-2.25 (m, 4H), 2.22-1.90 (m, 2H), 1.85-1.70 (m, 2H).

### Step-2: (R)-Perfluorophenyl 4-(2-chloro-4-(trifluoromethyl)phenoxy)spiro[chroman-2,1′-cyclobutane]-7-sulfonate

The title compound was prepared by following similar procedure as described for Intermediate-13a using (S)-perfluorophenyl 4-hydroxyspiro[chroman-2,1′-cyclobutane]-7-sulfonate and 2-chloro-4-(trifluoromethyl)phenol.

Yield 52%; ¹H NMR (400 MHz, Chloroform-d) δ 7.75 (d, J=2.3 Hz, 1H), 7.62-7.49 (m, 4H), 7.19 (d, J=8.6 Hz, 1H), 5.55 (t, J=6.0 Hz, 1H), 2.54-2.35 (m, 4H), 2.27-1.92 (m, 2H), 1.85-1.70 (m, 2H).

### Step-3: (R)-4-(2-Chloro-4-(trifluoromethyl)phenoxy)-N-(thiazol-2-yl)spiro[chroman-2,1′-cyclobutane]-7-sulfonamide

The title compound was prepared by following similar procedure as described in Example-34 using (R)-perfluorophenyl 4-(2-chloro-4-(trifluoromethyl)phenoxy)spiro[chroman-2,1′-cyclobutane]-7-sulfonate (Step-2) and 2-amino thiazole. Yield: 23%; LCMS(ESI): m/z 530.69 (M+H)⁺; ¹H NMR (400 MHz, DMSO-d₆) δ 12.83 (bs, 1H), 7.90 (d, J=2.2 Hz, 1H), 7.81-7.69 (m, 2H), 7.48 (d, J=8.1 Hz, 1H), 7.35 (dd, J=8.1, 1.8 Hz, 1H), 7.29 (d, J=4.6 Hz, 1H), 7.23 (d, J=1.8 Hz, 1H), 6.86 (d, J=4.6 Hz, 1H), 5.88 (t, J=5.2 Hz, 1H), 2.46-2.11 (m, 6H), 1.75-1.59 (m, 2H).

### Example-83: (R)-4-(2-(1-Methyl-1H-pyrazol-5-yl)-4-(trifluoromethyl)phenoxy)-N-(thiazol-2-yl)spiro[chroman-2,1′-cyclobutane]-7-sulfonamide

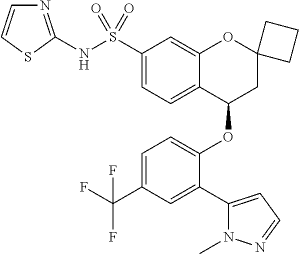 [SMILES: Cn1nccc1-c1cc(C(F)(F)F)ccc1O[C@@H]1CC2(CCC2)Oc2cc(S(=O)(=O)Nc3nccs3)ccc21]

### Step-1: (R)-Perfluorophenyl 4-(2-(1-methyl-1H-pyrazol-5-yl)-4-(trifluoromethyl) phenoxy)spiro[chroman-2,1′-cyclobutane]-7-sulfonate

Titled compound was prepared by following similar procedure as described in Intermediate-14a using (S)-perfluorophenyl 4-hydroxyspiro[chroman-2,1′-cyclobutane]-7-sulfonate (Step 1 of Example-82); Yield 65%; LCMS(ESI): m/z 661.00 (M+H)⁺.

### Step 2: (R)-4-(2-(1-Methyl-1H-pyrazol-5-yl)-4-(trifluoromethyl)phenoxy)-N-(thiazol-2-yl)spiro[chroman-2,1′-cyclobutane]-7-sulfonamide

The title compound was prepared by following the similar procedure as described in Example-41 using (R)-Perfluorophenyl 4-(2-(1-methyl-1H-pyrazol-5-yl)-4-(trifluoromethyl) phenoxy)spiro[chroman-2,1′-cyclobutane]-7-sulfonate (Step-1) and 2-amino thiazole. Yield: 29%; LCMS(ESI): m/z 576.57 (M+H)⁺; ¹H NMR (400 MHz, DMSO-d₆) δ 12.81 (bs, 1H), 7.90 (dd, J=8.9, 2.4 Hz, 1H), 7.78-7.72 (m, 1H), 7.64 (d, J=2.4 Hz, 1H), 7.41-7.35 (m, 2H), 7.31-7.25 (m, 2H), 7.16 (d, J=1.8 Hz, 1H), 6.86 (d, J=4.6 Hz, 1H), 6.25 (d, J=1.9 Hz, 1H), 5.86 (t, 1H), 3.50 (s, 3H), 2.45-2.28 (m, 2H), 2.25-2.15 (m, 2H), 1.97-1.85 (m, 2H), 1.85-1.52 (m, 2H).

### Example-84: (2R/S,4R)-4-(2-Chloro-4-(trifluoromethyl)phenoxy)-2-ethyl-N-(thiazol-2-yl)chroman-7-sulfonamide

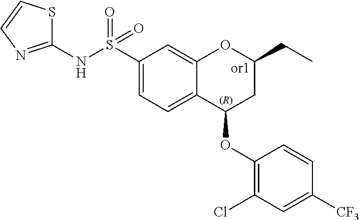

### Step-1: (2R/S,4R)-Perfluorophenyl 4-(2-chloro-4-(trifluoromethyl)phenoxy)-2-ethylchroman-7-sulfonate

To a solution of 2-Chloro-4-(trifluoromethyl)phenol (0.037 g, 0.189 mmol), Intermediate-20a (0.08 g, 0.189 mmol), and triphenylphosphine (0.064 g, 0.245 mmol) in THF (25 ml), DIAD (0.048 ml, 0.245 mmol) was added and stirred for 1 h at room temperature. The reaction mixture was diluted with ethyl acetate, washed with water, dried over Na₂SO₄ and concentrated under reduced pressure. The crude product was purified by flash column chromatography to obtain title compound as off white solid (0.08 g, 70%). ¹H NMR (400 MHz, Chloroform-d) δ 7.80-7.71 (m, 2H), 7.60-7.50 (m, 3H), 7.16 (d, J=8.6 Hz, 1H), 5.67 (dd, J=10.4, 5.9 Hz, 1H), 4.35-4.25 (m, 1H), 2.50 (ddd, J=13.3, 5.9, 2.0 Hz, 1H), 2.10 (dt, J=13.4, 10.9 Hz, 1H), 1.95-1.76 (m, 2H), 1.09 (t, J=7.4 Hz, 3H).

### Step-2: (2R/S,4R)-4-(2-Chloro-4-(trifluoromethyl)phenoxy)-2-ethyl-N-(thiazol-2-yl)chroman-7-sulfonamide

To a solution of (2R/S,4R)-perfluorophenyl 4-(2-Chloro-4-(trifluoromethyl)phenoxy)-2-ethylchroman-7-sulfonate (0.08 g, 0.133 mmol) and thiazol-2-amine (0.033 g, 0.332 mmol) in THF (10 ml) was added LiHMDS (1M in THF, 0.40 ml, 0.398 mmol) at 0° C. under nitrogen. The reaction mixture was stirred for 4 h at room temperature then poured into 2N HCl solution and extracted with ethyl acetate. The combined organic layers was washed with brine, dried over Na₂SO₄ and concentrated under vacuum. The crude was purified by column chromatography obtain title compound as white solid (0.018 g, 26%). LCMS(ESI): m/z 518.94 (M+H)⁺; ¹H NMR (400 MHz, DMSO-d₆) δ 12.81 (bs, 1H), 7.92 (d, J=2.2 Hz, 1H), 7.75 (dd, J=8.8, 2.3 Hz, 1H), 7.66 (d, J=8.8 Hz, 1H), 7.50 (d, J=8.2 Hz, 1H), 7.35 (dd, J=8.1, 1.8 Hz, 1H), 7.29 (d, J=4.6 Hz, 1H), 7.19 (d, J=1.8 Hz, 1H), 6.85 (d, J=4.6 Hz, 1H), 5.98 (dd, J=9.7, 6.0 Hz, 1H), 4.36-4.25 (m, 1H), 2.56 (ddd, J=6.6, 5.3, 2.0 Hz, 1H), 1.85-1.63 (m, 3H), 0.98 (t, J=7.4 Hz, 3H).

### Example-85: (2S/R,4R)-4-(2-Chloro-4-(trifluoromethyl)phenoxy)-2-ethyl-N-(thiazol-2-yl)chroman-7-sulfonamide

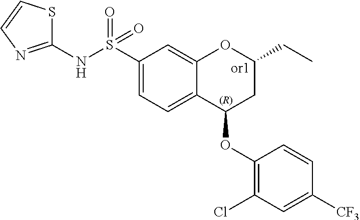

### Step-1: (2S/R,4R)-Perfluorophenyl 4-(2-chloro-4-(trifluoromethyl)phenoxy)-2-ethylchroman-7-sulfonate

The title compound was prepared by following similar procedure as described for Example-84 using Intermediate-20b.

Yield: 62%; ¹H NMR (400 MHz, Chloroform-d) δ 7.74-7.69 (m, 1H), 7.61-7.53 (m, 2H), 7.45 (dd, J=8.1, 1.9 Hz, 1H), 7.35 (d, J=8.1 Hz, 1H), 7.19 (d, J=8.7 Hz, 1H), 5.45 (t, J=2.9 Hz, 1H), 4.50-4.39 (m, 1H), 2.37 (dt, J=14.6, 2.1 Hz, 1H), 2.04-1.76 (m, 3H), 1.12 (t, J=7.4 Hz, 3H).

### Step-2: (2S/R,4R)-4-(2-Chloro-4-(trifluoromethyl)phenoxy)-2-ethyl-N-(thiazol-2-yl)chroman-7-sulfonamide

The title compound was prepared by following similar procedure as described in Example-84. Yield: 30%; LCMS(ESI): m/z 518.69 (M+H)⁺; ¹H NMR (400 MHz, DMSO-d₆) δ 12.82 (bs, 1H), 7.88 (d, J=2.2 Hz, 1H), 7.81-7.68 (m, 2H), 7.52 (d, J=8.1 Hz, 1H), 7.36-7.21 (m, 3H), 6.86 (d, J=4.6 Hz, 1H), 5.80 (t, J=2.7 Hz, 1H), 4.24-4.13 (m, 1H), 2.21 (dt, J=15.1, 2.2 Hz, 1H), 1.92 (ddd, J=15.1, 11.9, 3.3 Hz, 1H), 1.73 (p, J=7.3 Hz, 2H), 0.99 (t, J=7.4 Hz, 3H).

### Example-86: (2S/R,4R)-2-Ethyl-4-(2-(1-methyl-1H-pyrazol-5-yl)-4-(trifluoromethyl) phenoxy)-N-(thiazol-2-yl)chroman-7-sulfonamide

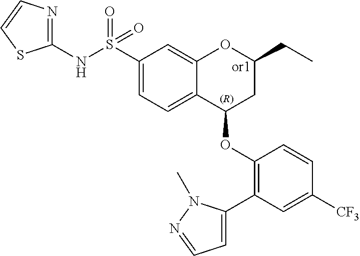

### Step-1: (2S/R,4R)-Perfluorophenyl 4-(2-bromo-4-(trifluoromethyl)phenoxy)-2-ethylchroman-7-sulfonate

The title compound was prepared by following similar procedure as described for Step-1 of Example-84 using 2-Bromo-4-(trifluoromethyl)phenol and Intermediate-20a.

Yield: 59%, ¹H NMR (400 MHz, Chloroform-d) δ 7.92 (dd, J=2.2, 0.8 Hz, 1H), 7.75 (dd, J=8.1, 0.9 Hz, 1H), 7.65-7.49 (m, 3H), 7.12 (d, J=8.6 Hz, 1H), 5.68 (dd, J=10.4, 5.9 Hz, 1H), 4.36-4.25 (m, 1H), 2.51 (ddd, J=13.3, 5.9, 2.1 Hz, 1H), 2.22-2.03 (m, 1H), 1.98-1.73 (m, 2H), 1.10 (t, J=7.4 Hz, 3H).

### Step-2: (2S/R,4R)-Perfluorophenyl 2-ethyl-4-(2-(1-methyl-1H-pyrazol-5-yl)-4-(trifluoro methyl)phenoxy)chroman-7-sulfonate

A solution of (2S/R,4R)-Perfluorophenyl 4-(2-bromo-4-(trifluoromethyl)phenoxy)-2-ethylchroman-7-sulfonate (0.1 g, 0.154 mmol), 1-Methyl-5-(4,4,5,5-tetramethyl-1,3,2-dioxaborolan-2-yl)-1H-pyrazole (0.052 g, 0.247 mmol) and K₃PO₄ (0.065 g, 0.309 mmol) in 1,4-dioxane:water (8 ml: 2 ml) was purged with N₂ for 10 minute then amphos (0.001 g, 0.015 mmol) was added and heated at 90° C. for 15 min under microwave irradiation. After completion of reaction, The reaction mixture was evaporated under vacuum and purified by flash column chromatography to obtain title compound as off-white solid (0.065 g, 64%). ¹H NMR (400 MHz, Chloroform-d) δ 7.76 (dd, J=8.7, 2.4 Hz, 1H), 7.67-7.62 (m, 1H), 7.57 (d, J=2.0 Hz, 1H), 7.50-7.39 (m, 2H), 7.39-7.21 (m, 2H), 6.33 (d, J=1.9 Hz, 1H), 5.59 (dd, J=10.2, 5.8 Hz, 1H), 4.24 (dt, J=11.7, 5.8 Hz, 1H), 3.76 (s, 3H), 2.42 (ddd, J=13.3, 5.9, 2.1 Hz, 1H), 2.01-1.64 (m, 3H), 1.05 (t, J=7.4 Hz, 3H).

### Step-3: (2S/R,4R)-2-Ethyl-4-(2-(1-methyl-1H-pyrazol-5-yl)-4-(trifluoro methyl)phenoxy)-N-(thiazol-2-yl)chroman-7-sulfonamide

To a solution of thiazol-2-amine (0.023 g, 0.231 mmol), (2S/R,4R)-perfluorophenyl 2-ethyl-4-(2-(1-methyl-1H-pyrazol-5-yl)-4-(trifluoromethyl)phenoxy)chroman-7-sulfonate (0.06 g, 0.093 mmol) in THF (10 ml) was added LiHMDS (1M in THF, 0.278 ml, 0.278 mmol) at 0° C. under nitrogen then stirred at room temperature for 4 h. The reaction mixture was poured into 2N HCl solution and extracted with ethyl acetate. The combined organic layers were washed with brine, dried over Na₂SO₄ and concentrated under vacuum. The crude product was purified by flash column chromatography to obtain title compound as white solid (0.015 g, 29%). LCMS(ESI): m/z 564.70 (M+H)⁺; ¹H NMR (400 MHz, DMSO-d₆) δ 12.79 (bs, 1H), 7.88 (dd, J=8.9, 2.4 Hz, 1H), 7.77-7.65 (m, 2H), 7.44 (d, J=1.9 Hz, 1H), 7.31-7.20 (m, 3H), 7.12 (s, 1H), 6.85 (d, J=4.6 Hz, 1H), 6.35 (d, J=1.9 Hz, 1H), 5.95 (dd, J=9.7, 5.9 Hz, 1H), 4.28 (dt, J=11.6, 6.0 Hz, 1H), 3.61 (s, 3H), 2.63-2.46 (m, 1H), 1.75-1.57 (m, 3H), 0.94 (t, J=7.4 Hz, 3H).

### Example-87: (2R/S,4R)-2-Ethyl-4-(2-(1-methyl-1H-pyrazol-5-yl)-4-(trifluoromethyl) phenoxy)-N-(thiazol-2-yl)chroman-7-sulfonamide

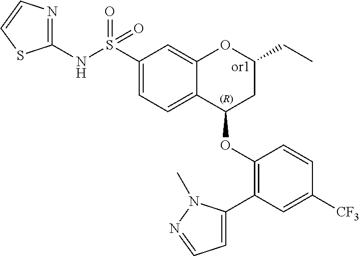

### Step-1: (2R/S,4R)-Perfluorophenyl 4-(2-bromo-4-(trifluoromethyl)phenoxy)-2-ethylchroman-7-sulfonate

The title compound was prepared by following the similar procedure as described in step-2 of Example-86. Yield: 70%; ¹H NMR (400 MHz, Chloroform-d) δ 7.76 (dd, J=8.7, 2.4 Hz, 1H), 7.63 (d, J=2.3 Hz, 1H), 7.52 (dd, J=6.9, 1.9 Hz, 2H), 7.49-7.38 (m, 1H), 7.33 (d, J=8.6 Hz, 1H), 7.18 (d, J=8.1 Hz, 1H), 6.19 (d, J=1.9 Hz, 1H), 5.25 (t, J=2.8 Hz, 1H), 3.91-3.80 (m, 1H), 3.65 (s, 3H), 2.16-2.04 (m, 1H), 1.86-1.69 (m, 3H), 1.02 (t, J=7.4 Hz, 3H).

### Step-2: (2R/S,4R)-2-Ethyl-4-(2-(1-methyl-1H-pyrazol-5-yl)-4-(trifluoromethyl) phenoxy)-N-(thiazol-2-yl)chroman-7-sulfonamide

The title compound was prepared by following the similar procedure as described in Example-86 using (2R/S,4R)-Perfluorophenyl 4-(2-bromo-4-(trifluoromethyl)phenoxy)-2-ethylchroman-7-sulfonate (step-1), 1-methyl-5-(4,4,5,5-tetra methyl-1,3,2-dioxaborolan-2-yl)-1H-pyrazole and 2-aminothiazole.

Yield: 40%; LCMS(ESI): m/z 565.05 (M+H)⁺; ¹H NMR (400 MHz, DMSO-d₆) δ 12.81 (bs, 1H), 7.97-7.88 (m, 1H), 7.78 (d, J=8.8 Hz, 1H), 7.64 (d, J=2.4 Hz, 1H), 7.45 (d, J=8.0 Hz, 1H), 7.39 (d, J=1.8 Hz, 1H), 7.34-7.25 (m, 2H), 7.15 (d, J=1.8 Hz, 1H), 6.86 (d, J=4.6 Hz, 1H), 6.19 (d, J=1.9 Hz, 1H), 5.69 (d, J=2.7 Hz, 1H), 3.73-3.62 (m, 1H), 3.47 (s, 3H), 2.12-2.04 (m, 1H), 1.87-1.77 (m, 1H), 1.67-1.55 (m, 2H), 0.91 (t, J=7.4 Hz, 3H).

### Example-88: 5-(2-Chloro-4-(trifluoromethyl)phenoxy)-N-(thiazol-2-yl)-5,6,7,8-tetrahydro naphthalene-2-sulfonamide

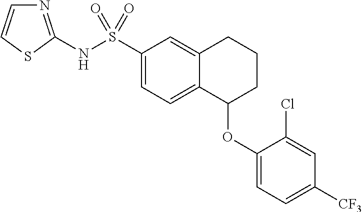 [SMILES: O=S(=O)(Nc1nccs1)c1ccc2c(c1)CCCC2Oc1ccc(C(F)(F)F)cc1Cl]

To a solution of N-(2,4-Dimethoxybenzyl)thiazol-2-amine (0.04 g, 0.16 mmol) in THF (5 ml) was added LiHMDS (1M in THF, 0.17 ml, 0.17 mmol) at −78° C. under nitrogen and allowed to stir at RT for 1 h. The reaction mixture was cooled again at −78° C. and perfluorophenyl 5-(2-chloro-4-(trifluoromethyl)phenoxy)-5,6,7,8-tetrahydronaphthalene-2-sulfonate (Intermediate-21) (0.09 g, 0.16 mmol) in THF was added and stirred at room temperature for 4 h. After completion of reaction, the reaction mixture was quenched with 2N HCl and the product was extracted with DCM. The combined organic layer was washed with brine, dried over Na₂SO₄, and concentrated under vacuum. The crude product was purified by flash column chromatography (30% ethyl acetate/petroleum ether) to obtain 5-(2-chloro-4-(trifluoromethyl)phenoxy)-N-(2,4-dimethoxybenzyl)-N-(thiazol-2-yl)-5,6,7,8-tetrahydro naphthalene-2-sulfonamide as a white solid (0.035 g, 35%). The above product was taken in DCM (1 ml) and added TFA (0.2 ml) and stirred for 1 h. Solvent was removed under reduced pressure and triturated with ether. The solvent was decanted and dried under vacuum to obtain the title compound as off-white solid. LCMS(ESI): m/z 488.99 (M+H)⁺; ¹H NMR (400 MHz, Chloroform-d) δ 7.79-7.72 (m, 2H), 7.68 (d, J=2.2, 1H), 7.60-7.45 (m, 2H), 7.20-7.12 (m, 2H), 6.56 (d, J=4.6 Hz, 1H), 5.50-5.47 (m, 1H), 3.05-2.93 (m, 1H), 2.92-2.81 (m, 1H), 2.15-2.05 (m, 3H), 1.91-1.82 (m, 1H).

### Example-89: (R)-5-(2-Chloro-4-(trifluoromethyl)phenoxy)-N-(thiazol-2-yl)-5,6,7,8-tetrahydronaphthalene-2-sulfonamide

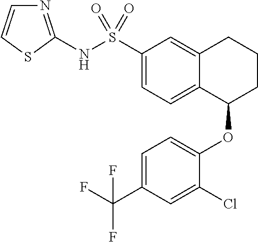 [SMILES: O=S(=O)(Nc1nccs1)c1ccc2c(c1)CCC[C@H]2Oc1ccc(C(F)(F)F)cc1Cl]

To a solution of thiazol-2-amine (0.050 g, 0.50 mmol) and (R)-perfluorophenyl 5-(2-chloro-4-(trifluoromethyl)phenoxy)-5,6,7,8-tetrahydronaphthalene-2-sulfonate (Intermediate-21a) (0.22 g, 0.38 mmol) in THF (5 ml) was added LiHMDS (1M in THF, 0.5 ml, 0.499 mmol) at 0° C. under N₂. The reaction mixture was stirred for 4 h at ambient temperature. The reaction mixture was poured into 2N HCl solution and extracted with ethyl acetate. The combined organic layer was washed with brine, dried over Na₂SO₄ and concentrated under vacuum. The crude product was purified by preparative HPLC to obtain title compound as white solid (0.083 g, 44%). LCMS(ESI): m/z 488.93 (M+H)⁺. ¹H NMR (400 MHz, Chloroform-d) δ 7.79-7.72 (m, 2H), 7.68 (d, J=2.2 Hz, 1H), 7.60-7.46 (m, 2H), 7.20-7.13 (m, 2H), 6.56 (d, J=4.5 Hz, 1H), 5.47 (t, J=4.8 Hz, 1H), 2.99 (dt, J=17.4, 5.4 Hz, 1H), 2.87 (dd, J=15.2, 9.4 Hz, 1H), 2.15-2.04 (m, 3H), 1.95-1.80 (m, 1H).

### Example-90: 5-(2-Bromo-4-(trifluoromethyl)phenoxy)-N-(thiazol-2-yl)-5,6,7,8-tetrahydro naphthalene-2-sulfonamide

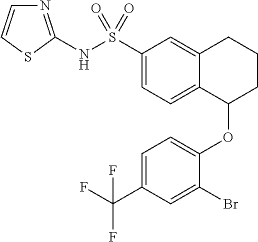 [SMILES: O=S(=O)(Nc1nccs1)c1ccc2c(c1)CCCC2Oc1ccc(C(F)(F)F)cc1Br]

The title compound was prepared by following similar procedure as described in Example-88 using Intermediate-22. Yield: 12%; LCMS(ESI): m/z 532.93 & 534.90 (M+H)⁺; ¹H NMR (400 MHz, Chloroform-d) δ 7.85 (d, J=2.5 Hz, 1H), 7.79-7.72 (m, 2H), 7.61-7.49 (m, 2H), 7.17-7.09 (m, 2H), 6.56 (d, J=4.6 Hz, 1H), 5.48 (t, J=5.0 Hz, 1H), 2.70-2.50 (m, 2H), 2.20-1.57 (m, 4H)

### Example-91: 5-(2-Chloro-4-(trifluoromethyl)phenoxy)-N-(1,2,4-thiadiazol-5-yl)-5,6,7,8-tetrahydronaphthalene-2-sulfonamide

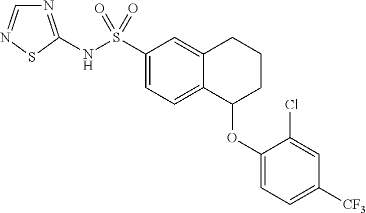 [SMILES: O=S(=O)(Nc1ncns1)c1ccc2c(c1)CCCC2Oc1ccc(C(F)(F)F)cc1Cl]

The title compound was prepared by following the similar procedure as described in Example-88 using Intermediate-21 and 1,2,4-thiadiazol-5-amine. Yield: 37%; LCMS(ESI): m/z 490.0 (M+H)⁺; ¹H NMR (400 MHz, Chloroform-d) δ 8.03 (s, 1H), 7.78-7.66 (m, 3H), 7.60-7.49 (m, 2H), 7.17 (d, J=8.6 Hz, 1H), 5.47 (t, J=4.9 Hz, 1H), 3.05-2.95 (m, 1H), 2.93-2.82 (m, 1H), 2.16-2.00 (m, 3H), 1.89 (d, J=6.9 Hz, 1H).

### Example-92: 5-(2-Bromo-4-(trifluoromethyl)phenoxy)-N-(1,2,4-thiadiazol-5-yl)-5,6,7,8-tetrahydronaphthalene-2-sulfonamide

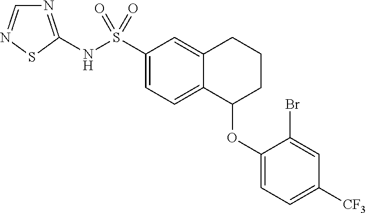 [SMILES: O=S(=O)(Nc1ncns1)c1ccc2c(c1)CCCC2Oc1ccc(C(F)(F)F)cc1Br]

The title compound was prepared by following the similar procedure as described in Example-88 using Intermediate 22 and 1,2,4-thiadiazol-5-amine. Yield: 10%; LCMS(ESI): m/z: 533.93 & 535.87 (M+&M+2)⁺; ¹H NMR (400 MHz, Chloroform-d) δ 8.03 (s, 1H), 7.90-7.71 (m, 3H), 7.69-7.54 (m, 2H), 7.14 (dd, J=8.8, 4.4 Hz, 1H), 5.54-5.44 (m, 1H), 3.05-2.95 (m, 1H), 2.90-2.80 (m, 1H), 2.20-2.05 (m, 3H), 1.95-1.85 (m, 1H).

### Example-93: N-(Thiazol-2-yl)-5-(4-(trifluoromethyl)phenoxy)-5,6,7,8-tetrahydro naphthalene-2-sulfonamide

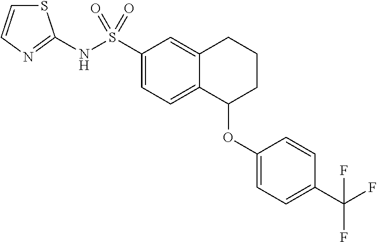 [SMILES: O=S(=O)(Nc1nccs1)c1ccc2c(c1)CCCC2Oc1ccc(C(F)(F)F)cc1]

The title compound was prepared by following the similar procedure as described in Example-88 using 4-(trifluoromethyl)phenol. Yield: 12%; LCMS(ESI): m/z 454.98 (M+H)⁺; ¹H NMR (400 MHz, Chloroform-d) δ 7.83-7.69 (m, 2H), 7.59 (d, J=8.7 Hz, 2H), 7.46 (d, J=8.7 Hz, 1H), 7.17 (d, J=4.6 Hz, 1H), 7.06 (d, J=8.5 Hz, 2H), 6.55 (d, J=4.6 Hz, 1H), 5.51-5.39 (m, 1H), 3.07-2.76 (m, 2H), 2.23-1.78 (m, 4H).

### Example-94: 5-(2-(1-Methyl-1H-pyrazol-5-yl)-4-(trifluoromethyl)phenoxy)-N-(thiazol-2-yl)-5,6,7,8-tetrahydronaphthalene-2-sulfonamide

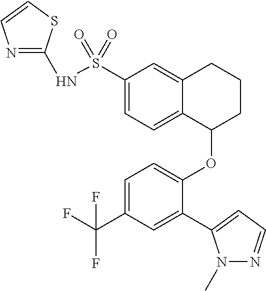 [SMILES: Cn1nccc1-c1cc(C(F)(F)F)ccc1OC1CCCc2cc(S(=O)(=O)Nc3nccs3)ccc21]

The title compound was prepared by following the similar procedure as described in Example-88 using Intermediate-23. Yield: 12%; LCMS(ESI): m/z 535.02 (M+H)⁺; ¹H NMR (400 MHz, Chloroform-d) δ 7.82-7.63 (m, 4H), 7.56-7.52 (m, 2H), 7.30 (d, J=9.2 Hz, 1H), 7.17-7.13 (m, 2H), 6.56 (d, J=3.9 Hz, 1H), 5.40-5.32 (m, 1H), 3.63 (s, 3H), 2.81 (d, J=20.7 Hz, 2H), 2.05-1.85 (m, 2H), 1.87-1.70 (m, 2H).

### Example-95: (R)-5-(2-(1-Methyl-1H-pyrazol-5-yl)-4-(trifluoromethyl)phenoxy)-N-(thiazol-2-yl)-5,6,7,8-tetrahydronaphthalene-2-sulfonamide

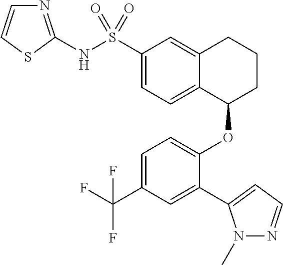 [SMILES: Cn1nccc1-c1cc(C(F)(F)F)ccc1O[C@@H]1CCCc2cc(S(=O)(=O)Nc3nccs3)ccc21]

The title compound was prepared by following the similar procedure as described in Example-88 using Intermediate-23a. Yield: 32%; LCMS(ESI): m/z 535.01 (M+H)⁺; ¹H NMR (400 MHz, DMSO-d₆) δ 12.75 (bs, 1H), 7.86 (dd, J=8.9, 2.4 Hz, 1H), 7.68 (d, J=8.8 Hz, 1H), 7.61 (d, J=2.5 Hz, 1H), 7.57-7.53 (m, 2H), 7.43-7.33 (m, 2H), 7.26 (d, J=4.6 Hz, 1H), 6.84 (d, J=4.6 Hz, 1H), 6.24 (d, J=1.9 Hz, 1H), 5.73 (t, J=4.9 Hz, 1H), 3.50 (s, 3H), 2.86-2.70 (m, 2H), 2.05-1.85 (m, 2H), 1.80-1.60 (m, 2H).

### Example-96: 5-(2-(1,2,3,6-Tetrahydropyridin-4-yl)-4-(trifluoromethyl)phenoxy)-N-(thiazol-2-yl)-5,6,7,8-tetrahydronaphthalene-2-sulfonamide trifluoroaceticacid

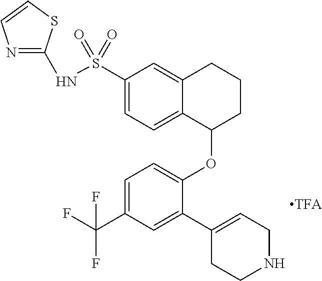 [SMILES: O=S(=O)(Nc1nccs1)c1ccc2c(c1)CCCC2Oc1ccc(C(F)(F)F)cc1C1=CCNCC1]

To a solution of tert-butyl 4-(2-((6-((perfluorophenoxy)sulfonyl)-1,2,3,4-tetrahydronaphthalen-1-yl)oxy)-5-(trifluoromethyl)phenyl)-5,6-dihydropyridine-1(2H)-carboxylate (Intermediate-24) (0.05 g, 0.07 mmol) and thiazol-2-amine (0.007 g, 0.07 mmol) in THF (2 ml) was added LiHMDS (1M in THF, 0.172 ml, 0.172 mmol) at 0° C. and slowly warm to ambient temperature and stirred for 2 h. TLC indicated completion of reaction. The reaction mixture was poured into saturated ammonium chloride solution and the product was extracted with DCM. The combined organic layer was washed with brine, dried over Na₂SO₄, concentrated under vacuum and purified by prep HPLC. Boc group was cleaved by taking the above compound in DCM (1 ml) and treated with TFA (0.2 ml) for 1 h. TFA was removed under reduced pressure and triturated with ether. The solvent was decanted and dried under vacuum to obtain the title compound as off-white solid (0.010 g, 27%). LCMS(ESI): m/z 536.1 (M+H)⁺; ¹H NMR (400 MHz, Methanol-d₄) δ 7.74-7.65 (m, 3H), 7.51-7.38 (m, 3H), 7.13 (d, J=4.6 Hz, 1H), 6.75 (d, J=4.7 Hz, 1H), 5.78-5.76 (m, 1H), 5.70 (t, J=4.7 Hz, 1H), 3.75-3.70 (m, 2H), 3.30-3.20 (m, 2H), 3.04-2.79 (m, 2H), 2.71-2.53 (m, 2H), 2.20-2.10 (m, 2H), 2.07-1.86 (m, 2H).

Below Example 97 and 98 were prepared by following the similar procedure as described in Example-96 using Intermediates 24a and 24b respectively.

### Example 97: (R/S)-5-(2-(1,2,3,6-tetrahydropyridin-4-yl)-4-(trifluoromethyl)phenoxy)-N-(thiazol-2-yl)-5,6,7,8-tetrahydronaphthalene-2-sulfonamide

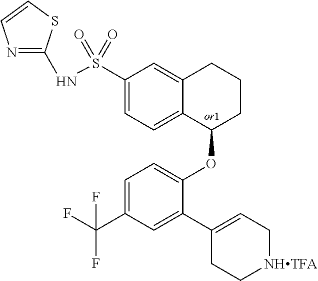

Chiral HPLC: Retention time 8.07 min.; Chiral column IA; Mobile Phase:n-hexane:01% DEA:0.1 TFA):(ETOH:DCM, 1:1) 80:20.

### Example 98: (R/S)-5-(2-(1,2,3,6-Tetrahydropyridin-4-yl)-4-(trifluoromethyl)phenoxy)-N-(thiazol-2-yl)-5,6,7,8-tetrahydronaphthalene-2-sulfonamide

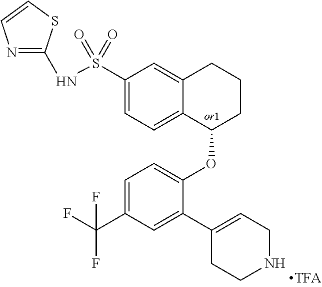 [SMILES: O=S(=O)(Nc1nccs1)c1ccc2c(c1)CCC[C@@H]2Oc1ccc(C(F)(F)F)cc1C1=CCNCC1]

Chiral HPLC: Retention time 10.10 min.; Chiral column IA; Mobile Phase:n-hexane:01% DEA:0.1 TFA):(ETOH:DCM, 1:1) 80:20

### Example-99: 5-(2-(Piperidin-4-yl)-4-(trifluoromethyl)phenoxy)-N-(thiazol-2-yl)-5,6,7,8-tetrahydronaphthalene-2-sulfonamide trifluoroacetic acid

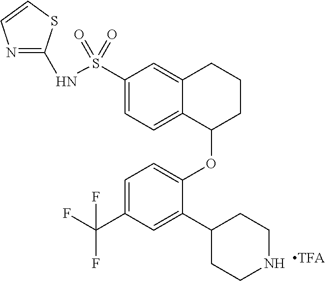 [SMILES: O=S(=O)(Nc1nccs1)c1ccc2c(c1)CCCC2Oc1ccc(C(F)(F)F)cc1C1CCNCC1]

To a solution of tert-butyl 4-(2-((6-((Perfluorophenoxy)sulfonyl)-1,2,3,4-tetrahydro naphthalen-1-yl)oxy)-5-(trifluoromethyl)phenyl)piperidine-1-carboxylate (Intermediate-25) (0.45 g, 0.624 mmol) and thiazol-2-amine (0.094 g, 0.935 mmol) in THF (5 ml) was added LiHMDS (1M in THF, 0.172 ml, 0.172 mmol) at 0° C. and stirred at ambient temperature for 2 h. The reaction mixture was poured into saturated aqueous ammonium chloride solution and extracted with dichloromethane. Crude product was purified by flash column chromatography (40% ethyl acetate/Petroleum ether) to obtain Boc protected intermediate. Boc group was cleaved by taking above compound in DCM (5 ml) and treated with TFA (1 ml) for 1 h. TFA was removed under reduced pressure and triturated with MeOH. It was filtered, filtrate was evaporated and again triturated with ether and decanted the solvent and dried under vacuum to obtain title compound as off white solid (0.17 g, 42%). LCMS(ESI): m/z 537.82 (M+H)⁺; ¹H NMR (400 MHz, Methanol-d₄) δ 7.74 (d, J=1.9 Hz, 1H), 7.69 (dd, J=8.1, 2.0 Hz, 1H), 7.64-7.59 (m, 1H), 7.49 (d, J=2.3 Hz, 1H), 7.45-7.40 (m, 2H), 7.13 (d, J=4.7 Hz, 1H), 6.75 (d, J=4.6 Hz, 1H), 5.71 (t, J=4.8 Hz, 1H), 3.44-3.36 (m, 1H), 3.25-3.15 (m, 2H), 3.10-2.86 (m, 4H), 2.20-2.14 (m, 2H), 2.09-1.70 (m, 6H).

The enantiomers of Example-99 were separated using chiral prep (Column: Chiral pak IA; mobile phase: n-hexane:0.1% DEA:0.1 TFA):(ETOH:DCM, 1:1) 85:15, affording Example-100 (retention time 6.77 minutes) and Example-101 (retention time 7.88 min).

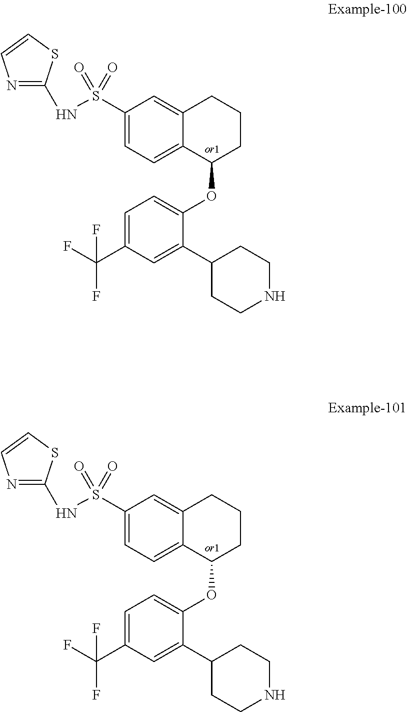 [SMILES: O=S(=O)(Nc1nccs1)c1ccc2c(c1)CCC[C@H]2Oc1ccc(C(F)(F)F)cc1C1CCNCC1 ; O=S(=O)(Nc1nccs1)c1ccc2c(c1)CCC[C@@H]2Oc1ccc(C(F)(F)F)cc1C1CCNCC1]

Below Examples-102 and 103 were prepared by following the similar procedure as described in Example-99 using Intermediate-25.

### Example-102: 5-(2-(Piperidin-4-yl)-4-(trifluoromethyl)phenoxy)-N-(1,2,4-thiadiazol-5-yl)-5,6,7,8-tetrahydronaphthalene-2-sulfonamide

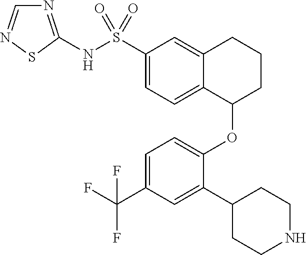 [SMILES: O=S(=O)(Nc1ncns1)c1ccc2c(c1)CCCC2Oc1ccc(C(F)(F)F)cc1C1CCNCC1]

Yield: 10%; LCMS(ESI): m/z 538.95 (M+H)⁺; ¹H NMR (400 MHz, Methanol-d₄) δ 7.95 (s, 1H), 7.72 (d, J=1.9 Hz, 1H), 7.66-7.60 (m, 2H), 7.45-7.40 (m, 2H), 7.27 (d, J=8.2 Hz, 1H), 5.66 (t, J=4.4 Hz, 1H), 3.45-3.35 (m, 1H), 3.33-3.20 (m, 1H), 3.06-2.80 (m, 5H), 2.24-1.80 (m, 6H), 1.75-1.65 (m, 2H).

### Example-103: 5-(2-(Piperidin-4-yl)-4-(trifluoromethyl)phenoxy)-N-(1,3,4-thiadiazol-2-yl)-5,6,7,8-tetrahydronaphthalene-2-sulfonamide trifluoroacetic acid

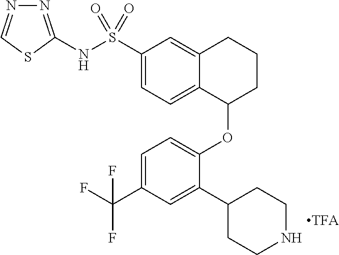 [SMILES: O=S(=O)(Nc1nncs1)c1ccc2c(c1)CCCC2Oc1ccc(C(F)(F)F)cc1C1CCNCC1]

Yield: 17%; LCMS(ESI): m/z 538.82 (M+H)⁺; ¹H NMR (400 MHz, Methanol-d4) δ 8.55 (s, 1H), 7.72 (d, J=1.9 Hz, 1H), 7.67 (dd, J=8.1, 2.0 Hz, 1H), 7.62 (dd, J=8.6, 2.3 Hz, 1H), 7.53-7.47 (m, 1H), 7.48-7.41 (m, 2H), 5.72 (t, 1H), 3.50-3.35 (m, 1H), 3.25-3.15 (m, 1H), 3.11-2.85 (m, 5H), 2.20-2.10 (m, 2H), 2.07-1.80 (m, 6H),

### Example-104: 5-(2-(1-Methylpiperidin-4-yl)-4-(trifluoromethyl)phenoxy)-N-(thiazol-2-yl)-5,6,7,8-tetrahydronaphthalene-2-sulfonamide

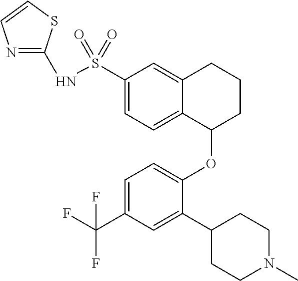 [SMILES: CN1CCC(c2cc(C(F)(F)F)ccc2OC2CCCc3cc(S(=O)(=O)Nc4nccs4)ccc32)CC1]

To a solution of 5-(2-(piperidin-4-yl)-4-(trifluoromethyl)phenoxy)-N-(thiazol-2-yl)-5,6,7,8-tetrahydronaphthalene-2-sulfonamide (Example-99)(0.015 g, 0.028 mmol) in MeOH (1 ml):DCM (10 ml) was added formaldehyde solution (0.082 ml, 0.837 mmol) and acetic acid (0.080 ml, 1.395 mmol) and stirred for 30 min. at ambient temperature, then sodium triacetoxyborohydride (0.030 g, 0.140 mmol) was added portion wise. The reaction mixture was stirred further 30 min. The reaction mixture was poured into saturated solution of sodium carbonate and extracted with chloroform. The combined organic layer was washed with brine, dried over Na₂SO₄ and concentrated under vacuum. The crude solid was triturated with ether and decanted the solvent and dried under vacuum to obtain title compound as white solid (0.005 g, 32%). LCMS(ESI): m/z 552.13 (M+H)⁺; 1H NMR (400 MHz, Methanol-d₄) δ 7.73 (s, 1H), 7.67 (d, J=8.2 Hz, 1H), 7.63-7.56 (m, 1H), 7.47 (d, J=2.3 Hz, 1H), 7.43 (s, 1H), 7.35 (d, J=8.1 Hz, 1H), 7.11 (d, J=4.5 Hz, 1H), 6.71 (d, J=4.5 Hz, 1H), 5.68 (t, J=4.5 Hz, 1H), 3.40-3.35 (m, 1H), 3.30-3.24 (m, 1H), 3.11-2.73 (m, 5H), 2.68 (s, 3H), 2.20-2.10 (m, 2H), 2.05-1.70 (m, 6H), Below Examples-105 and 106 were prepared by following similar procedure as described in Example-104 using Example 102, 103 respectively.

### Example-105: 5-(2-(1-Methylpiperidin-4-yl)-4-(trifluoromethyl)phenoxy)-N-(1,2,4-thiadiazol-5-yl)-5,6,7,8-tetrahydronaphthalene-2-sulfonamide

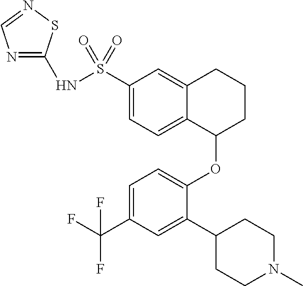 [SMILES: CN1CCC(c2cc(C(F)(F)F)ccc2OC2CCCc3cc(S(=O)(=O)Nc4ncns4)ccc32)CC1]

Yield: 38%; LCMS(ESI): m/z 552.82 (M+H)⁺; ¹H NMR (400 MHz, Methanol-d₄) δ 7.96 (s, 1H), 7.76-7.58 (m, 3H), 7.50-7.40 (m, 2H), 7.28 (d, J=8.1 Hz, 1H), 5.67 (t, J=4.2 Hz, 1H), 3.60-3.40 (m, 1H), 3.30-3.20 (m, 1H), 3.11-2.89 (m, 5H), 2.82 (s, 3H), 2.30-1.82 (m, 8H).

### Example-106: 5-(2-(1-Methylpiperidin-4-yl)-4-(trifluoromethyl)phenoxy)-N-(1,2,4-thiadiazol-5-yl)-5,6,7,8-tetrahydronaphthalene-2-sulfonamide

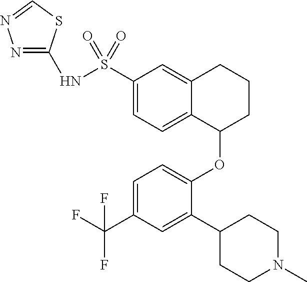 [SMILES: CN1CCC(c2cc(C(F)(F)F)ccc2OC2CCCc3cc(S(=O)(=O)Nc4nncs4)ccc32)CC1]

Yield: 25%; LCMS(ESI): m/z 553.10 (M+H)⁺; ¹H NMR (400 MHz, methanol-d4) δ 8.57 (s, 1H), 7.74-7.68 (m, 1H), 7.82-7.58 (m, 2H), 7.46-7.40 (m, 2H), 7.24 (d, J=8.1 Hz, 1H), 5.65 (t, J=4.1 Hz, 1H), 3.50-3.40 (m, 1H), 3.37-3.30 (m, 1H), 3.05-2.80 (m, 5H), 2.78 (s, 3H), 2.28-2.20 (m, 2H), 12.10-1.80 (m, 6H).

### Example-107: (R)-4-(2-(2-Fluoroethyl)-4-(trifluoromethyl)phenoxy)-N-(thiazol-2-yl)chroman-7-sulfonamide

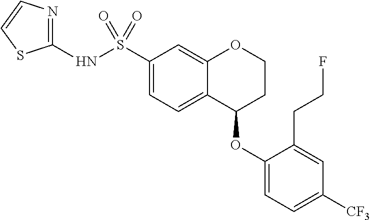 [SMILES: O=S(=O)(Nc1nccs1)c1ccc2c(c1)OCC[C@H]2Oc1ccc(C(F)(F)F)cc1CCF]

The title compound was prepared by following similar procedure as described for Example-34 using Intermediate-39 and Intermediate-12a LCMS(ESI): m/z 502.94 (M+H)⁺; ¹H NMR (400 MHz, DMSO-d6) δ 12.82 (s, 1H), 7.66 (dd, J=8.8, 2.4 Hz, 1H), 7.61 (d, J=2.4 Hz, 1H), 7.52-7.47 (m, 2H), 7.33 (dd, J=8.0, 1.8 Hz, 1H), 7.28 (d, J=4.6 Hz, 1H), 7.23 (d, J=1.8 Hz, 1H), 6.86 (d, J=4.6 Hz, 1H), 5.76 (t, J=4.1 Hz, 1H), 4.58 (td, J=6.3, 2.3 Hz, 1H), 4.47 (td, J=6.3, 2.4 Hz, 1H), 4.40-4.32 (m, 1H), 4.20 (td, J=10.8, 2.8 Hz, 1H), 2.98 (t, J=6.2 Hz, 1H), 2.91 (t, J=6.2 Hz, 1H), 2.31-2.21 (m, 1H), 2.21-2.09 (m, 1H).

### Example-108: (R)-4-(2-(1-Ethyl-1H-pyrazol-5-yl)-4-(trifluoromethyl)phenoxy)-N-(thiazol-2-yl)chroman-7-sulfonamide

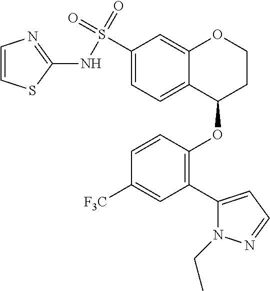 [SMILES: CCn1nccc1-c1cc(C(F)(F)F)ccc1O[C@@H]1CCOc2cc(S(=O)(=O)Nc3nccs3)ccc21]

The title compound was prepared by following similar procedure as described for Example-34 using Intermediate-38 and Intermediate-12a LCMS(ESI): m/z 550.82 (M+H)⁺; ¹H NMR (400 MHz, DMSO-d₆) δ 12.82 (s, 1H), 7.99-7.84 (m, 1H), 7.75 (d, J=8.8 Hz, 1H), 7.60 (d, J=2.4 Hz, 1H), 7.43 (d, J=1.8 Hz, 1H), 7.36 (d, J=8.1 Hz, 1H), 7.31-7.22 (m, 2H), 7.14 (d, J=1.8 Hz, 1H), 6.86 (d, J=4.6 Hz, 1H), 6.18 (d, J=1.8 Hz, 1H), 5.75 (t, 1H), 4.32-4.17 (m, 1H), 3.89-3.80 (m, 1H), 3.73 (q, J=7.0 Hz, 2H), 2.25-2.01 (m, 2H), 1.01 (t, J=7.2 Hz, 3H).

### Example-109: (R)-2-(2-((2,2-Dimethyl-7-(N-(thiazol-2-yl)sulfamoyl)chroman-4-yl)oxy)-5-(trifluoromethyl)phenyl)pyridine 1-oxide

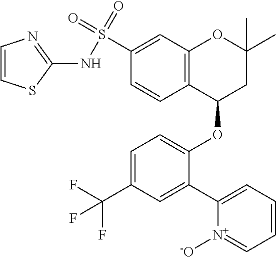

The title compound was prepared by following similar procedure as described in Example-61 using Intermediate-17a.

LCMS(ESI): m/z 577.93 (M+H)⁺; ¹H NMR (400 MHz, DMSO-d6) δ 12.78 (s, 1H), 8.36-8.29 (m, 1H), 7.91-7.80 (m, 1H), 7.74 (d, J=2.4 Hz, 1H), 7.59 (d, J=8.2 Hz, 2H), 7.49 (dd, J=7.7, 2.2 Hz, 1H), 7.41-7.34 (m, 1H), 7.33-7.29 (m, 1H), 7.29-7.21 (m, 2H), 7.06 (d, J=1.8 Hz, 1H), 6.85 (d, J=4.6 Hz, 1H), 5.76 (d, J=2.7 Hz, 1H), 2.46-2.36 (m, 1H), 1.94-1.82 (m, 1H), 1.35 (s, 3H), 1.22 (s, 3H).

### Example-110: (R)-3-(2-((7-(N-(Thiazol-2-yl)sulfamoyl)chroman-4-yl)oxy)-5-(trifluoro methyl)phenyl)pyridine 1-oxide

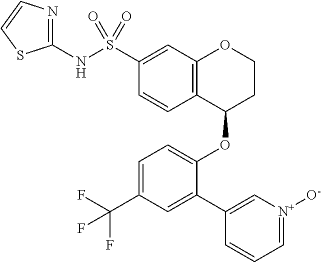 [SMILES: O=S(=O)(Nc1nccs1)c1ccc2c(c1)OCC[C@H]2Oc1ccc(C(F)(F)F)cc1-c1ccc[n+]([O-])c1]

### Step-1: (R)-perfluorophenyl 4-(2-(pyridin-3-yl)-4-(trifluoromethyl)phenoxy)chroman-7-sulfonate

The title compound was prepared similarly as described in Example-41 using (R)-perfluorophenyl 4-(2-bromo-4-(trifluoromethyl)phenoxy)chroman-7-sulfonate (Step-1, Intermediate-14a). Yield: 52%; LCMS(ESI): m/z 617.58 (M+H)⁺.

### Step-2: (R)-3-(2-((7-((perfluorophenoxy)sulfonyl)chroman-4-yl)oxy)-5-(trifluoromethyl) phenyl)pyridine 1-oxide

The title compound was prepared by following similar procedure as described in Example-61 (step-2).

Yield: 75%; LCMS(ESI): m/z 633.58 (M+H)⁺; ¹H NMR (400 MHz, Chloroform-d) δ 8.63 (s, 1H), 8.40 (d, J=6.2 Hz, 1H), 7.85-7.77 (m, 1H), 7.64 (d, J=2.3 Hz, 1H), 7.56-7.29 (m, 6H), 5.58-5.51 (m, 1H), 4.47-4.37 (m, 1H), 4.29-4.17 (m, 1H), 2.36 (dd, J=10.3, 4.5 Hz, 2H).

### Step-3: (R)-3-(2-((7-(N-(thiazol-2-yl)sulfamoyl)chroman-4-yl)oxy)-5-(trifluoro methyl) phenyl)pyridine 1-oxide

The title compound was prepared by following similar procedure as described in Example-34 using step-2 intermediate.

Yield: 28%; LCMS(ESI): m/z 549.70 (M+H)⁺; ¹H NMR (400 MHz, DMSO-d₆) δ 12.83 (s, 1H), 8.33 (dt, J=2.0, 1.0 Hz, 1H), 8.19-8.11 (m, 1H), 7.91-7.83 (m, 1H), 7.77 (d, J=2.4 Hz, 1H), 7.72 (d, J=8.8 Hz, 1H), 7.45 (d, J=8.1 Hz, 1H), 7.40-7.34 (m, 2H), 7.31-7.23 (m, 2H), 7.16 (d, J=1.8 Hz, 1H), 6.85 (d, J=4.6 Hz, 1H), 5.79 (t, 1H), 4.40-4.22 (m, 1H), 4.04 (td, J=10.9, 2.8 Hz, 1H), 2.41-2.03 (m, 2H).

### Example-111: (R)-4-(2-((7-(N-(Thiazol-2-yl)sulfamoyl)chroman-4-yl)oxy)-5-(trifluoromethyl)phenyl)pyridine 1-oxide

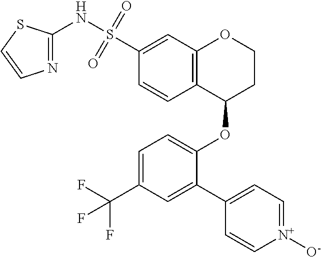 [SMILES: O=S(=O)(Nc1nccs1)c1ccc2c(c1)OCC[C@H]2Oc1ccc(C(F)(F)F)cc1-c1cc[n+]([O-])cc1]

The title compound was prepared by following similar procedure as described in Example-110.

LCMS(ESI): m/z 549.97 (M+H)⁺; ¹H NMR (400 MHz, DMSO-d₆) δ 12.83 (s, 1H), 8.22-8.10 (m, 2H), 7.83 (dd, J=8.8, 2.4 Hz, 1H), 7.78-7.69 (m, 2H), 7.55-7.48 (m, 2H), 7.41 (d, J=8.1 Hz, 1H), 7.32-7.25 (m, 2H), 7.16 (d, J=1.8 Hz, 1H), 6.86 (d, J=4.6 Hz, 1H), 5.79 (t, 1H), 4.31 (dd, J=10.3, 5.5 Hz, 1H), 4.13-4.05 (m, 1H), 2.30-2.12 (m, 2H).

### Pharmacological Activity

Certain illustrative compounds within the scope of the invention are screened for Nay activity according to the procedure given below. The screening of the compounds may be carried by other methods and procedures known to skilled in the art.

### Analysis of Inhibition of Sodium Channels by Test Compounds:

HEK-293 cells overexpressing the channel of interest were seeded in a 96-well plate at a density of 30000 cells/well and incubated at 37° C./5% CO₂ for 48 hr. The assay was carried out using the Red Membrane Potential Dye (Molecular Devices) following the manufacturer's instructions. Briefly, the cells were incubated with 1× red membrane potential dye for 1.5 hour. The cells were then treated with various concentrations of the test compounds for 15-20 min followed by depolarization with 10-30 μM Veratridine. The fluorescence was read following excitation at 510-545 nm and emission at 565-625 nm in FLIPR. The “max-min” fluorescence values were used to calculate the % inhibition. IC₅₀ values were calculated by plotting % inhibition against concentration and curve fitting into a sigmoidal dose response.

Certain compounds of the present invention are shown to have functional activity as inhibitors of Na_{V}1.7 and Na_{V}1.5 in vitro.

TABLE 1

| Example | Nav1.7 | Nav1.5 IC₅₀ (nM)/ |
|---|---|---|
| No. | IC₅₀ (nM) | % inhibition @ 10 μM |
| 01 | 319 | 3910 |
| 02 | 572 | 1333 |
| 03 | 919 | 6615 |
| 04 | 164 | 74% |
| 05 | 60 | 585 |
| 06 | 1692 | 65% |
| 07 | 13 | 39% |
| 08 | 8 | 49% |
| 09 | 62 | 51% |
| 10 | 3585 | 31% |
| 11 | 13 | 61% |
| 12 | 33 | 40% |
| 13 | 23 | 54% |
| 14 | 1163 | 26% |
| 15 | 798 | 39% |
| 16 | 101 | 2311 |
| 17 | 1432 | 5948 |
| 18 | 113 | 47% |
| 19 | 933 | 32% |
| 20 | >10 μM | >10 μM |
| 21 | 9 | 2492 |
| 22 | 52 | 1308 |
| 23 | 9 | 931 |
| 24 | 500 | >10 μM |
| 25 | 4 | 3003 |
| 26 | 146 | 2021 |
| 27 | 27 | 1989 |
| 28 | 1 | 484 |
| 29 | 2 | 1060 |
| 30 | 4 | 8296 |
| 31 | 2 | 486 |
| 32 | 4 | 108 |
| 33 | 305 | 2974 |
| 34 | 77 | 1625 |
| 35 | 261 | 58% |
| 36 | 472 | 2033 |
| 37 | 614 | 2445 |
| 38 | 1053 | 2965 |
| 39 | 33 | 3446 |
| 40 | 35 | 63% |
| 41 | 9 | 75% |
| 42 | 418 | 61% |
| 43 | 18 | 3737 |
| 44 | 75 | 3822 |
| 45 | 4 | 1949 |
| 46 | 43 | 1645 |
| 47 | 972 | 58% |
| 48 | 199 | 2718 |
| 49 | 29 | 2123 |
| 50 | 1789 | 40% |
| 51 | 170 | 611 |
| 52 | 61 | 4906 |
| 53 | 15 | 3150 |
| 54 | 223 | 2710 |
| 55 | 11 | 75% |
| 56 | 2895 | >10 μM |
| 57 | 56 | 72% |
| 58 | 93 | 84% |
| 59 | 122 | 62% |
| 60 | 122 | >10 μM |
| 61 | 81 | >10 μM |
| 62 | 58 | 50% |
| 63 | 45 | >10 μM |
| 64 | 299 | >10 μM |
| 65 | 171 | 69% |
| 66 | 911 | 4406 |
| 67 | 460 | 1693 |
| 68 | 447 | 3724 |
| 69 | 393 | ND |
| 70 | 533 | 45% |
| 71 | 117 | 2591 |
| 72 | 66 | 4963 |
| 73 | 390 | 3014 |
| 74 | 54 | >10 μM |
| 75 | 98 | 56% |
| 76 | 54 | 69% |
| 77 | 576 | 64% |
| 78 | 589 | 60% |
| 79 | 25 | 69% |
| 80 | 33 | 820 |
| 81 | 138 | 1264 |
| 82 | 341 | 2702 |
| 83 | 66 | 1285 |
| 84 | 201 | 5429 |
| 85 | 208 | 3802 |
| 86 | 14 | 50% |
| 87 | 13 | 2855 |
| 88 | 176 | 2718 |
| 89 | 111 | 2374 |
| 90 | 289 | 3717 |
| 91 | 989 | 601 |
| 92 | 13 | 3585 |
| 93 | 698 | ND |
| 94 | 49 | 2778 |
| 95 | 25 | 2277 |
| 96 | 4 | 1652 |
| 97 | 5 | 3564 |
| 98 | 38 | 3190 |
| 99 | 14 | 6149 |
| 100 | 11 | 3414 |
| 101 | 276 | 4598 |
| 102 | 41 | >10 μM |
| 103 | 570 | 51% |
| 104 | 318 | 92% |
| 105 | 912 | 27% |
| 106 | 2014 | 54% |
| 107 | 19 | 386 |
| 108 | 20 | 54% |
| 109 | 45 | >10 μM |
| 110 | 13 | >10 μM |
| 111 | 13 | >10 μM |

1.-30. (canceled)

31. A compound having the Formula (I): 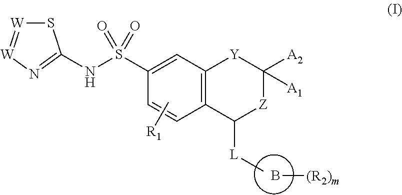
wherein,
Y is selected from CH₂, O and NR;
L is a bond or O;
R is hydrogen or substituted or unsubstituted alkyl;
A₁ and A₂ are independently hydrogen or substituted or unsubstituted alkyl; or
A₁ and A₂, together with the carbon atom to which they are attached, form a substituted or unsubstituted 3 to 6 membered cycloalkyl ring or 4-6 membered heterocyclyl ring;
Z is selected from CH₂ or —CH₂—CH₂—;
R₁ is selected from hydrogen, halogen, cyano, substituted or unsubstituted alkyl and substituted or unsubstituted alkoxy;
ring B is aryl or heteroaryl;
R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;
W at each occurrence is independently selected from N or CR₃;
R₃ is selected from hydrogen, halogen, cyano, substituted or unsubstituted alkyl and substituted or unsubstituted alkoxy;
‘m’ is an integer ranging from 0 to 3, both inclusive;
wherein the substituents for substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are one or more same or different and independently selected from the group consisting of hydroxy, halogen, carboxy, cyano, nitro, oxo (═O), alkyl, haloalkyl, hydroxyalkyl, alkoxyalkyl, alkenyl, alkynyl, aryl, arylalkyl, cycloalkyl, cycloalkylalkyl, cycloalkenyl, heteroaryl, heterocyclic ring, heterocyclylalkyl, heteroarylalkyl, —C(O)ORₓ, —C(O)R_{y}, —C(S)R_{y}, —C(O)NRₓR_{z}, —NRₓC(O)NRₓR_{z}, —N(Rₓ)S(O)₂R_{y}, —NRₓR_{z}, —NRₓC(O)R_{y}, —S(O)₂NRₓR_{z}, —ORₓ, —OC(O)R_{y}, and —S(O)₀₋₂R_{y};
Rₓ, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl;
R_{y}, which may be same or different at each occurrence, is independently selected from alkyl, haloalkyl, alkenyl, alkynyl, aryl, arylalkyl, cycloalkyl, cycloalkenyl, heteroaryl, heterocyclic ring, heterocyclylalkyl and heteroarylalkyl; and
R_{z}, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl; or Rₓ and R_{z} together with the nitrogen atom to which they are attached form a substituted or unsubstituted, saturated or unsaturated 4 to 8 membered cyclic ring, wherein the unsaturated cyclic ring may have one or two double bonds;
or N-oxides thereof or a pharmaceutically acceptable salt thereof.

32. The compound of claim 31, having the Formula (Ia): 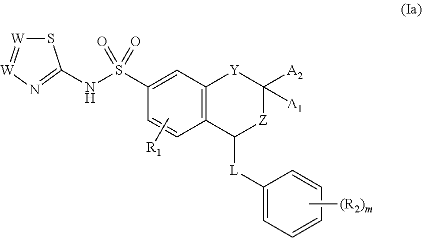
wherein,
Y is O or CH₂;
L is a bond or O;
Z is —CH₂—;
A₁ and A₂ are independently hydrogen or substituted or unsubstituted alkyl; or
A₁ and A₂, together with the carbon atom to which they are attached, form a substituted or unsubstituted 3 to 6 membered cycloalkyl ring or 4-6 membered heterocyclyl ring;
R₁ is selected from hydrogen, halogen, cyano, substituted or unsubstituted alkyl and substituted or unsubstituted alkoxy;
R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;
W at each occurrence is independently selected from N or CR₃;
R₃ is selected from hydrogen, halogen, cyano, substituted or unsubstituted alkyl and substituted or unsubstituted alkoxy;
‘m’ is an integer ranging from 0 to 3, both inclusive;
wherein the substituents for substituted or unsubstituted alkyl, substituted or unsubstituted alkoxy, substituted or unsubstituted cycloalkyl, substituted or unsubstituted aryl, substituted or unsubstituted heteroaryl, substituted or unsubstituted heterocyclyl, substituted or unsubstituted hydroxyalkyl, substituted or unsubstituted alkoxyalkyl, substituted or unsubstituted haloalkyl are one or more same or different and independently selected from the group consisting of hydroxy, halogen, carboxy, cyano, nitro, oxo (═O), alkyl, haloalkyl, hydroxyalkyl, alkoxyalkyl, alkenyl, alkynyl, aryl, arylalkyl, cycloalkyl, cycloalkylalkyl, cycloalkenyl, heteroaryl, heterocyclic ring, heterocyclylalkyl, heteroarylalkyl, —C(O)ORₓ, —C(O)R_{y}, —C(S)R_{y}, —C(O)NRₓR_{z}, —NRₓC(O)NRₓR_{z}, —N(Rₓ)S(O)₂R_{y}, —NRₓR_{z}, —NRₓC(O)R_{y}, —S(O)₂NRₓR_{z}, —ORₓ, —OC(O)R_{y}, and —S(O)₀₋₂R_{y};
Rₓ, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl;
R_{y}, which may be same or different at each occurrence, is independently selected from alkyl, haloalkyl, alkenyl, alkynyl, aryl, arylalkyl, cycloalkyl, cycloalkenyl, heteroaryl, heterocyclic ring, heterocyclylalkyl and heteroarylalkyl; and
R_{z}, which may be same or different at each occurrence, is independently selected from hydrogen, alkyl, haloalkyl, alkenyl, alkynyl, aryl, arylalkyl, cycloalkyl, cycloalkenyl, heteroaryl, heterocyclic ring, heterocyclylalkyl and heteroarylalkyl; or Rₓ and R_{z} together with the nitrogen atom to which they are attached form a substituted or unsubstituted, saturated or unsaturated 4 to 8 membered cyclic ring, wherein the unsaturated cyclic ring may have one or two double bonds;
or N-oxides thereof or a pharmaceutically acceptable salt thereof.

33. The compound according to claim 31 wherein Y is CH₂ or O.

34. The compound according to claim 31 wherein L is a bond or O.

35. The compound of claim 31, having the Formula (II): 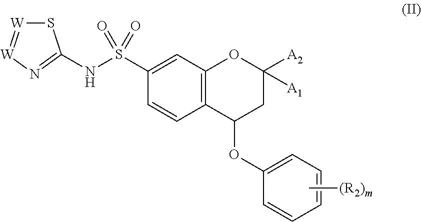
wherein,
A₁ and A₂ are independently hydrogen or substituted or unsubstituted alkyl; or
A₁ and A₂, together with the carbon atom to which they are attached, form a substituted or unsubstituted 3 to 6 membered cycloalkyl ring;
R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;
W at each occurrence is independently selected from N or CR₃;
R₃ is selected from hydrogen, halogen, cyano, substituted or unsubstituted alkyl and substituted or unsubstituted alkoxy; and
‘m’ is an integer ranging from 1 to 3, both inclusive;
or N-oxides thereof or a pharmaceutically acceptable salt thereof.

36. The compound of claim 35, wherein m is 2, R₃ is H, A₁ is H, A₂ is H, one R₂ is CF₃, the W adjacent to S is CR₃ and the W adjacent to N is CR₃.

37. The compound of claim 35, wherein m is 2, R₃ is H, A₁ is Me, A₂ is Me, one R₂ is CF₃, the W adjacent to S is CR₃ and the W adjacent to N is CR₃.

38. The compound of claim 36, wherein the other R₂ is selected from halogen and substituted or unsubstituted heteroaryl.

39. The compound of claim 31, having the Formula (III): 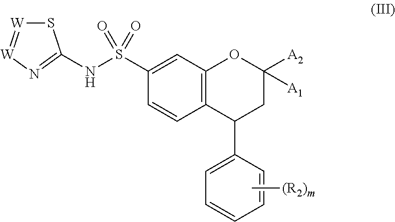
wherein,
A₁ and A₂ are independently hydrogen or substituted or unsubstituted alkyl; or
A₁ and A₂, together with the carbon atom to which they are attached, form a substituted or unsubstituted 3 to 6 membered cycloalkyl ring;
R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;
W at each occurrence is independently selected from N or CR₃;
R₃ is selected from hydrogen, halogen, cyano, substituted or unsubstituted alkyl and substituted or unsubstituted alkoxy; and
‘m’ is an integer ranging from 1 to 3, both inclusive;
or N-oxides thereof or a pharmaceutically acceptable salt thereof.

40. The compound of claim 31, having the Formula (IV): 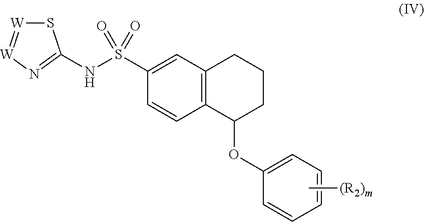
wherein,
R₂, which may be same or different at each occurrence, is independently selected from halogen, cyano, substituted or unsubstituted alkyl, substituted or unsubstituted haloalkyl, substituted or unsubstituted alkoxy, substituted or unsubstituted hydroxyalkyl, substituted or unsubstituted alkoxyalkyl, substituted or unsubstituted cycloalkyl, substituted or unsubstituted aryl, substituted or unsubstituted heteroaryl and substituted or unsubstituted heterocyclyl;
W at each occurrence is independently selected from N or CR₃;
R₃ is selected from hydrogen, halogen, cyano, substituted or unsubstituted alkyl and substituted or unsubstituted alkoxy; and
‘m’ is an integer ranging from 1 to 3, both inclusive;
or N-oxides thereof or a pharmaceutically acceptable salt thereof.

41. The compound according to claim 31 wherein m is 1 to 3.

42. The compound according to claim 31 wherein R₂ is same or different at each occurrence and independently selected from halogen, cyano, substituted or unsubstituted (C₁-C₆)alkyl, substituted or unsubstituted (C₁-C₆)haloalkyl, substituted or unsubstituted alkoxy, substituted or unsubstituted (C₃-C₁₂)cycloalkyl, substituted or unsubstituted C₆-aryl, substituted or unsubstituted heteroaryl and substituted or unsubstituted heterocyclyl.

43. The compound of claim 42, wherein R₂ is same or different at each occurrence and independently selected from halogen, haloalkyl, alkoxy, substituted or unsubstituted heteroaryl and substituted or unsubstituted heterocyclyl.

44. The compound according to claim 42 wherein m is 2; one R₂ is CF₃ and the other R₂ moiety is selected from the group consisting of: halogen, alkoxy, substituted or unsubstituted heteroaryl and substituted or unsubstituted heterocyclyl.

45. The compound according to claim 31 wherein A₁ and A₂ are independently H or substituted or unsubstituted (C₁-C₆)alkyl.

46. The compound according to claim 31 wherein A₁ and A₂ are Me.

47. The compound according to claim 31 wherein A₁ and A₂, together with the atoms to which they are attached, form a 4 membered cycloalkyl ring.

48. The compound according to claim 31 wherein W at each occurrence is independently selected from N or CR₃; where R₃ is selected from hydrogen, halogen and substituted or unsubstituted (C₁-C₆)alkyl.

49. The compound according to claim 31 having the Formula (Ia): 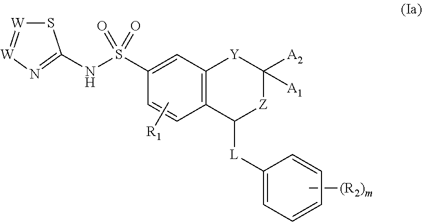
wherein Y is O or CH₂; L is O or bond; R₁ is hydrogen; A₁ and A₂ are hydrogen or substituted or unsubstituted (C₁-C₆)alkyl; Z is CH₂; or A₁ and A₂ together form a substituted or unsubstituted 3 to 6 membered cycloalkyl ring; at each occurrence R₂ is independently selected from halogen, cyano, substituted or unsubstituted (C₁-C₆)alkyl, substituted or unsubstituted (C₁-C₆)haloalkyl, substituted or unsubstituted (C₁-C₆)alkoxy, substituted or unsubstituted (C₃-C₁₂)cycloalkyl, substituted or unsubstituted C₆-aryl, substituted or unsubstituted heteroaryl and substituted or unsubstituted heterocyclyl; ‘m’ is 1 to 3 and W at each occurrence is independently selected from N or CR₃; where R₃ is hydrogen or halogen or substituted or unsubstituted (C₁-C₆)alkyl.

50. The compound of claim 49, wherein R₂ is independently selected from halogen, haloalkyl, alkoxy, substituted or unsubstituted heteroaryl and substituted or unsubstituted heterocyclyl; ‘m’ is 1 or 2.

51. The compound of which is selected from:
4-(2-Chloro-4-(trifluoromethyl)phenyl)-2,2-dimethyl-N-(1,2,4-thiadiazol-5-yl)chroman-7-sulfonamide,
4-(2-Chloro-4-(trifluoromethyl)phenyl)-2,2-dimethyl-N-(thiazol-2-yl)chroman-7-sulfonamide,
4-(2-Chloro-4-(trifluoromethyl)phenyl)-2,2-dimethyl-N-(1,3,4-thiadiazol-2-yl)chroman-7-sulfonamide,
4-(2-Methoxy-4-(trifluoromethyl)phenyl)-2,2-dimethyl-N-(1,2,4-thiadiazol-5-yl)chroman-7-sulfonamide,
4-(2-Methoxy-4-(trifluoromethyl)phenyl)-2,2-dimethyl-N-(thiazol-2-yl)chroman-7-sulfonamide,
4-(2-Methoxy-4-(trifluoromethyl)phenyl)-2,2-dimethyl-N-(1,3,4-thiadiazol-2-yl)chroman-7-sulfonamide,
2,2-Dimethyl-4-(2-(1,2,3,6-tetrahydropyridin-4-yl)-4-(trifluoromethyl)phenyl)-N-(thiazol-2-yl)chroman-7-sulfonamide hydrochloride,
2,2-Dimethyl-4-(2-(1,2,3,6-tetrahydropyridin-4-yl)-4-(trifluoromethyl)phenyl)-N-(1,2,4-thiadiazol-5-yl)chroman-7-sulfonamide hydrochloride,
2,2-Dimethyl-4-(2-(piperidin-4-yl)-4-(trifluoromethyl)phenyl)-N-(thiazol-2-yl)chroman-7-sulfonamide hydrochloride,
(R/S)-2,2-Dimethyl-4-(2-(piperidin-4-yl)-4-(trifluoromethyl)phenyl)-N-(thiazol-2-yl)chroman-7-sulfonamide hydrochloride,
2,2-Dimethyl-4-(2-(piperidin-4-yl)-4-(trifluoromethyl)phenyl)-N-(1,2,4-thiadiazol-5-yl)chroman-7-sulfonamide hydrochloride,
(R/S)-2,2-Dimethyl-4-(2-(piperidin-4-yl)-4-(trifluoromethyl)phenyl)-N-(1,2,4-thiadiazol-5-yl)chroman-7-sulfonamide,
2,2-Dimethyl-4-(2-(piperidin-4-yl)-4-(trifluoromethyl)phenyl)-N-(1,3,4-thiadiazol-2-yl)chroman-7-sulfonamide hydrochloride,
2,2-Dimethyl-4-(2-(1-methylpiperidin-4-yl)-4-(trifluoromethyl)phenyl)-N-(thiazol-2-yl)chroman-7-sulfonamide,
(R/S)-2,2-Dimethyl-4-(2-(1-methylpiperidin-4-yl)-4-(trifluoromethyl)phenyl)-N-(thiazol-2-yl)chroman-7-sulfonamide,
2,2-Dimethyl-4-(2-(1-methylpiperidin-4-yl)-4-(trifluoromethyl)phenyl)-N-(1,2,4-thiadiazol-5-yl)chroman-7-sulfonamide hydrochloride,
(R/S)-2,2-Dimethyl-4-(2-(1-methylpiperidin-4-yl)-4-(trifluoromethyl)phenyl)-N-(1,2,4-thiadiazol-5-yl)chroman-7-sulfonamide,
2,2-Dimethyl-4-(2-(1-methylpiperidin-4-yl)-4-(trifluoromethyl)phenyl)-N-(1,3,4-thiadiazol-2-yl)chroman-7-sulfonamide hydrochloride,
4-(2-(3-Fluoroazetidin-3-yl)-4-(trifluoromethyl)phenyl)-2,2-dimethyl-N-(thiazol-2-yl)chroman-7-sulfonamide hydrochloride,
4-(2-(3-Fluoroazetidin-3-yl)-4-(trifluoromethyl)phenyl)-2,2-dimethyl-N-(1,2,4-thiadiazol-5-yl)chroman-7-sulfonamide hydrochloride,
4-(2-Chloro-4-(trifluoromethyl)phenyl)-N-(1,2,4-thiadiazol-5-yl)chroman-7-sulfonamide,
4-(2-Chloro-4-(trifluoromethyl)phenyl)-N-(1,3,4-thiadiazol-2-yl)chroman-7-sulfonamide,
4-(2-Chloro-4-(trifluoromethyl)phenyl)-N-(thiazol-2-yl)chroman-7-sulfonamide,
(R/S)-4-(2-Chloro-4-(trifluoromethyl)phenyl)-N-(thiazol-2-yl)chroman-7-sulfonamide,
4-(2-(1,2,3,6-Tetrahydropyridin-4-yl)-4-(trifluoromethyl)phenyl)-N-(thiazol-2-yl) chroman-7-sulfonamide hydrochloride,
4-(2-(1,2,3,6-Tetrahydropyridin-4-yl)-4-(trifluoromethyl)phenyl)-N-(1,2,4-thiadiazol-5-yl)chroman-7-sulfonamide hydrochloride,
4-(2-(1,2,3,6-Tetrahydropyridin-4-yl)-4-(trifluoromethyl)phenyl)-N-(1,3,4-thiadiazol-2-yl)chroman-7-sulfonamide hydrochloride,
4-(2-(3-Fluoroazetidin-3-yl)-4-(trifluoromethyl)phenyl)-N-(thiazol-2-yl)chroman-7-sulfonamide hydrochloride,
4-(2-(1-Methyl-1H-pyrazol-5-yl)-4-(trifluoromethyl)phenyl)-N-(thiazol-2-yl)chroman-7-sulfonamide,
4-(2-Chloro-4-(trifluoromethyl)phenoxy)-N-(thiazol-2-yl)chroman-7-sulfonamide,
(R)-4-(2-Chloro-4-(trifluoromethyl)phenoxy)-N-(thiazol-2-yl)chroman-7-sulfonamide,
(R)-4-(2-Cyano-4-(trifluoromethyl)phenoxy)-N-(thiazazol-2-yl)chroman-7-sulfonamide,
(R)-4-(2,4-Dichlorophenoxy)-N-(thiazol-2-yl)chroman-7-sulfonamide,
(R)-4-(3,4-Dichlorophenoxy)-N-(thiazol-2-yl)chroman-7-sulfonamide,
(R)—N-(Thiazol-2-yl)-4-(2,4,6-trifluorophenoxy)chroman-7-sulfonamide,
4-(2-Chloro-4-(trifluoromethyl)phenoxy)-N-(1,2,4-thiadiazol-5-yl)chroman-7-sulfonamide,
(R)-4-(2-Chloro-4-(trifluoromethyl)phenoxy)-N-(1,2,4-thiadiazol-5-yl)chroman-7-sulfonamide,
(R)-4-(2-(1-Methyl-1H-pyrazol-5-yl)-4-(trifluoromethyl)phenoxy)-N-(thiazol-2-yl)chroman-7-sulfonamide,
(S)-4-(2-(1-Methyl-1H-pyrazol-5-yl)-4-(trifluoromethyl)phenoxy)-N-(thiazol-2-yl)chroman-7-sulfonamide,
(R)-4-(2-(Pyridin-3-yl)-4-(trifluoromethyl)phenoxy)-N-(thiazol-2-yl)chroman-7-sulfonamide,
(R)-4-(2-(Pyridin-4-yl)-4-(trifluoromethyl)phenoxy)-N-(thiazol-2-yl)chroman-7-sulfonamide,
(R)-4-(2-(5-Fluoropyridin-2-yl)-4-(trifluoromethyl)phenoxy)-N-(thiazol-2-yl)chroman-7-sulfonamide,
(R)-4-(2-(6-Methylpyridin-3-yl)-4-(trifluoromethyl)phenoxy)-N-(thiazol-2-yl)chroman-7-sulfonamide,
(R)-4-(2-(2-Methylpyrimidin-5-yl)-4-(trifluoromethyl)phenoxy)-N-(thiazol-2-yl)chroman-7-sulfonamide,
(R)-4-(2-(1-Methyl-1H-pyrazol-4-yl)-4-(trifluoromethyl)phenoxy)-N-(thiazol-2-yl)chroman-7-sulfonamide,
(R)-4-(2-Ethyl-4-(trifluoromethyl)phenoxy)-N-(thiazol-2-yl)chroman-7-sulfonamide,
(R)-4-(2-Isopropyl-4-(trifluoromethyl)phenoxy)-N-(thiazol-2-yl)chroman-7-sulfonamide,
(R)-4-(2-Cyclopropyl-4-(trifluoromethyl)phenoxy)-N-(thiazol-2-yl)chroman-7-sulfonamide,
(R)-4-(2-(1-Isopropyl-1H-pyrazol-5-yl)-4-(trifluoromethyl)phenoxy)-N-(thiazol-2-yl)chroman-7-sulfonamide,
(R)-4-(2-(1-(2-Fluoroethyl)-1H-pyrazol-5-yl)-4-(trifluoromethyl)phenoxy)-N-(thiazol-2-yl)chroman-7-sulfonamide,
(R)-4-((4′-Fluoro-5-(trifluoromethyl)-[1,1′-biphenyl]-2-yl)oxy)-N-(thiazol-2-yl)chroman-7-sulfonamide,
(R)-4-(2-(Pyridin-2-yl)-4-(trifluoromethyl)phenoxy)-N-(thiazol-2-yl)chroman-7-sulfonamide,
(R)-4-(2-(4-Cyclopropyl-1H-1,2,3-triazol-1-yl)-4-(trifluoromethyl)phenoxy)-N-(thiazol-2-yl)chroman-7-sulfonamide,
(R)-4-(2-(1H-Pyrazol-1-yl)-4-(trifluoromethyl)phenoxy)-N-(thiazol-2-yl)chroman-7-sulfonamide,
(R)-4-(2-(2-Methylthiazol-4-yl)-4-(trifluoromethyl)phenoxy)-N-(thiazol-2-yl)chroman-7-sulfonamide,
(R)-4-(2-(2-Methyloxazol-4-yl)-4-(trifluoromethyl)phenoxy)-N-(thiazol-2-yl)chroman-7-sulfonamide,
(R)—N,N-Dimethyl-2-(2-((7-(N-(thiazol-2-yl)sulfamoyl)chroman-4-yl)oxy)-5-(trifluoromethyl)phenyl)acetamide,
(R)-2-(2-((7-(N-(Thiazol-2-yl)sulfamoyl)chroman-4-yl)oxy)-5-(trifluoromethyl)phenyl)pyridine 1-oxide,
4-(2-(1,2,3,6-Tetrahydropyridin-4-yl)-4-(trifluoromethyl)phenoxy)-N-(thiazol-2-yl)chroman-7-sulfonamide trifluoroacetic acid,
4-(2-(Piperidin-4-yl)-4-(trifluoromethyl)phenoxy)-N-(thiazol-2-yl)chroman-7-sulfonamide hydrochloride,
4-(2-(piperidin-4-yl)-4-(trifluoromethyl)phenoxy)-N-(1,2,4-thiadiazol-5-yl)chroman-7-sulfonamide trifluoroacetic acid,
(R)-4-(2-Chloro-4-(trifluoromethyl)phenoxy)-2,2-dimethyl-N-(thiazol-2-yl)chroman-7-sulfonamide,
(S)-4-(2-Chloro-4-(trifluoromethyl)phenoxy)-2,2-dimethyl-N-(thiazol-2-yl)chroman-7-sulfonamide,
(R)-2,2-Dimethyl-N-(thiazol-2-yl)-4-(4-(trifluoromethyl)phenoxy)chroman-7-sulfonamide,
(R)-2,2-Dimethyl-N-(thiazol-2-yl)-4-(3-(trifluoromethyl)phenoxy)chroman-7-sulfonamide,
(R)-4-(2,4-Dichlorophenoxy)-2,2-dimethyl-N-(thiazol-2-yl)chroman-7-sulfonamide,
(R)-4-(2-Cyano-4-(trifluoromethyl)phenoxy)-2,2-dimethyl-N-(thiazol-2-yl)chroman-7-sulfonamide,
(R)-4-(2-Ethyl-4-(trifluoromethyl)phenoxy)-2,2-dimethyl-N-(thiazol-2-yl)chroman-7-sulfonamide,
(R)-4-(2-(1-Isopropyl-1H-pyrazol-5-yl)-4-(trifluoromethyl)phenoxy)-2,2-dimethyl-N-(thiazol-2-yl)chroman-7-sulfonamide,
(R)-4-((4′-Fluoro-5-(trifluoromethyl)-[1,1′-biphenyl]-2-yl)oxy)-2,2-dimethyl-N-(thiazol-2-yl)chroman-7-sulfonamide,
(R)-4-(2-(4-Cyclopropyl-1H-1,2,3-triazol-1-yl)-4-(trifluoromethyl)phenoxy)-2,2-dimethyl-N-(thiazol-2-yl)chroman-7-sulfonamide,
(R)-4-(2-(1H-Pyrazol-1-yl)-4-(trifluoromethyl)phenoxy)-2,2-dimethyl-N-(thiazol-2-yl)chroman-7-sulfonamide,
(R)-2,2-Dimethyl-4-(2-(pyridin-2-yl)-4-(trifluoromethyl)phenoxy)-N-(thiazol-2-yl)chroman-7-sulfonamide,
(R)-2,2-Dimethyl-4-(2-(2-methylthiazol-4-yl)-4-(trifluoromethyl)phenoxy)-N-(thiazol-2-yl)chroman-7-sulfonamide,
(R)-2,2-Dimethyl-4-(2-(2-methyloxazol-4-yl)-4-(trifluoromethyl)phenoxy)-N-(thiazol-2-yl)chroman-7-sulfonamide,
(R)-2,2-Dimethyl-4-(2-(1-methyl-1H-pyrazol-5-yl)-4-(trifluoromethyl)phenoxy)-N-(thiazol-2-yl)chroman-7-sulfonamide,
(R)-2,2-Dimethyl-4-(2-(1-methyl-1H-pyrazol-5-yl)-5-(trifluoromethyl)phenoxy)-N-(thiazol-2-yl)chroman-7-sulfonamide,
(R)-2,2-Dimethyl-4-(2-(pyridin-4-yl)-4-(trifluoromethyl)phenoxy)-N-(thiazol-2-yl)chroman-7-sulfonamide,
(R)-4-(2-Chloro-4-(trifluoromethyl)phenoxy)-N-(thiazol-2-yl)spiro[chroman-2, 1′-cyclobutane]-7-sulfonamide,
(R)-4-(2-(1-Methyl-1H-pyrazol-5-yl)-4-(trifluoromethyl)phenoxy)-N-(thiazol-2-yl)spiro[chroman-2,1′-cyclobutane]-7-sulfonamide,
(2R/S,4R)-4-(2-Chloro-4-(trifluoromethyl)phenoxy)-2-ethyl-N-(thiazol-2-yl)chroman-7-sulfonamide,
(2S/R,4R)-4-(2-Chloro-4-(trifluoromethyl)phenoxy)-2-ethyl-N-(thiazol-2-yl)chroman-7-sulfonamide,
(2S/R,4R)-2-Ethyl-4-(2-(1-methyl-1H-pyrazol-5-yl)-4-(trifluoromethyl) phenoxy)-N-(thiazol-2-yl)chroman-7-sulfonamide,
(2R/S,4R)-2-Ethyl-4-(2-(1-methyl-1H-pyrazol-5-yl)-4-(trifluoromethyl) phenoxy)-N-(thiazol-2-yl)chroman-7-sulfonamide,
5-(2-Chloro-4-(trifluoromethyl)phenoxy)-N-(thiazol-2-yl)-5,6,7,8-tetrahydronaphthalene-2-sulfonamide,
(R)-5-(2-Chloro-4-(trifluoromethyl)phenoxy)-N-(thiazol-2-yl)-5,6,7,8-tetrahydronaphthalene-2-sulfonamide,
5-(2-Bromo-4-(trifluoromethyl)phenoxy)-N-(thiazol-2-yl)-5,6,7,8-tetrahydronaphthalene-2-sulfonamide,
5-(2-Chloro-4-(trifluoromethyl)phenoxy)-N-(1,2,4-thiadiazol-5-yl)-5,6,7,8-tetrahydronaphthalene-2-sulfonamide,
5-(2-Bromo-4-(trifluoromethyl)phenoxy)-N-(1,2,4-thiadiazol-5-yl)-5,6,7,8-tetrahydronaphthalene-2-sulfonamide,
N-(Thiazol-2-yl)-5-(4-(trifluoromethyl)phenoxy)-5,6,7,8-tetrahydronaphthalene-2-sulfonamide,
5-(2-(1-Methyl-1H-pyrazol-5-yl)-4-(trifluoromethyl)phenoxy)-N-(thiazol-2-yl)-5,6,7,8-tetrahydronaphthalene-2-sulfonamide,
(R)-5-(2-(1-Methyl-1H-pyrazol-5-yl)-4-(trifluoromethyl)phenoxy)-N-(thiazol-2-yl)-5,6,7,8-tetrahydronaphthalene-2-sulfonamide,
5-(2-(1,2,3,6-Tetrahydropyridin-4-yl)-4-(trifluoromethyl)phenoxy)-N-(thiazol-2-yl)-5,6,7,8-tetrahydronaphthalene-2-sulfonamide trifluoroacetic acid,
(R/S)-5-(2-(1,2,3,6-Tetrahydropyridin-4-yl)-4-(trifluoromethyl)phenoxy)-N-(thiazol-2-yl)-5,6,7,8-tetrahydronaphthalene-2-sulfonamide trifluoroacetic acid,
5-(2-(Piperidin-4-yl)-4-(trifluoromethyl)phenoxy)-N-(thiazol-2-yl)-5,6,7,8-tetrahydronaphthalene-2-sulfonamide trifluoroacetic acid,
(R/S)-5-(2-(Piperidin-4-yl)-4-(trifluoromethyl)phenoxy)-N-(thiazol-2-yl)-5,6,7,8-tetrahydronaphthalene-2-sulfonamide,
5-(2-(Piperidin-4-yl)-4-(trifluoromethyl)phenoxy)-N-(1,2,4-thiadiazol-5-yl)-5,6,7,8-tetrahydronaphthalene-2-sulfonamide,
5-(2-(Piperidin-4-yl)-4-(trifluoromethyl)phenoxy)-N-(1,3,4-thiadiazol-2-yl)-5,6,7,8-tetrahydronaphthalene-2-sulfonamide trifluoroacetic acid,
5-(2-(1-Methylpiperidin-4-yl)-4-(trifluoromethyl)phenoxy)-N-(thiazol-2-yl)-5,6,7,8-tetrahydronaphthalene-2-sulfonamide,
5-(2-(1-Methylpiperidin-4-yl)-4-(trifluoromethyl)phenoxy)-N-(1,2,4-thiadiazol-5-yl)-5,6,7,8-tetrahydronaphthalene-2-sulfonamide,
5-(2-(1-Methylpiperidin-4-yl)-4-(trifluoromethyl)phenoxy)-N-(1,3,4-thiadiazol-2-yl)-5,6,7,8-tetrahydronaphthalene-2-sulfonamide,
(R)-4-(2-(2-Fluoroethyl)-4-(trifluoromethyl)phenoxy)-N-(thiazol-2-yl)chroman-7-sulfonamide,
(R)-4-(2-(1-Ethyl-1H-pyrazol-5-yl)-4-(trifluoromethyl)phenoxy)-N-(thiazol-2-yl)chroman-7-sulfonamide,
(R)-2-(2-((2,2-Dimethyl-7-(N-(thiazol-2-yl)sulfamoyl)chroman-4-yl)oxy)-5-(trifluoromethyl)phenyl)pyridine 1-oxide,
(R)-3-(2-((7-(N-(Thiazol-2-yl)sulfamoyl)chroman-4-yl)oxy)-5-(trifluoromethyl)phenyl)pyridine 1-oxide and
(R)-4-(2-((7-(N-(Thiazol-2-yl)sulfamoyl)chroman-4-yl)oxy)-5-(trifluoromethyl)phenyl)pyridine 1-oxide
or a free base thereof, N-oxide thereof, or stereoisomers thereof or a pharmaceutically acceptable salt thereof.

52. A pharmaceutical composition comprising one or more compounds according to claim 31, and one or more pharmaceutically acceptable excipients.

53. A method of treating, managing and/or lessening diseases or disorders, syndromes or conditions associated with the modulation of Na_{V}1.7 function in a subject in need thereof wherein the method comprises administering to the subject a therapeutically effective amount of a compound according to claim 31, or a pharmaceutically acceptable salt thereof.

54. The method of claim 53, wherein the diseases, disorders, syndromes or conditions associated with the modulation of Na_{V}1.7 functions are selected from the group consisting of pain and erythromelalgia.

55. The method of claim 53, wherein the diseases, disorders, syndromes or conditions associated with the modulation of Na_{V}1.7 functions are selected from the group consisting of neurological disorders, cardiovascular conditions, neuromascular conditions, multiple sclerosis, cancer, pruritis or benign prostatic hyperplasia (BPH).

56. The method of claim 54, wherein the pain is neuropathic pain.

57. The method of claim 54, wherein the pain is inflammatory pain.

58. The method of claim 53, wherein the diseases, disorders, syndromes or conditions associated with the modulation of Na_{V}1.7 function are selected from the group consisting of postoperative pain, arthritis pain, osteoarthritis pain, pain associated with cancer including chemotherapy pain, neuropathic pain secondary to metastatic inflammation, neuralgic, orofacial pain, burn pain, somatic pain, dental pain, sciatica pain, intestinal obstruction pain, visceral pain, coliky pain, myofacial pain, trauma pain, labour pain, trigeminal neuralgia, glossopharangyl neuralgia, adiposis dolorosa, acute herpetic and postherpetic neuralgia, diabetic neuropathy, causalgia, brachial plexus avulsion, occipital neuralgia, reflux sympathetic dystrophy, fibromyalgia, gout, phantom limb pain, pain following stroke, thalamic lesions, radiculopathy, chronic headache, migraine pain, familial hemiplegic migraine, conditions associated with cephalic pain, sinus headache, tension headache, cardiac pain arising from an ischemic myocardium, pain following stroke, neuropathy secondary to metastatic inflammation, pain due to connective tissue damage, and other forms of neuralgic, neuropathic, and idiopathic pain syndromes in a subject in need thereof comprising administering to the subject with a therapeutically effective amount.

59. A process for the preparation of compound of Formula (Ib): 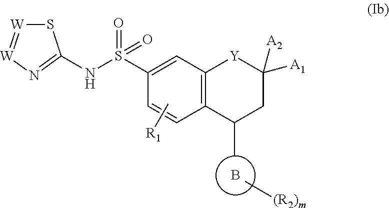
wherein ring B, Y, W, A₁, A₂, R₁, R₂ and m are as defined in claim 31;
the process comprising the steps of:
a) reducing a double bond of compound of formula (5) followed by deprotection using suitable reagents to obtain compound of formula (6) 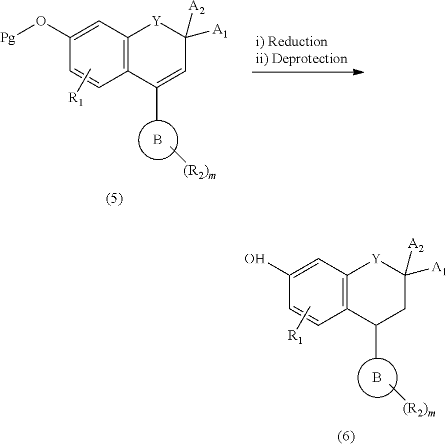
b) reacting a hydroxy group in compound of formula (6) with suitable triflating agents to get compound of formula (7) 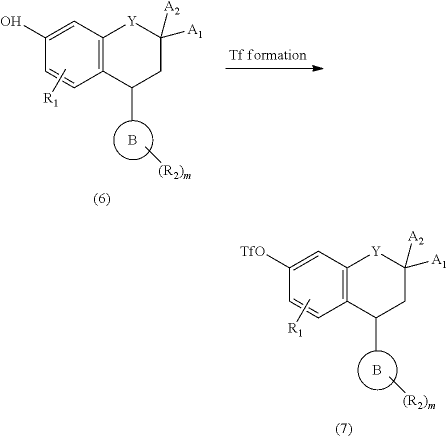
c) reacting a triflate compound of formula (7) with benzyl mercaptan in the presence of Pd catalyst to give the compound of formula (8) 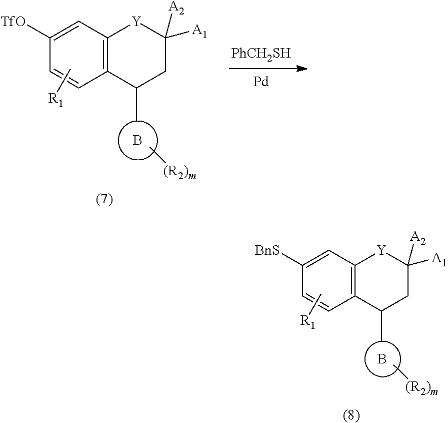
d) converting a compound of formula (8) to compound of formula (9) using pentafluorophenol 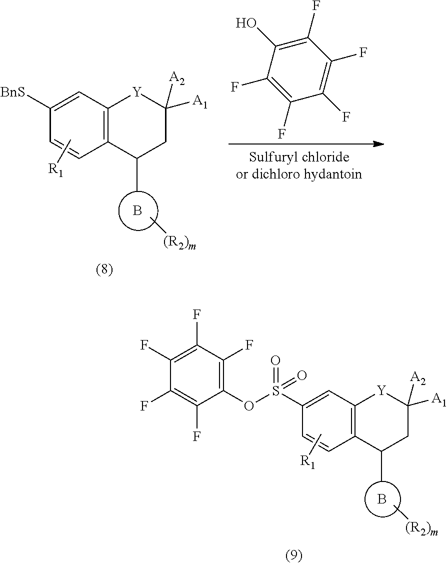
e) reacting a compound of formula (9) with an amino compound of formula (10) in presence of suitable base to afford compound of formula (Ib)

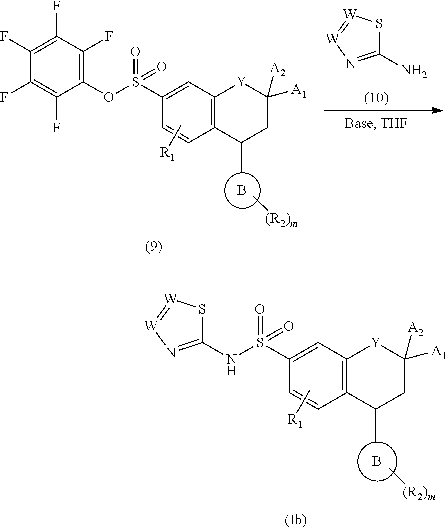

60. A process for the preparation of compound of Formula (Ic): 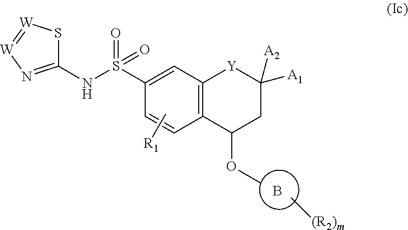
wherein ring B, W, Y, A₁, A₂, R₁, R₂ and m are as defined in claim 31;
the process comprising the steps of:
a) reducing a keto group of compound of formula (13) using suitable reducing agents to give compound of formula (18) 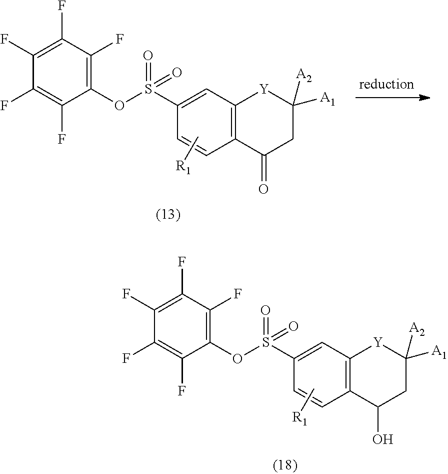
b) reacting a hydroxyl group in compound of formula (18) with phenol of formula (19) under Mitsunobu reaction conditions gives the compound of formula (20) 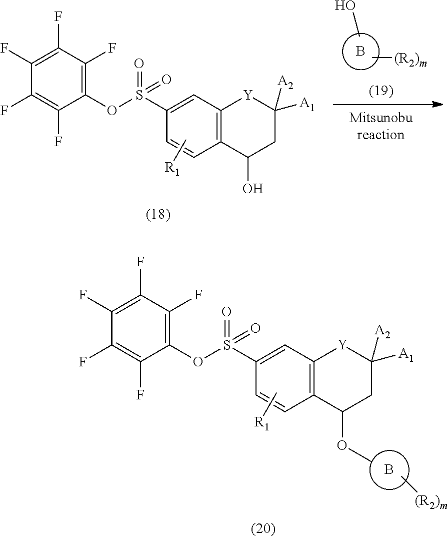
c) reacting a compound of formula (20) with an amino compound of formula (10) in presence of suitable base to afford compound of formula (Ic) [chemical structure]
